(19) United States
(12) Patent Application Publication
(10) Pub. No.: US 2026/0112486 A1
(43) Pub. Date: Apr. 23, 2026

(54) PROTEIN PREDICTORS FOR LUNG CANCER

(71) Applicant: Janssen Pharmaceutica NV, Beerse (BE)

(72) Inventors: Takahiro Sato, Philadelphia, PA (US); Robert Yunchuan Yang, Belmont, MA (US); Duncan H. Whitney, Boston, MA (US)

(21) Appl. No.: 18/865,075

(22) PCT Filed: Jun. 13, 2023
(86) PCT No.: PCT/EP2023/065832

§ 371 Date: Nov. 12, 2024

Related U.S. Application Data
(60) Provisional application No. 63/351,689, filed on Jun. 13, 2022.

(51) Int. Cl. G16H 50/20; G01N 33/575; G01N 33/5752; G16B 20/00

(52) U.S. Cl. CPC G16H 50/20; G01N 33/5752; G01N 33/5758; G16B 20/00

(57) ABSTRACT

Disclosed herein are methods for analyzing predictors including quantitative values of biomarkers (e.g., protein biomarkers) for predicting risk of cancer in a human subject. Further disclosed herein are kits for measuring quantitative values of the markers as well as computer systems and software embodiments for predicting risk of cancer in a human subject based on the quantitative values of the biomarkers (e.g., protein biomarkers).

### CROSS REFERENCE TO RELATED APPLICATIONS

This application is the U.S. national stage of PCT Application No. PCT/EP2023/065832, filed Jun. 13, 2023, which claims priority to U.S. Provisional Patent Application No. 63/351,689, filed Jun. 13, 2022, the entire contents of which are each expressly incorporated herein by reference.

### FIELD

The field relates to predictive models that are useful for predicting risk of cancer (e.g., lung cancer). These predictive models are based at least on the measurement of protein profiles from samples (e.g., blood plasma samples).

### BACKGROUND

Lung cancer is the leading cause of cancer deaths worldwide. This is largely due to its advanced stage at the time of diagnosis, with 5-year survival of only 15% or less. It is difficult to identify people who have early stage lung cancer in a cost-efficient manner. Hence, people are often referred to hospital clinics with late stage disease, which leads to poor curative opportunities and outlook.

### SUMMARY

Disclosed herein are methods for predicting risk of cancer (e.g., future risk of cancer or presence or absence of cancer) in a subject using plasma proteomics data derived from the subject. Further disclosed are methods, such as recursive feature elimination, for selecting a subset of protein biomarkers for predicting risk of cancer. Additionally disclosed herein are non-transitory computer readable mediums for predicting risk of cancer in a subject using predictive models. Additionally disclosed herein are kits containing one or more sets of reagents for determining quantitative values of protein predictors for predicting risk of cancer. In various embodiments, the prediction for risk of cancer for the subject is a prediction of presence or absence of cancer in the subject, or a prediction of whether the subject is likely to develop cancer in the future (e.g., within 1-20 years). In various embodiments, the terms “levels” and “values”, such as the levels or values of metabolites, biomarkers, markers or predictors, are synonymous and may be used interchangeably. Therefore, in these embodiments, any reference to “values”, such as the values of metabolites, biomarkers, markers or predictors, may equally be construed as “levels”, such as the levels of those metabolites, biomarkers, markers or predictors. Similarly, in these embodiments, any reference herein to “levels”, such as the levels of metabolites, biomarkers, markers or predictors, may equally be construed as “values”, such as the values of those metabolites, biomarkers, markers or predictors.

Advantageously, the methods, non-transitory computer readable mediums, and/or kits as described herein can lead to early detection of lung cancer (e.g., before diagnosis), which may result in early intervention and treatment. This informs which patients to target with disease interception strategies, and thus improve the survival and decreased mortality rates due to lung cancer.

Disclosed herein is a method for predicting risk of cancer in a subject, the method comprising: obtaining or having obtained a dataset derived from the subject comprising quantitative levels of a plurality of biomarkers, wherein the plurality of biomarkers comprises protein biomarkers comprising two or more of TSPAN1, CD28, SCN3B, ADGRB3, and IGFBP6, and generating a prediction of risk of cancer for the subject by applying a predictive model to the quantitative values of the plurality of biomarkers.

In various embodiments, the protein biomarkers comprise three or more of TSPAN1, CD28, SCN3B, ADGRB3, and IGFBP6.

In various embodiments, the protein biomarkers comprise four or more of TSPAN1, CD28, SCN3B, ADGRB3, and IGFBP6.

In various embodiments, the protein biomarkers comprise each of TSPAN1, CD28, SCN3B, ADGRB3, and IGFBP6.

In various embodiments, the protein biomarkers further comprise one or more of NRTN, AIF1L, HSPB6, MB, TNFRSF19, IL5RA, TNR, CDNF, CST1, FGFBP2, S100A16, CD248, GFRA3, LMOD1, and POF1B.

In various embodiments, the protein biomarkers further comprise five or more of NRTN, AIF1L, HSPB6, MB, TNFRSF19, IL5RA, TNR, CDNF, CST1, FGFBP2, S100A16, CD248, GFRA3, LMOD1, and POF1B.

In various embodiments, the protein biomarkers further comprise ten or more of NRTN, AIF1L, HSPB6, MB, TNFRSF19, IL5RA, TNR, CDNF, CST1, FGFBP2, S100A16, CD248, GFRA3, LMOD1, and POF1B.

In various embodiments, the protein biomarkers further comprise each of NRTN, AIF1L, HSPB6, MB, TNFRSF19, IL5RA, TNR, CDNF, CST1, FGFBP2, S100A16, CD248, GFRA3, LMOD1, and POF1B.

In various embodiments, the protein biomarkers further comprise one or more of DENND2B, COMP, CNTN2, SCARA5, CSPG4, ITGAV, SOST, SERPINA4, LILRA4, SPINK5, PINLYP, ACTN2, JAM2, FAP, TMOD4, GUCA2A, MFAP3L, DKK4, LAMA1, BAG3, SNCG, SEPTIN3, VWC2, KLRC1, ATRAID, ART3, SLITRK2, SIGLEC6, TMED4, and SLAMF7.

In various embodiments, the protein biomarkers further comprise five or more of DENND2B, COMP, CNTN2, SCARA5, CSPG4, ITGAV, SOST, SERPINA4, LILRA4, SPINK5, PINLYP, ACTN2, JAM2, FAP, TMOD4, GUCA2A, MFAP3L, DKK4, LAMA1, BAG3, SNCG, SEPTIN3, VWC2, KLRC1, ATRAID, ART3, SLITRK2, SIGLEC6, TMED4, and SLAMF7.

In various embodiments, the protein biomarkers further comprise ten or more of DENND2B, COMP, CNTN2, SCARA5, CSPG4, ITGAV, SOST, SERPINA4, LILRA4, SPINK5, PINLYP, ACTN2, JAM2, FAP, TMOD4, GUCA2A, MFAP3L, DKK4, LAMA1, BAG3, SNCG, SEPTIN3, VWC2, KLRC1, ATRAID, ART3, SLITRK2, SIGLEC6, TMED4, and SLAMF7.

In various embodiments, the protein biomarkers further comprise twenty or more of DENND2B, COMP, CNTN2, SCARA5, CSPG4, ITGAV, SOST, SERPINA4, LILRA4, SPINK5, PINLYP, ACTN2, JAM2, FAP, TMOD4, GUCA2A, MFAP3L, DKK4, LAMA1, BAG3, SNCG, SEPTIN3, VWC2, KLRC1, ATRAID, ART3, SLITRK2, SIGLEC6, TMED4, and SLAMF7.

In various embodiments, the protein biomarkers further comprise each of DENND2B, COMP, CNTN2, SCARA5, CSPG4, ITGAV, SOST, SERPINA4, LILRA4, SPINK5, PINLYP, ACTN2, JAM2, FAP, TMOD4, GUCA2A, MFAP3L, DKK4, LAMA1, BAG3, SNCG, SEPTIN3, VWC2, KLRC1, ATRAID, ART3, SLITRK2, SIGLEC6, TMED4, and SLAMF7.

In various embodiments, the protein biomarkers further comprise one or more of CKMT1A, SEMA6C, CD2, CST5, PBXIP1, LECT2, PYY, AGRN, INSL5, CD38, PI16, CCN5, TNFRSF17, LY9, GPC1, CLMP, MEP1B, CCN1, PCDH7, SPARCL1, CRNN, PM20D1, TNFRSF12A, DSCAM, PALM, CX3CL1, MEP1A, SLURP1, APOA4, ADAMTSL5, MEPE, WFDC1, RPS10, CD300C, RIPK4, CALCB, RTBDN, ENO3, NTF3, PTPRZ1, LRP2BP, CPE, MCAM, BGN, PLB1, YAP1, TGFBI, CYB5A, EDDM3B, and SELENOP.

In various embodiments, the protein biomarkers further comprise five or more of CKMT1A, SEMA6C, CD2, CST5, PBXIP1, LECT2, PYY, AGRN, INSL5, CD38, PI16, CCN5, TNFRSF17, LY9, GPC1, CLMP, MEP1B, CCN1, PCDH7, SPARCL1, CRNN, PM20D1, TNFRSF12A, DSCAM, PALM, CX3CL1, MEP1A, SLURP1, APOA4, ADAMTSL5, MEPE, WFDC1, RPS10, CD300C, RIPK4, CALCB, RTBDN, ENO3, NTF3, PTPRZ1, LRP2BP, CPE, MCAM, BGN, PLB1, YAP1, TGFBI, CYB5A, EDDM3B, and SELENOP.

In various embodiments, the protein biomarkers further comprise ten or more of CKMT1A, SEMA6C, CD2, CST5, PBXIP1, LECT2, PYY, AGRN, INSL5, CD38, PI16, CCN5, TNFRSF17, LY9, GPC1, CLMP, MEP1B, CCN1, PCDH7, SPARCL1, CRNN, PM20D1, TNFRSF12A, DSCAM, PALM, CX3CL1, MEP1A, SLURP1, APOA4, ADAMTSL5, MEPE, WFDC1, RPS10, CD300C, RIPK4, CALCB, RTBDN, ENO3, NTF3, PTPRZ1, LRP2BP, CPE, MCAM, BGN, PLB1, YAP1, TGFBI, CYB5A, EDDM3B, and SELENOP.

In various embodiments, the protein biomarkers further comprise twenty or more of CKMT1A, SEMA6C, CD2, CST5, PBXIP1, LECT2, PYY, AGRN, INSL5, CD38, PI16, CCN5, TNFRSF17, LY9, GPC1, CLMP, MEP1B, CCN1, PCDH7, SPARCL1, CRNN, PM20D1, TNFRSF12A, DSCAM, PALM, CX3CL1, MEP1A, SLURP1, APOA4, ADAMTSL5, MEPE, WFDC1, RPS10, CD300C, RIPK4, CALCB, RTBDN, ENO3, NTF3, PTPRZ1, LRP2BP, CPE, MCAM, BGN, PLB1, YAP1, TGFBI, CYB5A, EDDM3B, and SELENOP.

In various embodiments, the protein biomarkers further comprise thirty or more of CKMT1A, SEMA6C, CD2, CST5, PBXIP1, LECT2, PYY, AGRN, INSL5, CD38, PI16, CCN5, TNFRSF17, LY9, GPC1, CLMP, MEP1B, CCN1, PCDH7, SPARCL1, CRNN, PM20D1, TNFRSF12A, DSCAM, PALM, CX3CL1, MEP1A, SLURP1, APOA4, ADAMTSL5, MEPE, WFDC1, RPS10, CD300C, RIPK4, CALCB, RTBDN, ENO3, NTF3, PTPRZ1, LRP2BP, CPE, MCAM, BGN, PLB1, YAP1, TGFBI, CYB5A, EDDM3B, and SELENOP.

In various embodiments, the protein biomarkers further comprise forty or more of CKMT1A, SEMA6C, CD2, CST5, PBXIP1, LECT2, PYY, AGRN, INSL5, CD38, PI16, CCN5, TNFRSF17, LY9, GPC1, CLMP, MEP1B, CCN1, PCDH7, SPARCL1, CRNN, PM20D1, TNFRSF12A, DSCAM, PALM, CX3CL1, MEP1A, SLURP1, APOA4, ADAMTSL5, MEPE, WFDC1, RPS10, CD300C, RIPK4, CALCB, RTBDN, ENO3, NTF3, PTPRZ1, LRP2BP, CPE, MCAM, BGN, PLB1, YAP1, TGFBI, CYB5A, EDDM3B, and SELENOP.

In various embodiments, the protein biomarkers further comprise each of CKMT1A, SEMA6C, CD2, CST5, PBXIP1, LECT2, PYY, AGRN, INSL5, CD38, PI16, CCN5, TNFRSF17, LY9, GPC1, CLMP, MEP1B, CCN1, PCDH7, SPARCL1, CRNN, PM20D1, TNFRSF12A, DSCAM, PALM, CX3CL1, MEP1A, SLURP1, APOA4, ADAMTSL5, MEPE, WFDC1, RPS10, CD300C, RIPK4, CALCB, RTBDN, ENO3, NTF3, PTPRZ1, LRP2BP, CPE, MCAM, BGN, PLB1, YAP1, TGFBI, CYB5A, EDDM3B, and SELENOP.

In various embodiments, the protein biomarkers further comprise one or more of ENPP6, TMEM25, GIP, CSPG5, SCGN, TMPRSS15, LAIR2, KIRREL1, NTF4, TSPAN7, ENDOU, KLK10, CCL24, GPR37, CD3D, TJP3, DKKL1, CFC1, LRRC38, GCG, AGBL2, FASLG, AHNAK2, WFIKKN2, ANXA10, HS6ST1, DUSP29, CA14, CLEC7A, PHLDB2, SCRG1, RSPO3, TOP1, TINAGL1, NCAM1, FAM3D, FLT3LG, ZP3, AGRP, ASAH2, PDGFRB, AFM, NPY, PPY, XG, MFGE8, PROS1, MEGF11, CTSO, CTLA4, CSF3R, FCAR, CTAG1A, SCPEP1, PRSS53, CRELD2, PILRA, PROC, VASH1, NOS3, BPIFB2, UPK3BL1, NOP56, JAM3, HLA-DRA, SIL1, TRPV3, EDEM2, POLR2A, CBLN1, FKBP7, CCL20, PILRB, SIRPB1, VSTM1, BST2, DLL4, C1RL, RNASET2, KCNH2, IL12RB2, FZD10, OXCT1, TREML2, GRIN2B, GFRAL, RGS8, LRPAP1, LRP2, IGSF21, DPT, HEPACAM2, MATN3, UXS1, PTTG1, BTN1A1, IL17C, SCIN, TK1, FKBP14, VWA5A, PRKG1, SV2A, PMCH, NEXN, CDCP1, DDX53, THSD1, PAK4, MMP12, FCN1, UMOD, PDIA4, IL6, BRK1, LILRA2, RBPMS2, SERPIND1, TPSG1, CEACAM5, FGF9, PPIF, RNF43, SIGLEC9, TOMM20, PDE5A, NELL1, GBA, PAEP, ERN1, PCSK7, CHCHD6, MARCO, SFTPA1, IL9, KYNU, SPINT1, LRFN2, NECTIN1, OSCAR, PZP, BPIFB1, LILRA5, CALY, RRAS, GADD45GIP1, ISM2, SCGB3A2, CEACAM6, LPP, GKN1, LRIG1, CLSPN, CXCL13, SFTPA2, COX6B1, PTGR1, RBPMS, PPT1, AOC1, PDLIM5, L3HYPDH, LONP1, APOL1, CEACAM18, FGF7, and KRT14.

In various embodiments, the predictive model comprises a elastic net regression model, and wherein the predictive model achieves an area under a curve (AUC) value of at least 0.85.

In various embodiments, the predictive model comprises a support vector machine, and wherein the predictive model achieves an area under a curve (AUC) value of at least 0.84.

In various embodiments, the predictive model comprises a random forest model, and wherein the predictive model achieves an area under a curve (AUC) value of at least 0.72.

In various embodiments, the predictive model comprises a XGBoost model, and wherein the predictive model achieves an area under a curve (AUC) value of at least 0.73.

Additionally disclosed herein is a method for predicting risk of cancer in a subject, the method comprising: obtaining or having obtained a dataset derived from the subject comprising quantitative levels of a plurality of biomarkers, wherein the plurality of biomarkers comprises protein biomarkers comprising two or more of GAST, ENPP2, FZD8, FGF23, and TFF1, and generating a prediction of risk of cancer for the subject by applying a predictive model to the quantitative values of the plurality of biomarkers.

In various embodiments, the protein biomarkers comprise three or more of GAST, ENPP2, FZD8, FGF23, and TFF1.

In various embodiments, the protein biomarkers comprise four or more of GAST, ENPP2, FZD8, FGF23, and TFF1.

In various embodiments, the protein biomarkers comprise each of VWA5A, GAST, ENPP2, FZD8, FGF23, and TFF1.

In various embodiments, the protein biomarkers further comprise one or more of MAPT, FGF16, OXT, BRD1, MFAP4, WNT9A, FLRT2, CRTAC1, PAPPA, POMC, NGF, IDI2, TPT1, EPHA10, and MFAP3.

In various embodiments, the protein biomarkers further comprise five or more of MAPT, FGF16, OXT, BRD1, MFAP4, WNT9A, FLRT2, CRTAC1, PAPPA, POMC, NGF, IDI2, TPT1, EPHA10, and MFAP3.

In various embodiments, the protein biomarkers further comprise ten or more of MAPT, FGF16, OXT, BRD1, MFAP4, WNT9A, FLRT2, CRTAC1, PAPPA, POMC, NGF, IDI2, TPT1, EPHA10, and MFAP3.

In various embodiments, the protein biomarkers further comprise each of MAPT, FGF16, OXT, BRD1, MFAP4, WNT9A, FLRT2, CRTAC1, PAPPA, POMC, NGF, IDI2, TPT1, EPHA10, and MFAP3.

In various embodiments, the protein biomarkers further comprise one or more of SOWAHA, RARRES1, DUSP3, SEMA3F, CNTN3, LPA, KLK11, RPGR, EPO, TDGF1, IL17A, CD160, TNPO1, GAMT, ENPP6, TMEM25, GIP, CSPG5, SCGN, TMPRSS15, LAIR2, KIRREL1, NTF4, TSPAN7, ENDOU, KLK10, CCL24, GPR37, CD3D, and TJP3.

In various embodiments, the protein biomarkers further comprise five or more of SOWAHA, RARRES1, DUSP3, SEMA3F, CNTN3, LPA, KLK11, RPGR, EPO, TDGF1, IL17A, CD160, TNPO1, GAMT, ENPP6, TMEM25, GIP, CSPG5, SCGN, TMPRSS15, LAIR2, KIRREL1, NTF4, TSPAN7, ENDOU, KLK10, CCL24, GPR37, CD3D, and TJP3.

In various embodiments, the protein biomarkers further comprise ten or more of SOWAHA, RARRES1, DUSP3, SEMA3F, CNTN3, LPA, KLK11, RPGR, EPO, TDGF1, IL17A, CD160, TNPO1, GAMT, ENPP6, TMEM25, GIP, CSPG5, SCGN, TMPRSS15, LAIR2, KIRREL1, NTF4, TSPAN7, ENDOU, KLK10, CCL24, GPR37, CD3D, and TJP3.

In various embodiments, the protein biomarkers further comprise twenty or more of SOWAHA, RARRES1, DUSP3, SEMA3F, CNTN3, LPA, KLK11, RPGR, EPO, TDGF1, IL17A, CD160, TNPO1, GAMT, ENPP6, TMEM25, GIP, CSPG5, SCGN, TMPRSS15, LAIR2, KIRREL1, NTF4, TSPAN7, ENDOU, KLK10, CCL24, GPR37, CD3D, and TJP3.

The method of any one of claims 26-33, wherein the protein biomarkers further comprise each of SOWAHA, RARRES1, DUSP3, SEMA3F, CNTN3, LPA, KLK11, RPGR, EPO, TDGF1, IL17A, CD160, TNPO1, GAMT, ENPP6, TMEM25, GIP, CSPG5, SCGN, TMPRSS15, LAIR2, KIRREL1, NTF4, TSPAN7, ENDOU, KLK10, CCL24, GPR37, CD3D, and TJP3.

In various embodiments, the protein biomarkers further comprise one or more of DKKL1, CFC1, LRRC38, GCG, AGBL2, FASLG, AHNAK2, WFIKKN2, ANXA10, HS6ST1, DUSP29, CA14, CLEC7A, PHLDB2, SCRG1, RSPO3, TOP1, TINAGL1, NCAM1, FAM3D, FLT3LG, ZP3, AGRP, ASAH2, PDGFRB, AFM, NPY, PPY, XG, MFGE8, PROS1, MEGF11, SCT, CFB, F11, ANK2, ENOPH1, UGDH, ASAH1, ERBB4, IL36A, FGA, C5, OSMR, SSBP1, RICTOR, LRG1, C4BPB, AIDA, and SSC4D.

In various embodiments, the protein biomarkers further comprise five or more of DKKL1, CFC1, LRRC38, GCG, AGBL2, FASLG, AHNAK2, WFIKKN2, ANXA10, HS6ST1, DUSP29, CA14, CLEC7A, PHLDB2, SCRG1, RSPO3, TOP1, TINAGL1, NCAM1, FAM3D, FLT3LG, ZP3, AGRP, ASAH2, PDGFRB, AFM, NPY, PPY, XG, MFGE8, PROS1, MEGF11, SCT, CFB, F11, ANK2, ENOPH1, UGDH, ASAH1, ERBB4, IL36A, FGA, C5, OSMR, SSBP1, RICTOR, LRG1, C4BPB, AIDA, and SSC4D.

In various embodiments, the protein biomarkers further comprise ten or more of DKKL1, CFC1, LRRC38, GCG, AGBL2, FASLG, AHNAK2, WFIKKN2, ANXA10, HS6ST1, DUSP29, CA14, CLEC7A, PHLDB2, SCRG1, RSPO3, TOP1, TINAGL1, NCAM1, FAM3D, FLT3LG, ZP3, AGRP, ASAH2, PDGFRB, AFM, NPY, PPY, XG, MFGE8, PROS1, MEGF11, SCT, CFB, F11, ANK2, ENOPH1, UGDH, ASAH1, ERBB4, IL36A, FGA, C5, OSMR, SSBP1, RICTOR, LRG1, C4BPB, AIDA, and SSC4D.

In various embodiments, the protein biomarkers further comprise twenty or more of DKKL1, CFC1, LRRC38, GCG, AGBL2, FASLG, AHNAK2, WFIKKN2, ANXA10, HS6ST1, DUSP29, CA14, CLEC7A, PHLDB2, SCRG1, RSPO3, TOP1, TINAGL1, NCAM1, FAM3D, FLT3LG, ZP3, AGRP, ASAH2, PDGFRB, AFM, NPY, PPY, XG, MFGE8, PROS1, MEGF11, SCT, CFB, F11, ANK2, ENOPH1, UGDH, ASAH1, ERBB4, IL36A, FGA, C5, OSMR, SSBP1, RICTOR, LRG1, C4BPB, AIDA, and SSC4D.

In various embodiments, the protein biomarkers further comprise thirty or more of DKKL1, CFC1, LRRC38, GCG, AGBL2, FASLG, AHNAK2, WFIKKN2, ANXA10, HS6ST1, DUSP29, CA14, CLEC7A, PHLDB2, SCRG1, RSPO3, TOP1, TINAGL1, NCAM1, FAM3D, FLT3LG, ZP3, AGRP, ASAH2, PDGFRB, AFM, NPY, PPY, XG, MFGE8, PROS1, MEGF11, SCT, CFB, F11, ANK2, ENOPH1, UGDH, ASAH1, ERBB4, IL36A, FGA, C5, OSMR, SSBP1, RICTOR, LRG1, C4BPB, AIDA, and SSC4D.

In various embodiments, the protein biomarkers further comprise forty or more of DKKL1, CFC1, LRRC38, GCG, AGBL2, FASLG, AHNAK2, WFIKKN2, ANXA10, HS6ST1, DUSP29, CA14, CLEC7A, PHLDB2, SCRG1, RSPO3, TOP1, TINAGL1, NCAM1, FAM3D, FLT3LG, ZP3, AGRP, ASAH2, PDGFRB, AFM, NPY, PPY, XG, MFGE8, PROS1, MEGF11, SCT, CFB, F11, ANK2, ENOPH1, UGDH, ASAH1, ERBB4, IL36A, FGA, C5, OSMR, SSBP1, RICTOR, LRG1, C4BPB, AIDA, and SSC4D.

In various embodiments, the protein biomarkers further comprise each of DKKL1, CFC1, LRRC38, GCG, AGBL2, FASLG, AHNAK2, WFIKKN2, ANXA10, HS6ST1, DUSP29, CA14, CLEC7A, PHLDB2, SCRG1, RSPO3, TOP1, TINAGL1, NCAM1, FAM3D, FLT3LG, ZP3, AGRP, ASAH2, PDGFRB, AFM, NPY, PPY, XG, MFGE8, PROS1, MEGF11, SCT, CFB, F11, ANK2, ENOPH1, UGDH, ASAH1, ERBB4, IL36A, FGA, C5, OSMR, SSBP1, RICTOR, LRG1, C4BPB, AIDA, and SSC4D.

In various embodiments, the protein biomarkers further comprise one or more of GRN, IFNAR1, ENPEP, ACADSB, MAN1A2, GBP4, SERPING1, COL4A4, SOX2, GRSF1, PRAME, KIR2DS4, ADAMTS1, ITPRIP, CRISP3, DSG4, ITIH4, MRC1, GABRA4, SERPINA3, MILR1, PLIN1, SHH, KLKB1, IL17RA, MMP10, LBP, SMAD5, ADRA2A, SESTD1, CFI, AKR7L, CTSH, LYPD3, CBLIF, SMTN, CFH, SERPINC1, GDF15, PDZD2, ALDH2, IZUMO1, DNM3, CCL19, CSF2, MCEE, FDX1, SDC1, POSTN, GP2, CST7, CD14, NEK7, SHC1, CRELD1, TCN2, CMIP, CRHBP, C9, PXDNL, NRCAM, DLG4, TRAF3IP2, SULT2A1, GSTT2B, ITIH1, MRPL24, MUC16, IL3, CLU, FHIP2A, TK1, FKBP14, VWA5A, PRKG1, SV2A, PMCH, NEXN, CDCP1, DDX53, THSD1, PAK4, MMP12, FCN1, UMOD, PDIA4, IL6, BRK1, LILRA2, RBPMS2, SERPIND1, TPSG1, CEACAM5, FGF9, PPIF, RNF43, SIGLEC9, TOMM20, PDE5A, NELL1, GBA, PAEP, ERN1, PCSK7, CHCHD6, MARCO, SFTPA1, IL9, KYNU, SPINT1, LRFN2, NECTIN1, OSCAR, PZP, BPIFB1, LILRA5, CALY, RRAS, GADD45GIP1, ISM2, SCGB3A2, CEACAM6, LPP, GKN1, LRIG1, CLSPN, CXCL13, SFTPA2, COX6B1, PTGR1, RBPMS, PPT1, AOC1, PDLIM5, L3HYPDH, LONP1, APOL1, CEACAM18, FGF7, and KRT14.

In various embodiments, the predictive model comprises a elastic net regression model, and wherein the predictive model achieves an area under a curve (AUC) value of at least 0.79.

In various embodiments, the predictive model comprises a support vector machine, and wherein the predictive model achieves an area under a curve (AUC) value of at least 0.81.

In various embodiments, the predictive model comprises a random forest model, and wherein the predictive model achieves an area under a curve (AUC) value of at least 0.71.

In various embodiments, the predictive model comprises a XGBoost model, and wherein the predictive model achieves an area under a curve (AUC) value of at least 0.70.

Additionally disclosed herein is a method for predicting risk of cancer in a subject, the method comprising: obtaining or having obtained a dataset derived from the subject comprising quantitative levels of a plurality of biomarkers, wherein the plurality of biomarkers comprises protein biomarkers comprising two or more of TGFA, MMP12, TNFRSF13B, TNFSF14, and MASP1, and generating a prediction of risk of cancer for the subject by applying a predictive model to the quantitative values of the plurality of biomarkers.

In various embodiments, the protein biomarkers comprise three or more of TGFA, MMP12, TNFRSF13B, TNFSF14, and MASP1.

In various embodiments, the protein biomarkers comprise four or more of TGFA, MMP12, TNFRSF13B, TNFSF14, and MASP1.

In various embodiments, the protein biomarkers comprise each of TGFA, MMP12, TNFRSF13B, TNFSF14, and MASP1.

In various embodiments, the protein biomarkers further comprise one or more of THBS2, GDNF, FLT1, FXYD5, CST5, ARNT, CDCP1, CCL20, FLT3LG, CLEC7A, PRKCQ, SCGN, IL5, NPY, and S100A16.

In various embodiments, the protein biomarkers further comprise five or more of THBS2, GDNF, FLT1, FXYD5, CST5, ARNT, CDCP1, CCL20, FLT3LG, CLEC7A, PRKCQ, SCGN, IL5, NPY, and S100A16.

In various embodiments, the protein biomarkers further comprise ten or more of THBS2, GDNF, FLT1, FXYD5, CST5, ARNT, CDCP1, CCL20, FLT3LG, CLEC7A, PRKCQ, SCGN, IL5, NPY, and S100A16.

In various embodiments, the protein biomarkers further comprise each of THBS2, GDNF, FLT1, FXYD5, CST5, ARNT, CDCP1, CCL20, FLT3LG, CLEC7A, PRKCQ, SCGN, IL5, NPY, and S100A16.

In various embodiments, the protein biomarkers further comprise one or more of IL1B, CD84, STC1, PRDX3, LAP3, GAMT, CASP2, ITGA6, DECR1, and YTHDF3.

In various embodiments, the protein biomarkers further comprise five or more of IL1B, CD84, STC1, PRDX3, LAP3, GAMT, CASP2, ITGA6, DECR1, and YTHDF3.

In various embodiments, the protein biomarkers further comprise each of IL1B, CD84, STC1, PRDX3, LAP3, GAMT, CASP2, ITGA6, DECR1, and YTHDF3.

In various embodiments, the predictive model comprises a elastic net regression model, and wherein the predictive model achieves an area under a curve (AUC) value of at least 0.65.

In various embodiments, the predictive model comprises a support vector machine, and wherein the predictive model achieves an area under a curve (AUC) value of at least 0.70.

In various embodiments, the predictive model comprises a random forest model, and wherein the predictive model achieves an area under a curve (AUC) value of at least 0.67.

In various embodiments, the predictive model comprises a XGBoost model, and wherein the predictive model achieves an area under a curve (AUC) value of at least 0.68.

In various embodiments, the cancer is lung cancer.

In various embodiments, the risk of cancer is a level of risk of the subject developing cancer within 1 year, within 2 years, within 3 years, within 4 years, within 5 years, within 6 years, within 7 years, within 8 years, within 9 years, or within 10 years.

In various embodiments, the risk of cancer is a presence or absence of cancer.

In various embodiments, the dataset is derived from a test sample obtained from the subject.

In various embodiments, the test sample is a blood, serum or plasma sample.

In various embodiments, obtaining or having obtained the dataset comprises performing one or more assays.

In various embodiments, performing the one or more assays comprises performing an immunoassay to determine the expression levels of the plurality of biomarkers.

In various embodiments, the immunoassay is a Proximity Extension Assay (PEA) or LUMINEX xMAP Multiplex Assay.

In various embodiments, the dataset comprises plasma proteomics data.

In various embodiments, the method further comprises: selecting a therapy for providing to the subject based on the prediction of cancer.

Additionally disclosed herein is a non-transitory computer readable medium comprising instructions that, when executed by a processor, cause the processor to: obtain or have obtained a dataset derived from the subject comprising quantitative levels of a plurality of biomarkers, wherein the plurality of biomarkers comprises protein biomarkers comprising two or more of TSPAN1, CD28, SCN3B, ADGRB3, and IGFBP6, and generate a prediction of risk of cancer for the subject by applying a predictive model to the quantitative values of the plurality of biomarkers.

In various embodiments, the protein biomarkers comprise three or more of TSPAN1, CD28, SCN3B, ADGRB3, and IGFBP6.

In various embodiments, the protein biomarkers comprise four or more of TSPAN1, CD28, SCN3B, ADGRB3, and IGFBP6.

In various embodiments, the protein biomarkers comprise each of TSPAN1, CD28, SCN3B, ADGRB3, and IGFBP6.

In various embodiments, the protein biomarkers further comprise one or more of NRTN, AIF1L, HSPB6, MB, TNFRSF19, IL5RA, TNR, CDNF, CST1, FGFBP2, S100A16, CD248, GFRA3, LMOD1, and POF1B.

In various embodiments, the protein biomarkers further comprise five or more of NRTN, AIF1L, HSPB6, MB, TNFRSF19, IL5RA, TNR, CDNF, CST1, FGFBP2, S100A16, CD248, GFRA3, LMOD1, and POF1B.

In various embodiments, the protein biomarkers further comprise ten or more of NRTN, AIF1L, HSPB6, MB, TNFRSF19, IL5RA, TNR, CDNF, CST1, FGFBP2, S100A16, CD248, GFRA3, LMOD1, and POF1B.

In various embodiments, the protein biomarkers further comprise each of NRTN, AIF1L, HSPB6, MB, TNFRSF19, IL5RA, TNR, CDNF, CST1, FGFBP2, S100A16, CD248, GFRA3, LMOD1, and POF1B.

In various embodiments, the protein biomarkers further comprise one or more of DENND2B, COMP, CNTN2, SCARA5, CSPG4, ITGAV, SOST, SERPINA4, LILRA4, SPINK5, PINLYP, ACTN2, JAM2, FAP, TMOD4, GUCA2A, MFAP3L, DKK4, LAMA1, BAG3, SNCG, SEPTIN3, VWC2, KLRC1, ATRAID, ART3, SLITRK2, SIGLEC6, TMED4, and SLAMF7.

In various embodiments, the protein biomarkers further comprise five or more of DENND2B, COMP, CNTN2, SCARA5, CSPG4, ITGAV, SOST, SERPINA4, LILRA4, SPINK5, PINLYP, ACTN2, JAM2, FAP, TMOD4, GUCA2A, MFAP3L, DKK4, LAMA1, BAG3, SNCG, SEPTIN3, VWC2, KLRC1, ATRAID, ART3, SLITRK2, SIGLEC6, TMED4, and SLAMF7.

In various embodiments, the protein biomarkers further comprise ten or more of DENND2B, COMP, CNTN2, SCARA5, CSPG4, ITGAV, SOST, SERPINA4, LILRA4, SPINK5, PINLYP, ACTN2, JAM2, FAP, TMOD4, GUCA2A, MFAP3L, DKK4, LAMA1, BAG3, SNCG, SEPTIN3, VWC2, KLRC1, ATRAID, ART3, SLITRK2, SIGLEC6, TMED4, and SLAMF7.

In various embodiments, the protein biomarkers further comprise twenty or more of DENND2B, COMP, CNTN2, SCARA5, CSPG4, ITGAV, SOST, SERPINA4, LILRA4, SPINK5, PINLYP, ACTN2, JAM2, FAP, TMOD4, GUCA2A, MFAP3L, DKK4, LAMA1, BAG3, SNCG, SEPTIN3, VWC2, KLRC1, ATRAID, ART3, SLITRK2, SIGLEC6, TMED4, and SLAMF7.

In various embodiments, the protein biomarkers further comprise each of DENND2B, COMP, CNTN2, SCARA5, CSPG4, ITGAV, SOST, SERPINA4, LILRA4, SPINK5, PINLYP, ACTN2, JAM2, FAP, TMOD4, GUCA2A, MFAP3L, DKK4, LAMA1, BAG3, SNCG, SEPTIN3, VWC2, KLRC1, ATRAID, ART3, SLITRK2, SIGLEC6, TMED4, and SLAMF7.

In various embodiments, the protein biomarkers further comprise one or more of CKMT1A, SEMA6C, CD2, CST5, PBXIP1, LECT2, PYY, AGRN, INSL5, CD38, PI16, CCN5, TNFRSF17, LY9, GPC1, CLMP, MEP1B, CCN1, PCDH7, SPARCL1, CRNN, PM20D1, TNFRSF12A, DSCAM, PALM, CX3CL1, MEP1A, SLURP1, APOA4, ADAMTSL5, MEPE, WFDC1, RPS10, CD300C, RIPK4, CALCB, RTBDN, ENO3, NTF3, PTPRZ1, LRP2BP, CPE, MCAM, BGN, PLB1, YAP1, TGFBI, CYB5A, EDDM3B, and SELENOP.

In various embodiments, the protein biomarkers further comprise five or more of CKMT1A, SEMA6C, CD2, CST5, PBXIP1, LECT2, PYY, AGRN, INSL5, CD38, PI16, CCN5, TNFRSF17, LY9, GPC1, CLMP, MEP1B, CCN1, PCDH7, SPARCL1, CRNN, PM20D1, TNFRSF12A, DSCAM, PALM, CX3CL1, MEP1A, SLURP1, APOA4, ADAMTSL5, MEPE, WFDC1, RPS10, CD300C, RIPK4, CALCB, RTBDN, ENO3, NTF3, PTPRZ1, LRP2BP, CPE, MCAM, BGN, PLB1, YAP1, TGFBI, CYB5A, EDDM3B, and SELENOP.

In various embodiments, the protein biomarkers further comprise ten or more of CKMT1A, SEMA6C, CD2, CST5, PBXIP1, LECT2, PYY, AGRN, INSL5, CD38, PI16, CCN5, TNFRSF17, LY9, GPC1, CLMP, MEP1B, CCN1, PCDH7, SPARCL1, CRNN, PM20D1, TNFRSF12A, DSCAM, PALM, CX3CL1, MEP1A, SLURP1, APOA4, ADAMTSL5, MEPE, WFDC1, RPS10, CD300C, RIPK4, CALCB, RTBDN, ENO3, NTF3, PTPRZ1, LRP2BP, CPE, MCAM, BGN, PLB1, YAP1, TGFBI, CYB5A, EDDM3B, and SELENOP.

In various embodiments, the protein biomarkers further comprise twenty or more of CKMT1A, SEMA6C, CD2, CST5, PBXIP1, LECT2, PYY, AGRN, INSL5, CD38, PI16, CCN5, TNFRSF17, LY9, GPC1, CLMP, MEP1B, CCN1, PCDH7, SPARCL1, CRNN, PM20D1, TNFRSF12A, DSCAM, PALM, CX3CL1, MEP1A, SLURP1, APOA4, ADAMTSL5, MEPE, WFDC1, RPS10, CD300C, RIPK4, CALCB, RTBDN, ENO3, NTF3, PTPRZ1, LRP2BP, CPE, MCAM, BGN, PLB1, YAP1, TGFBI, CYB5A, EDDM3B, and SELENOP.

In various embodiments, the protein biomarkers further comprise thirty or more of CKMT1A, SEMA6C, CD2, CST5, PBXIP1, LECT2, PYY, AGRN, INSL5, CD38, PI16, CCN5, TNFRSF17, LY9, GPC1, CLMP, MEP1B, CCN1, PCDH7, SPARCL1, CRNN, PM20D1, TNFRSF12A, DSCAM, PALM, CX3CL1, MEP1A, SLURP1, APOA4, ADAMTSL5, MEPE, WFDC1, RPS10, CD300C, RIPK4, CALCB, RTBDN, ENO3, NTF3, PTPRZ1, LRP2BP, CPE, MCAM, BGN, PLB1, YAP1, TGFBI, CYB5A, EDDM3B, and SELENOP.

In various embodiments, the protein biomarkers further comprise forty or more of CKMT1A, SEMA6C, CD2, CST5, PBXIP1, LECT2, PYY, AGRN, INSL5, CD38, PI16, CCN5, TNFRSF17, LY9, GPC1, CLMP, MEP1B, CCN1, PCDH7, SPARCL1, CRNN, PM20D1, TNFRSF12A, DSCAM, PALM, CX3CL1, MEP1A, SLURP1, APOA4, ADAMTSL5, MEPE, WFDC1, RPS10, CD300C, RIPK4, CALCB, RTBDN, ENO3, NTF3, PTPRZ1, LRP2BP, CPE, MCAM, BGN, PLB1, YAP1, TGFBI, CYB5A, EDDM3B, and SELENOP.

In various embodiments, the protein biomarkers further comprise each of CKMT1A, SEMA6C, CD2, CST5, PBXIP1, LECT2, PYY, AGRN, INSL5, CD38, PI16, CCN5, TNFRSF17, LY9, GPC1, CLMP, MEP1B, CCN1, PCDH7, SPARCL1, CRNN, PM20D1, TNFRSF12A, DSCAM, PALM, CX3CL1, MEP1A, SLURP1, APOA4, ADAMTSL5, MEPE, WFDC1, RPS10, CD300C, RIPK4, CALCB, RTBDN, ENO3, NTF3, PTPRZ1, LRP2BP, CPE, MCAM, BGN, PLB1, YAP1, TGFBI, CYB5A, EDDM3B, and SELENOP.

In various embodiments, the protein biomarkers further comprise one or more of ENPP6, TMEM25, GIP, CSPG5, SCGN, TMPRSS15, LAIR2, KIRREL1, NTF4, TSPAN7, ENDOU, KLK10, CCL24, GPR37, CD3D, TJP3, DKKL1, CFC1, LRRC38, GCG, AGBL2, FASLG, AHNAK2, WFIKKN2, ANXA10, HS6ST1, DUSP29, CA14, CLEC7A, PHLDB2, SCRG1, RSPO3, TOP1, TINAGL1, NCAM1, FAM3D, FLT3LG, ZP3, AGRP, ASAH2, PDGFRB, AFM, NPY, PPY, XG, MFGE8, PROS1, MEGF11, CTSO, CTLA4, CSF3R, FCAR, CTAG1A, SCPEP1, PRSS53, CRELD2, PILRA, PROC, VASH1, NOS3, BPIFB2, UPK3BL1, NOP56, JAM3, HLA-DRA, SIL1, TRPV3, EDEM2, POLR2A, CBLN1, FKBP7, CCL20, PILRB, SIRPB1, VSTM1, BST2, DLL4, C1RL, RNASET2, KCNH2, IL12RB2, FZD10, OXCT1, TREML2, GRIN2B, GFRAL, RGS8, LRPAP1, LRP2, IGSF21, DPT, HEPACAM2, MATN3, UXS1, PTTG1, BTN1A1, IL17C, SCIN, TK1, FKBP14, VWA5A, PRKG1, SV2A, PMCH, NEXN, CDCP1, DDX53, THSD1, PAK4, MMP12, FCN1, UMOD, PDIA4, IL6, BRK1, LILRA2, RBPMS2, SERPIND1, TPSG1, CEACAM5, FGF9, PPIF, RNF43, SIGLEC9, TOMM20, PDE5A, NELL1, GBA, PAEP, ERN1, PCSK7, CHCHD6, MARCO, SFTPA1, IL9, KYNU, SPINT1, LRFN2, NECTIN1, OSCAR, PZP, BPIFB1, LILRA5, CALY, RRAS, GADD45GIP1, ISM2, SCGB3A2, CEACAM6, LPP, GKN1, LRIG1, CLSPN, CXCL13, SFTPA2, COX6B1, PTGR1, RBPMS, PPT1, AOC1, PDLIM5, L3HYPDH, LONP1, APOL1, CEACAM18, FGF7, KRT14.

In various embodiments, the predictive model comprises a elastic net regression model, and wherein the predictive model achieves an area under a curve (AUC) value of at least 0.85.

In various embodiments, the predictive model comprises a support vector machine, and wherein the predictive model achieves an area under a curve (AUC) value of at least 0.84.

In various embodiments, the predictive model comprises a random forest model, and wherein the predictive model achieves an area under a curve (AUC) value of at least 0.72.

In various embodiments, the predictive model comprises a XGBoost model, and wherein the predictive model achieves an area under a curve (AUC) value of at least 0.73.

Additionally disclosed herein is a non-transitory computer readable medium comprising instructions that, when executed by a processor, cause the processor to: obtain or have obtained a dataset derived from the subject comprising quantitative levels of a plurality of biomarkers, wherein the plurality of biomarkers comprises protein biomarkers comprising two or more of GAST, ENPP2, FZD8, FGF23, and TFF1, and generate a prediction of risk of cancer for the subject by applying a predictive model to the quantitative values of the plurality of biomarkers.

In various embodiments, the protein biomarkers comprise three or more of GAST, ENPP2, FZD8, FGF23, and TFF1.

In various embodiments, the protein biomarkers comprise four or more of GAST, ENPP2, FZD8, FGF23, and TFF1.

In various embodiments, the protein biomarkers comprise each of GAST, ENPP2, FZD8, FGF23, and TFF1.

In various embodiments, the protein biomarkers further comprise one or more of MAPT, FGF16, OXT, BRD1, MFAP4, WNT9A, FLRT2, CRTAC1, PAPPA, POMC, NGF, IDI2, TPT1, EPHA10, and MFAP3.

In various embodiments, the protein biomarkers further comprise five or more of MAPT, FGF16, OXT, BRD1, MFAP4, WNT9A, FLRT2, CRTAC1, PAPPA, POMC, NGF, IDI2, TPT1, EPHA10, and MFAP3.

In various embodiments, the protein biomarkers further comprise ten or more of MAPT, FGF16, OXT, BRD1, MFAP4, WNT9A, FLRT2, CRTAC1, PAPPA, POMC, NGF, IDI2, TPT1, EPHA10, and MFAP3.

In various embodiments, the protein biomarkers further comprise each of MAPT, FGF16, OXT, BRD1, MFAP4, WNT9A, FLRT2, CRTAC1, PAPPA, POMC, NGF, IDI2, TPT1, EPHA10, and MFAP3.

In various embodiments, the protein biomarkers further comprise one or more of SOWAHA, RARRES1, DUSP3, SEMA3F, CNTN3, LPA, KLK11, RPGR, EPO, TDGF1, IL17A, CD160, TNPO1, GAMT, ENPP6, TMEM25, GIP, CSPG5, SCGN, TMPRSS15, LAIR2, KIRREL1, NTF4, TSPAN7, ENDOU, KLK10, CCL24, GPR37, CD3D, and TJP3.

In various embodiments, the protein biomarkers further comprise five or more of SOWAHA, RARRES1, DUSP3, SEMA3F, CNTN3, LPA, KLK11, RPGR, EPO, TDGF1, IL17A, CD160, TNPO1, GAMT, ENPP6, TMEM25, GIP, CSPG5, SCGN, TMPRSS15, LAIR2, KIRREL1, NTF4, TSPAN7, ENDOU, KLK10, CCL24, GPR37, CD3D, and TJP3.

In various embodiments, the protein biomarkers further comprise ten or more of SOWAHA, RARRES1, DUSP3, SEMA3F, CNTN3, LPA, KLK11, RPGR, EPO, TDGF1, IL17A, CD160, TNPO1, GAMT, ENPP6, TMEM25, GIP, CSPG5, SCGN, TMPRSS15, LAIR2, KIRREL1, NTF4, TSPAN7, ENDOU, KLK10, CCL24, GPR37, CD3D, and TJP3.

In various embodiments, the protein biomarkers further comprise twenty or more of SOWAHA, RARRES1, DUSP3, SEMA3F, CNTN3, LPA, KLK11, RPGR, EPO, TDGF1, IL17A, CD160, TNPO1, GAMT, ENPP6, TMEM25, GIP, CSPG5, SCGN, TMPRSS15, LAIR2, KIRREL1, NTF4, TSPAN7, ENDOU, KLK10, CCL24, GPR37, CD3D, and TJP3.

In various embodiments, the protein biomarkers further comprise each of SOWAHA, RARRES1, DUSP3, SEMA3F, CNTN3, LPA, KLK11, RPGR, EPO, TDGF1, IL17A, CD160, TNPO1, GAMT, ENPP6, TMEM25, GIP, CSPG5, SCGN, TMPRSS15, LAIR2, KIRREL1, NTF4, TSPAN7, ENDOU, KLK10, CCL24, GPR37, CD3D, and TJP3.

In various embodiments, the protein biomarkers further comprise one or more of DKKL1, CFC1, LRRC38, GCG, AGBL2, FASLG, AHNAK2, WFIKKN2, ANXA10, HS6ST1, DUSP29, CA14, CLEC7A, PHLDB2, SCRG1, RSPO3, TOP1, TINAGL1, NCAM1, FAM3D, FLT3LG, ZP3, AGRP, ASAH2, PDGFRB, AFM, NPY, PPY, XG, MFGE8, PROS1, MEGF11, SCT, CFB, F11, ANK2, ENOPH1, UGDH, ASAH1, ERBB4, IL36A, FGA, C5, OSMR, SSBP1, RICTOR, LRG1, C4BPB, AIDA, and SSC4D.

In various embodiments, the protein biomarkers further comprise five or more of DKKL1, CFC1, LRRC38, GCG, AGBL2, FASLG, AHNAK2, WFIKKN2, ANXA10, HS6ST1, DUSP29, CA14, CLEC7A, PHLDB2, SCRG1, RSPO3, TOP1, TINAGL1, NCAM1, FAM3D, FLT3LG, ZP3, AGRP, ASAH2, PDGFRB, AFM, NPY, PPY, XG, MFGE8, PROS1, MEGF11, SCT, CFB, F11, ANK2, ENOPH1, UGDH, ASAH1, ERBB4, IL36A, FGA, C5, OSMR, SSBP1, RICTOR, LRG1, C4BPB, AIDA, and SSC4D.

In various embodiments, the protein biomarkers further comprise ten or more of DKKL1, CFC1, LRRC38, GCG, AGBL2, FASLG, AHNAK2, WFIKKN2, ANXA10, HS6ST1, DUSP29, CA14, CLEC7A, PHLDB2, SCRG1, RSPO3, TOP1, TINAGL1, NCAM1, FAM3D, FLT3LG, ZP3, AGRP, ASAH2, PDGFRB, AFM, NPY, PPY, XG, MFGE8, PROS1, MEGF11, SCT, CFB, F11, ANK2, ENOPH1, UGDH, ASAH1, ERBB4, IL36A, FGA, C5, OSMR, SSBP1, RICTOR, LRG1, C4BPB, AIDA, and SSC4D.

In various embodiments, the protein biomarkers further comprise twenty or more of DKKL1, CFC1, LRRC38, GCG, AGBL2, FASLG, AHNAK2, WFIKKN2, ANXA10, HS6ST1, DUSP29, CA14, CLEC7A, PHLDB2, SCRG1, RSPO3, TOP1, TINAGL1, NCAM1, FAM3D, FLT3LG, ZP3, AGRP, ASAH2, PDGFRB, AFM, NPY, PPY, XG, MFGE8, PROS1, MEGF11, SCT, CFB, F11, ANK2, ENOPH1, UGDH, ASAH1, ERBB4, IL36A, FGA, C5, OSMR, SSBP1, RICTOR, LRG1, C4BPB, AIDA, and SSC4D.

In various embodiments, the protein biomarkers further comprise thirty or more of DKKL1, CFC1, LRRC38, GCG, AGBL2, FASLG, AHNAK2, WFIKKN2, ANXA10, HS6ST1, DUSP29, CA14, CLEC7A, PHLDB2, SCRG1, RSPO3, TOP1, TINAGL1, NCAM1, FAM3D, FLT3LG, ZP3, AGRP, ASAH2, PDGFRB, AFM, NPY, PPY, XG, MFGE8, PROS1, MEGF11, SCT, CFB, F11, ANK2, ENOPH1, UGDH, ASAH1, ERBB4, IL36A, FGA, C5, OSMR, SSBP1, RICTOR, LRG1, C4BPB, AIDA, and SSC4D.

In various embodiments, the protein biomarkers further comprise forty or more of DKKL1, CFC1, LRRC38, GCG, AGBL2, FASLG, AHNAK2, WFIKKN2, ANXA10, HS6ST1, DUSP29, CA14, CLEC7A, PHLDB2, SCRG1, RSPO3, TOP1, TINAGL1, NCAM1, FAM3D, FLT3LG, ZP3, AGRP, ASAH2, PDGFRB, AFM, NPY, PPY, XG, MFGE8, PROS1, MEGF11, SCT, CFB, F11, ANK2, ENOPH1, UGDH, ASAH1, ERBB4, IL36A, FGA, C5, OSMR, SSBP1, RICTOR, LRG1, C4BPB, AIDA, and SSC4D.

In various embodiments, the protein biomarkers further comprise each of DKKL1, CFC1, LRRC38, GCG, AGBL2, FASLG, AHNAK2, WFIKKN2, ANXA10, HS6ST1, DUSP29, CA14, CLEC7A, PHLDB2, SCRG1, RSPO3, TOP1, TINAGL1, NCAM1, FAM3D, FLT3LG, ZP3, AGRP, ASAH2, PDGFRB, AFM, NPY, PPY, XG, MFGE8, PROS1, MEGF11, SCT, CFB, F11, ANK2, ENOPH1, UGDH, ASAH1, ERBB4, IL36A, FGA, C5, OSMR, SSBP1, RICTOR, LRG1, C4BPB, AIDA, and SSC4D.

In various embodiments, the protein biomarkers further comprise one or more of GRN, IFNAR1, ENPEP, ACADSB, MAN1A2, GBP4, SERPING1, COL4A4, SOX2, GRSF1, PRAME, KIR2DS4, ADAMTS1, ITPRIP, CRISP3, DSG4, ITIH4, MRC1, GABRA4, SERPINA3, MILR1, PLIN1, SHH, KLKB1, IL17RA, MMP10, LBP, SMAD5, ADRA2A, SESTD1, CFI, AKR7L, CTSH, LYPD3, CBLIF, SMTN, CFH, SERPINC1, GDF15, PDZD2, ALDH2, IZUMO1, DNM3, CCL19, CSF2, MCEE, FDX1, SDC1, POSTN, GP2, CST7, CD14, NEK7, SHC1, CRELD1, TCN2, CMIP, CRHBP, C9, PXDNL, NRCAM, DLG4, TRAF3IP2, SULT2A1, GSTT2B, ITIH1, MRPL24, MUC16, IL3, CLU, FHIP2A, TK1, FKBP14, VWA5A, PRKG1, SV2A, PMCH, NEXN, CDCP1, DDX53, THSD1, PAK4, MMP12, FCN1, UMOD, PDIA4, IL6, BRK1, LILRA2, RBPMS2, SERPIND1, TPSG1, CEACAM5, FGF9, PPIF, RNF43, SIGLEC9, TOMM20, PDE5A, NELL1, GBA, PAEP, ERN1, PCSK7, CHCHD6, MARCO, SFTPA1, IL9, KYNU, SPINT1, LRFN2, NECTIN1, OSCAR, PZP, BPIFB1, LILRA5, CALY, RRAS, GADD45GIP1, ISM2, SCGB3A2, CEACAM6, LPP, GKN1, LRIG1, CLSPN, CXCL13, SFTPA2, COX6B1, PTGR1, RBPMS, PPT1, AOC1, PDLIM5, L3HYPDH, LONP1, APOL1, CEACAM18, FGF7, and KRT14.

In various embodiments, the predictive model comprises a elastic net regression model, and wherein the predictive model achieves an area under a curve (AUC) value of at least 0.79.

In various embodiments, the predictive model comprises a support vector machine, and wherein the predictive model achieves an area under a curve (AUC) value of at least 0.81.

In various embodiments, the predictive model comprises a random forest model, and wherein the predictive model achieves an area under a curve (AUC) value of at least 0.71.

In various embodiments, the predictive model comprises a XGBoost model, and wherein the predictive model achieves an area under a curve (AUC) value of at least 0.70.

In various embodiments, the cancer is lung cancer.

In various embodiments, the risk of cancer is a level of risk of the subject developing cancer within 1 year, within 2 years, within 3 years, within 4 years, within 5 years, within 6 years, within 7 years, within 8 years, within 9 years, or within 10 years.

In various embodiments, the risk of cancer is a presence or absence of cancer.

In various embodiments, the dataset is derived from a test sample obtained from the subject.

In various embodiments, the test sample is a blood, serum or plasma sample.

In various embodiments, the dataset is obtained from having performed one or more assays.

In various embodiments, the one or more assays comprises an immunoassay to determine the expression levels of the plurality of biomarkers.

In various embodiments, the immunoassay is a Proximity Extension Assay (PEA) or LUMINEX xMAP Multiplex Assay.

Additionally disclosed herein is a non-transitory computer readable medium comprising instructions that, when executed by a processor, cause the processor to: obtain or have obtained a dataset derived from the subject comprising quantitative levels of a plurality of biomarkers, wherein the plurality of biomarkers comprises protein biomarkers comprising two or more of TGFA, MMP12, TNFRSF13B, TNFSF14, and MASP1, and generate a prediction of risk of cancer for the subject by applying a predictive model to the quantitative values of the plurality of biomarkers.

In various embodiments, the protein biomarkers comprise three or more of TGFA, MMP12, TNFRSF13B, TNFSF14, and MASP1.

In various embodiments, the protein biomarkers comprise four or more of TGFA, MMP12, TNFRSF13B, TNFSF14, and MASP1.

In various embodiments, the protein biomarkers comprise each of TGFA, MMP12, TNFRSF13B, TNFSF14, and MASP1.

In various embodiments, the protein biomarkers further comprise one or more of THBS2, GDNF, FLT1, FXYD5, CST5, ARNT, CDCP1, CCL20, FLT3LG, CLEC7A, PRKCQ, SCGN, IL5, NPY, and S100A16.

In various embodiments, the protein biomarkers further comprise five or more of THBS2, GDNF, FLT1, FXYD5, CST5, ARNT, CDCP1, CCL20, FLT3LG, CLEC7A, PRKCQ, SCGN, IL5, NPY, and S100A16.

In various embodiments, the protein biomarkers further comprise ten or more of THBS2, GDNF, FLT1, FXYD5, CST5, ARNT, CDCP1, CCL20, FLT3LG, CLEC7A, PRKCQ, SCGN, IL5, NPY, and S100A16.

In various embodiments, the protein biomarkers further comprise each of THBS2, GDNF, FLT1, FXYD5, CST5, ARNT, CDCP1, CCL20, FLT3LG, CLEC7A, PRKCQ, SCGN, IL5, NPY, and S100A16.

In various embodiments, the protein biomarkers further comprise one or more of IL1B, CD84, STC1, PRDX3, LAP3, GAMT, CASP2, ITGA6, DECR1, and YTHDF3.

In various embodiments, the protein biomarkers further comprise five or more of IL1B, CD84, STC1, PRDX3, LAP3, GAMT, CASP2, ITGA6, DECR1, and YTHDF3.

In various embodiments, the protein biomarkers further comprise each of IL1B, CD84, STC1, PRDX3, LAP3, GAMT, CASP2, ITGA6, DECR1, and YTHDF3.

In various embodiments, the predictive model comprises a elastic net regression model, and wherein the predictive model achieves an area under a curve (AUC) value of at least 0.65.

In various embodiments, the predictive model comprises a support vector machine, and wherein the predictive model achieves an area under a curve (AUC) value of at least 0.70.

In various embodiments, the predictive model comprises a random forest model, and wherein the predictive model achieves an area under a curve (AUC) value of at least 0.67.

In various embodiments, the predictive model comprises a XGBoost model, and wherein the predictive model achieves an area under a curve (AUC) value of at least 0.68.

In various embodiments, the dataset comprises plasma proteomics data.

In various embodiments, a therapy is selected for providing to the subject based on the prediction of cancer.

### BRIEF DESCRIPTION OF THE DRAWINGS

These and other features, aspects, and advantages of the present invention will become better understood with regard to the following description and accompanying drawings.

FIG. 1A depicts an overview of an environment for predicting risk of cancer in a subject via a cancer prediction system, in accordance with an embodiment.

FIG. 1B depicts a block diagram of the cancer prediction system, in accordance with an embodiment.

FIG. 2 depicts example training data for training a prediction model, in accordance with an embodiment.

FIG. 3 depicts implementation of an example prediction model, in accordance with an embodiment.

FIG. 4 illustrates an example computer for implementing the entities shown in FIG. 1A, 1i, 2, and 3.

FIGS. 5A-5C show the performance of predictive models using various machine learning algorithms in an Olink® Target 96 platform, in accordance with the embodiments of the prediction model shown in FIGS. 1-3.

FIGS. 6A-6B show the performance of predictive models using various machine learning algorithms in an Olink® Explore 3072 platform, in accordance with the embodiments of the prediction model shown in FIGS. 1-3.

FIGS. 7A-7B show the performance of predictive models using various machine learning algorithms in an Olink® Explore 3072 platform, in accordance with the embodiments of the prediction model shown in FIGS. 1-3.

FIGS. 8A-8E illustrate circulating plasma proteins prediction of future lung cancer using the 240 proteins in the 1-3Y cohort (as identified in Table 13). FIG. 8A illustrates a boxplot of training AUC values from four different machine learning models (e.g., Elastic Net, Random Forest, Support Vector Machine, XGBoost, 5-fold CV repeated 5 times) trained on the LLP cohort to predict lung cancer in patients 1-3 years before diagnosis (53 cancer and 109 control samples). FIG. 8B illustrates combined z-scores plotted over time in the LLP cohort for 1-3Y proteins, where protein levels in LLP subjects were transformed using the z-score method and combined to generate one score. FIG. 8C illustrates AUROC (Area Under the Receiver Operating Characteristic Curve) of 1-3Y SVM model trained in Liverpool tested in UK Biobank samples 1-3 years before lung cancer diagnosis (62 cancer and 5500 control samples). FIG. 8D illustrates performance of the 1-3Y SVM model in the UK Biobank across different years of diagnosis of lung cancer. Samples taken at different times prior to lung cancer were segregated by year (2-12 years) and the SVM model for 1-3Y was tested by ROC analysis. FIG. 8E illustrates Barplot for AUROC values for SVM model predicting future development of cancer for several cancer types from UK Biobank 1-3 years before diagnosis, where the same approach as taken for lung cancer was taken to identify plasma samples at least 2 years prior to other first cancer diagnosis (number of cases labelled on bar chart) and the AUC for ROC analysis shown.

FIGS. 9A-9B illustrate combined z-score from 1-3Y in relation to cancer stage and pack years of smoking, where protein levels in LLP subjects were transformed using the z-score method and combined to generate one score. FIG. 9A illustrates combined z-scores plotted in time-frame categories (5-10 years, 3-5 years, 1-3 years prior to diagnosis or at diagnosis) for healthy subjects and cases of different lung cancer stage for 1-3Y proteins with P-values generated using Wilcoxon signed-rank test. FIG. 9B illustrates z-scores correlated with pack years of smoking at time of sample in the same time frame categories, where the correlation was measured using Pearson correlation coefficient.

FIGS. 10A-10C illustrate circulating plasma proteins prediction of long-term future lung cancer. FIG. 10A illustrates a boxplot of training AUC values from four different machine learning models (Elastic Net, Random Forest, Support Vector Machine, XGBoost, 5-fold CV repeated 5 times) trained on the LLP cohort to predict lung cancer in patients 1-5 years before diagnosis (110 Cancer, 215 control samples). FIG. 10B illustrates combined z-scores plotted over time in the LLP cohort for 1-5Y proteins, where protein levels in LLP subjects were transformed using the z-score method and combined to generate one score. FIG. 10C illustrates z-scores correlated with age at time of sample in the same time frame categories; correlation was measured using Pearson correlation coefficient.

FIG. 11 illustrates Gene Enrichment Analysis including top 20 pathways over- or under-represented in plasma samples from 1-3Y or 1-5Y models. FIG. 11 demonstrates pathways for predictive panels, including three shared over-represented and three shared under-represented pathways.

FIG. 12 illustrates an example Study Design.

FIG. 13 illustrates identification of future lung cancer cases and relevant matched controls from the UK Biobank.

FIG. 14 illustrates correlation between plasma protein measurements utilizing the Olink Target 96 platform (“old”) and the Olink Explore 3072 platform (“new”).

FIG. 15 illustrates longitudinal changes in z score for 1-3Y and 1-5Y proteins.

FIG. 16A-16F illustrate combined z-scores from 1-3Y and 1-5Y in relation to histology, history of COPD, age, and stage.

FIG. 17 illustrates examples of time-dependent levels for selected plasma proteins.

### DETAILED DESCRIPTION

### I. Definitions

Terms used in the claims and specification are defined as set forth below unless otherwise specified.

The term “subject” encompasses a cell, tissue, or organism, human or non-human, whether in vivo, ex vivo, or in vitro, male or female.

The term “mammal” encompasses both humans and non-humans and includes but is not limited to humans, non-human primates, canines, felines, murines, bovines, equines, and porcines.

The term “sample” can include a single cell or multiple cells or fragments of cells or an aliquot of body fluid, such as a blood sample, taken from a subject, by means including venipuncture, excretion, ejaculation, massage, biopsy, needle aspirate, lavage sample, scraping, surgical incision, or intervention or other means known in the art. Examples of an aliquot of body fluid include amniotic fluid, aqueous humor, bile, lymph, breast milk, interstitial fluid, blood, blood plasma, cerumen (earwax), Cowper's fluid (pre-ejaculatory fluid), chyle, chyme, female ejaculate, menses, mucus, saliva, urine, vomit, tears, vaginal lubrication, sweat, serum, semen, sebum, pus, pleural fluid, cerebrospinal fluid, synovial fluid, intracellular fluid, and vitreous humour.

The term “predictor” or “predictors” refers to variables, such as markers or biomarkers, analyzed by a prediction model, or one or more panels of a prediction model. In various embodiments, a “predictor” refers to biomarkers, such as protein biomarkers.

The terms “marker,” “markers,” “biomarker,” and “biomarkers” encompass, without limitation, lipids, lipoproteins, proteins, cytokines, chemokines, growth factors, peptides, nucleic acids (e.g., DNA, mRNA, or micro-RNA (miRNA)), genes, and oligonucleotides, together with their related complexes, metabolites, mutations, variants, polymorphisms, modifications, fragments, subunits, degradation products, elements, and other analytes or sample-derived measures. A marker can also include mutated proteins, mutated nucleic acids, variations in copy numbers, and/or transcript variants, in circumstances in which such mutations, variations in copy number and/or transcript variants are useful for generating a prediction model, or are useful in prediction models developed using related markers (e.g., non-mutated versions of the proteins or nucleic acids, alternative transcripts, etc.). In particular embodiments, a marker or biomarker refers to a protein biomarker. In particular embodiments, a marker or biomarker refers to a non-invasive protein biomarker.

The term “antibody” is used in the broadest sense and specifically covers monoclonal antibodies (including full length monoclonal antibodies), polyclonal antibodies, multispecific antibodies (e.g., bispecific antibodies), and antibody fragments that are antigen-binding so long as they exhibit the desired biological activity, e.g., an antibody or an antigen-binding fragment thereof.

“Antibody fragment”, and all grammatical variants thereof, as used herein are defined as a portion of an intact antibody comprising the antigen binding site or variable region of the intact antibody, wherein the portion is free of the constant heavy chain domains (i.e. CH2, CH3, and CH4, depending on antibody isotype) of the Fc region of the intact antibody. Examples of antibody fragments include Fab, Fab′, Fab′-SH, F(ab′)₂, and Fv fragments; diabodies; any antibody fragment that is a polypeptide having a primary structure consisting of one uninterrupted sequence of contiguous amino acid residues (referred to herein as a “single-chain antibody fragment” or “single chain polypeptide”).

A “predictive model” or “prediction model” refers to a model that analyzes values for a plurality of predictors and determines a prediction of risk of cancer. In various embodiments, a prediction model includes one panel. In various embodiments, a prediction model includes more than one panel, such as two panels, three panels, four panels, five panels, six panels, seven panels, eight panels, nine panels, or ten panels. The two or more panels can provide combinable information for predicting risk of cancer for the subject.

The term “panel” refers to a set of predictors that are informative for predicting risk of cancer. In one example, quantitative values of biomarkers in a panel can be informative for predicting risk of cancer. In various embodiments, a panel can include two, three, four, five, six, seven, eight, nine, ten, eleven, twelve, thirteen, fourteen, fifteen, sixteen, seventeen, eighteen, nineteen, twenty, twenty one, twenty two, twenty three, twenty four, twenty five, twenty six, twenty seven, twenty eight, twenty nine, thirty, thirty one, thirty two, thirty three, thirty four, thirty five, thirty six, thirty seven, thirty eight, thirty nine, forty, forty one, forty two, forty three, forty four, forty five, forty six, forty seven, forty eight, forty nine, fifty, fifty one, fifty two, fifty three, fifty four, fifty five, fifty six, fifty seven, fifty eight, fifty nine, sixty, sixty one, sixty two, sixty three, sixty four, sixty five, sixty six, sixty seven, sixty eight, sixty nine, seventy, seventy one, seventy two, seventy three, seventy four, seventy five, seventy six, seventy seven, seventy eight, seventy night, eighty, eighty one, eighty two, eighty three, eighty four, eighty five, eighty six, eighty seven, eighty eight, eighty nine, ninety, ninety one, ninety two, ninety three, ninety four, ninety five, ninety six, ninety seven, ninety eight, ninety nine, and one hundred predictors.

In various embodiments, a panel can include at least one hundred, at least two hundred, at least three hundred, at least four hundred, at least five hundred, at least six hundred, at least seven hundred, at least eight hundred, at least nine hundred, or at least one thousand predictors.

The term “obtaining a dataset associated with a sample” encompasses obtaining a set of data determined from at least one sample. Obtaining a dataset encompasses obtaining a sample and processing the sample to experimentally determine the data. The phrase also encompasses receiving a set of data, e.g., from a third party that has processed the sample to experimentally determine the dataset. Additionally, the phrase encompasses mining data from at least one database or at least one publication or a combination of databases and publications. A dataset can be obtained by one of skill in the art via a variety of known ways including stored on a storage memory.

It must be noted that, as used in the specification, the singular forms “a,” “an” and “the” include plural referents unless the context clearly dictates otherwise.

### II. System Environment Overview

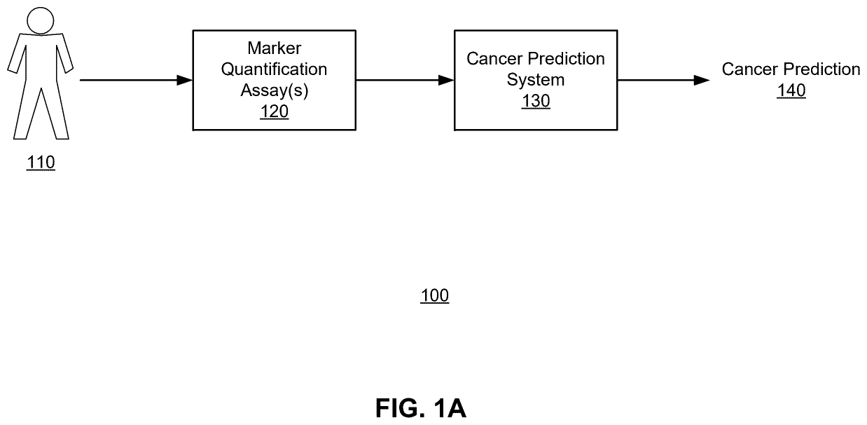

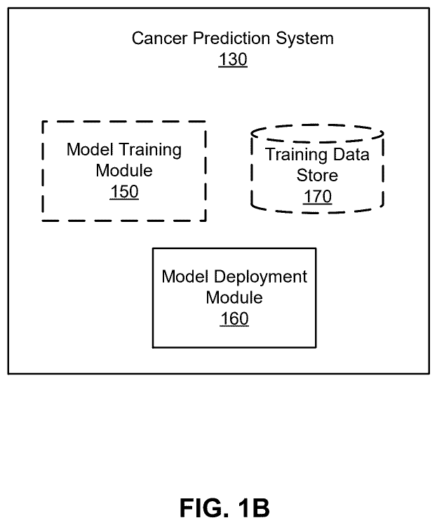

FIG. 1A depicts an overview of an environment 100 for predicting risk of cancer in a subject 110 via a cancer prediction system 130. The system environment 100 provides context in order to introduce a marker quantification assay 120 and a cancer prediction system 130 for determining a cancer prediction 140.

In various embodiments, a test sample is obtained from the subject 110. The sample can be obtained by the individual or by a third party, e.g., a medical professional. Examples of medical professionals include physicians, emergency medical technicians, nurses, first responders, psychologists, phlebotomist, medical physics personnel, nurse practitioners, surgeons, dentists, and any other medical professional as would be known to one skilled in the art.

The test sample is tested to determine values of one or more biomarkers (e.g., protein biomarkers) by performing one or more marker quantification assays 120. A marker quantification assay 120 determines quantitative values of one or more biomarkers from the test sample. In various embodiments, more than one marker quantification assay 120 can be performed to determine values of one or more biomarkers. In particular embodiments, the marker quantification assay 120 is a protein quantification assay. Therefore, by performing the marker quantification assay 120, quantitative values of one or more protein biomarkers are determined.

In various embodiments, the marker quantification assay 120 may be an assay useful for detecting and/or quantifying proteins in a biological sample. Example assays useful for detecting and/or quantifying proteins in a biological sample include an immunoassay (e.g., Proximity Extension Assay (PEA) or LUMINEX xMAP Multiplex Assay) to determine the expression levels of the plurality of biomarkers. In various embodiments, the quantitative values of various biomarkers can be obtained in a single run using a single test sample obtained from the subject 110. In some embodiments, the quantitative values of biomarkers are obtained through multiple test samples obtained from the subject 110 (e.g., a blood sample). The quantified values of the biomarkers are provided to the cancer prediction system 130.

Generally, the cancer prediction system 130 analyzes the quantitative values of biomarkers (e.g., protein biomarkers) determined by the marker quantification assay(s) 120 and generates the cancer prediction 140. In various embodiments, the cancer prediction 140 represents a prediction of presence or absence of cancer in the subject. In various embodiments, the cancer prediction 140 can be a future risk of cancer prediction for the subject 110 (e.g., a likelihood of the subject developing cancer within a time period e.g., within 1-5 years, within 1-3 years, or within 2-5 years). In various embodiments, the cancer prediction 140 can be a current risk of cancer prediction for the subject 110 (e.g., a current presence or absence of cancer in the subject 110). In various embodiments, the cancer prediction 140 can be informative for identifying a therapeutic that is likely to be effective in treating a cancer that is present or is predicted to occur within a predetermined time. In various embodiments, the therapeutic can serve as a prophylactic to delay or prevent the onset of the cancer within the predetermined time.

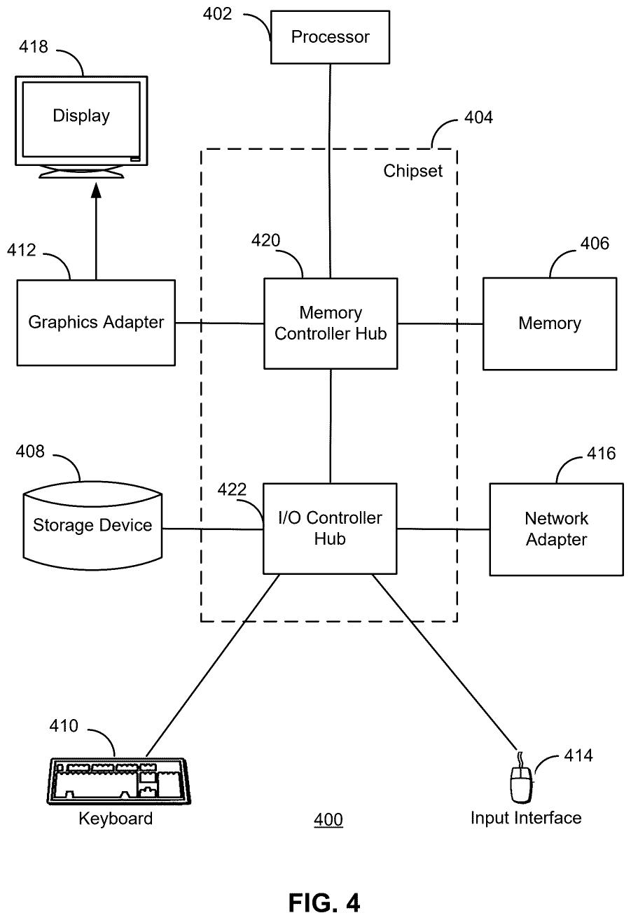

The cancer prediction system 130 can include one or more computers, embodied as a computer system 400 as discussed below with respect to FIG. 4. Therefore, in various embodiments, the steps described in reference to the cancer prediction system 130 are performed in silico.

In various embodiments, the marker quantification assay 120 and the cancer prediction system 130 can be employed by different parties. For example, a first party performs the marker quantification assay 120 and then provides the determined quantitative values to a second party which implements the cancer prediction system 130. For example, the first party may be a clinical laboratory that obtains test samples from subjects 110 and performs marker quantification assay(s) 120 on the test samples. The second party receives the quantitative values of biomarkers resulting from performed marker quantification assay(s) 120 and analyzes the quantitative values using the cancer prediction system 130.

Reference is now made to FIG. 1B which depicts a block diagram illustrating the computer logic components of the cancer prediction system 130, in accordance with an embodiment. Specifically, the cancer prediction system 130 may include a model training module 150, a model deployment module 160, and a training data store 170.

Each of the components of the cancer prediction system 130 is hereafter described in reference to two phases: 1) a training phase and 2) a deployment phase. More specifically, the training phase refers to the building and training of one or more prediction models based on training data that includes quantitative values of biomarkers obtained from individuals that are known to be healthy (e.g., absence of cancer), known to have cancer (e.g., previously diagnosed with cancer), or known to develop cancer within a certain amount of time (e.g., within 1-5 years). Therefore, the prediction models are trained to predict a risk of cancer in a subject based on at least quantitative biomarker values.

During the deployment phase, a prediction model is applied to quantitative biomarker values (e.g., protein biomarker values) from a test sample obtained from a subject of interest to predict risk of cancer for the subject of interest. In various embodiments, the prediction model only analyzes quantitative biomarker values from a test sample obtained from the subject.

In some embodiments, the components of the cancer prediction system 130 are applied during one of the training phase and the deployment phase. For example, the model training module 150 and training data store 170 (indicated by the dotted lines in FIG. 1B) are applied during the training phase whereas the model deployment module 160 is applied during the deployment phase. In various embodiments, the components of the cancer prediction system 130 can be performed by different parties depending on whether the components are applied during the training phase or the deployment phase. In such scenarios, the training and deployment of the prediction model are performed by different parties. For example, the model training module 150 and training data store 170 applied during the training phase can be employed by a first party (e.g., to train a prediction model) and the model deployment module 160 applied during the deployment phase can be performed by a second party (e.g., to deploy the prediction model).

### III. Prediction Model

### I.A. Training a Prediction Model

During the training phase, the model training module 150 trains one or more prediction models using training data. In various embodiments, the training data can be derived from samples obtained from individuals. In various embodiments, the training data includes quantitative values of biomarkers (e.g., protein biomarkers) derived from the samples obtained from individuals. Such individuals can be healthy individuals, individuals known to have cancer (e.g., individuals previously diagnosed with cancer), or individuals that are known to develop cancer within a particular timeframe (e.g., within 1-3 years, within 1-5 years, or within 2-5 years). In various embodiments, the individuals from which training data are derived are clinical subjects. For example, the training data can include quantitative values of biomarkers (e.g., protein biomarkers) that were measured from test samples obtained from clinical subjects, such as subjects that were enrolled in a clinical study or clinical trial.

Referring to FIG. 1B, the training data may be stored in the training data store 170. In various embodiments, the cancer prediction system 130 generates the training data and analyzes quantitative values of biomarkers from test samples. In various embodiments, the cancer prediction system 130 obtains the training data from a third party. The third party may have analyzed test samples to determine the quantitative biomarker values from the individuals.

In various embodiments, the training data includes reference ground truths that indicate information about a cancer. As an example, the training data can include a reference ground truth that indicates a presence or absence of cancer. As another example, the training data can include a reference ground truth that indicates development of cancer within a certain time. For example, the training data can include a reference ground truth that indicates that a subject developed cancer within a particular time period. In various embodiments, the time period can be any one of 1 month, 2 months, 3 months, 4 months, 5 months, 6 months, 7 months, 8 months, 9 months, 10 months, 11 months, 1 year, 1.5 years, 2 years, 2.5 years, 3 years, 3.5 years, 4 years, 4.5 years, 5 years, 5.5 years, 6 years, 6.5 years, 7 years, 7.5 years, 8 years, 8.5 years, 9 years, 9.5 years, 10 years, 10.5 years, 11 years, 11.5 years, 12 years, 12.5 years, 13 years, 13.5 years, 14 years, 14.5 years, 15 years, 15.5 years, 16 years, 16.5 years, 17 years, 17.5 years, 18 years, 18.5 years, 19 years, 19.5 years, or 20 years. In various embodiments, the training data can include two or more reference ground truths, each reference ground truth indicating development of cancer within a particular timeframe. For example, the training data can include a first reference ground truth indicating whether the individual developed cancer within 1 year and can further include a second reference ground truth indicating whether the individual developed cancer within 3 years.

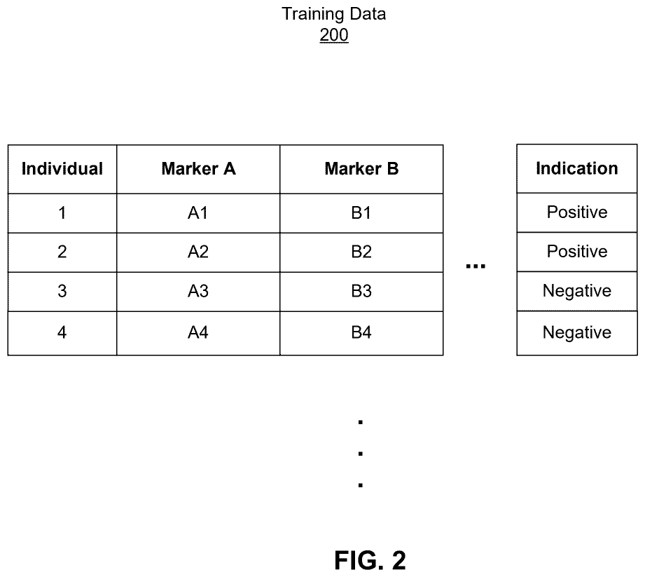

Reference is made to FIG. 2, which depicts an example set of training data 200, in accordance with an embodiment. As shown in FIG. 2, the training data 200 includes data corresponding to multiple individuals (e.g., column 1 depicting individual 1, 2, 3, 4 . . . ). For each individual, the training data 200 includes quantitative values (e.g., A1, B1, A2, B2, etc.) for different markers (e.g., protein biomarkers) obtained from the corresponding individual. In some embodiments, the quantitative values are determined by the marker quantification assay 120 shown in FIG. 1A. Although FIG. 2 explicitly depicts four individuals and two different markers (marker A and marker B), the training data 200 may include tens, hundreds, or thousands of individuals, tens, hundreds, or thousands of markers.

As shown in FIG. 2, a first training example (e.g., first row) of the training data refers to individual 1, corresponding quantitative values of marker A (e.g., A1) and marker B (e.g., B1).

Similarly, the second training example (e.g., second row) of the training data refers to individual 2, corresponding quantitative values of marker A (e.g., A2) and marker B (e.g., B2). Individuals 3 and 4 have similar corresponding marker values as shown in FIG. 2.

The training data 200 further includes a reference ground truth (e.g., column titled “Indication”) that indicates cancer information pertaining to the corresponding individual. As an example, an indication may be a current presence or current absence of cancer in the individual. As another example, an indication may be a presence or absence of cancer in the individual within a time period. For example, referring to the first training example (e.g., first row), a “Positive” indication under the column titled “Time” can indicate that the individual 1 developed cancer within the time period (e.g., within any one of 1 month, 2 months, 3 months, 4 months, 5 months, 6 months, 7 months, 8 months, 9 months, 10 months, 11 months, 1 year, 1.5 years, 2 years, 2.5 years, 3 years, 3.5 years, 4 years, 4.5 years, 5 years, 5.5 years, 6 years, 6.5 years, 7 years, 7.5 years, 8 years, 8.5 years, 9 years, 9.5 years, 10 years, 10.5 years, 11 years, 11.5 years, 12 years, 12.5 years, 13 years, 13.5 years, 14 years, 14.5 years, 15 years, 15.5 years, 16 years, 16.5 years, 17 years, 17.5 years, 18 years, 18.5 years, 19 years, 19.5 years, or 20 years).

Referring to the second training example (e.g., second row), the second training example includes an indication of “Positive” under the column titled “Indication” which indicates that the second individual developed cancer within the time period. The third and fourth training examples corresponding to Individual 3 and Individual 4, respectively, include reference ground truths with an indication of “Negative” which indicates that the individuals do not develop cancer within the time period.

Although the training data 200 in FIG. 2 depicts one reference ground truth (e.g., “Indication”), in various embodiments, training data 200 can include more reference ground truths (e.g., two indications or more). As one example, the training data 200 can additionally include reference ground truth values that indicate whether the individual developed cancer within two, three, four, five, six, seven, eight, nine, ten, eleven, twelve, thirteen, fourteen, fifteen, sixteen, seventeen, eighteen, nineteen, or twenty other time periods.

In some embodiments, for training the prediction model, the model training module 150 retrieves the training data from the training data store 170 and randomly partitions the training data into a training set and a test set. As an example, 66% of the training data may be partitioned into the training set and the other 33% can be partitioned into the test set. Other proportions of training set and test set may be implemented. As such, the training set is used to train prediction models whereas the test set is used to validate the prediction models.

In various embodiments, the prediction model is any one of a regression model (e.g., linear regression, logistic regression, Cox regression, elastic net regression, Cox Elastic regression model, ridge regression, or polynomial regression), decision tree, random forest, support vector machine, elastic net regulation, Naïve Bayes model, k-means cluster, or neural network (e.g., feed-forward networks, convolutional neural networks (CNN), deep neural networks (DNN), autoencoder neural networks, generative adversarial networks, or recurrent networks (e.g., long short-term memory networks (LSTM), bi-directional recurrent networks, deep bi-directional recurrent networks), or any combination thereof. In particular embodiments, the prediction model is any one of an elastic net logistic regression model, random forest model, support vector machine, or XGBoost model. In particular embodiments, the prediction model is an elastic net logistic regression model. In particular embodiments, the prediction model is a random forest model. In particular embodiments, the prediction model is a support vector machine. In particular embodiments, the prediction model is a XGBoost model.

The prediction model can be trained using a machine learning implemented method, such as any one of a linear regression algorithm, logistic regression algorithm, decision tree algorithm, support vector machine classification, elastic net regulation, Naïve Bayes classification, K-Nearest Neighbor classification, random forest algorithm, deep learning algorithm, gradient boosting algorithm, and dimensionality reduction techniques such as manifold learning, principal component analysis, factor analysis, autoencoder regularization, and independent component analysis, or combinations thereof. In various embodiments, the prediction model is trained using supervised learning algorithms, unsupervised learning algorithms, semi-supervised learning algorithms (e.g., partial supervision), weak supervision, transfer, multi-task learning, or any combination thereof.

In various embodiments, the prediction model has one or more parameters, such as hyperparameters or model parameters. Hyperparameters are generally established prior to training. Examples of hyperparameters include the learning rate, depth or leaves of a decision tree, number of hidden layers in a deep neural network, number of clusters in a k-means cluster, penalty in a regression model, and a regularization parameter associated with a cost function. Model parameters are generally adjusted during training. Examples of model parameters include weights associated with nodes in layers of neural network, support vectors in a support vector machine, and coefficients in a regression model. The model parameters of the prediction model are trained (e.g., adjusted) using the training data to improve the predictive capacity of the prediction model.

The model training module 150 trains a prediction model using the training data. In various embodiments, the model training module 150 constructs a prediction model that receives, as input, two or more predictors (e.g., values of biomarkers). In various embodiments, the model training module 150 constructs a prediction model that receives, as input, three predictors. In various embodiments, the model training module 150 constructs a prediction model that receives, as input, four predictors. In various embodiments, the model training module 150 constructs a prediction model that receives, as input, two, three, four, five, six, seven, eight, nine, ten, eleven, twelve, thirteen, fourteen, fifteen, sixteen, seventeen, eighteen, nineteen, twenty, twenty one, twenty two, twenty three, twenty four, twenty five, twenty six, twenty seven, twenty eight, twenty nine, thirty, thirty one, thirty two, thirty three, thirty four, thirty five, thirty six, thirty seven, thirty eight, thirty nine, forty, forty one, forty two, forty three, forty four, forty five, forty six, forty seven, forty eight, forty nine, fifty, fifty one, fifty two, fifty three, fifty four, fifty five, fifty six, fifty seven, fifty eight, fifty nine, sixty, sixty one, sixty two, sixty three, sixty four, sixty five, sixty six, sixty seven, sixty eight, sixty nine, seventy, seventy one, seventy two, seventy three, seventy four, seventy five, seventy six, seventy seven, seventy eight, seventy night, eighty, eighty one, eighty two, eighty three, eighty four, eighty five, eighty six, eighty seven, eighty eight, eighty nine, ninety, ninety one, ninety two, ninety three, ninety four, ninety five, ninety six, ninety seven, ninety eight, ninety nine, and one hundred predictors. In various embodiments, a panel can include at least one hundred, at least two hundred, at least three hundred, at least four hundred, at least five hundred, at least six hundred, at least seven hundred, at least eight hundred, at least nine hundred, or at least one thousand predictors.

In various embodiments, the model training module 150 constructs a prediction model that receives, as input, quantitative values of three biomarkers. In various embodiments, the model training module 150 constructs a prediction model that receives, as input, quantitative values of four biomarkers. In some embodiments, the model training module 150 constructs a prediction model that receives, as input, quantitative values for more than four biomarkers. In various embodiments, the model training module 150 constructs a prediction model that receives as input, quantitative values for five, six, seven, eight, nine, ten, eleven, twelve, thirteen, fourteen, fifteen, sixteen, seventeen, eighteen, nineteen, twenty, twenty one, twenty two, twenty three, twenty four, twenty five, twenty six, twenty seven, twenty eight, twenty nine, thirty, thirty one, thirty two, thirty three, thirty four, thirty five, thirty six, thirty seven, thirty eight, thirty nine, forty, forty one, forty two, forty three, forty four, forty five, forty six, forty seven, forty eight, forty nine, fifty, one hundred, two hundred, three hundred, four hundred, five hundred, six hundred, seven hundred, eight hundred, nine hundred, one thousand, or more markers. In particular embodiments, the model training module 150 constructs a prediction model that receives as input, quantitative values for 5 markers. In particular embodiments, the model training module 150 constructs a prediction model that receives as input, quantitative values for at least 10 markers. In particular embodiments, the model training module 150 constructs a prediction model that receives as input, quantitative values for at least 20 markers. In particular embodiments, the model training module 150 constructs a prediction model that receives as input, quantitative values for at least 30 markers. In particular embodiments, the model training module 150 constructs a prediction model that receives as input, quantitative values for at least 40 markers. In particular embodiments, the model training module 150 constructs a prediction model that receives as input, quantitative values for at least 50 markers. In particular embodiments, the model training module 150 constructs a prediction model that receives as input, quantitative values for at least 100 markers. In particular embodiments, the model training module 150 constructs a prediction model that receives as input, quantitative values for at least 400 markers. In particular embodiments, the model training module 150 constructs a prediction model that receives as input, quantitative values for at least any of 5, 10, 15, 20, 30, 50, 100, 425, or 493 biomarkers.

In various embodiments, the model training module 150 identifies a set of biomarkers that are to be used to train a prediction model. The model training module 150 may begin with a list of candidate biomarkers that are promising for diagnosing a cancer. In various embodiment, the model training module 150 performs a feature selection process to identify the set of biomarkers to be included for the prediction model. For example, candidate biomarkers that are determined to be highly correlated with a presence of cancer would be deemed important are therefore likely to be included in the panel in comparison to other biomarkers that are not highly correlated.

In various embodiments, each prediction model is iteratively trained using, as input, the quantitative values of the markers for each individual. For example, referring again to FIG. 2, one iteration involves providing a training example (e.g., a row of the training data). Each prediction model is trained on reference ground truth data that includes the indication(s). In various embodiments, over training iterations, the prediction model is trained (e.g., the parameters are tuned) to minimize a prediction error between a prediction outputted by the prediction model and the ground truth data. In various embodiments, the prediction error is calculated based on a loss function, examples of which include a L1 regularization (Lasso Regression) loss function, a L2 regularization (Ridge Regression) loss function, or a combination of L1 and L2 regularization (ElasticNet).

In various embodiments, a penalty factor is employed to lower the risk of false-positive selection of predictive biomarkers arising from their low levels. In various embodiments, a penalty factor is added to the general Elastic Net penalty based on the proportion of values of each biomarker at or below a lower limit of quantitation (LLOQ).

III.B. Deploying a Prediction model

During the deployment phase, the model deployment module 160 (as shown in FIG. 1B) applies a trained prediction model to generate a prediction for risk of cancer in the subject. In various embodiments, the prediction for risk of cancer for the subject is a prediction of presence of absence of cancer in the subject. In particular embodiments, the subject has not previously been diagnosed with a disease. Therefore, the deployment of the prediction model enables in silico prediction of whether the subject is likely to develop cancer in the future (e.g., within 1-20 years). In various embodiments, the model deployment module 160 applies a trained prediction model that analyzes quantitative values of biomarkers to determine a risk of cancer in a subject.

In various embodiments, the trained prediction model includes a single panel that includes one or more biomarkers. Thus, the trained prediction model outputs a prediction based on the one or more biomarkers of the single panel.

In various embodiments, the trained prediction model includes two or more panels, each panel comprising one or more biomarkers. In various embodiments, a panel includes a set of biomarkers that are distinct from a set of biomarkers of another panel in the prediction model. In various embodiments, one or more biomarkers of one panel can overlap with one or more biomarkers of another panel. In other words, two panels may share one or more biomarkers. In various embodiments, two panels may share at least one, at least two, at least three, at least four, at least five, at least six, at least seven, at least eight, at least nine, at least ten, at least fifteen, at least twenty, at least thirty, at least fifty, at least one hundred, at least two hundred, at least three hundred, at least four hundred, at least five hundred, at least six hundred, at least seven hundred, at least eight hundred, at least nine hundred, or at least one thousand biomarkers.

In such embodiments where the trained prediction model includes two or more panels, the trained prediction model outputs a prediction based on the biomarkers of each of the two or more panels. To generate an overall prediction, the trained prediction model combines an output of a first panel with an output of a second panel. Thus, the one or more biomarkers of the first panel as well as the one or more biomarkers of the second panel contribute towards the overall prediction outputted by the trained prediction model.

In various embodiments, the output of each of the panels of the prediction model is a score (e.g., an indication of how likely it is that the subject has cancer or will develop cancer). Thus, the trained prediction model combines scores outputted by the individual panels to generate an overall prediction. In various embodiments, the trained prediction model combines the scores outputted by the individual panels by comparing the scores outputted by the individual panels and selecting one of the scores. Thus, the selected score serves as the basis for the overall prediction of the prediction model. In various embodiments, the trained prediction model combines the scores outputted by the individual panels by comparing the scores outputted by the individual panels and selecting the higher score.

In various embodiments, the trained prediction model combines the supplemented scores by comparing the supplemented scores and selecting one of the supplemented scores. In various embodiments, the prediction model selects the highest supplemented score. In such embodiments, the overall prediction outputted by the prediction model can be the selected score or can be derived from the selected score (e.g., overall prediction is generated based on the comparison between the selected score and a reference score as described above).

In various embodiments, prior to comparing the scores and selecting a score, the prediction model normalizes each score outputted by a panel to a corresponding reference score. Thus, normalized scores are compared to one another to select the score.

In various embodiments, the overall prediction outputted by the prediction model is the selected score that is selected from the scores outputted the panels. In various embodiments, the prediction model generates the overall prediction by comparing the selected score to one or more reference scores. In various embodiments, the reference score can be a score corresponding to healthy patients (e.g., a “healthy score”), a baseline score at a prior timepoint (e.g., longitudinal analysis), a score corresponding to patients clinically diagnosed with cancer (e.g., a “reference cancer score”), a score corresponding to patients diagnosed with a particular subtype of cancer (e.g., a cancer subtype score), a score corresponding to patients who are known to develop cancer within a particular time period (e.g., a time to event score), or a threshold score (e.g., a cutoff).

In particular embodiments, the reference score can be a “healthy score” corresponding to healthy patients and can be generated by implementing a prediction model to analyze quantitative values of biomarkers. In particular embodiments, the reference score is a time to event score corresponding to patients who are known to develop cancer within a time period (e.g., within any one of 1 month, 2 months, 3 months, 4 months, 5 months, 6 months, 7 months, 8 months, 9 months, 10 months, 11 months, 1 year, 1.5 years, 2 years, 2.5 years, 3 years, 3.5 years, 4 years, 4.5 years, 5 years, 5.5 years, 6 years, 6.5 years, 7 years, 7.5 years, 8 years, 8.5 years, 9 years, 9.5 years, 10 years, 10.5 years, 11 years, 11.5 years, 12 years, 12.5 years, 13 years, 13.5 years, 14 years, 14.5 years, 15 years, 15.5 years, 16 years, 16.5 years, 17 years, 17.5 years, 18 years, 18.5 years, 19 years, 19.5 years, or 20 years).

In various embodiments, the overall prediction is generated based on the comparison between a score of the prediction model and one or more reference scores. The overall prediction is informative for predicting risk of cancer for the subject within one or more time periods. To provide an example, the score can be from a panel of the prediction model. The score is compared to a healthy score (e.g., reference score derived from healthy patients). If the score is significantly different (e.g., p<0.05) from the healthy score, the overall prediction can indicate that the subject has cancer, or will likely develop cancer. As another example, the score from the prediction model can be compared to one or more time to event scores of patients who are known to develop cancer within a particular time period. If the score is significantly different (e.g., p<0.05) from a time to event score, then the overall prediction can indicate that the subject is unlikely to develop cancer within a period of time corresponding to the time to event score. If the score is not significantly different (e.g., p>0.05) from a time to event score, then the overall prediction can indicate that the subject is likely to develop cancer within a period of time corresponding to the time to event score. As described herein, a period of time can be any of within any one of 1 month, 2 months, 3 months, 4 months, 5 months, 6 months, 7 months, 8 months, 9 months, 10 months, 11 months, 1 year, 1.5 years, 2 years, 2.5 years, 3 years, 3.5 years, 4 years, 4.5 years, 5 years, 5.5 years, 6 years, 6.5 years, 7 years, 7.5 years, 8 years, 8.5 years, 9 years, 9.5 years, 10 years, 10.5 years, 11 years, 11.5 years, 12 years, 12.5 years, 13 years, 13.5 years, 14 years, 14.5 years, 15 years, 15.5 years, 16 years, 16.5 years, 17 years, 17.5 years, 18 years, 18.5 years, 19 years, 19.5 years, or 20 years.

In various embodiments, the subject can undergo treatment depending on the overall prediction. For example, if the subject is predicted to likely develop cancer within a particular period of time, the subject can be administered a therapeutic intervention. Here, the therapeutic intervention can serve as a prophylactic treatment to delay or prevent the onset of the cancer.

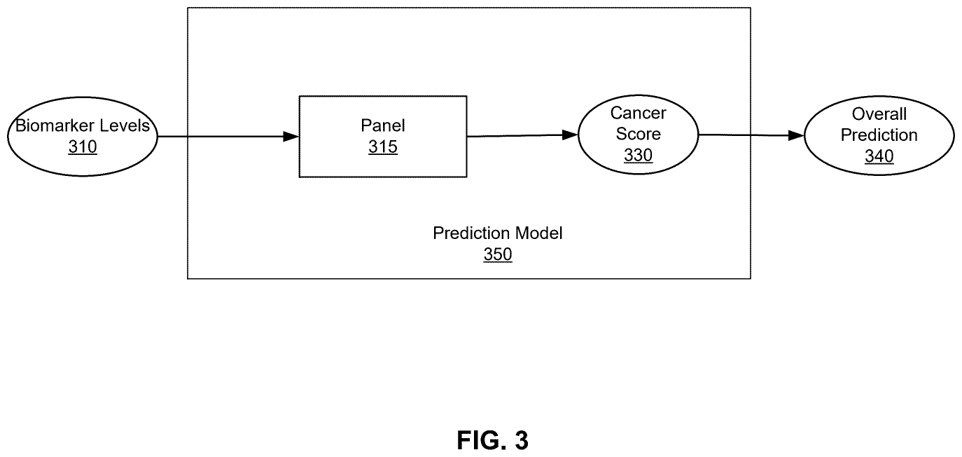

Reference is now made to FIG. 3, which depicts implementation of an example prediction model, in accordance with a fourth embodiment. Here, the prediction model 350 may include a single panel 315. Thus, single panel 315 of the prediction model analyzes the quantitative biomarker levels 310.

Based on the analysis of the quantitative biomarker levels 310, the prediction model 350 generates a cancer score 330. The cancer score 330 is compared to one or more reference scores. In various embodiments, the cancer score 330 can be compared to a time to event score. If the cancer score 330 is not significantly different (e.g., p>0.05) from the time to event score, then the overall prediction 340 can indicate that the individual is likely to develop cancer within a time period corresponding to the time to event score. Alternatively, if the cancer score 330 is significantly different (e.g., p<0.05) from the time to event score, then the overall prediction 340 can indicate that individual is not likely to develop cancer within the time period corresponding to the time to event score. The cancer score 330 can be compared to multiple time to event scores corresponding to different time periods to predict whether the individual is likely to develop cancer within any of the time periods corresponding to the time to event scores.

As shown and described in reference to FIG. 3, the prediction model 350 can generate a cancer score (e.g., cancer score 330) that is informative for determining an overall prediction 340. In various embodiments, the cancer score represents an aggregate score of the levels (e.g., altered or dysregulated levels) of the biomarkers of the prediction model 350. This means that it is not necessary to know how the level of any individual marker has changed to obtain the cancer score. For example, assuming a prediction model of 20 biomarkers, the upregulation or downregulation of any one biomarker represents one component that results in the cancer score. Thus, even though a first patient and second patient may both exhibit upregulation of a biomarker, the final aggregate cancer scores may indicate that the first patient is likely to develop cancer within a certain timeframe, whereas the second patient is unlikely to develop cancer within the certain timeframe.

As further shown in FIG. 3, the output of the prediction model 350 is an overall prediction 340. In particular embodiments, the overall prediction 340 represents a prediction of risk of cancer (e.g., lung cancer) for the subject. In particular embodiments, the overall prediction 340 represents a prediction of whether the subject is likely to develop lung cancer within a particular time period. In various embodiments, the time period is any one of 1 month, 2 months, 3 months, 4 months, 5 months, 6 months, 7 months, 8 months, 9 months, 10 months, 11 months, 1 year, 1.5 years, 2 years, 2.5 years, 3 years, 3.5 years, 4 years, 4.5 years, 5 years, 5.5 years, 6 years, 6.5 years, 7 years, 7.5 years, 8 years, 8.5 years, 9 years, 9.5 years, 10 years, 10.5 years, 11 years, 11.5 years, 12 years, 12.5 years, 13 years, 13.5 years, 14 years, 14.5 years, 15 years, 15.5 years, 16 years, 16.5 years, 17 years, 17.5 years, 18 years, 18.5 years, 19 years, 19.5 years, or 20 years. In various embodiments, the overall prediction 340 can represent multiple predictions of whether the subject is likely to develop lung cancer within N different time periods. In various embodiments, N is 1, 2, 3, 4, 5, 6, 7, 8, 9, 10, 11, 12, 13, 14, 15, 16, 17, 18, 19, or 20 different time periods.

In various embodiments, the prediction model 350 achieves e.g., an area under the curve (AUC) performance metric (e.g., minimum, median, mean, maximum, first quartile, second quartile, third quartile, or fourth quartile AUC value) of at least 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, or 0.99. In various embodiments, the prediction model 350 achieves e.g., an AUC performance metric (e.g., minimum, median, mean, maximum, first quartile, second quartile, third quartile, or fourth quartile AUC value) of about 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, or 0.99.

### IV. Panel(s) of a Prediction Model

Embodiments described herein involve implementing a prediction model that includes one or more panels. Each panel includes one or more predictors, examples of which include biomarkers (e.g., protein biomarkers).

In various embodiments, multiple panels can be included in a prediction model. The implementation of multiple panels is informative for generating an overall prediction for risk of cancer in a subject. In various embodiments, a panel of the prediction model is a univariate panel. In such embodiments, the univariate panel includes one predictor. In other embodiments, a panel is a multivariate panel. In such embodiments, the multivariate panel includes more than one predictor. In various embodiments, the multivariate panel includes two predictors. In various embodiments, the multivariate panel includes 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 predictors. In various embodiments, the multivariate panel includes at least 100, at least 200, at least 300, at least 400, at least 500, at least 600, at least 700, at least 800, at least 900, at least 1000, or more predictors. In particular embodiments, the multivariate panel includes five predictors. In particular embodiments, the multivariate panel includes ten predictors. In particular embodiments, the multivariate panel includes fifteen predictors. In particular embodiments, the multivariate panel includes twenty predictors. In particular embodiments, the multivariate panel includes thirty predictors. In particular embodiments, the multivariate panel includes fifty predictors. In particular embodiments, the multivariate panel includes at least one hundred predictors. In particular embodiments, the multivariate panel includes at least two hundred predictors. In particular embodiments, the multivariate panel includes at least three hundred predictors. In particular embodiments, the multivariate panel includes at least four hundred predictors. In particular embodiments, the multivariate panel includes at least five hundred predictors. In particular embodiments, the multivariate panel includes at least six hundred predictors. In particular embodiments, the multivariate panel includes at least seven hundred predictors. In particular embodiments, the multivariate panel includes at least eight hundred predictors. In particular embodiments, the multivariate panel includes at least nine hundred predictors. In particular embodiments, the multivariate panel includes at least one thousand predictors. In particular embodiments, the multivariate panel includes 425 predictors. In particular embodiments, the multivariate panel includes 493 predictors.

In various embodiments, the prediction model (such as the prediction model in FIG. 3) includes between 1 and 1000 biomarkers. In various embodiments, the prediction model (such as the prediction model in FIG. 3) includes between 1 and 500 biomarkers. In various embodiments, the prediction model (such as the prediction model in FIG. 3) includes between 1 and 100 biomarkers. In various embodiments, the prediction model (such as the prediction model in FIG. 3) includes between 1 and 60 biomarkers. In various embodiments, the prediction model includes between 10 and 50 biomarkers. In various embodiments, the prediction model includes between 20 and 40 biomarkers. In various embodiments, the prediction model includes between 25 and 38 biomarkers. In various embodiments, the prediction model includes between 30 and 35 biomarkers. In various embodiments, the prediction model includes between 20 and 30 biomarkers. In various embodiments, the prediction model includes between 30 and 40 biomarkers. In various embodiments, the prediction model includes between 40 and 50 biomarkers. In particular embodiments, the prediction model includes 5 biomarkers. In particular embodiments, the prediction model includes 10 biomarkers. In particular embodiments, the prediction model includes 15 biomarkers. In particular embodiments, the prediction model includes 20 biomarkers. In particular embodiments, the prediction model includes 30 biomarkers. In particular embodiments, the prediction model includes 50 biomarkers.

In various embodiments, a panel of the prediction model (such as the panel of the prediction model shown in any of FIG. 3) includes one or more protein biomarkers. Example protein biomarkers included in panels of the prediction model or the prediction model include protein biomarkers shown below in Tables 1-3.

In particular embodiments, a panel of the prediction model (such as the panel of the prediction model shown in any of FIG. 3) includes one or more, two or more, three or more, four or more, or each protein biomarker selected from TGFA, MMP12, TNFRSF13B, TNFSF14, and MASP1.

In particular embodiments, a panel of the prediction model (such as the panel of the prediction model shown in any of FIG. 3) includes one or more, two or more, three or more, four or more, five or more, six or more, seven or more, eight or more, nine or more, ten or more, eleven or more, twelve or more, thirteen or more, fourteen or more, or each protein biomarker selected from THBS2, GDNF, FLT1, FXYD5, CST5, ARNT, CDCP1, CCL20, FLT3LG, CLEC7A, PRKCQ, SCGN, IL5, NPY, and S100A16.

In particular embodiments, a panel of the prediction model (such as the panel of the prediction model shown in any of FIG. 3) includes one or more, two or more, three or more, four or more, five or more, six or more, seven or more, eight or more, nine or more, or each protein biomarker selected from IL1B, CD84, STC1, PRDX3, LAP3, GAMT, CASP2, ITGA6, DECR1, and YTHDF3.

In particular embodiments, a panel of the prediction model (such as the panel of the prediction model shown in any of FIG. 3) includes one or more, two or more, three or more, four or more, or each protein biomarker selected from CEACAM5, TOP1, NCAM1, SCGB3A2, and CALY.

In particular embodiments, a panel of the prediction model (such as the panel of the prediction model shown in any of FIG. 3) includes one or more, two or more, three or more, four or more, five or more, six or more, seven or more, eight or more, nine or more, ten or more, eleven or more, twelve or more, thirteen or more, fourteen or more, or each protein biomarker selected from TGFBI, CABP2, ENPP6, KRT14, HEPACAM2, TMEM25, SGSH, MFAP3L, TNFSF14, CD3D, TMED4, ZP3, MMP12, GCG, and AFM.

In particular embodiments, a panel of the prediction model (such as the panel of the prediction model shown in any of FIG. 3) includes one or more, two or more, three or more, four or more, five or more, six or more, seven or more, eight or more, nine or more, ten or more, eleven or more, twelve or more, thirteen or more, fourteen or more, fifteen or more, sixteen is more, seventeen or more, eighteen or more, nineteen or more, twenty or more, twenty one or more, twenty two or more, twenty three or more, twenty four or more, twenty five or more twenty six or more, twenty seven or more, twenty eight or more, twenty nine or more, or each protein biomarker selected from SPINT1, LILRA4, FLT3LG, AGBL2, PAEP, SCGB3A1, LRFN2, TJP3, FGF7, LRIG1, CA14, CEACAM18, CST1, ANXA10, CDCP1, GPC5, OSCAR, CEACAM6, CD2, SNCG, GPR37, SEPTIN3, RAB10, DKK4, DKKL1, SOST, CSF3, VWA5A, TSPAN7, and PAK4.

In particular embodiments, a panel of the prediction model (such as the panel of the prediction model shown in any of FIG. 3) includes one or more, two or more, three or more, four or more, five or more, six or more, seven or more, eight or more, nine or more, ten or more, eleven or more, twelve or more, thirteen or more, fourteen or more, fifteen or more, sixteen is more, seventeen or more, eighteen or more, nineteen or more, twenty or more, twenty one or more, twenty two or more, twenty three or more, twenty four or more, twenty five or more twenty six or more, twenty seven or more, twenty eight or more, twenty nine or more, thirty or more, thirty one or more, thirty two or more, thirty three or more, thirty four or more, thirty five or more, thirty six or more, thirty seven or more, thirty eight or more, thirty nine or more, forty or more, forty one or more, forty two or more, forty three or more, forty four or more, forty five or more, forty six or more, forty seven or more, forty eight or more, forty nine or more, or each protein biomarker selected from BPIFB1, SIGLEC9, ZNRD2, PM20D1, TK1, RPS10, PMCH, RNF43, MEP1B, BGN, NELL1, CD101, LRP2BP, PRSS53, MFGE8, THSD1, CKMT1A, MEPE, APOL1, RBPMS, MARCO, KLRC1, FGFBP2, TPSG1, SELENOP, CLEC7A, UPK3BL1, HS6ST1, ENDOU, IL12RB2, CYB5A, GKN1, NRTN, CCL26, CRNN, PINLYP, LAIR2, BAG3, SCPEP1, RIPK4, CTSE, TMOD4, SFTPA1, SEMA4D, IL17C, GFRA3, DPEP2, EDEM2, CD84, and KIRREL2.

In particular embodiments, a panel of the prediction model (such as the panel of the prediction model shown in any of FIG. 3) includes one or more protein biomarker selected from NECTIN1, CBLN1, NTF3, PYY, XG, NPY, CCL20, SIL1, PLB1, DUSP29, UMOD, ATXN2L, LEO1, PROS1, EDDM3B, ENO3, DCBLD2, MMP9, KIF22, DENND2B, C1RL, PVALB, CXCL8, PPY, CCN1, KLK10, RRAS, SCN3B, BPIFB2, ITGAL, DDX1, MEGF11, NOP56, NTF4, HNMT, IL9, SCRIB, UXS1, MEP1A, ACTN2, NECAP2, CLEC10A, DDX53, SV2A, ATXN10, PI16, KCNH2, TNR, PDGFRB, SERPINA4, CDC27, MICALL2, CD28, BRK1, SLC16A1, DSCAM, PBXIP1, MATN3, SFTPA2, PTTG1, ASAH2, SCG2, PTGR1, GBA, PTPRZ1, ERN1, LECT2, SCGN, HLA-DRA, IL5RA, LRPAP1, CXCL13, NEXN, CD248, KYNU, ADAMTS15, WFIKKN2, CLEC14A, FZD10, PROC, LY9, LRP2, CX3CL1, RNASET2, CTSS, MCEMP1, COMP, SIGLEC6, CCL24, AOC1, PLXNB3, TMPRSS15, FCAR, SCIN, IFI30, KIRREL1, FXYD5, S100A16, LILRA5, CLSPN, AHNAK2, CTLA4, INSL5, WDR46, CST5, PHLDB2, TREML2, GUCA2A, PFDN2, PDIA4, LAMA1, SLAMF7, RGS8, IL6, PSG1, PZP, RRM2, GFRAL, AIF1L, LGMN, C1QTNF9, TSPAN1, DLL4, CRELD2, SCARF1, FGF9, JAM3, LPP, HSPB1, PPT1, PPIF, TRPV3, APOA4, LYSMD3, TGFA, ATP6V1D, LRRC38, CTAG1A, TINAGL1, POLR2A, EDIL3, LAP3, SORD, ARHGAP30, CSPG4, ART3, GADD45GIP1, SLURP1, LILRA2, GZMH, FKBP7, SLC27A4, CALCB, GIT1, CTSO, PCBD1, CSF3R, EIF1AX, CSPG5, CD93, ADAMTSL5, ISM2, CPE, WFDC1, VWC2, SPINK5, BTN1A1, DPT, FCN1, AIF1, GPC1, FAP, CLNS1A, CFC1, FASLG, NCS1, PRKAR1A, RCOR1, SLITRK2, SPARCL1, HSPB6, TNFRSF12A, IL6, SERPIND1, CEBPB, CASC3, AMPD3, YTHDF3, AAMDC, STX7, AGRP, ICA1, CHCHD6, IGSF21, VSTM1, PCDH7, VNN2, GP6, ITGAV, CD40LG, GIP, MB, TPD52L2, HPSE, GRIN2B, TREML1, C3, TNFRSF17, IL6, CD226, PALM, FKBP14, RBPMS2, CLEC6A, DAAM1, FAM3D, WASF1, HS1BP3, NOS3, POF1B, PLXNA4, MITD1, ERMAP, SYAP1, LRRC59, CNTN2, RAB2B, PENK, MCAM, EIF2S2, EGF, PTPN6, NID2, EHD3, IGFBP6, LMOD1, PAGR1, CD300C, SKAP2, PRKG1, SYTL4, GYS1, CASP3, PILRA, CD69, CCN5, PCBP2, LMOD1, PDIA5, PCSK7, SCARA5, METAP1D, ADGRB3, MPIG6B, NUMB, L3HYPDH, DENR, AGRN, COX6B1, JAM2, TIA1, CACYBP, SEMA6C, VAT1, SUSD1, RSPO3, TWF2, BOLA1, OXCT1, ITGA6, BST2, F2R, PILRB, RTBDN, ENOX2, DOK1, VASH1, DTD1, DDHD2, TBC1D23, GLRX5, CDNF, SIRPB1, NMT1, STK11, RPL14, PSTPIP2, FHIT, CLMP, LMOD1, ERP29, BECN1, CD38, YAP1, CA13, CRKL, PPP1R9B, FLI1, CMC1, CDC37, ARHGAP45, PDAP1, NUDC, CLEC1B, USO1, SNAP23, HGS, FUS, PIK3AP1, F11R, TBC1D17, ITPA, IL1B, ENO1, THTPA, SAFB2, JPT2, GIMAP7, NIT2, RILPL2, PRTFDC1, TADA3, TOMM20, HPCAL1, LONP1, CALCOCO1, ATRAID, TYMP, TNFRSF19, DNPEP, NRGN, STK4, SSNA1, CRYGD, LZTFL1, SNAP29, PDLIM5, CASP2, MANF, BACH1, DAPP1, AKR1B1, EREG, DAG1, HSBP1, DUT, AKT2, PLA2G4A, TXLNA, PIKFYVE, FYB1, CSDE1, RHOC, HNRNPK, DCTD, SCRG1, LACTB2, RGCC, GIMAP8, GRHPR, SNX5, NCK2, EIF4G1, BNIP3L, ACOT13, MECR, MAP2K6, SEC31A, MGLL, MESD, NUDT16, SULTIA1, GOPC, VTA1, PDLIM7, ANXA2, GGACT, PMVK, USP8, SNCA, CAMSAP1, HEXIMI, SHMT1, LGALS8, APPL2, MAP2K1, EHBP1, MAP4K5, PDE5A, HARS1, SRC, TACC3, and RAB27B.

In particular embodiments, a panel of the prediction model (such as the panel of the prediction model shown in any of FIG. 3) includes one or more, two or more, three or more, four or more, or each protein biomarker selected from VWA5A, ENPP6, TMEM25, ALDH2, and LEO1.

In particular embodiments, a panel of the prediction model (such as the panel of the prediction model shown in any of FIG. 3) includes one or more, two or more, three or more, four or more, five or more, six or more, seven or more, eight or more, nine or more, ten or more, eleven or more, twelve or more, thirteen or more, fourteen or more, or each protein biomarker selected from GAMT, TPSG1, ANK2, SCT, TSPAN7, GPC5, PGLYRP1, PAK4, TNFSF14, CLEC6A, TMPRSS15, PMCH, KRT14, SFTPA1, and LRFN2.

In particular embodiments, a panel of the prediction model (such as the panel of the prediction model shown in any of FIG. 3) includes one or more, two or more, three or more, four or more, five or more, six or more, seven or more, eight or more, nine or more, ten or more, eleven or more, twelve or more, thirteen or more, fourteen or more, fifteen or more, sixteen is more, seventeen or more, eighteen or more, nineteen or more, twenty or more, twenty one or more, twenty two or more, twenty three or more, twenty four or more, twenty five or more twenty six or more, twenty seven or more, twenty eight or more, twenty nine or more, or each protein biomarker selected from MMP12, TNPO1, GAST, CD3D, TK1, DLGAP5, SCGN, CCL24, PSG1, CLU, CFB, LBP, CRYM, LAIR2, TCN2, SV2A, CRHBP, C5, SCGB3A2, ANXA10, GCG, RPGR, PAPPA, FZD8, CSPG5, BRK1, OXT, FDX1, ENPEP, and LRG1.

In particular embodiments, a panel of the prediction model (such as the panel of the prediction model shown in any of FIG. 3) includes one or more, two or more, three or more, four or more, five or more, six or more, seven or more, eight or more, nine or more, ten or more, eleven or more, twelve or more, thirteen or more, fourteen or more, fifteen or more, sixteen is more, seventeen or more, eighteen or more, nineteen or more, twenty or more, twenty one or more, twenty two or more, twenty three or more, twenty four or more, twenty five or more twenty six or more, twenty seven or more, twenty eight or more, twenty nine or more, thirty or more, thirty one or more, thirty two or more, thirty three or more, thirty four or more, thirty five or more, thirty six or more, thirty seven or more, thirty eight or more, thirty nine or more, forty or more, forty one or more, forty two or more, forty three or more, forty four or more, forty five or more, forty six or more, forty seven or more, forty eight or more, forty nine or more, or each protein biomarker selected from PRAME, KIRREL1, KIF22, SPINT1, FGA, C1QTNF9, KIR2DS4, MMP9, NEXN, FCN1, MFGE8, ZNRD2, PDGFRB, HS6ST1, DUSP3, CABP2, DNM3, FGL1, TOP1, CDCP1, RAB10, THSD1, FASLG, MCEMP1, COL4A4, ENO1, BRD1, GP5, ZP3, SERPIND1, NCAM1, ATXN10, MUC16, GABRA4, POSTN, MAEA, SHH, DDX53, PRKG1, PAEP, RICTOR, IL6, FKBP14, CCL26, AIDA, GIP, TGFA, ITIH4, PCSK7, and RARRES1.

In particular embodiments, a panel of the prediction model (such as the panel of the prediction model shown in any of FIG. 3) includes one or more protein marker selected from SLC27A4, IL6, DKKL1, MFAP3, STX7, SSBP1, AKR7L, UGDH, IGHMBP2, GBP4, RBPMS, ST6GAL1, LILRA5, LILRA2, SOWAHA, ACADSB, CAMLG, CRTAC1, SUSD1, IL6, KLK10, GRSF1, MFAP4, NMT1, CNTN3, IL36A, EHD3, MAPT, AGBL2, ERN1, POMC, PDIA4, LGMN, EPHA10, PCBP2, PTGR1, GIT1, TREML1, GALNT2, TDGF1, INSR, OSCAR, MMP10, MRPL24, EIF1AX, AHNAK2, TP53, GBA, LRRC38, CLEC12A, TPT1, PPP1CC, BPIFB1, CFC1, SIGLEC9, CALY, OSM, ADAMTS1, OSMR, TYMP, GPR37, CLEC7A, SMAD5, SFTPA2, CTSS, HNMT, BATF, CCL19, SHC1, CST7, S100A12, ASAH2, PPIB, LYPD3, APOL1, AFM, SSC4D, FGF7, TDRKH, SCG2, ENPP2, PRKAR1A, FAM3D, GADD45GIP1, SEMA4D, PPP1R14A, EGF, NTF4, SERPING1, COX6B1, NECAP2, TFF1, IDI2, TJP3, CA14, PZP, PLIN1, ERBB4, TBC1D23, CRISP3, IFI30, ITIH1, C9, LAP3, PDIA5, ENDOU, FLT3LG, VNN2, MILR1, SDC1, CEACAM18, FHIP2A, CEACAM5, F11, WFIKKN2, USO1, CD40LG, GSTT2B, DUSP29, ATXN2L, IL6, RRM2, FGF23, ARHGAP30, SERPINA3, CXCL13, MMP8, NUDC, ENOPH1, NEK7, MAN1A2, ASAH1, STX5, IZUMO1, SERPINC1, IL9, PVALB, GZMH, FGF16, TFF2, WASF1, TMEM106A, GP2, PLXNA4, GNE, LGALS8, AOC1, FLRT2, CHCHD6, RNF43, TPD52L2, CSDE1, GPD1, PLA2G4A, LRIG1, NGF, RAB27B, VAT1, NUDT16, TRAF3IP2, MARCO, UMOD, PIK3AP1, MEGF11, NEDD4L, PKD2, CEBPB, RILPL2, IL3, RGCC, SARG, SMAD2, CTSH, KLKB1, ERP44, SULT2A1, SORD, IFNAR1, KLK11, TOMM20, C3, ADRA2A, NCK2, KIRREL2, CACNB3, SKAP2, CEACAM6, DNAJC21, PROS1, NRCAM, NPY, FYB1, RAB2B, MANF, MECR, LPA, DAAM1, DCTD, FXYD5, CRELD1, PLEKHO1, TINAGL1, ZBTB16, PROK1, MAP2K1, DAPP1, DSG4, PPP1R9B, RILP, EIF4G1, SESTD1, KIFBP, HGS, CD14, ANKMY2, WNT9A, CA13, GP1BB, CLIP2, BANK1, WDR46, HSPB1, CSF2, SNCA, RRAS, PRTFDC1, RBPMS2, LARP1, KAZN, CLSPN, RHOC, PPT1, DPEP2, METAP1D, STK11, CFH, PDE5A, MRC1, BIN2, IL17A, PXDNL, GP6, EPO, MAP3K5, MCEE, DDHD2, PHLDB2, NECTIN1, CCDC50, GKN1, MPIG6B, CBLIF, SYTL4, SSH3, PDZD2, SULTIA1, DLG4, HPCAL1, ICA1, GDF15, CD160, APPL2, GRN, IL17RA, CDC42BPB, C4BPB, DAG1, CMIP, KYNU, NUMB, PPY, PPIF, CFI, DTD1, LDLRAP1, FGF9, STXBP1, CMC1, GOPC, SMTN, PTPN6, L3HYPDH, PDAP1, LPP, THTPA, XG, AGRP, RAB11FIP3, F11R, BCR, LONP1, BNIP3L, SELP, GYS1, MGLL, PDLIM5, MESD, DNPEP, SRC, PMVK, ITPRIP, CD69, CALCOCO1, PAFAH2, GIPC3, SNAP23, STAT5B, RSPO3, AKT1S1, SNAP29, CASP2, AKT2, NELL1, MCTS1, TIA1, SCRG1, CIRBP, SEMA3F, SOX2, NRGN, PSTPIP2, ISM2, EHBP1, VTA1, and DUT.

In various embodiments, the panel of biomarkers include one or more proteins identified in Table 13 under the column “Gene Name”. In various embodiments, the panel of biomarkers include one or more proteins identified in Table 13 under the column “Gene Name” and differentially expressed in 1-5Y cohort (identified as “1-5Y only” or “Both” under the column “Cohort”). In various embodiments, the panel of biomarkers include two or more, five or more, ten or more, twenty or more, thirty or more, forty or more, fifty or more, one hundred or more, two hundred or more, or each of proteins identified in Table 13 under the column “Gene Name” and differentially expressed in 1-5Y cohort (identified as “1-5Y only” or “Both” under the column “Cohort”).

In particular embodiments, a panel of the prediction model (such as the panel of the prediction model shown in any of FIG. 3) includes one or more, two or more, three or more, four or more, or each protein biomarker selected from TSPAN1, CD28, SCN3B, ADGRB3, and IGFBP6.

In particular embodiments, a panel of the prediction model (such as the panel of the prediction model shown in any of FIG. 3) includes one or more, two or more, three or more, four or more, five or more, six or more, seven or more, eight or more, nine or more, ten or more, eleven or more, twelve or more, thirteen or more, fourteen or more, or each protein biomarker selected from NRTN, AIF1L, HSPB6, MB, TNFRSF19, IL5RA, TNR, CDNF, CST1, FGFBP2, S100A16, CD248, GFRA3, LMOD1, and POF1B.

In particular embodiments, a panel of the prediction model (such as the panel of the prediction model shown in any of FIG. 3) includes one or more, two or more, three or more, four or more, five or more, six or more, seven or more, eight or more, nine or more, ten or more, eleven or more, twelve or more, thirteen or more, fourteen or more, fifteen or more, sixteen is more, seventeen or more, eighteen or more, nineteen or more, twenty or more, twenty one or more, twenty two or more, twenty three or more, twenty four or more, twenty five or more twenty six or more, twenty seven or more, twenty eight or more, twenty nine or more, or each protein biomarker selected from DENND2B, COMP, CNTN2, SCARA5, CSPG4, ITGAV, SOST, SERPINA4, LILRA4, SPINK5, PINLYP, ACTN2, JAM2, FAP, TMOD4, GUCA2A, MFAP3L, DKK4, LAMA1, BAG3, SNCG, SEPTIN3, VWC2, KLRC1, ATRAID, ART3, SLITRK2, SIGLEC6, TMED4, and SLAMF7.

In particular embodiments, a panel of the prediction model (such as the panel of the prediction model shown in any of FIG. 3) includes one or more, two or more, three or more, four or more, five or more, six or more, seven or more, eight or more, nine or more, ten or more, eleven or more, twelve or more, thirteen or more, fourteen or more, fifteen or more, sixteen is more, seventeen or more, eighteen or more, nineteen or more, twenty or more, twenty one or more, twenty two or more, twenty three or more, twenty four or more, twenty five or more twenty six or more, twenty seven or more, twenty eight or more, twenty nine or more, thirty or more, thirty one or more, thirty two or more, thirty three or more, thirty four or more, thirty five or more, thirty six or more, thirty seven or more, thirty eight or more, thirty nine or more, forty or more, forty one or more, forty two or more, forty three or more, forty four or more, forty five or more, forty six or more, forty seven or more, forty eight or more, forty nine or more, or each protein biomarker selected from CKMT1A, SEMA6C, CD2, CST5, PBXIP1, LECT2, PYY, AGRN, INSL5, CD38, PI16, CCN5, TNFRSF17, LY9, GPC1, CLMP, MEP1B, CCN1, PCDH7, SPARCL1, CRNN, PM20D1, TNFRSF12A, DSCAM, PALM, CX3CL1, MEP1A, SLURP1, APOA4, ADAMTSL5, MEPE, WFDC1, RPS10, CD300C, RIPK4, CALCB, RTBDN, ENO3, NTF3, PTPRZ1, LRP2BP, CPE, MCAM, BGN, PLB1, YAP1, TGFBI, CYB5A, EDDM3B, and SELENOP.

In particular embodiments, a panel of the prediction model (such as the panel of the prediction model shown in any of FIG. 3) includes one or more protein marker selected from ENPP6, TMEM25, GIP, CSPG5, SCGN, TMPRSS15, LAIR2, KIRREL1, NTF4, TSPAN7, ENDOU, KLK10, CCL24, GPR37, CD3D, TJP3, DKKL1, CFC1, LRRC38, GCG, AGBL2, FASLG, AHNAK2, WFIKKN2, ANXA10, HS6ST1, DUSP29, CA14, CLEC7A, PHLDB2, SCRG1, RSPO3, TOP1, TINAGL1, NCAM1, FAM3D, FLT3LG, ZP3, AGRP, ASAH2, PDGFRB, AFM, NPY, PPY, XG, MFGE8, PROS1, MEGF11, CTSO, CTLA4, CSF3R, FCAR, CTAG1A, SCPEP1, PRSS53, CRELD2, PILRA, PROC, VASH1, NOS3, BPIFB2, UPK3BL1, NOP56, JAM3, HLA-DRA, SIL1, TRPV3, EDEM2, POLR2A, CBLN1, FKBP7, CCL20, PILRB, SIRPB1, VSTM1, BST2, DLL4, C1RL, RNASET2, KCNH2, IL12RB2, FZD10, OXCT1, TREML2, GRIN2B, GFRAL, RGS8, LRPAP1, LRP2, IGSF21, DPT, HEPACAM2, MATN3, UXS1, PTTG1, BTN1A1, IL17C, SCIN, TK1, FKBP14, VWA5A, PRKG1, SV2A, PMCH, NEXN, CDCP1, DDX53, THSD1, PAK4, MMP12, FCN1, UMOD, PDIA4, IL6, BRK1, LILRA2, RBPMS2, SERPIND1, TPSG1, CEACAM5, FGF9, PPIF, RNF43, SIGLEC9, TOMM20, PDE5A, NELL1, GBA, PAEP, ERN1, PCSK7, CHCHD6, MARCO, SFTPA1, IL9, KYNU, SPINT1, LRFN2, NECTIN1, OSCAR, PZP, BPIFB1, LILRA5, CALY, RRAS, GADD45GIP1, ISM2, SCGB3A2, CEACAM6, LPP, GKN1, LRIG1, CLSPN, CXCL13, SFTPA2, COX6B1, PTGR1, RBPMS, PPT1, AOC1, PDLIM5, L3HYPDH, LONP1, APOL1, CEACAM18, FGF7, and KRT14.

In various embodiments, the panel of biomarkers include one or more proteins identified in Table 13 under the column “Gene Name”. In various embodiments, the panel of biomarkers include one or more proteins identified in Table 13 under the column “Gene Name” and differentially expressed in 1-3Y cohort (identified as “1-3Y only” or “Both” under the column “Cohort”). In various embodiments, the panel of biomarkers include two or more, five or more, ten or more . . . two hundred or more proteins identified in Table 13 under the column “Gene Name” and differentially expressed in 1-3Y cohort (identified as “1-3Y only” or “Both” under the column “Cohort”).

In particular embodiments, a panel of the prediction model (such as the panel of the prediction model shown in any of FIG. 3) includes one or more, two or more, three or more, four or more, or each protein biomarker selected from GAST, ENPP2, FZD8, FGF23, and TFF1.

In particular embodiments, a panel of the prediction model (such as the panel of the prediction model shown in any of FIG. 3) includes one or more, two or more, three or more, four or more, five or more, six or more, seven or more, eight or more, nine or more, ten or more, eleven or more, twelve or more, thirteen or more, fourteen or more, or each protein biomarker selected from MAPT, FGF16, OXT, BRD1, MFAP4, WNT9A, FLRT2, CRTAC1, PAPPA, POMC, NGF, IDI2, TPT1, EPHA10, and MFAP3.

In particular embodiments, a panel of the prediction model (such as the panel of the prediction model shown in any of FIG. 3) includes one or more, two or more, three or more, four or more, five or more, six or more, seven or more, eight or more, nine or more, ten or more, eleven or more, twelve or more, thirteen or more, fourteen or more, fifteen or more, sixteen is more, seventeen or more, eighteen or more, nineteen or more, twenty or more, twenty one or more, twenty two or more, twenty three or more, twenty four or more, twenty five or more twenty six or more, twenty seven or more, twenty eight or more, twenty nine or more, or each protein biomarker selected from SOWAHA, RARRES1, DUSP3, SEMA3F, CNTN3, LPA, KLK11, RPGR, EPO, TDGF1, IL17A, CD160, TNPO1, GAMT, ENPP6, TMEM25, GIP, CSPG5, SCGN, TMPRSS15, LAIR2, KIRREL1, NTF4, TSPAN7, ENDOU, KLK10, CCL24, GPR37, CD3D, and TJP3.

In particular embodiments, a panel of the prediction model (such as the panel of the prediction model shown in any of FIG. 3) includes one or more, two or more, three or more, four or more, five or more, six or more, seven or more, eight or more, nine or more, ten or more, eleven or more, twelve or more, thirteen or more, fourteen or more, fifteen or more, sixteen is more, seventeen or more, eighteen or more, nineteen or more, twenty or more, twenty one or more, twenty two or more, twenty three or more, twenty four or more, twenty five or more twenty six or more, twenty seven or more, twenty eight or more, twenty nine or more, thirty or more, thirty one or more, thirty two or more, thirty three or more, thirty four or more, thirty five or more, thirty six or more, thirty seven or more, thirty eight or more, thirty nine or more, forty or more, forty one or more, forty two or more, forty three or more, forty four or more, forty five or more, forty six or more, forty seven or more, forty eight or more, forty nine or more, or each protein biomarker selected from DKKL1, CFC1, LRRC38, GCG, AGBL2, FASLG, AHNAK2, WFIKKN2, ANXA10, HS6ST1, DUSP29, CA14, CLEC7A, PHLDB2, SCRG1, RSPO3, TOP1, TINAGL1, NCAM1, FAM3D, FLT3LG, ZP3, AGRP, ASAH2, PDGFRB, AFM, NPY, PPY, XG, MFGE8, PROS1, MEGF11, SCT, CFB, F11, ANK2, ENOPH1, UGDH, ASAH1, ERBB4, IL36A, FGA, C5, OSMR, SSBP1, RICTOR, LRG1, C4BPB, AIDA, and SSC4D.

In particular embodiments, a panel of the prediction model (such as the panel of the prediction model shown in any of FIG. 3) includes one or more protein marker selected from GRN, IFNAR1, ENPEP, ACADSB, MAN1A2, GBP4, SERPING1, COL4A4, SOX2, GRSF1, PRAME, KIR2DS4, ADAMTS1, ITPRIP, CRISP3, DSG4, ITIH4, MRC1, GABRA4, SERPINA3, MILR1, PLIN1, SHH, KLKB1, IL17RA, MMP10, LBP, SMAD5, ADRA2A, SESTD1, CFI, AKR7L, CTSH, LYPD3, CBLIF, SMTN, CFH, SERPINC1, GDF15, PDZD2, ALDH2, IZUMO1, DNM3, CCL19, CSF2, MCEE, FDX1, SDC1, POSTN, GP2, CST7, CD14, NEK7, SHC1, CRELD1, TCN2, CMIP, CRHBP, C9, PXDNL, NRCAM, DLG4, TRAF3IP2, SULT2A1, GSTT2B, ITIH1, MRPL24, MUC16, IL3, CLU, FHIP2A, TK1, FKBP14, VWA5A, PRKG1, SV2A, PMCH, NEXN, CDCP1, DDX53, THSD1, PAK4, MMP12, FCN1, UMOD, PDIA4, IL6, BRK1, LILRA2, RBPMS2, SERPIND1, TPSG1, CEACAM5, FGF9, PPIF, RNF43, SIGLEC9, TOMM20, PDE5A, NELL1, GBA, PAEP, ERN1, PCSK7, CHCHD6, MARCO, SFTPA1, IL9, KYNU, SPINT1, LRFN2, NECTIN1, OSCAR, PZP, BPIFB1, LILRA5, CALY, RRAS, GADD45GIP1, ISM2, SCGB3A2, CEACAM6, LPP, GKN1, LRIG1, CLSPN, CXCL13, SFTPA2, COX6B1, PTGR1, RBPMS, PPT1, AOC1, PDLIM5, L3HYPDH, LONP1, APOL1, CEACAM18, FGF7, and KRT14.

### V. Assays

As shown in FIG. 1A, the system environment 100 involves implementing a marker quantification assay 120 for evaluating quantitative values of one or more biomarkers. Examples of an assay (e.g., marker quantification assay 120) for one or more markers include DNA assays, microarrays, polymerase chain reaction (PCR), RT-PCR, Southern blots, Northern blots, antibody-binding assays, enzyme-linked immunosorbent assays (ELISAs), flow cytometry, protein assays, Western blots, nephelometry, turbidimetry, chromatography, mass spectrometry, immunoassays, including, by way of example, but not limitation, RIA, immunofluorescence, immunochemiluminescence, immunoelectrochemiluminescence, or competitive immunoassays, immunoprecipitation, and the assays described in the Examples section below. The information from the assay can be quantitative and sent to a computer system of the invention. The information can also be qualitative, such as observing patterns or fluorescence, which can be translated into a quantitative measure by a user or automatically by a reader or computer system.

Various immunoassays designed to quantitate markers can be used in screening including multiplex assays. Measuring the concentration of a target marker in a sample or fraction thereof can be accomplished by a variety of specific assays. For example, a conventional sandwich type assay can be used in an array, ELISA, RIA, etc. format. Other immunoassays include Ouchterlony plates that provide a simple determination of antibody binding. Additionally, Western blots can be performed on protein gels or protein spots on filters, using a detection system specific for the markers as desired, conveniently using a labeling method.

Protein based analysis, using an antibody that specifically binds to a polypeptide (e.g. marker), can be used to quantify the marker level in a test sample obtained from a subject. In various embodiments, an antibody that binds to a marker can be a monoclonal antibody. In various embodiments, an antibody that binds to a marker can be a polyclonal antibody. For multiplex analysis of markers, arrays containing one or more marker affinity reagents, e.g. antibodies can be generated. Such an array can be constructed comprising antibodies against markers. Detection can utilize one or a panel of marker affinity reagents, e.g. a panel or cocktail of affinity reagents specific for one, two, three, four, five, six, seven, eight, nine, ten, eleven, twelve, thirteen, fourteen, fifteen, sixteen, seventeen, eighteen, nineteen, twenty, twenty one, or more markers.

In various embodiments, the multiplex assay involves the use of oligonucleotide labeled antibody probes that bind to target biomarkers and allow for subsequent quantification of biomarkers. One example of a multiplex assay that involves oligonucleotide labeled antibody probes is the Proximity Extension Assay (PEA) technology (Olink® Proteomics). Briefly, a pair of oligonucleotide labeled antibodies bind to a biomarker, wherein the two oligonucleotide sequences are complementary to one another. Thus, only when both antibodies bind to the target biomarker will the oligonucleotide sequences hybridize with one another. Mismatched oligonucleotide sequences (which occurs due to non-specific binding of antibodies or cross-reactivity of antibodies) will not hybridize and therefore, will not result in a readout. Hybridized oligonucleotide sequences undergo nucleic acid extension and amplification, followed by quantification using microfluidic qPCR. The quantified levels correlate to the quantitative expression values of the respective biomarkers.

In various embodiments, the multiplex assay involves the use of bead conjugated antibodies (e.g., capture antibodies) that enable the binding and detection of biomarkers. One example of a multiplex assay involving bead conjugated antibodies is Luminex's xMAP® Technology. Here, bead conjugated antibodies are added to the sample along with biotinylated detection antibodies. Both antibodies are specific to the biomarkers of interest and therefore, form an antibody-antigen sandwich. Streptavidin is further added, which binds to the biotinylated detection antibodies and enables detection of the complex. The Luminex 200™ or FlexMap® analyzer are employed to identify and quantify the amount of the biomarker in the sample. In various embodiments, the multiplex assay represents an improvement over Luminex's xMAP® technology, such as the Multi-Analyte Profile (MAP) technology by Myriad Rules Based Medicine (RBM), Inc.

The information from the assay can be quantitative and sent to a computer system of the invention. The information can also be qualitative, such as observing patterns or fluorescence, which can be translated into a quantitative measure by a user or automatically by a reader or computer system.

In various embodiments, prior to implementation of a marker quantification assay 120, a sample obtained from a subject can be processed. In various embodiments, processing the sample enables the implementation of the marker quantification assay 120 to more accurately evaluate quantitative values of one or more biomarkers in the sample.

In various embodiments, the sample from a subject can be processed to extract biomarkers from the sample. In one embodiment, the sample can undergo phase separation to separate the biomarkers from other portions of the sample. For example, the sample can undergo centrifugation (e.g., pelleting or density gradient centrifugation) to separate larger and/or more dense entities in the sample (e.g., cells and other macromolecules) from the biomarkers. Other examples include filtration (e.g., ultrafiltration) to phase separate the biomarkers from other portions of the sample.

In various embodiments, the sample from a subject can be processed to produce a sub-sample with a fraction of biomarkers that were in the sample. In various embodiments, producing a fraction of biomarkers can involve performing a fractionation procedure. One example of fractionation procedures include chromatography (e.g., gel filtration, ion exchange, hydrophobic chromatography, liquid chromatography or affinity chromatography). In particular embodiments, the protein fractionation procedure involves affinity purification or immunoprecipitation where biomarkers are bound by specific antibodies. Such antibodies can be immobilized on a support, such as a magnetic particle or nanoparticle or a plate.

### VI. Therapeutic Agents and Compositions for Therapeutic Agents

In various embodiments, a therapeutic agent can be provided to a subject subsequent to obtaining the sample from the subject and determining quantitative values of one or more markers in the obtained sample. As one example, a prediction model that analyzes predictors including quantitative values of one or more markers predicts that an individual is likely to develop cancer within a time period. In various embodiments, the prediction model may generate a prediction that is informative for selecting a therapeutic agent to be provided to the subject, the therapeutic agent likely to delay or prevent the onset of the cancer within the time period. For example, if the prediction model predicts that the subject has a presence of cancer, the prediction from the prediction model can be used to select a therapeutic agent for treating the currently present cancer. As another example, if the prediction model predicts that the subject is likely to develop cancer within a future timeframe, the prediction from the prediction model can be used to select a therapeutic agent that can be administered prophylactically (e.g., to prevent or to slow the onset of the future development of the cancer).

In various embodiments the therapeutic agent is a biologic, e.g. a cytokine, antibody, soluble cytokine receptor, anti-sense oligonucleotide, siRNA, RNA/DNA based vaccine, immune cell based therapies (e.g., adoptive cell therapy), and the like. Such biologic agents encompass muteins and derivatives of the biological agent, which derivatives can include, for example, fusion proteins, PEGylated derivatives, cholesterol conjugated derivatives, and the like as known in the art. Also included are antagonists of cytokines and cytokine receptors, e.g. traps and monoclonal antagonists. Also included are biosimilar or bioequivalent drugs to the active agents set forth herein. In various embodiments, the therapeutic agent can be radiotherapy or a surgical intervention.

Therapeutic agents for lung cancer can include chemotherapeutics such as docetaxel, doxorubicin hydrocholoride, methotrexate, cisplatin, carboplatin, gemcitabine, Nab-paclitaxel, paclitaxel, pemetrexed, gefitinib, erlotinib, brigatinib (Alunbrig®), capmatinib (Tabrecta®), selpercatinib (Retevmo®), entrectinib (Rozlytrek®), lorlatinib (Lorbrena®), larotrectinib (Vitrakvi®), dacomitinib (Vizimpro®), everolimus (Afinitor®), vinorelbine, pralsetinib (Gavreto®), dabrafenib (Tafinlar®), trametinib (Mekinist®), crizotinib (Xalkori®), alectinib (Alecensa®), ceritinib (Zykadia®), osimertinib (Tagrisso®). Afatinib (Gilotrif®), dacomitinib (Vizimpro®), and nintedanib (Vargatef®). Therapeutic agents for lung cancer can include antibody therapies such as durvalumab (Imfinzi®), nivolumab (Opdivo®), pembrolizumab (Keytruda®), atezolizumab (Tecentriq®), ramucirumab, bevacizumab (Avastin®, Mvasi®, Zirabev®), necitumumab (Portrazza®), and ipilimumab (Yervoy®).

A pharmaceutical composition administered to an individual includes an active agent such as the therapeutic agent described above. The active ingredient is present in a therapeutically effective amount, i.e., an amount sufficient when administered to treat a disease or medical condition mediated thereby. The compositions can also include various other agents to enhance delivery and efficacy, e.g. to enhance delivery and stability of the active ingredients.

Thus, for example, the compositions can also include, depending on the formulation desired, pharmaceutically-acceptable, non-toxic carriers or diluents, which are defined as vehicles commonly used to formulate pharmaceutical compositions for animal or human administration.

The diluent is selected so as not to affect the biological activity of the combination. Examples of such diluents are distilled water, buffered water, physiological saline, PBS, Ringer's solution, dextrose solution, and Hank's solution. In addition, the pharmaceutical composition or formulation can include other carriers, adjuvants, or non-toxic, nontherapeutic, nonimmunogenic stabilizers, excipients and the like. The compositions can also include additional substances to approximate physiological conditions, such as pH adjusting and buffering agents, toxicity adjusting agents, wetting agents and detergents. The composition can also include any of a variety of stabilizing agents, such as an antioxidant.

The pharmaceutical compositions described herein can be administered in a variety of different ways. Examples include administering a composition containing a pharmaceutically acceptable carrier via oral, intranasal, rectal, topical, intraperitoneal, intravenous, intramuscular, subcutaneous, subdermal, transdermal, intrathecal, or intracranial method.

Such a pharmaceutical composition may be administered for treatment (e.g., after diagnosis of a patient with lung cancer) purposes. Preventing, prophylaxis or prevention of a disease or disorder as used in the context of this invention refers to the administration of a composition to prevent the occurrence, onset, progression, or recurrence of lung cancer some or all of the symptoms of lung cancer or to lessen the likelihood of the onset of lung cancer. Treating, treatment, or therapy of lung cancer shall mean slowing, stopping or reversing the cancer's progression by administration of treatment according to the present invention. In the preferred embodiment, treating lung cancer means reversing the cancer's progression, ideally to the point of eliminating the cancer itself.

### VII. Cancers

Methods described herein involve diagnosing a cancer in a subject. In various embodiments, the cancer in the subject can include one or more of: lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, kidney cancer, lung cancer, neuroblastoma/glioblastoma, ovarian cancer, pancreatic cancer, prostate cancer, skin cancer, liver cancer, melanoma, squamous cell carcinomas of the mouth, throat, larynx, and lung, colon cancer, cervical cancer, cervical carcinoma, breast cancer, and epithelial cancer, renal cancer, genitourinary cancer, pulmonary cancer, esophageal carcinoma, head and neck carcinoma, large bowel cancer, hematopoietic cancer, testicular cancer, colon and/or rectal cancer, prostatic cancer, or pancreatic cancer.

In various embodiments, the cancer in the subject can be a particular subtype of a lung cancer. Example lung cancer subtypes include, but are not limited to: small cell lung cancer, non-small cell lung cancer, adenocarcinoma, squamous cell cancer, large cell carcinoma, small cell carcinoma, combined small cell carcinoma, lung sarcoma, lung lymphoma, bronchial carcinoids, and a stage of lung cancer (e.g., stage 1, stage 2, stage 3, or stage 4).

In various embodiments, the methods disclosed herein involve predicting a future risk of cancer, such as lung cancer, in a subject, In various embodiments, the methods disclosed herein involve predicting a future risk of a subtype of lung cancer, such as one of adenocarcinoma, squamous cell cancer, or large cell carcinoma.

### VIII. Computer Implementation

The methods of the invention, including the methods of predicting risk of cancer in an individual, are, in some embodiments, performed on one or more computers.

For example, the building and deployment of a prediction model and database storage can be implemented in hardware or software, or a combination of both. In one embodiment of the invention, a machine-readable storage medium is provided, the medium comprising a data storage material encoded with machine readable data which, when using a machine programmed with instructions for using said data, is capable of displaying any of the datasets and execution and results of a prediction model. Such data can be used for a variety of purposes, such as patient monitoring, treatment considerations, and the like. The invention can be implemented in computer programs executing on programmable computers, comprising a processor, a data storage system (including volatile and non-volatile memory and/or storage elements), a graphics adapter, a pointing device, a network adapter, at least one input device, and at least one output device. A display is coupled to the graphics adapter. Program code is applied to input data to perform the functions described above and generate output information. The output information is applied to one or more output devices, in known fashion. The computer can be, for example, a personal computer, microcomputer, or workstation of conventional design.

Each program can be implemented in a high level procedural or object oriented programming language to communicate with a computer system. However, the programs can be implemented in assembly or machine language, if desired. In any case, the language can be a compiled or interpreted language. Each such computer program is preferably stored on a storage media or device (e.g., ROM or magnetic diskette) readable by a general or special purpose programmable computer, for configuring and operating the computer when the storage media or device is read by the computer to perform the procedures described herein. The system can also be considered to be implemented as a computer-readable storage medium, configured with a computer program, where the storage medium so configured causes a computer to operate in a specific and predefined manner to perform the functions described herein.

The signature patterns and databases thereof can be provided in a variety of media to facilitate their use. “Media” refers to a manufacture that contains the signature pattern information of the present invention. The databases of the present invention can be recorded on computer readable media, e.g. any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. One of skill in the art can readily appreciate how any of the presently known computer readable mediums can be used to create a manufacture comprising a recording of the present database information. “Recorded” refers to a process for storing information on computer readable medium, using any such methods as known in the art. Any convenient data storage structure can be chosen, based on the means used to access the stored information. A variety of data processor programs and formats can be used for storage, e.g. word processing text file, database format, etc.

In some embodiments, the methods of the invention, including the methods of predicting risk of cancer in an individual, are performed on one or more computers in a distributed computing system environment (e.g., in a cloud computing environment). In this description, “cloud computing” is defined as a model for enabling on-demand network access to a shared set of configurable computing resources. Cloud computing can be employed to offer on-demand access to the shared set of configurable computing resources. The shared set of configurable computing resources can be rapidly provisioned via virtualization and released with low management effort or service provider interaction, and then scaled accordingly. A cloud-computing model can be composed of various characteristics such as, for example, on-demand self-service, broad network access, resource pooling, rapid elasticity, measured service, and so forth. A cloud-computing model can also expose various service models, such as, for example, Software as a Service (“SaaS”), Platform as a Service (“PaaS”), and Infrastructure as a Service (“IaaS”). A cloud-computing model can also be deployed using different deployment models such as private cloud, community cloud, public cloud, hybrid cloud, and so forth. In this description and in the claims, a “cloud-computing environment” is an environment in which cloud computing is employed.

### VIII.A. Example Computer

FIG. 4 illustrates an example computer for implementing the entities shown in FIG. 1A, 1i, 2, and 3. The computer 400 includes at least one processor 402 coupled to a chipset 404. The chipset 404 includes a memory controller hub 420 and an input/output (I/O) controller hub 422. A memory 406 and a graphics adapter 412 are coupled to the memory controller hub 420, and a display 418 is coupled to the graphics adapter 412. A storage device 408, an input interface 414, and network adapter 416 are coupled to the I/O controller hub 422. Other embodiments of the computer 400 have different architectures.

The storage device 408 is a non-transitory computer-readable storage medium such as a hard drive, compact disk read-only memory (CD-ROM), DVD, or a solid-state memory device. The memory 406 holds instructions and data used by the processor 402. The input interface 414 is a touch-screen interface, a mouse, track ball, or other type of pointing device, a keyboard 410, or some combination thereof, and is used to input data into the computer 400. In some embodiments, the computer 400 may be configured to receive input (e.g., commands) from the input interface 414 via gestures from the user. The graphics adapter 412 displays images and other information on the display 418. The network adapter 416 couples the computer 400 to one or more computer networks.

The computer 400 is adapted to execute computer program modules for providing functionality described herein. As used herein, the term “module” refers to computer program logic used to provide the specified functionality. Thus, a module can be implemented in hardware, firmware, and/or software. In one embodiment, program modules are stored on the storage device 408, loaded into the memory 406, and executed by the processor 402.

The types of computers 400 used by the entities of FIGS. 1A, 1, and 2 can vary depending upon the embodiment and the processing power required by the entity. For example, the cancer prediction system 130 can run in a single computer 400 or multiple computers 400 communicating with each other through a network such as in a server farm. The computers 400 can lack some of the components described above, such as graphics adapters 412, and displays 418.

### IX. Kit Implementation

Also disclosed herein are kits for predicting risk of a cancer in an individual. Such kits can include reagents for detecting quantitative values of one or biomarkers and instructions for predicting risk of cancer based on at least the detected quantitative values of the biomarkers.

The detection reagents can be provided as part of a kit. Thus, the invention further provides kits for detecting the presence of a panel of biomarkers of interest in a biological test sample. A kit can comprise one or more sets of reagents for generating a dataset via at least one detection assay that analyzes the test sample from the subject. In various embodiments, the set of reagents enables detection of quantitative values of protein biomarkers, such as any of the protein biomarkers described herein and in particular, any of the protein biomarkers identified in Tables 1-3.

A kit can include instructions for use of one or more sets of reagents. For example, a kit can include instructions for performing at least one marker quantification assay, examples of which are described herein. In various embodiments, the kits include instructions for practicing the methods disclosed herein (e.g., methods for training or deploying a prediction model to predict risk of cancer). These instructions can be present in the subject kits in a variety of forms, one or more of which can be present in the kit. One form in which these instructions can be present is as printed information on a suitable medium or substrate, e.g., a piece or pieces of paper on which the information is printed, in the packaging of the kit, in a package insert, etc. Yet another means would be a computer readable medium, e.g., diskette, CD, hard-drive, network data storage, etc., on which the information has been recorded. Yet another means that can be present is a website address which can be used via the internet to access the information at a removed site. Any convenient means can be present in the kits.

### X. Systems

Further disclosed herein are systems for predicting risk of cancer in a subject. In various embodiments, such a system can include one or more sets of reagents for detecting quantitative values of biomarkers in one or more panels of a prediction model, an apparatus configured to receive a mixture of the one or more sets of reagents and a test sample obtained from a subject to measure the quantitative values of the biomarkers, and a computer system communicatively coupled to the apparatus to obtain the measured quantitative values and to implement the prediction model to predict risk of cancer in a subject.

The one or more sets of reagents enable the detection of quantitative levels of the biomarkers in the biomarker panel. In various embodiments, the one or more sets of reagents involve reagents used to perform one or more assays more measuring levels of protein biomarkers. For example, the reagents include one or more antibodies that bind to one or more of the biomarkers. The antibodies may be monoclonal antibodies or polyclonal antibodies. As another example, the reagents can include reagents for performing ELISA including buffers and detection agents.

The apparatus is configured to detect quantitative levels of biomarkers in a mixture of a reagent and test sample. As an example, the apparatus can determine quantitative levels of biomarkers through a protein detection assay (e.g., a protein detection assay that uses one of NMR spectroscopy or LC-MS).

The mixture of the reagent and test sample may be presented to the apparatus through various conduits, examples of which include wells of a well plate (e.g., 96 well plate), a vial, a tube, and integrated fluidic circuits. As such, the apparatus may have an opening (e.g., a slot, a cavity, an opening, a sliding tray) that can receive the container including the reagent test sample mixture and perform a reading to generate quantitative values of biomarkers. Examples of an apparatus include a plate reader (e.g., a luminescent plate reader, absorbance plate reader, fluorescence plate reader), a spectrometer, and a spectrophotometer. Further examples of an apparatus include an NMR spectroscopy system or a LC-MS system.

The computer system, such as example computer 400 described in FIG. 4, communicates with the apparatus to receive the quantitative values of biomarkers. The computer system implements, in silico, a prediction model to analyze the quantitative values of the biomarkers and predict risk of cancer for the subject.

### Additional Embodiments

Disclosed herein are methods for predicting risk of cancer in a subject, the method comprising: obtaining or having obtained a dataset derived from the subject comprising quantitative levels of a plurality of biomarkers, wherein the plurality of biomarkers comprises protein biomarkers comprising two or more of TGFA, MMP12, TNFRSF13B, TNFSF14, and MASP1, and generating a prediction of risk of cancer for the subject by applying a predictive model to the quantitative values of the plurality of biomarkers.

In various embodiments, the protein biomarkers comprise three or more of TGFA, MMP12, TNFRSF13B, TNFSF14, and MASP1.

In various embodiments, the protein biomarkers comprise four or more of TGFA, MMP12, TNFRSF13B, TNFSF14, and MASP1.

In various embodiments, the protein biomarkers comprise each of TGFA, MMP12, TNFRSF13B, TNFSF14, and MASP1.

In various embodiments, the protein biomarkers further comprise one or more of THBS2, GDNF, FLT1, FXYD5, CST5, ARNT, CDCP1, CCL20, FLT3LG, CLEC7A, PRKCQ, SCGN, IL5, NPY, and S100A16.

In various embodiments, the protein biomarkers further comprise five or more of THBS2, GDNF, FLT1, FXYD5, CST5, ARNT, CDCP1, CCL20, FLT3LG, CLEC7A, PRKCQ, SCGN, IL5, NPY, and S100A16.

In various embodiments, the protein biomarkers further comprise ten or more of THBS2, GDNF, FLT1, FXYD5, CST5, ARNT, CDCP1, CCL20, FLT3LG, CLEC7A, PRKCQ, SCGN, IL5, NPY, and S100A16.

In various embodiments, the protein biomarkers further comprise each of THBS2, GDNF, FLT1, FXYD5, CST5, ARNT, CDCP1, CCL20, FLT3LG, CLEC7A, PRKCQ, SCGN, IL5, NPY, and S100A16.

In various embodiments, the protein biomarkers further comprise one or more, five or more, or each of IL1B, CD84, STC1, PRDX3, LAP3, GAMT, CASP2, ITGA6, DECR1, and YTHDF3.

In various embodiments, the protein biomarkers further comprise one or more of IL1B, CD84, STC1, PRDX3, LAP3, GAMT, CASP2, ITGA6, DECR1, and YTHDF3.

In various embodiments, the protein biomarkers further comprise five or more of IL1B, CD84, STC1, PRDX3, LAP3, GAMT, CASP2, ITGA6, DECR1, and YTHDF3.

In various embodiments, the protein biomarkers further comprise each of IL1B, CD84, STC1, PRDX3, LAP3, GAMT, CASP2, ITGA6, DECR1, and YTHDF3.

In various embodiments, the predictive model comprises a elastic net regression model, and the predictive model achieves an area under a curve (AUC) value of at least 0.65. In various embodiments, the predictive model comprises a support vector machine, and the predictive model achieves an area under a curve (AUC) value of at least 0.70. In various embodiments, the predictive model comprises a random forest model, and the predictive model achieves an area under a curve (AUC) value of at least 0.67. In various embodiments, the predictive model comprises a XGBoost model, and the predictive model achieves an area under a curve (AUC) value of at least 0.68.

Additionally disclosed herein is a method for predicting risk of cancer in a subject, the method comprising: obtaining or having obtained a dataset derived from the subject comprising quantitative levels of a plurality of biomarkers, wherein the plurality of biomarkers comprises protein biomarkers comprising two or more of CEACAM5, TOP1, NCAM1, SCGB3A2, and CALY, and generating a prediction of risk of cancer for the subject by applying a predictive model to the quantitative values of the plurality of biomarkers.

In various embodiments, the protein biomarkers comprise three or more of CEACAM5, TOP1, NCAM1, SCGB3A2, and CALY.

In various embodiments, the protein biomarkers comprise four or more of CEACAM5, TOP1, NCAM1, SCGB3A2, and CALY.

In various embodiments, the protein biomarkers comprise each of CEACAM5, TOP1, NCAM1, SCGB3A2, and CALY.

In various embodiments, the protein biomarkers further comprise one or more of TGFBI, CABP2, ENPP6, KRT14, HEPACAM2, TMEM25, SGSH, MFAP3L, TNFSF14, CD3D, TMED4, ZP3, MMP12, GCG, and AFM.

In various embodiments, the protein biomarkers further comprise five or more of TGFBI, CABP2, ENPP6, KRT14, HEPACAM2, TMEM25, SGSH, MFAP3L, TNFSF14, CD3D, TMED4, ZP3, MMP12, GCG, and AFM.

In various embodiments, the protein biomarkers further comprise ten or more of TGFBI, CABP2, ENPP6, KRT14, HEPACAM2, TMEM25, SGSH, MFAP3L, TNFSF14, CD3D, TMED4, ZP3, MMP12, GCG, and AFM.

In various embodiments, the protein biomarkers further comprise each of TGFBI, CABP2, ENPP6, KRT14, HEPACAM2, TMEM25, SGSH, MFAP3L, TNFSF14, CD3D, TMED4, ZP3, MMP12, GCG, and AFM.

In various embodiments, the protein biomarkers further comprise one or more of SPINT1, LILRA4, FLT3LG, AGBL2, PAEP, SCGB3A1, LRFN2, TJP3, FGF7, LRIG1, CA14, CEACAM18, CST1, ANXA10, CDCP1, GPC5, OSCAR, CEACAM6, CD2, SNCG, GPR37, SEPTIN3, RAB10, DKK4, DKKL1, SOST, CSF3, VWA5A, TSPAN7, and PAK4.

In various embodiments, the protein biomarkers further comprise five or more of SPINT1, LILRA4, FLT3LG, AGBL2, PAEP, SCGB3A1, LRFN2, TJP3, FGF7, LRIG1, CA14, CEACAM18, CST1, ANXA10, CDCP1, GPC5, OSCAR, CEACAM6, CD2, SNCG, GPR37, SEPTIN3, RAB10, DKK4, DKKL1, SOST, CSF3, VWA5A, TSPAN7, and PAK4.

In various embodiments, the protein biomarkers further comprise ten or more of SPINT1, LILRA4, FLT3LG, AGBL2, PAEP, SCGB3A1, LRFN2, TJP3, FGF7, LRIG1, CA14, CEACAM18, CST1, ANXA10, CDCP1, GPC5, OSCAR, CEACAM6, CD2, SNCG, GPR37, SEPTIN3, RAB10, DKK4, DKKL1, SOST, CSF3, VWA5A, TSPAN7, and PAK4.

In various embodiments, the protein biomarkers further comprise twenty or more of SPINT1, LILRA4, FLT3LG, AGBL2, PAEP, SCGB3A1, LRFN2, TJP3, FGF7, LRIG1, CA14, CEACAM18, CST1, ANXA10, CDCP1, GPC5, OSCAR, CEACAM6, CD2, SNCG, GPR37, SEPTIN3, RAB10, DKK4, DKKL1, SOST, CSF3, VWA5A, TSPAN7, and PAK4.

In various embodiments, the protein biomarkers further comprise each of SPINT1, LILRA4, FLT3LG, AGBL2, PAEP, SCGB3A1, LRFN2, TJP3, FGF7, LRIG1, CA14, CEACAM18, CST1, ANXA10, CDCP1, GPC5, OSCAR, CEACAM6, CD2, SNCG, GPR37, SEPTIN3, RAB10, DKK4, DKKL1, SOST, CSF3, VWA5A, TSPAN7, and PAK4.

In various embodiments, the protein biomarkers further comprise one or more of BPIFB1, SIGLEC9, ZNRD2, PM20D1, TK1, RPS10, PMCH, RNF43, MEP1B, BGN, NELL1, CD101, LRP2BP, PRSS53, MFGE8, THSD1, CKMT1A, MEPE, APOL1, RBPMS, MARCO, KLRC1, FGFBP2, TPSG1, SELENOP, CLEC7A, UPK3BL1, HS6ST1, ENDOU, IL12RB2, CYB5A, GKN1, NRTN, CCL26, CRNN, PINLYP, LAIR2, BAG3, SCPEP1, RIPK4, CTSE, TMOD4, SFTPA1, SEMA4D, IL17C, GFRA3, DPEP2, EDEM2, CD84, and KIRREL2.

In various embodiments, the protein biomarkers further comprise five or more of BPIFB1, SIGLEC9, ZNRD2, PM20D1, TK1, RPS10, PMCH, RNF43, MEP1B, BGN, NELL1, CD101, LRP2BP, PRSS53, MFGE8, THSD1, CKMT1A, MEPE, APOL1, RBPMS, MARCO, KLRC1, FGFBP2, TPSG1, SELENOP, CLEC7A, UPK3BL1, HS6ST1, ENDOU, IL12RB2, CYB5A, GKN1, NRTN, CCL26, CRNN, PINLYP, LAIR2, BAG3, SCPEP1, RIPK4, CTSE, TMOD4, SFTPA1, SEMA4D, IL17C, GFRA3, DPEP2, EDEM2, CD84, and KIRREL2.

In various embodiments, the protein biomarkers further comprise ten or more of BPIFB1, SIGLEC9, ZNRD2, PM20D1, TK1, RPS10, PMCH, RNF43, MEP1B, BGN, NELL1, CD101, LRP2BP, PRSS53, MFGE8, THSD1, CKMT1A, MEPE, APOL1, RBPMS, MARCO, KLRC1, FGFBP2, TPSG1, SELENOP, CLEC7A, UPK3BL1, HS6ST1, ENDOU, IL12RB2, CYB5A, GKN1, NRTN, CCL26, CRNN, PINLYP, LAIR2, BAG3, SCPEP1, RIPK4, CTSE, TMOD4, SFTPA1, SEMA4D, IL17C, GFRA3, DPEP2, EDEM2, CD84, and KIRREL2.

In various embodiments, the protein biomarkers further comprise twenty or more of BPIFB1, SIGLEC9, ZNRD2, PM20D1, TK1, RPS10, PMCH, RNF43, MEP1B, BGN, NELL1, CD101, LRP2BP, PRSS53, MFGE8, THSD1, CKMT1A, MEPE, APOL1, RBPMS, MARCO, KLRC1, FGFBP2, TPSG1, SELENOP, CLEC7A, UPK3BL1, HS6ST1, ENDOU, IL12RB2, CYB5A, GKN1, NRTN, CCL26, CRNN, PINLYP, LAIR2, BAG3, SCPEP1, RIPK4, CTSE, TMOD4, SFTPA1, SEMA4D, IL17C, GFRA3, DPEP2, EDEM2, CD84, and KIRREL2.

In various embodiments, the protein biomarkers further comprise thirty or more of BPIFB1, SIGLEC9, ZNRD2, PM20D1, TK1, RPS10, PMCH, RNF43, MEP1B, BGN, NELL1, CD101, LRP2BP, PRSS53, MFGE8, THSD1, CKMT1A, MEPE, APOL1, RBPMS, MARCO, KLRC1, FGFBP2, TPSG1, SELENOP, CLEC7A, UPK3BL1, HS6ST1, ENDOU, IL12RB2, CYB5A, GKN1, NRTN, CCL26, CRNN, PINLYP, LAIR2, BAG3, SCPEP1, RIPK4, CTSE, TMOD4, SFTPA1, SEMA4D, IL17C, GFRA3, DPEP2, EDEM2, CD84, and KIRREL2.

In various embodiments, the protein biomarkers further comprise forty or more of BPIFB1, SIGLEC9, ZNRD2, PM20D1, TK1, RPS10, PMCH, RNF43, MEP1B, BGN, NELL1, CD101, LRP2BP, PRSS53, MFGE8, THSD1, CKMT1A, MEPE, APOL1, RBPMS, MARCO, KLRC1, FGFBP2, TPSG1, SELENOP, CLEC7A, UPK3BL1, HS6ST1, ENDOU, IL12RB2, CYB5A, GKN1, NRTN, CCL26, CRNN, PINLYP, LAIR2, BAG3, SCPEP1, RIPK4, CTSE, TMOD4, SFTPA1, SEMA4D, IL17C, GFRA3, DPEP2, EDEM2, CD84, and KIRREL2.

In various embodiments, the protein biomarkers further comprise each of BPIFB1, SIGLEC9, ZNRD2, PM20D1, TK1, RPS10, PMCH, RNF43, MEP1B, BGN, NELL1, CD101, LRP2BP, PRSS53, MFGE8, THSD1, CKMT1A, MEPE, APOL1, RBPMS, MARCO, KLRC1, FGFBP2, TPSG1, SELENOP, CLEC7A, UPK3BL1, HS6ST1, ENDOU, IL12RB2, CYB5A, GKN1, NRTN, CCL26, CRNN, PINLYP, LAIR2, BAG3, SCPEP1, RIPK4, CTSE, TMOD4, SFTPA1, SEMA4D, IL17C, GFRA3, DPEP2, EDEM2, CD84, and KIRREL2.

In various embodiments, the protein biomarkers further comprise one or more of NECTIN1, CBLN1, NTF3, PYY, XG, NPY, CCL20, SIL1, PLB1, DUSP29, UMOD, ATXN2L, LEO1, PROS1, EDDM3B, ENO3, DCBLD2, MMP9, KIF22, DENND2B, C1RL, PVALB, CXCL8, PPY, CCN1, KLK10, RRAS, SCN3B, BPIFB2, ITGAL, DDX1, MEGF11, NOP56, NTF4, HNMT, IL9, SCRIB, UXS1, MEP1A, ACTN2, NECAP2, CLEC1OA, DDX53, SV2A, ATXN10, PI16, KCNH2, TNR, PDGFRB, SERPINA4, CDC27, MICALL2, CD28, BRK1, SLC16A1, DSCAM, PBXIP1, MATN3, SFTPA2, PTTG1, ASAH2, SCG2, PTGR1, GBA, PTPRZ1, ERN1, LECT2, SCGN, HLA-DRA, IL5RA, LRPAP1, CXCL13, NEXN, CD248, KYNU, ADAMTS15, WFIKKN2, CLEC14A, FZD10, PROC, LY9, LRP2, CX3CL1, RNASET2, CTSS, MCEMP1, COMP, SIGLEC6, CCL24, AOC1, PLXNB3, TMPRSS15, FCAR, SCIN, IFI30, KIRREL1, FXYD5, S100A16, LILRA5, CLSPN, AHNAK2, CTLA4, INSL5, WDR46, CST5, PHLDB2, TREML2, GUCA2A, PFDN2, PDIA4, LAMA1, SLAMF7, RGS8, IL6, PSG1, PZP, RRM2, GFRAL, AIF1L, LGMN, C1QTNF9, TSPAN1, DLL4, CRELD2, SCARF1, FGF9, JAM3, LPP, HSPB1, PPT1, PPIF, TRPV3, APOA4, LYSMD3, TGFA, ATP6V1D, LRRC38, CTAG1A, TINAGL1, POLR2A, EDIL3, LAP3, SORD, ARHGAP30, CSPG4, ART3, GADD45GIP1, SLURP1, LILRA2, GZMH, FKBP7, SLC27A4, CALCB, GIT1, CTSO, PCBD1, CSF3R, EIF1AX, CSPG5, CD93, ADAMTSL5, ISM2, CPE, WFDC1, VWC2, SPINK5, BTN1A1, DPT, FCN1, AIF1, GPC1, FAP, CLNS1A, CFC1, FASLG, NCS1, PRKAR1A, RCOR1, SLITRK2, SPARCL1, HSPB6, TNFRSF12A, IL6, SERPIND1, CEBPB, CASC3, AMPD3, YTHDF3, AAMDC, STX7, AGRP, ICA1, CHCHD6, IGSF21, VSTM1, PCDH7, VNN2, GP6, ITGAV, CD40LG, GIP, MB, TPD52L2, HPSE, GRIN2B, TREML1, C3, TNFRSF17, IL6, CD226, PALM, FKBP14, RBPMS2, CLEC6A, DAAM1, FAM3D, WASF1, HS1BP3, NOS3, POF1B, PLXNA4, MITD1, ERMAP, SYAP1, LRRC59, CNTN2, RAB2B, PENK, MCAM, EIF2S2, EGF, PTPN6, NID2, EHD3, IGFBP6, LMOD1, PAGR1, CD300C, SKAP2, PRKG1, SYTL4, GYS1, CASP3, PILRA, CD69, CCN5, PCBP2, LMOD1, PDIA5, PCSK7, SCARA5, METAP1D, ADGRB3, MPIG6B, NUMB, L3HYPDH, DENR, AGRN, COX6B1, JAM2, TIA1, CACYBP, SEMA6C, VAT1, SUSD1, RSPO3, TWF2, BOLA1, OXCT1, ITGA6, BST2, F2R, PILRB, RTBDN, ENOX2, DOK1, VASH1, DTD1, DDHD2, TBC1D23, GLRX5, CDNF, SIRPB1, NMT1, STK11, RPL14, PSTPIP2, FHIT, CLMP, LMOD1, ERP29, BECN1, CD38, YAP1, CA13, CRKL, PPP1R9B, FLI1, CMC1, CDC37, ARHGAP45, PDAP1, NUDC, CLEC1B, USO1, SNAP23, HGS, FUS, PIK3AP1, F11R, TBC1D17, ITPA, IL1B, ENO1, THTPA, SAFB2, JPT2, GIMAP7, NIT2, RILPL2, PRTFDC1, TADA3, TOMM20, HPCAL1, LONP1, CALCOCO1, ATRAID, TYMP, TNFRSF19, DNPEP, NRGN, STK4, SSNA1, CRYGD, LZTFL1, SNAP29, PDLIM5, CASP2, MANF, BACH1, DAPP1, AKR1B1, EREG, DAG1, HSBP1, DUT, AKT2, PLA2G4A, TXLNA, PIKFYVE, FYB1, CSDE1, RHOC, HNRNPK, DCTD, SCRG1, LACTB2, RGCC, GIMAP8, GRHPR, SNX5, NCK2, EIF4G1, BNIP3L, ACOT13, MECR, MAP2K6, SEC31A, MGLL, MESD, NUDT16, SULTIA1, GOPC, VTA1, PDLIM7, ANXA2, GGACT, PMVK, USP8, SNCA, CAMSAP1, HEXIMI, SHMT1, LGALS8, APPL2, MAP2K1, EHBP1, MAP4K5, PDE5A, HARS1, SRC, TACC3, and RAB27B.

In various embodiments, the predictive model comprises a elastic net regression model, and the predictive model achieves an area under a curve (AUC) value of at least 0.85. In various embodiments, the predictive model comprises a support vector machine, and the predictive model achieves an area under a curve (AUC) value of at least 0.84. In various embodiments, the predictive model comprises a random forest model, and the predictive model achieves an area under a curve (AUC) value of at least 0.72. In various embodiments, the predictive model comprises a XGBoost model, and the predictive model achieves an area under a curve (AUC) value of at least 0.73.

Additionally disclosed herein is a method for predicting risk of cancer in a subject, the method comprising: obtaining or having obtained a dataset derived from the subject comprising quantitative levels of a plurality of biomarkers, wherein the plurality of biomarkers comprises protein biomarkers comprising two or more of VWA5A, ENPP6, TMEM25, ALDH2, and LEO1, and generating a prediction of risk of cancer for the subject by applying a predictive model to the quantitative values of the plurality of biomarkers.

In various embodiments, the protein biomarkers comprise three or more of VWA5A, ENPP6, TMEM25, ALDH2, and LEO1.

In various embodiments, the protein biomarkers comprise four or more of VWA5A, ENPP6, TMEM25, ALDH2, and LEO1.

In various embodiments, the protein biomarkers comprise each of VWA5A, ENPP6, TMEM25, ALDH2, and LEO1.

In various embodiments, the protein biomarkers further comprise one or more of GAMT, TPSG1, ANK2, SCT, TSPAN7, GPC5, PGLYRP1, PAK4, TNFSF14, CLEC6A, TMPRSS15, PMCH, KRT14, SFTPA1, and LRFN2.

In various embodiments, the protein biomarkers further comprise five or more of GAMT, TPSG1, ANK2, SCT, TSPAN7, GPC5, PGLYRP1, PAK4, TNFSF14, CLEC6A, TMPRSS15, PMCH, KRT14, SFTPA1, and LRFN2.

In various embodiments, the protein biomarkers further comprise ten or more of GAMT, TPSG1, ANK2, SCT, TSPAN7, GPC5, PGLYRP1, PAK4, TNFSF14, CLEC6A, TMPRSS15, PMCH, KRT14, SFTPA1, and LRFN2.

In various embodiments, the protein biomarkers further comprise each of GAMT, TPSG1, ANK2, SCT, TSPAN7, GPC5, PGLYRP1, PAK4, TNFSF14, CLEC6A, TMPRSS15, PMCH, KRT14, SFTPA1, and LRFN2.

In various embodiments, the protein biomarkers further comprise one or more of MMP12, TNPO1, GAST, CD3D, TK1, DLGAP5, SCGN, CCL24, PSG1, CLU, CFB, LBP, CRYM, LAIR2, TCN2, SV2A, CRHBP, C5, SCGB3A2, ANXA10, GCG, RPGR, PAPPA, FZD8, CSPG5, BRK1, OXT, FDX1, ENPEP, and LRG1.

In various embodiments, the protein biomarkers further comprise five or more of MMP12, TNPO1, GAST, CD3D, TK1, DLGAP5, SCGN, CCL24, PSG1, CLU, CFB, LBP, CRYM, LAIR2, TCN2, SV2A, CRHBP, C5, SCGB3A2, ANXA10, GCG, RPGR, PAPPA, FZD8, CSPG5, BRK1, OXT, FDX1, ENPEP, and LRG1.

In various embodiments, the protein biomarkers further comprise ten or more of MMP12, TNPO1, GAST, CD3D, TK1, DLGAP5, SCGN, CCL24, PSG1, CLU, CFB, LBP, CRYM, LAIR2, TCN2, SV2A, CRHBP, C5, SCGB3A2, ANXA10, GCG, RPGR, PAPPA, FZD8, CSPG5, BRK1, OXT, FDX1, ENPEP, and LRG1.

In various embodiments, the protein biomarkers further comprise twenty or more of MMP12, TNPO1, GAST, CD3D, TK1, DLGAP5, SCGN, CCL24, PSG1, CLU, CFB, LBP, CRYM, LAIR2, TCN2, SV2A, CRHBP, C5, SCGB3A2, ANXA10, GCG, RPGR, PAPPA, FZD8, CSPG5, BRK1, OXT, FDX1, ENPEP, and LRG1.

In various embodiments, the protein biomarkers further comprise each of MMP12, TNPO1, GAST, CD3D, TK1, DLGAP5, SCGN, CCL24, PSG1, CLU, CFB, LBP, CRYM, LAIR2, TCN2, SV2A, CRHBP, C5, SCGB3A2, ANXA10, GCG, RPGR, PAPPA, FZD8, CSPG5, BRK1, OXT, FDX1, ENPEP, and LRG1.

In various embodiments, the protein biomarkers further comprise one or more of PRAME, KIRREL1, KIF22, SPINT1, FGA, C1QTNF9, KIR2DS4, MMP9, NEXN, FCN1, MFGE8, ZNRD2, PDGFRB, HS6ST1, DUSP3, CABP2, DNM3, FGL1, TOP1, CDCP1, RAB10, THSD1, FASLG, MCEMP1, COL4A4, ENO1, BRD1, GP5, ZP3, SERPIND1, NCAM1, ATXN10, MUC16, GABRA4, POSTN, MAEA, SHH, DDX53, PRKG1, PAEP, RICTOR, IL6, FKBP14, CCL26, AIDA, GIP, TGFA, ITIH4, PCSK7, and RARRES1.

In various embodiments, the protein biomarkers further comprise five or more of PRAME, KIRREL1, KIF22, SPINT1, FGA, C1QTNF9, KIR2DS4, MMP9, NEXN, FCN1, MFGE8, ZNRD2, PDGFRB, HS6ST1, DUSP3, CABP2, DNM3, FGL1, TOP1, CDCP1, RAB10, THSD1, FASLG, MCEMP1, COL4A4, ENO1, BRD1, GP5, ZP3, SERPIND1, NCAM1, ATXN10, MUC16, GABRA4, POSTN, MAEA, SHH, DDX53, PRKG1, PAEP, RICTOR, IL6, FKBP14, CCL26, AIDA, GIP, TGFA, ITIH4, PCSK7, and RARRES1.

In various embodiments, the protein biomarkers further comprise ten or more of PRAME, KIRREL1, KIF22, SPINT1, FGA, C1QTNF9, KIR2DS4, MMP9, NEXN, FCN1, MFGE8, ZNRD2, PDGFRB, HS6ST1, DUSP3, CABP2, DNM3, FGL1, TOP1, CDCP1, RAB10, THSD1, FASLG, MCEMP1, COL4A4, ENO1, BRD1, GP5, ZP3, SERPIND1, NCAM1, ATXN10, MUC16, GABRA4, POSTN, MAEA, SHH, DDX53, PRKG1, PAEP, RICTOR, IL6, FKBP14, CCL26, AIDA, GIP, TGFA, ITIH4, PCSK7, and RARRES1.

In various embodiments, the protein biomarkers further comprise twenty or more of PRAME, KIRREL1, KIF22, SPINT1, FGA, C1QTNF9, KIR2DS4, MMP9, NEXN, FCN1, MFGE8, ZNRD2, PDGFRB, HS6ST1, DUSP3, CABP2, DNM3, FGL1, TOP1, CDCP1, RAB10, THSD1, FASLG, MCEMP1, COL4A4, ENO1, BRD1, GP5, ZP3, SERPIND1, NCAM1, ATXN10, MUC16, GABRA4, POSTN, MAEA, SHH, DDX53, PRKG1, PAEP, RICTOR, IL6, FKBP14, CCL26, AIDA, GIP, TGFA, ITIH4, PCSK7, and RARRES1.

In various embodiments, the protein biomarkers further comprise thirty or more of PRAME, KIRREL1, KIF22, SPINT1, FGA, C1QTNF9, KIR2DS4, MMP9, NEXN, FCN1, MFGE8, ZNRD2, PDGFRB, HS6ST1, DUSP3, CABP2, DNM3, FGL1, TOP1, CDCP1, RAB10, THSD1, FASLG, MCEMP1, COL4A4, ENO1, BRD1, GP5, ZP3, SERPIND1, NCAM1, ATXN10, MUC16, GABRA4, POSTN, MAEA, SHH, DDX53, PRKG1, PAEP, RICTOR, IL6, FKBP14, CCL26, AIDA, GIP, TGFA, ITIH4, PCSK7, and RARRES1.

In various embodiments, the protein biomarkers further comprise forty or more of PRAME, KIRREL1, KIF22, SPINT1, FGA, C1QTNF9, KIR2DS4, MMP9, NEXN, FCN1, MFGE8, ZNRD2, PDGFRB, HS6ST1, DUSP3, CABP2, DNM3, FGL1, TOP1, CDCP1, RAB10, THSD1, FASLG, MCEMP1, COL4A4, ENO1, BRD1, GP5, ZP3, SERPIND1, NCAM1, ATXN10, MUC16, GABRA4, POSTN, MAEA, SHH, DDX53, PRKG1, PAEP, RICTOR, IL6, FKBP14, CCL26, AIDA, GIP, TGFA, ITIH4, PCSK7, and RARRES1.

In various embodiments, the protein biomarkers further comprise each of PRAME, KIRREL1, KIF22, SPINT1, FGA, C1QTNF9, KIR2DS4, MMP9, NEXN, FCN1, MFGE8, ZNRD2, PDGFRB, HS6ST1, DUSP3, CABP2, DNM3, FGL1, TOP1, CDCP1, RAB10, THSD1, FASLG, MCEMP1, COL4A4, ENO1, BRD1, GP5, ZP3, SERPIND1, NCAM1, ATXN10, MUC16, GABRA4, POSTN, MAEA, SHH, DDX53, PRKG1, PAEP, RICTOR, IL6, FKBP14, CCL26, AIDA, GIP, TGFA, ITIH4, PCSK7, and RARRES1.

In various embodiments, the protein biomarkers further comprise one or more of SLC27A4, IL6, DKKL1, MFAP3, STX7, SSBP1, AKR7L, UGDH, IGHMBP2, GBP4, RBPMS, ST6GAL1, LILRA5, LILRA2, SOWAHA, ACADSB, CAMLG, CRTAC1, SUSD1, IL6, KLK10, GRSF1, MFAP4, NMT1, CNTN3, IL36A, EHD3, MAPT, AGBL2, ERN1, POMC, PDIA4, LGMN, EPHA10, PCBP2, PTGR1, GIT1, TREML1, GALNT2, TDGF1, INSR, OSCAR, MMP10, MRPL24, EIF1AX, AHNAK2, TP53, GBA, LRRC38, CLEC12A, TPT1, PPP1CC, BPIFB1, CFC1, SIGLEC9, CALY, OSM, ADAMTS1, OSMR, TYMP, GPR37, CLEC7A, SMAD5, SFTPA2, CTSS, HNMT, BATF, CCL19, SHC1, CST7, S100A12, ASAH2, PPIB, LYPD3, APOL1, AFM, SSC4D, FGF7, TDRKH, SCG2, ENPP2, PRKAR1A, FAM3D, GADD45GIP1, SEMA4D, PPP1R14A, EGF, NTF4, SERPING1, COX6B1, NECAP2, TFF1, IDI2, TJP3, CA14, PZP, PLIN1, ERBB4, TBC1D23, CRISP3, IFI30, ITIH1, C9, LAP3, PDIA5, ENDOU, FLT3LG, VNN2, MILR1, SDC1, CEACAM18, FHIP2A, CEACAM5, F11, WFIKKN2, USO1, CD40LG, GSTT2B, DUSP29, ATXN2L, IL6, RRM2, FGF23, ARHGAP30, SERPINA3, CXCL13, MMP8, NUDC, ENOPH1, NEK7, MAN1A2, ASAH1, STX5, IZUMO1, SERPINC1, IL9, PVALB, GZMH, FGF16, TFF2, WASF1, TMEM106A, GP2, PLXNA4, GNE, LGALS8, AOC1, FLRT2, CHCHD6, RNF43, TPD52L2, CSDE1, GPD1, PLA2G4A, LRIG1, NGF, RAB27B, VAT1, NUDT16, TRAF3IP2, MARCO, UMOD, PIK3AP1, MEGF11, NEDD4L, PKD2, CEBPB, RILPL2, IL3, RGCC, SARG, SMAD2, CTSH, KLKB1, ERP44, SULT2A1, SORD, IFNAR1, KLK11, TOMM20, C3, ADRA2A, NCK2, KIRREL2, CACNB3, SKAP2, CEACAM6, DNAJC21, PROS1, NRCAM, NPY, FYB1, RAB2B, MANF, MECR, LPA, DAAM1, DCTD, FXYD5, CRELD1, PLEKHO1, TINAGL1, ZBTB16, PROK1, MAP2K1, DAPP1, DSG4, PPP1R9B, RILP, EIF4G1, SESTD1, KIFBP, HGS, CD14, ANKMY2, WNT9A, CA13, GP1BB, CLIP2, BANK1, WDR46, HSPB1, CSF2, SNCA, RRAS, PRTFDC1, RBPMS2, LARP1, KAZN, CLSPN, RHOC, PPT1, DPEP2, METAP1D, STK11, CFH, PDE5A, MRC1, BIN2, IL17A, PXDNL, GP6, EPO, MAP3K5, MCEE, DDHD2, PHLDB2, NECTIN1, CCDC50, GKN1, MPIG6B, CBLIF, SYTL4, SSH3, PDZD2, SULTIA1, DLG4, HPCAL1, ICA1, GDF15, CD160, APPL2, GRN, IL17RA, CDC42BPB, C4BPB, DAG1, CMIP, KYNU, NUMB, PPY, PPIF, CFI, DTD1, LDLRAP1, FGF9, STXBP1, CMC1, GOPC, SMTN, PTPN6, L3HYPDH, PDAP1, LPP, THTPA, XG, AGRP, RAB11FIP3, F11R, BCR, LONP1, BNIP3L, SELP, GYS1, MGLL, PDLIM5, MESD, DNPEP, SRC, PMVK, ITPRIP, CD69, CALCOCO1, PAFAH2, GIPC3, SNAP23, STAT5B, RSPO3, AKT1S1, SNAP29, CASP2, AKT2, NELL1, MCTS1, TIA1, SCRG1, CIRBP, SEMA3F, SOX2, NRGN, PSTPIP2, ISM2, EHBP1, VTA1, and DUT.

In various embodiments, the predictive model comprises a elastic net regression model, and the predictive model achieves an area under a curve (AUC) value of at least 0.79. In various embodiments, the predictive model comprises a support vector machine, and the predictive model achieves an area under a curve (AUC) value of at least 0.81. In various embodiments, the predictive model comprises a random forest model, and the predictive model achieves an area under a curve (AUC) value of at least 0.71. In various embodiments, the predictive model comprises a XGBoost model, and the predictive model achieves an area under a curve (AUC) value of at least 0.70.

In various embodiments, the cancer is lung cancer. In various embodiments, the risk of cancer is a level of risk of the subject developing cancer within 1 year, within 2 years, within 3 years, within 4 years, within 5 years, within 6 years, within 7 years, within 8 years, within 9 years, or within 10 years. In various embodiments, the risk of cancer is a presence or absence of cancer. In various embodiments, the dataset is derived from a test sample obtained from the subject. In various embodiments, the test sample is a blood, serum or plasma sample. In various embodiments, obtaining or having obtained the dataset comprises performing one or more assays. In various embodiments, performing the one or more assays comprises performing an immunoassay to determine the expression levels of the plurality of biomarkers. In various embodiments, the immunoassay is a Proximity Extension Assay (PEA) or LUMINEX xMAP Multiplex Assay. In various embodiments, the dataset comprises plasma proteomics data. In various embodiments, methods disclosed herein further comprise: selecting a therapy for providing to the subject based on the prediction of cancer.

Additionally disclosed herein is a non-transitory computer readable medium comprising instructions that, when executed by a processor, cause the processor to: obtain or have obtained a dataset derived from the subject comprising quantitative levels of a plurality of biomarkers, wherein the plurality of biomarkers comprises protein biomarkers comprising two or more of TGFA, MMP12, TNFRSF13B, TNFSF14, and MASP1, and generate a prediction of risk of cancer for the subject by applying a predictive model to the quantitative values of the plurality of biomarkers.

In various embodiments, the protein biomarkers comprise three or more of TGFA, MMP12, TNFRSF13B, TNFSF14, and MASP1.

In various embodiments, the protein biomarkers comprise four or more of TGFA, MMP12, TNFRSF13B, TNFSF14, and MASP1.

In various embodiments, the protein biomarkers comprise each of TGFA, MMP12, TNFRSF13B, TNFSF14, and MASP1.

In various embodiments, the protein biomarkers further comprise one or more of THBS2, GDNF, FLT1, FXYD5, CST5, ARNT, CDCP1, CCL20, FLT3LG, CLEC7A, PRKCQ, SCGN, IL5, NPY, and S100A16.

In various embodiments, the protein biomarkers further comprise five or more of THBS2, GDNF, FLT1, FXYD5, CST5, ARNT, CDCP1, CCL20, FLT3LG, CLEC7A, PRKCQ, SCGN, IL5, NPY, and S100A16.

In various embodiments, the protein biomarkers further comprise ten or more of THBS2, GDNF, FLT1, FXYD5, CST5, ARNT, CDCP1, CCL20, FLT3LG, CLEC7A, PRKCQ, SCGN, IL5, NPY, and S100A16.

In various embodiments, the protein biomarkers further comprise each of THBS2, GDNF, FLT1, FXYD5, CST5, ARNT, CDCP1, CCL20, FLT3LG, CLEC7A, PRKCQ, SCGN, IL5, NPY, and S100A16.

In various embodiments, the protein biomarkers further comprise one or more, five or more, or each of IL1B, CD84, STC1, PRDX3, LAP3, GAMT, CASP2, ITGA6, DECR1, and YTHDF3.

In various embodiments, the protein biomarkers further comprise one or more of IL1B, CD84, STC1, PRDX3, LAP3, GAMT, CASP2, ITGA6, DECR1, and YTHDF3.

In various embodiments, the protein biomarkers further comprise five or more of IL1B, CD84, STC1, PRDX3, LAP3, GAMT, CASP2, ITGA6, DECR1, and YTHDF3.

In various embodiments, the protein biomarkers further comprise each of IL1B, CD84, STC1, PRDX3, LAP3, GAMT, CASP2, ITGA6, DECR1, and YTHDF3.

In various embodiments, the predictive model comprises an elastic net regression model, and the predictive model achieves an area under a curve (AUC) value of at least 0.65. In various embodiments, the predictive model comprises a support vector machine, and the predictive model achieves an area under a curve (AUC) value of at least 0.70. In various embodiments, the predictive model comprises a random forest model, and the predictive model achieves an area under a curve (AUC) value of at least 0.67. In various embodiments, the predictive model comprises a XGBoost model, and the predictive model achieves an area under a curve (AUC) value of at least 0.68.

Additionally disclosed herein is a non-transitory computer readable medium comprising instructions that, when executed by a processor, cause the processor to: obtain or have obtained a dataset derived from the subject comprising quantitative levels of a plurality of biomarkers, wherein the plurality of biomarkers comprises protein biomarkers comprising two or more of CEACAM5, TOP1, NCAM1, SCGB3A2, and CALY, and generate a prediction of risk of cancer for the subject by applying a predictive model to the quantitative values of the plurality of biomarkers.

In various embodiments, the protein biomarkers comprise three or more of CEACAM5, TOP1, NCAM1, SCGB3A2, and CALY.

In various embodiments, the protein biomarkers comprise four or more of CEACAM5, TOP1, NCAM1, SCGB3A2, and CALY.

In various embodiments, the protein biomarkers comprise each of CEACAM5, TOP1, NCAM1, SCGB3A2, and CALY.

In various embodiments, the protein biomarkers further comprise one or more of TGFBI, CABP2, ENPP6, KRT14, HEPACAM2, TMEM25, SGSH, MFAP3L, TNFSF14, CD3D, TMED4, ZP3, MMP12, GCG, and AFM.

In various embodiments, the protein biomarkers further comprise five or more of TGFBI, CABP2, ENPP6, KRT14, HEPACAM2, TMEM25, SGSH, MFAP3L, TNFSF14, CD3D, TMED4, ZP3, MMP12, GCG, and AFM.

In various embodiments, the protein biomarkers further comprise ten or more of TGFBI, CABP2, ENPP6, KRT14, HEPACAM2, TMEM25, SGSH, MFAP3L, TNFSF14, CD3D, TMED4, ZP3, MMP12, GCG, and AFM.

In various embodiments, the protein biomarkers further comprise each of TGFBI, CABP2, ENPP6, KRT14, HEPACAM2, TMEM25, SGSH, MFAP3L, TNFSF14, CD3D, TMED4, ZP3, MMP12, GCG, and AFM.

In various embodiments, the protein biomarkers further comprise one or more of SPINT1, LILRA4, FLT3LG, AGBL2, PAEP, SCGB3A1, LRFN2, TJP3, FGF7, LRIG1, CA14, CEACAM18, CST1, ANXA10, CDCP1, GPC5, OSCAR, CEACAM6, CD2, SNCG, GPR37, SEPTIN3, RAB10, DKK4, DKKL1, SOST, CSF3, VWA5A, TSPAN7, and PAK4.

In various embodiments, the protein biomarkers further comprise five or more of SPINT1, LILRA4, FLT3LG, AGBL2, PAEP, SCGB3A1, LRFN2, TJP3, FGF7, LRIG1, CA14, CEACAM18, CST1, ANXA10, CDCP1, GPC5, OSCAR, CEACAM6, CD2, SNCG, GPR37, SEPTIN3, RAB10, DKK4, DKKL1, SOST, CSF3, VWA5A, TSPAN7, and PAK4.

In various embodiments, the protein biomarkers further comprise ten or more of SPINT1, LILRA4, FLT3LG, AGBL2, PAEP, SCGB3A1, LRFN2, TJP3, FGF7, LRIG1, CA14, CEACAM18, CST1, ANXA10, CDCP1, GPC5, OSCAR, CEACAM6, CD2, SNCG, GPR37, SEPTIN3, RAB10, DKK4, DKKL1, SOST, CSF3, VWA5A, TSPAN7, and PAK4.

In various embodiments, the protein biomarkers further comprise twenty or more of SPINT1, LILRA4, FLT3LG, AGBL2, PAEP, SCGB3A1, LRFN2, TJP3, FGF7, LRIG1, CA14, CEACAM18, CST1, ANXA10, CDCP1, GPC5, OSCAR, CEACAM6, CD2, SNCG, GPR37, SEPTIN3, RAB10, DKK4, DKKL1, SOST, CSF3, VWA5A, TSPAN7, and PAK4.

In various embodiments, the protein biomarkers further comprise each of SPINT1, LILRA4, FLT3LG, AGBL2, PAEP, SCGB3A1, LRFN2, TJP3, FGF7, LRIG1, CA14, CEACAM18, CST1, ANXA10, CDCP1, GPC5, OSCAR, CEACAM6, CD2, SNCG, GPR37, SEPTIN3, RAB10, DKK4, DKKL1, SOST, CSF3, VWA5A, TSPAN7, and PAK4.

In various embodiments, the protein biomarkers further comprise one or more of BPIFB1, SIGLEC9, ZNRD2, PM20D1, TK1, RPS10, PMCH, RNF43, MEP1B, BGN, NELL1, CD101, LRP2BP, PRSS53, MFGE8, THSD1, CKMT1A, MEPE, APOL1, RBPMS, MARCO, KLRC1, FGFBP2, TPSG1, SELENOP, CLEC7A, UPK3BL1, HS6ST1, ENDOU, IL12RB2, CYB5A, GKN1, NRTN, CCL26, CRNN, PINLYP, LAIR2, BAG3, SCPEP1, RIPK4, CTSE, TMOD4, SFTPA1, SEMA4D, IL17C, GFRA3, DPEP2, EDEM2, CD84, and KIRREL2.

In various embodiments, the protein biomarkers further comprise five or more of BPIFB1, SIGLEC9, ZNRD2, PM20D1, TK1, RPS10, PMCH, RNF43, MEP1B, BGN, NELL1, CD101, LRP2BP, PRSS53, MFGE8, THSD1, CKMT1A, MEPE, APOL1, RBPMS, MARCO, KLRC1, FGFBP2, TPSG1, SELENOP, CLEC7A, UPK3BL1, HS6ST1, ENDOU, IL12RB2, CYB5A, GKN1, NRTN, CCL26, CRNN, PINLYP, LAIR2, BAG3, SCPEP1, RIPK4, CTSE, TMOD4, SFTPA1, SEMA4D, IL17C, GFRA3, DPEP2, EDEM2, CD84, and KIRREL2.

In various embodiments, the protein biomarkers further comprise ten or more of BPIFB1, SIGLEC9, ZNRD2, PM20D1, TK1, RPS10, PMCH, RNF43, MEP1B, BGN, NELL1, CD101, LRP2BP, PRSS53, MFGE8, THSD1, CKMT1A, MEPE, APOL1, RBPMS, MARCO, KLRC1, FGFBP2, TPSG1, SELENOP, CLEC7A, UPK3BL1, HS6ST1, ENDOU, IL12RB2, CYB5A, GKN1, NRTN, CCL26, CRNN, PINLYP, LAIR2, BAG3, SCPEP1, RIPK4, CTSE, TMOD4, SFTPA1, SEMA4D, IL17C, GFRA3, DPEP2, EDEM2, CD84, and KIRREL2.

In various embodiments, the protein biomarkers further comprise twenty or more of BPIFB1, SIGLEC9, ZNRD2, PM20D1, TK1, RPS10, PMCH, RNF43, MEP1B, BGN, NELL1, CD101, LRP2BP, PRSS53, MFGE8, THSD1, CKMT1A, MEPE, APOL1, RBPMS, MARCO, KLRC1, FGFBP2, TPSG1, SELENOP, CLEC7A, UPK3BL1, HS6ST1, ENDOU, IL12RB2, CYB5A, GKN1, NRTN, CCL26, CRNN, PINLYP, LAIR2, BAG3, SCPEP1, RIPK4, CTSE, TMOD4, SFTPA1, SEMA4D, IL17C, GFRA3, DPEP2, EDEM2, CD84, and KIRREL2.

In various embodiments, the protein biomarkers further comprise thirty or more of BPIFB1, SIGLEC9, ZNRD2, PM20D1, TK1, RPS10, PMCH, RNF43, MEP1B, BGN, NELL1, CD101, LRP2BP, PRSS53, MFGE8, THSD1, CKMT1A, MEPE, APOL1, RBPMS, MARCO, KLRC1, FGFBP2, TPSG1, SELENOP, CLEC7A, UPK3BL1, HS6ST1, ENDOU, IL12RB2, CYB5A, GKN1, NRTN, CCL26, CRNN, PINLYP, LAIR2, BAG3, SCPEP1, RIPK4, CTSE, TMOD4, SFTPA1, SEMA4D, IL17C, GFRA3, DPEP2, EDEM2, CD84, and KIRREL2.

In various embodiments, the protein biomarkers further comprise forty or more of BPIFB1, SIGLEC9, ZNRD2, PM20D1, TK1, RPS10, PMCH, RNF43, MEP1B, BGN, NELL1, CD101, LRP2BP, PRSS53, MFGE8, THSD1, CKMT1A, MEPE, APOL1, RBPMS, MARCO, KLRC1, FGFBP2, TPSG1, SELENOP, CLEC7A, UPK3BL1, HS6ST1, ENDOU, IL12RB2, CYB5A, GKN1, NRTN, CCL26, CRNN, PINLYP, LAIR2, BAG3, SCPEP1, RIPK4, CTSE, TMOD4, SFTPA1, SEMA4D, IL17C, GFRA3, DPEP2, EDEM2, CD84, and KIRREL2.

In various embodiments, the protein biomarkers further comprise each of BPIFB1, SIGLEC9, ZNRD2, PM20D1, TK1, RPS10, PMCH, RNF43, MEP1B, BGN, NELL1, CD101, LRP2BP, PRSS53, MFGE8, THSD1, CKMT1A, MEPE, APOL1, RBPMS, MARCO, KLRC1, FGFBP2, TPSG1, SELENOP, CLEC7A, UPK3BL1, HS6ST1, ENDOU, IL12RB2, CYB5A, GKN1, NRTN, CCL26, CRNN, PINLYP, LAIR2, BAG3, SCPEP1, RIPK4, CTSE, TMOD4, SFTPA1, SEMA4D, IL17C, GFRA3, DPEP2, EDEM2, CD84, and KIRREL2.

In various embodiments, the protein biomarkers further comprise one or more of NECTIN1, CBLN1, NTF3, PYY, XG, NPY, CCL20, SIL1, PLB1, DUSP29, UMOD, ATXN2L, LEO1, PROS1, EDDM3B, ENO3, DCBLD2, MMP9, KIF22, DENND2B, C1RL, PVALB, CXCL8, PPY, CCN1, KLK10, RRAS, SCN3B, BPIFB2, ITGAL, DDX1, MEGF11, NOP56, NTF4, HNMT, IL9, SCRIB, UXS1, MEP1A, ACTN2, NECAP2, CLEC1OA, DDX53, SV2A, ATXN10, PI16, KCNH2, TNR, PDGFRB, SERPINA4, CDC27, MICALL2, CD28, BRK1, SLC16A1, DSCAM, PBXIP1, MATN3, SFTPA2, PTTG1, ASAH2, SCG2, PTGR1, GBA, PTPRZ1, ERN1, LECT2, SCGN, HLA-DRA, IL5RA, LRPAP1, CXCL13, NEXN, CD248, KYNU, ADAMTS15, WFIKKN2, CLEC14A, FZD10, PROC, LY9, LRP2, CX3CL1, RNASET2, CTSS, MCEMP1, COMP, SIGLEC6, CCL24, AOC1, PLXNB3, TMPRSS15, FCAR, SCIN, IFI30, KIRREL1, FXYD5, S100A16, LILRA5, CLSPN, AHNAK2, CTLA4, INSL5, WDR46, CST5, PHLDB2, TREML2, GUCA2A, PFDN2, PDIA4, LAMA1, SLAMF7, RGS8, IL6, PSG1, PZP, RRM2, GFRAL, AIF1L, LGMN, C1QTNF9, TSPAN1, DLL4, CRELD2, SCARF1, FGF9, JAM3, LPP, HSPB1, PPT1, PPIF, TRPV3, APOA4, LYSMD3, TGFA, ATP6V1D, LRRC38, CTAG1A, TINAGL1, POLR2A, EDIL3, LAP3, SORD, ARHGAP30, CSPG4, ART3, GADD45GIP1, SLURP1, LILRA2, GZMH, FKBP7, SLC27A4, CALCB, GIT1, CTSO, PCBD1, CSF3R, EIF1AX, CSPG5, CD93, ADAMTSL5, ISM2, CPE, WFDC1, VWC2, SPINK5, BTN1A1, DPT, FCN1, AIF1, GPC1, FAP, CLNS1A, CFC1, FASLG, NCS1, PRKAR1A, RCOR1, SLITRK2, SPARCL1, HSPB6, TNFRSF12A, IL6, SERPIND1, CEBPB, CASC3, AMPD3, YTHDF3, AAMDC, STX7, AGRP, ICA1, CHCHD6, IGSF21, VSTM1, PCDH7, VNN2, GP6, ITGAV, CD40LG, GIP, MB, TPD52L2, HPSE, GRIN2B, TREML1, C3, TNFRSF17, IL6, CD226, PALM, FKBP14, RBPMS2, CLEC6A, DAAM1, FAM3D, WASF1, HS1BP3, NOS3, POF1B, PLXNA4, MITD1, ERMAP, SYAP1, LRRC59, CNTN2, RAB2B, PENK, MCAM, EIF2S2, EGF, PTPN6, NID2, EHD3, IGFBP6, LMOD1, PAGR1, CD300C, SKAP2, PRKG1, SYTL4, GYS1, CASP3, PILRA, CD69, CCN5, PCBP2, LMOD1, PDIA5, PCSK7, SCARA5, METAP1D, ADGRB3, MPIG6B, NUMB, L3HYPDH, DENR, AGRN, COX6B1, JAM2, TIA1, CACYBP, SEMA6C, VAT1, SUSD1, RSPO3, TWF2, BOLA1, OXCT1, ITGA6, BST2, F2R, PILRB, RTBDN, ENOX2, DOK1, VASH1, DTD1, DDHD2, TBC1D23, GLRX5, CDNF, SIRPB1, NMT1, STK11, RPL14, PSTPIP2, FHIT, CLMP, LMOD1, ERP29, BECN1, CD38, YAP1, CA13, CRKL, PPP1R9B, FLI1, CMC1, CDC37, ARHGAP45, PDAP1, NUDC, CLEC1B, USO1, SNAP23, HGS, FUS, PIK3AP1, F11R, TBC1D17, ITPA, IL1B, ENO1, THTPA, SAFB2, JPT2, GIMAP7, NIT2, RILPL2, PRTFDC1, TADA3, TOMM20, HPCAL1, LONP1, CALCOCO1, ATRAID, TYMP, TNFRSF19, DNPEP, NRGN, STK4, SSNA1, CRYGD, LZTFL1, SNAP29, PDLIM5, CASP2, MANF, BACH1, DAPP1, AKR1B1, EREG, DAG1, HSBP1, DUT, AKT2, PLA2G4A, TXLNA, PIKFYVE, FYB1, CSDE1, RHOC, HNRNPK, DCTD, SCRG1, LACTB2, RGCC, GIMAP8, GRHPR, SNX5, NCK2, EIF4G1, BNIP3L, ACOT13, MECR, MAP2K6, SEC31A, MGLL, MESD, NUDT16, SULTIA1, GOPC, VTA1, PDLIM7, ANXA2, GGACT, PMVK, USP8, SNCA, CAMSAP1, HEXIMI, SHMT1, LGALS8, APPL2, MAP2K1, EHBP1, MAP4K5, PDE5A, HARS1, SRC, TACC3, and RAB27B.

In various embodiments, the predictive model comprises a elastic net regression model, and the predictive model achieves an area under a curve (AUC) value of at least 0.85. In various embodiments, the predictive model comprises a support vector machine, and the predictive model achieves an area under a curve (AUC) value of at least 0.84. In various embodiments, the predictive model comprises a random forest model, and the predictive model achieves an area under a curve (AUC) value of at least 0.72. In various embodiments, the predictive model comprises a XGBoost model, and the predictive model achieves an area under a curve (AUC) value of at least 0.73.

Additionally disclosed herein is a non-transitory computer readable medium comprising instructions that, when executed by a processor, cause the processor to: obtain or have obtained a dataset derived from the subject comprising quantitative levels of a plurality of biomarkers, wherein the plurality of biomarkers comprises protein biomarkers comprising two or more of VWA5A, ENPP6, TMEM25, ALDH2, and LEO1, and generate a prediction of risk of cancer for the subject by applying a predictive model to the quantitative values of the plurality of biomarkers.

In various embodiments, the protein biomarkers comprise three or more of VWA5A, ENPP6, TMEM25, ALDH2, and LEO1.

In various embodiments, the protein biomarkers comprise four or more of VWA5A, ENPP6, TMEM25, ALDH2, and LEO1.

In various embodiments, the protein biomarkers comprise each of VWA5A, ENPP6, TMEM25, ALDH2, and LEO1.

In various embodiments, the protein biomarkers further comprise one or more of GAMT, TPSG1, ANK2, SCT, TSPAN7, GPC5, PGLYRP1, PAK4, TNFSF14, CLEC6A, TMPRSS15, PMCH, KRT14, SFTPA1, and LRFN2.

In various embodiments, the protein biomarkers further comprise five or more of GAMT, TPSG1, ANK2, SCT, TSPAN7, GPC5, PGLYRP1, PAK4, TNFSF14, CLEC6A, TMPRSS15, PMCH, KRT14, SFTPA1, and LRFN2.

In various embodiments, the protein biomarkers further comprise ten or more of GAMT, TPSG1, ANK2, SCT, TSPAN7, GPC5, PGLYRP1, PAK4, TNFSF14, CLEC6A, TMPRSS15, PMCH, KRT14, SFTPA1, and LRFN2.

In various embodiments, the protein biomarkers further comprise each of GAMT, TPSG1, ANK2, SCT, TSPAN7, GPC5, PGLYRP1, PAK4, TNFSF14, CLEC6A, TMPRSS15, PMCH, KRT14, SFTPA1, and LRFN2.

In various embodiments, the protein biomarkers further comprise one or more of MMP12, TNPO1, GAST, CD3D, TK1, DLGAP5, SCGN, CCL24, PSG1, CLU, CFB, LBP, CRYM, LAIR2, TCN2, SV2A, CRHBP, C5, SCGB3A2, ANXA10, GCG, RPGR, PAPPA, FZD8, CSPG5, BRK1, OXT, FDX1, ENPEP, and LRG1.

In various embodiments, the protein biomarkers further comprise five or more of MMP12, TNPO1, GAST, CD3D, TK1, DLGAP5, SCGN, CCL24, PSG1, CLU, CFB, LBP, CRYM, LAIR2, TCN2, SV2A, CRHBP, C5, SCGB3A2, ANXA10, GCG, RPGR, PAPPA, FZD8, CSPG5, BRK1, OXT, FDX1, ENPEP, and LRG1.

In various embodiments, the protein biomarkers further comprise ten or more of MMP12, TNPO1, GAST, CD3D, TK1, DLGAP5, SCGN, CCL24, PSG1, CLU, CFB, LBP, CRYM, LAIR2, TCN2, SV2A, CRHBP, C5, SCGB3A2, ANXA10, GCG, RPGR, PAPPA, FZD8, CSPG5, BRK1, OXT, FDX1, ENPEP, and LRG1.

In various embodiments, the protein biomarkers further comprise twenty or more of MMP12, TNPO1, GAST, CD3D, TK1, DLGAP5, SCGN, CCL24, PSG1, CLU, CFB, LBP, CRYM, LAIR2, TCN2, SV2A, CRHBP, C5, SCGB3A2, ANXA10, GCG, RPGR, PAPPA, FZD8, CSPG5, BRK1, OXT, FDX1, ENPEP, and LRG1.

In various embodiments, the protein biomarkers further comprise each of MMP12, TNPO1, GAST, CD3D, TK1, DLGAP5, SCGN, CCL24, PSG1, CLU, CFB, LBP, CRYM, LAIR2, TCN2, SV2A, CRHBP, C5, SCGB3A2, ANXA10, GCG, RPGR, PAPPA, FZD8, CSPG5, BRK1, OXT, FDX1, ENPEP, and LRG1.

In various embodiments, the protein biomarkers further comprise one or more of PRAME, KIRREL1, KIF22, SPINT1, FGA, C1QTNF9, KIR2DS4, MMP9, NEXN, FCN1, MFGE8, ZNRD2, PDGFRB, HS6ST1, DUSP3, CABP2, DNM3, FGL1, TOP1, CDCP1, RAB10, THSD1, FASLG, MCEMP1, COL4A4, ENO1, BRD1, GP5, ZP3, SERPIND1, NCAM1, ATXN10, MUC16, GABRA4, POSTN, MAEA, SHH, DDX53, PRKG1, PAEP, RICTOR, IL6, FKBP14, CCL26, AIDA, GIP, TGFA, ITIH4, PCSK7, and RARRES1.

In various embodiments, the protein biomarkers further comprise five or more of PRAME, KIRREL1, KIF22, SPINT1, FGA, C1QTNF9, KIR2DS4, MMP9, NEXN, FCN1, MFGE8, ZNRD2, PDGFRB, HS6ST1, DUSP3, CABP2, DNM3, FGL1, TOP1, CDCP1, RAB10, THSD1, FASLG, MCEMP1, COL4A4, ENO1, BRD1, GP5, ZP3, SERPIND1, NCAM1, ATXN10, MUC16, GABRA4, POSTN, MAEA, SHH, DDX53, PRKG1, PAEP, RICTOR, IL6, FKBP14, CCL26, AIDA, GIP, TGFA, ITIH4, PCSK7, and RARRES1.

In various embodiments, the protein biomarkers further comprise ten or more of PRAME, KIRREL1, KIF22, SPINT1, FGA, C1QTNF9, KIR2DS4, MMP9, NEXN, FCN1, MFGE8, ZNRD2, PDGFRB, HS6ST1, DUSP3, CABP2, DNM3, FGL1, TOP1, CDCP1, RAB10, THSD1, FASLG, MCEMP1, COL4A4, ENO1, BRD1, GP5, ZP3, SERPIND1, NCAM1, ATXN10, MUC16, GABRA4, POSTN, MAEA, SHH, DDX53, PRKG1, PAEP, RICTOR, IL6, FKBP14, CCL26, AIDA, GIP, TGFA, ITIH4, PCSK7, and RARRES1.

In various embodiments, the protein biomarkers further comprise twenty or more of PRAME, KIRREL1, KIF22, SPINT1, FGA, C1QTNF9, KIR2DS4, MMP9, NEXN, FCN1, MFGE8, ZNRD2, PDGFRB, HS6ST1, DUSP3, CABP2, DNM3, FGL1, TOP1, CDCP1, RAB10, THSD1, FASLG, MCEMP1, COL4A4, ENO1, BRD1, GP5, ZP3, SERPIND1, NCAM1, ATXN10, MUC16, GABRA4, POSTN, MAEA, SHH, DDX53, PRKG1, PAEP, RICTOR, IL6, FKBP14, CCL26, AIDA, GIP, TGFA, ITIH4, PCSK7, and RARRES1.

In various embodiments, the protein biomarkers further comprise thirty or more of PRAME, KIRREL1, KIF22, SPINT1, FGA, C1QTNF9, KIR2DS4, MMP9, NEXN, FCN1, MFGE8, ZNRD2, PDGFRB, HS6ST1, DUSP3, CABP2, DNM3, FGL1, TOP1, CDCP1, RAB10, THSD1, FASLG, MCEMP1, COL4A4, ENO1, BRD1, GP5, ZP3, SERPIND1, NCAM1, ATXN10, MUC16, GABRA4, POSTN, MAEA, SHH, DDX53, PRKG1, PAEP, RICTOR, IL6, FKBP14, CCL26, AIDA, GIP, TGFA, ITIH4, PCSK7, and RARRES1.

In various embodiments, the protein biomarkers further comprise forty or more of PRAME, KIRREL1, KIF22, SPINT1, FGA, C1QTNF9, KIR2DS4, MMP9, NEXN, FCN1, MFGE8, ZNRD2, PDGFRB, HS6ST1, DUSP3, CABP2, DNM3, FGL1, TOP1, CDCP1, RAB10, THSD1, FASLG, MCEMP1, COL4A4, ENO1, BRD1, GP5, ZP3, SERPIND1, NCAM1, ATXN10, MUC16, GABRA4, POSTN, MAEA, SHH, DDX53, PRKG1, PAEP, RICTOR, IL6, FKBP14, CCL26, AIDA, GIP, TGFA, ITIH4, PCSK7, and RARRES1.

In various embodiments, the protein biomarkers further comprise each of PRAME, KIRREL1, KIF22, SPINT1, FGA, C1QTNF9, KIR2DS4, MMP9, NEXN, FCN1, MFGE8, ZNRD2, PDGFRB, HS6ST1, DUSP3, CABP2, DNM3, FGL1, TOP1, CDCP1, RAB10, THSD1, FASLG, MCEMP1, COL4A4, ENO1, BRD1, GP5, ZP3, SERPIND1, NCAM1, ATXN10, MUC16, GABRA4, POSTN, MAEA, SHH, DDX53, PRKG1, PAEP, RICTOR, IL6, FKBP14, CCL26, AIDA, GIP, TGFA, ITIH4, PCSK7, and RARRES1.

In various embodiments, the protein biomarkers further comprise one or more of SLC27A4, IL6, DKKL1, MFAP3, STX7, SSBP1, AKR7L, UGDH, IGHMBP2, GBP4, RBPMS, ST6GAL1, LILRA5, LILRA2, SOWAHA, ACADSB, CAMLG, CRTAC1, SUSD1, IL6, KLK10, GRSF1, MFAP4, NMT1, CNTN3, IL36A, EHD3, MAPT, AGBL2, ERN1, POMC, PDIA4, LGMN, EPHA10, PCBP2, PTGR1, GIT1, TREML1, GALNT2, TDGF1, INSR, OSCAR, MMP10, MRPL24, EIF1AX, AHNAK2, TP53, GBA, LRRC38, CLEC12A, TPT1, PPP1CC, BPIFB1, CFC1, SIGLEC9, CALY, OSM, ADAMTS1, OSMR, TYMP, GPR37, CLEC7A, SMAD5, SFTPA2, CTSS, HNMT, BATF, CCL19, SHC1, CST7, S100A12, ASAH2, PPIB, LYPD3, APOL1, AFM, SSC4D, FGF7, TDRKH, SCG2, ENPP2, PRKAR1A, FAM3D, GADD45GIP1, SEMA4D, PPP1R14A, EGF, NTF4, SERPING1, COX6B1, NECAP2, TFF1, IDI2, TJP3, CA14, PZP, PLIN1, ERBB4, TBC1D23, CRISP3, IFI30, ITIH1, C9, LAP3, PDIA5, ENDOU, FLT3LG, VNN2, MILR1, SDC1, CEACAM18, FHIP2A, CEACAM5, F11, WFIKKN2, USO1, CD40LG, GSTT2B, DUSP29, ATXN2L, IL6, RRM2, FGF23, ARHGAP30, SERPINA3, CXCL13, MMP8, NUDC, ENOPH1, NEK7, MAN1A2, ASAH1, STX5, IZUMO1, SERPINC1, IL9, PVALB, GZMH, FGF16, TFF2, WASF1, TMEM106A, GP2, PLXNA4, GNE, LGALS8, AOC1, FLRT2, CHCHD6, RNF43, TPD52L2, CSDE1, GPD1, PLA2G4A, LRIG1, NGF, RAB27B, VAT1, NUDT16, TRAF3IP2, MARCO, UMOD, PIK3AP1, MEGF11, NEDD4L, PKD2, CEBPB, RILPL2, IL3, RGCC, SARG, SMAD2, CTSH, KLKB1, ERP44, SULT2A1, SORD, IFNAR1, KLK11, TOMM20, C3, ADRA2A, NCK2, KIRREL2, CACNB3, SKAP2, CEACAM6, DNAJC21, PROS1, NRCAM, NPY, FYB1, RAB2B, MANF, MECR, LPA, DAAM1, DCTD, FXYD5, CRELD1, PLEKHO1, TINAGL1, ZBTB16, PROK1, MAP2K1, DAPP1, DSG4, PPP1R9B, RILP, EIF4G1, SESTD1, KIFBP, HGS, CD14, ANKMY2, WNT9A, CA13, GP1BB, CLIP2, BANK1, WDR46, HSPB1, CSF2, SNCA, RRAS, PRTFDC1, RBPMS2, LARP1, KAZN, CLSPN, RHOC, PPT1, DPEP2, METAP1D, STK11, CFH, PDE5A, MRC1, BIN2, IL17A, PXDNL, GP6, EPO, MAP3K5, MCEE, DDHD2, PHLDB2, NECTIN1, CCDC50, GKN1, MPIG6B, CBLIF, SYTL4, SSH3, PDZD2, SULTIA1, DLG4, HPCAL1, ICA1, GDF15, CD160, APPL2, GRN, IL17RA, CDC42BPB, C4BPB, DAG1, CMIP, KYNU, NUMB, PPY, PPIF, CFI, DTD1, LDLRAP1, FGF9, STXBP1, CMC1, GOPC, SMTN, PTPN6, L3HYPDH, PDAP1, LPP, THTPA, XG, AGRP, RAB11FIP3, F11R, BCR, LONP1, BNIP3L, SELP, GYS1, MGLL, PDLIM5, MESD, DNPEP, SRC, PMVK, ITPRIP, CD69, CALCOCO1, PAFAH2, GIPC3, SNAP23, STAT5B, RSPO3, AKT1S1, SNAP29, CASP2, AKT2, NELL1, MCTS1, TIA1, SCRG1, CIRBP, SEMA3F, SOX2, NRGN, PSTPIP2, ISM2, EHBP1, VTA1, and DUT.

In various embodiments, the predictive model comprises an elastic net regression model, and the predictive model achieves an area under a curve (AUC) value of at least 0.79. In various embodiments, the predictive model comprises a support vector machine, and the predictive model achieves an area under a curve (AUC) value of at least 0.81. In various embodiments, the predictive model comprises a random forest model, and the predictive model achieves an area under a curve (AUC) value of at least 0.71. In various embodiments, the predictive model comprises a XGBoost model, and the predictive model achieves an area under a curve (AUC) value of at least 0.70.

In various embodiments, the cancer is lung cancer. In various embodiments, the risk of cancer is a level of risk of the subject developing cancer within 1 year, within 2 years, within 3 years, within 4 years, within 5 years, within 6 years, within 7 years, within 8 years, within 9 years, or within 10 years. In various embodiments, the risk of cancer is a presence or absence of cancer. In various embodiments, the dataset is derived from a test sample obtained from the subject. In various embodiments, the test sample is a blood, serum or plasma sample. In various embodiments, the dataset is obtained from having performed one or more assays. In various embodiments, the one or more assays comprises an immunoassay to determine the expression levels of the plurality of biomarkers. In various embodiments, the immunoassay is a Proximity Extension Assay (PEA) or LUMINEX xMAP Multiplex Assay. In various embodiments, the dataset comprises plasma proteomics data. In various embodiments, a therapy is selected for providing to the subject based on the prediction of cancer.

### EXAMPLES

Below are examples of specific embodiments for carrying out the present invention. The examples are offered for illustrative purposes only and are not intended to limit the scope of the present invention in any way. Efforts have been made to ensure accuracy with respect to numbers used (e.g., amounts, temperatures, etc.), but some experimental error and deviation should be allowed for.

In some scenarios as described herein, the proteins in Example 4 can be subsets of proteins described in Example 1 and/or identified in Tables 1-3 (e.g., 425 proteins for 1-3Y and 493 proteins for 1-5Y).

### Example 1: Study Methods

This study was performed using data and biospecimens collected as part of the Liverpool Lung Project (LLP) cohort, and were obtained following institutional review board approval, and patients provided written informed consent. Leveraging the Liverpool Lung Project (LLP), a unique 10-year observational cohort that followed subjects from healthy to lung cancer diagnoses, pre-diagnosis plasma proteomics were generated in a cross-sectional sub-cohort including 292 subjects e.g., with samples taken 1-5 years before their diagnosis, and a longitudinal sub-cohort including 246 samples from 144 subjects, e.g., taken 5-10 years before their diagnosis, 2-5 years before their diagnosis, and/or at time of their diagnosis.

In the study methods, plasma proteomics data were generated using two separate workflows or approaches. In one workflow (Example 2), 366 proteins were analyzed to develop predictive models incorporating 30 biomarkers (hereafter referred to as predictive models using the Olink® Target 96 platform). In another workflow (Examples 3 and 4), 2941 proteins were analyzed to develop predictive models for predicting future lung cancer development within 1-3 years and within 1-5 years. Such predictive models are hereafter referred to as predictive models using the Olink® Explore 3072 platform. Receiver operating characteristic (ROC) curves, area under curves (AUCs) (e.g., median AUC) from the models, and recursive feature elimination (RFE) using 5-fold cross validation repeated 5 times were reported.

For each approach or workflow, four machine learning algorithms (e.g., Elastic Net (“en”), Random Forest (“rf”), Support Vector Machine (“svm”), XGBoost (“xgb”)) were implemented to develop prediction models to predict cancer vs. healthy based on different biomarkers. Biomarkers for the Olink® Target 96 platform were selected based on differential expression between healthy and cancer subjects in “WP2” step (linear model, p<0.05). Biomarkers for the Olink® Explore 3072 platform were selected after performing differential expression on a random set of 50% of the dataset 1000 times, and significant proteins were defined as being differentially expressed (p<0.05) at least 100 times.

Tables 1-3 show the predictors that were included in the prediction models. Tables 1-3 further identify the rank of each protein biomarker in the corresponding workflow or model (e.g., “Olink Target 96 WP2 rank,” “1-5Y Rank,” or “1-3Y Rank”). Tables 1-3 further identify the biomarker name, pathway information, Biomarker symbol, Uniprot number, and/or protein name of each protein biomarker.

The proteins in Example 4 can be subsets of proteins described Tables 1-3 (e.g., 425 proteins for 1-3Y and 493 proteins for 1-5Y).

### Example 2: Example Results from Prediction Models Using Olink® Target 96 Platform

In this example, a prediction model including 30 protein biomarkers was constructed from the cross-sectional sub-cohort as described in Example 1 for predicting future lung cancer development within 1-5 years. Here, the prediction model was constructed using four separate machine learning algorithms (Elastic Net (“en”), Random Forest (“rf”), Support Vector Machine (“svm”), XGBoost (“xgb”)), followed by recursive feature elimination (RFE) from 5-fold cross-validation (CV) repeated for 5 times to reduce the total number of predictors in the model.

Here, the prediction model was constructed in accordance with the embodiment shown in FIG. 3. Thus, the prediction model analyzes biomarker levels and generates a cancer score that is informative for the overall prediction (e.g., presence or absence of cancer).

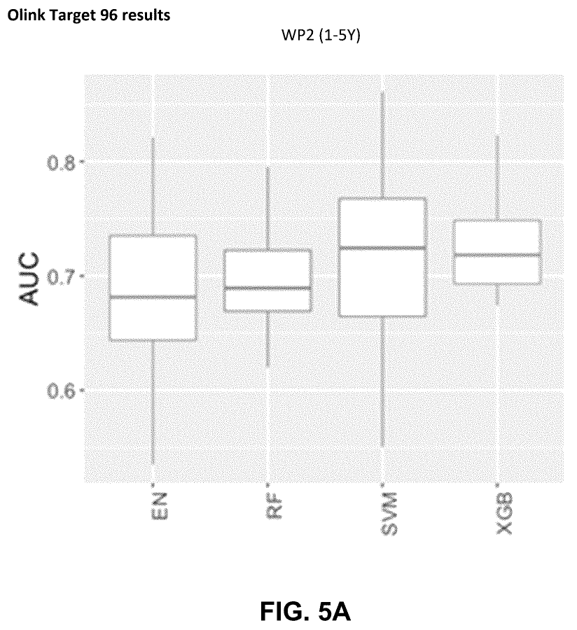

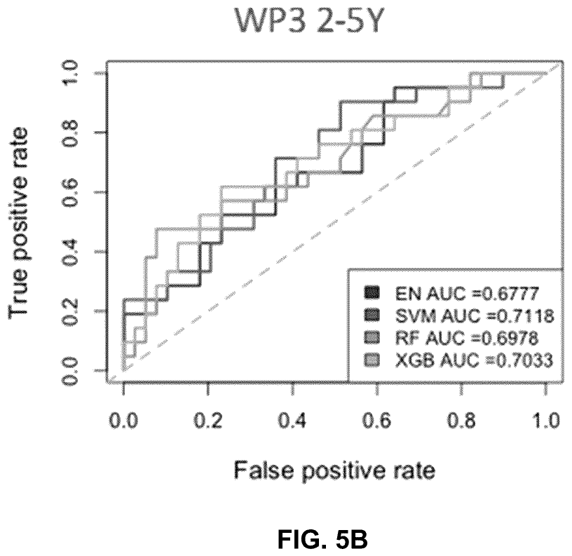

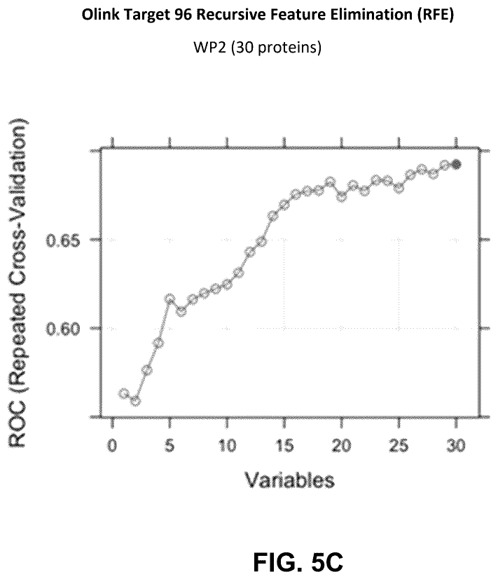

As shown in FIG. 5A, the four different prediction models successfully predicted future lung cancer development from 1-5 years before diagnosis with AUCs ranging from 0.68 to 0.74.

As shown in FIG. 5B and Table 4, in an independent validation set (longitudinal sub-cohort), the model predicted cancer development 2-5 years prior to diagnoses with AUCs ranging from 0.68 to 0.71.

FIG. 5C shows the performance of the predictive model (e.g., Random Forest) as a function of the number of predictors in the model, in accordance with the embodiment of the prediction model shown in FIG. 3. Beginning with the 30 initial protein biomarkers (30 biomarkers shown in Table 1), the performance of the predictive model was evaluated as protein biomarkers were iteratively removed via recursive feature elimination (RFE). For example, with the 30 initial protein biomarkers (indicated on the x-axis of FIG. 5C as “variables”), the predictive model achieved an AUC performance metric of nearly 0.7. As the number of protein biomarkers decreased, the predictive capacity of the model remained predictive. For example, at 20 protein biomarkers (which includes the biomarkers in Table 1 with corresponding “Olink Target 96 WP2 rank” between 1-20), the predictive model exhibited an AUC of ˜0.67. At 10 protein biomarkers (which includes the biomarkers in Table 1 with corresponding “Olink Target 96 WP2 rank” between 1-10), the predictive model exhibited an AUC of ˜0.63. At 5 protein biomarkers (which includes the biomarkers in Table 1 with corresponding “Olink Target 96 WP2 rank” between 1-5), the predictive model exhibited an AUC of ˜0.62.

### Example 3: Example Results from Prediction Models Using Olink® Explore 3072 Platform

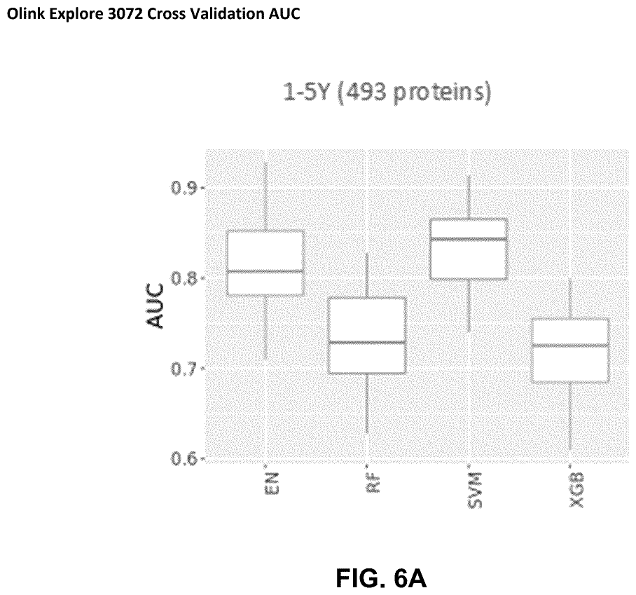

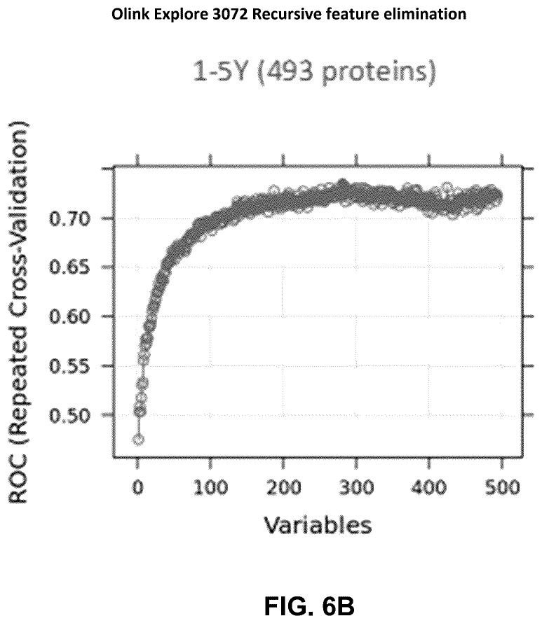

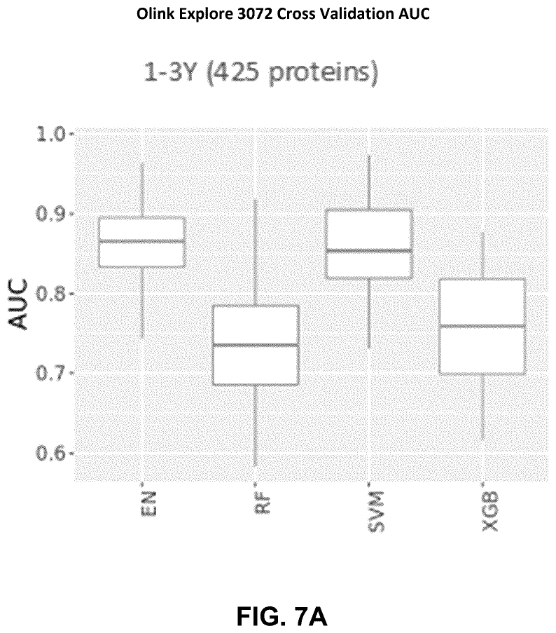

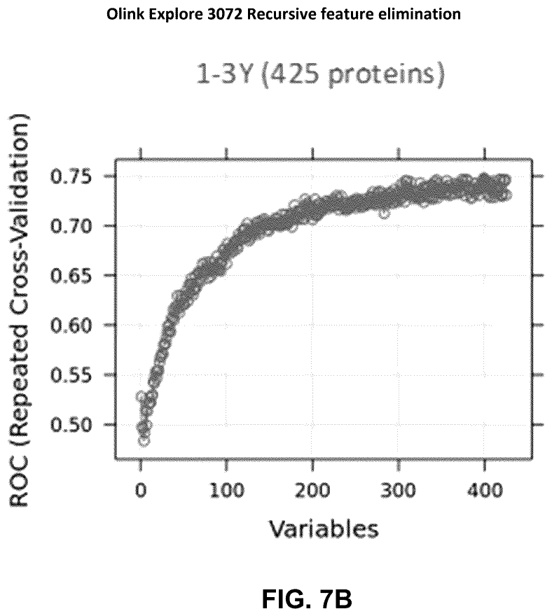

In this example, patient samples from the cross-sectional and longitudinal sub-cohorts were incorporated to construct a prediction model for predicting future lung cancer development within 1-5 year (“1-5Y”) (FIGS. 6A and 6B, and Table 5) and 1-3 year (“1-3Y”) (FIGS. 7A and 7B, and Table 6) before diagnosis. For 1-5Y before diagnosis, 493 protein biomarkers were derived. For 1-3Y before diagnosis, 425 protein biomarkers were derived.

Here, the prediction model was constructed using four separate machine learning algorithms (Elastic Net Regression (“en”), Random Forest (“rf”), Support Vector Machine (“svm”), XGBoost (“xgb”)), followed by recursive feature elimination (RFE) from 5-fold cross-validation (CV) repeated for 5 times to reduce the total number of predictors in the model.

Here, prediction models were constructed in accordance with the embodiment shown in FIG. 3. Thus, prediction models analyze biomarker levels and generate a cancer score that is informative for the overall prediction (e.g., future risk of cancer, or presence or absence of cancer).

As shown in FIG. 6A and Table 5, the four different prediction models successfully predicted future lung cancer development from 1-5 years before diagnosis with AUCs (e.g., median AUCs) ranging from 0.73 to 0.84.

Table 5 shows various AUC performance metrics, such as “Min.,” “1st. Qu.,” “Median,” “Mean,” “3rd. Qu,” “Max.” AUC from various “models” (e.g., logistic, svm, rv, xgb) or machine learning algorithms (e.g., “en,” “svm,” “rf,” or “xgb”) ranging from 0.60 to 0.93.

FIG. 6B shows the performance of the predictive model (e.g., Random Forest) as a function of the number of predictors in the model, in accordance with the embodiment of the prediction model shown in FIG. 3. Beginning with the 493 initial protein biomarkers (493 biomarkers shown in Table 2), the performance of the predictive model was evaluated as protein biomarkers were iteratively removed via RFE. For example, with the 493 initial protein biomarkers (indicated on the x-axis of FIG. 6B as “variables”), the predictive model achieved an AUC performance metric of nearly 0.73. As the number of protein biomarkers decreased, the predictive capacity of the model remained predictive. For example, at 100 protein biomarkers (which includes the biomarkers in Table 2 with corresponding “1-5Y rank” between 1-100), the predictive model exhibited an AUC of ˜0.70. At 10 protein biomarkers (which includes the biomarkers in Table 2 with corresponding “1-5Y rank” between 1-10), the predictive model exhibited an AUC of ˜0.57. At 5 protein biomarkers (which includes the biomarkers in Table 2 with corresponding “1-5Y rank” between 1-5), the predictive model exhibited an AUC of ˜0.53.

Table 6 shows various AUC model performance metrics, such as “Min.,” “1st. Qu.,” “Median,” “Mean,” “3rd. Qu,” “Max.” AUC from four different “models” (e.g., logistic, svm, rv, xgb) or machine learning algorithms (e.g., en, svm, rf, xgb) ranging from 0.58 to 0.99.

As shown in FIG. 7A and Table 6, the prediction models successfully predicted future lung cancer development from 1-3 years before diagnosis with AUCs (e.g., median AUCs) ranging from 0.74 to 0.87.

FIG. 7B shows the performance of the predictive model (e.g., Random Forest) as a function of the number of predictors in the model, in accordance with the embodiment of the prediction model shown in FIG. 3. Beginning with the 425 initial protein biomarkers (425 biomarkers shown in Table 3), the performance of the predictive model was evaluated as protein biomarkers were iteratively removed via RFE. For example, with the 425 initial protein biomarkers (indicated on the x-axis of FIG. 7B as “variables”), the predictive model achieved an AUC performance metric of nearly 0.75. As the number of protein biomarkers decreased, the predictive capacity of the model remained predictive. For example, at 100 protein biomarkers (which includes the biomarkers in Table 3 with corresponding “1-3Y rank” between 1-100), the predictive model exhibited an AUC of ˜0.68. At 10 protein biomarkers (which includes the biomarkers in Table 3 with corresponding “1-3Y rank” between 1-10), the predictive model exhibited an AUC of ˜0.55. At 5 protein biomarkers (which includes the biomarkers in Table 3 with corresponding “1-3Y rank” between 1-5), the predictive model exhibited an AUC of ˜0.53.

### Example 4: Example Early Prediction of Lung Cancer Using Plasma Protein Biomarkers from Prediction Models Using Olink® Explore 3072 and Target 96 Platforms

Individual plasma proteins have been identified as minimally invasive biomarkers for lung cancer diagnosis with potential utility in early detection. Differences in specific plasma protein levels have been previously shown to be indicative for lung cancer diagnosis, or related to imminent lung cancer. However, more comprehensive plasma protein profiling over longer time periods pre-diagnosis has not been studied.

In this example, the Olink® Explore-3072 platform quantitated 2941 proteins in 496 Liverpool Lung Project (LLP) plasma samples, including 131 cases taken 1-10 years prior to diagnosis, 237 controls, and 90 subjects at multiple times. 1112 proteins associated with haemolysis were excluded. Feature selection with bootstrapping identified differentially expressed proteins, subsequently modelled for lung cancer prediction and validated in UK Biobank data.

### Methods

EDTA plasma samples from LLP subjects were collected by standardized protocols (between 1998 and 2016), with a single cell depletion centrifugation (2200 g, 15 minutes) prior to storing at −80° C. and a further cell depletion spin after thawing, before being aliquoted for Olink studies and refrozen for shipment.

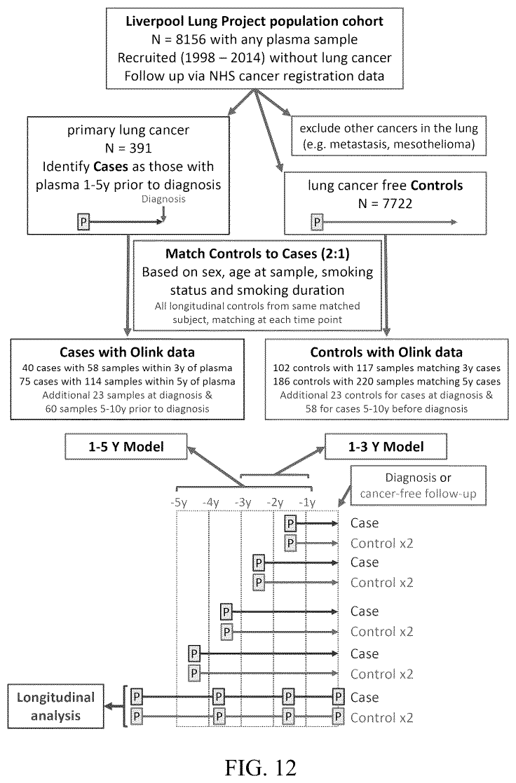

The cases and controls in this example were selected retrospectively as a nested case-control cohort from the LLP population cohort, as shown in FIG. 12.

As illustrated in Table 7, LLP population cohort subjects without lung cancer at the time of recruitment, but were identified with subsequent diagnosis of primary lung cancer within 5 years for the primary discovery cohort.

As illustrated in Table 9, non-small cell lung cancer cases included almost equal numbers of adenocarcinoma (n=53) and squamous cell carcinoma (n=49) and were either early stage (45%) or late stage (52%) at the time of diagnosis.

As illustrated in Table 10, samples at diagnosis (n=23), 1-3 years prior to diagnosis (n=21), 3-5 years prior to diagnosis (n=30) or 5-10 years prior to diagnosis (n=33), were identified for longitudinal studies from 42 cases, along with 110 longitudinal samples at the same time points from 48 controls.

For each case, sex (e.g., self-reported as sex assigned at birth) and age at plasma sample were used to match control subjects (2 per case for discovery cohort and 1 per case for longitudinal studies). Controls were selected to have substantially the same smoking status (e.g., current, former, or never) at the time of sampling and similar lifetime smoking duration (based on all forms of tobacco). Where multiple longitudinal bio-specimens were available from cases, controls were identified with multiple samples at approximately the same intervals. Most subjects were smokers at the time of initial blood collection, with 10 never smokers, and 24 had quit smoking at the time of the last sample used.

Pre-diagnosis plasma proteomics was assessed in a cross-sectional sub-cohort (292 subjects, 1-5 years before diagnosis), and a longitudinal sub-cohort (246 samples from 144 subjects, 5-10 years before diagnosis, 2-5 years before diagnosis, and at time of diagnosis).

Plasma proteomics data was generated using the Olink Explore 3072 platform (2941 proteins), which consists of 8 separate panels: Oncology, Oncology II, Cardiometabolic, Cardiometabolic II, Inflammation, Inflammation II, Neurology, and Neurology II. PCA plots with all proteins and samples were generated, and 6 samples with >5 standard deviations from the mean were filtered. PCA for each panel were generated separately, and an additional 5 samples with >5 standard deviations from the mean were filtered. Data was also generated using the Olink® Target 96 platform (panels: Cardiometabolic, Cardiovascular II, Cardiovascular III, Cell Regulation, Development, Immune Response, Inflammation, Metabolism, Neuro Exploratory, Neurology, Oncology II, Oncology III, Organ Damage).

Haemolysis is known to contribute to increased levels of some proteins in plasma. As shown in Table 11, to avoid potential false-positives results due to haemolysis-associated signals, proteins that were found to be significantly associated with haemolysis were systematically removed. Each sample in the LLP cohort had a haemolysis score assigned ranging from 0 to 4. A linear model was generated to identify proteins significantly associated with haemolysis, with 1112 proteins out of 2941 proteins measured by Olink Explore identified based on FDR<0·01. These proteins were filtered out from further analysis.

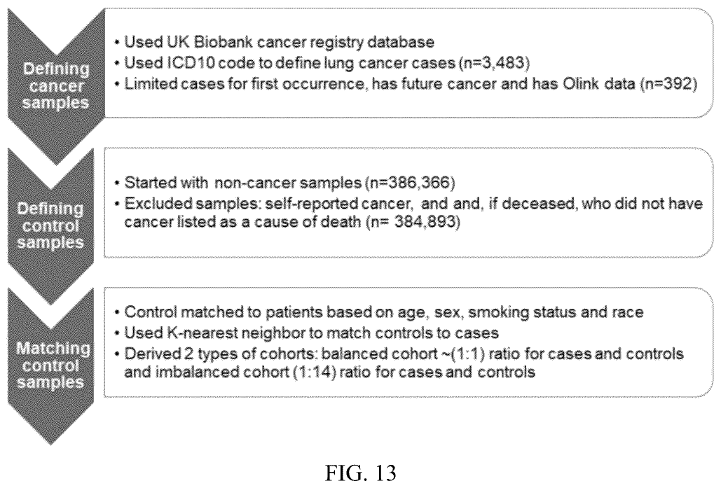

Olink data were generated in UK Biobank (UKB) data. UK Biobank population includes ages from 40 to 69 years, and LLP population includes ages from 48 to 84 years. The analysis involved initial batch of data which was generated using the Olink Explore 1536 platform (1472 proteins) on 54,306 UKB participants. Future cancer cases from UK Biobank cancer registry were extracted. Lung cancer cases using the ICD10 code of C34 were defined. Cancer cases were restricted to the first occurrence, have future cancer from the baseline blood draw, and have Olink data. After applying selection criteria, the total number of cases was 392, as shown in FIG. 13 and Table 12.

Controls were defined as individuals with no record of cancer, who did not self-report any previous cancer incidents, and if deceased cancer was not the cause of death. Controls to cancer cases by age, sex, smoking status and race, were matched using the K-nearest neighbor method to generate matching controls. Two patient-to-control ratios were implemented: one is a balanced ratio where the ratio of cancer to control is 1:1, and another represents the risk of getting lung cancer as 1 cancer:14 controls (392 cases and 5500 controls).

For pan-cancer analysis, the above process for each cancer type was repeated, followed by combining control samples from different cancer types into one pooled control sample; ICD 10 cancer codes: Prostate, C61; Breast, C50; Colorectal, C18 & C19; Uterine Cancer, C44; Kidney Cancer, C64; Pancreatic, C25; Bladder, C67; Stomach, C16; Liver, C22.

### Machine Learning

Feature selection was performed on the discovery cohorts as shown in Table 7 by bootstrapping differential expression on a random set of 50% of the dataset 1000 times using a linear model with age, sex, and pack years as covariates, and proteins were defined as being differentially expressed between cases and controls (P<0·05 linear model anova) at least 100 times. Proteins significantly associated with haemolysis were then filtered out. Four different machine learning algorithms (e.g., Elastic Net, Random Forest, Support Vector Machine, XGBoost) were trained as a binary model to predict cancer vs. control either at 1-3 years before diagnosis or 1-5 years before diagnosis of lung cancer. Receiver operating characteristic area under the curve values (AUCs) from the models are reported as the median AUC from 5-fold cross validation repeated 5 times. To predict future cancer in UKB individuals, the method involves intersecting selected proteins with proteins available in UKB data and trained Support Vector Machine (SVM) classifiers using this set of proteins.

For GO biological process pathways gene set enrichment, 7658 gene sets were obtained from msigdb (www.gsea-msigdb.org), and the list was filtered to only include proteins measured by the Olink Explore platform (2941 proteins). Hypergeometric tests were performed separately on proteins higher or lower in lung cancer cases from the 1-3 years and 1-5 years models, with the background as the 2941 proteins measured by Olink.

### Results

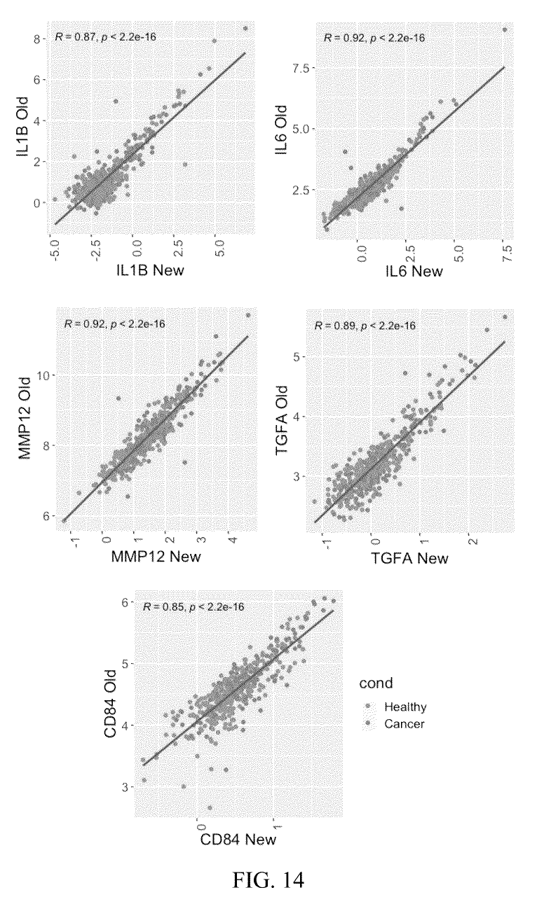

Patient samples taken 1-3 years before diagnosis (1-3Y) from the cross-sectional and longitudinal sub-cohorts were combined to build models to predict development of future lung cancer. 422 proteins that were differentially expressed between healthy subjects and future lung cancer cases 1-3Y prior to diagnosis were identified. 240/422 proteins were kept for further analysis (e.g., 158 up in cases and 82 down) after filtering out proteins that were significantly associated with haemolysis (as shown in Table 11). A subset of these proteins was measured on the Olink® Target 96 platform and these correlated well with the Olink® Explore platform. 262/265 of the overlapping proteins had a significant correlation with FDR<0·05 (FIG. 14 and Table 14).

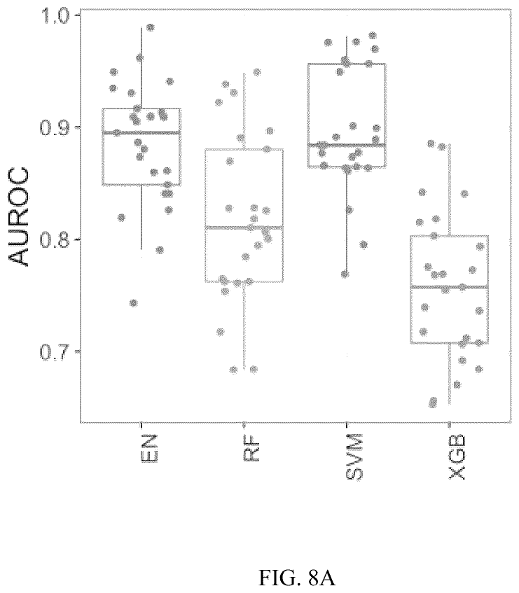

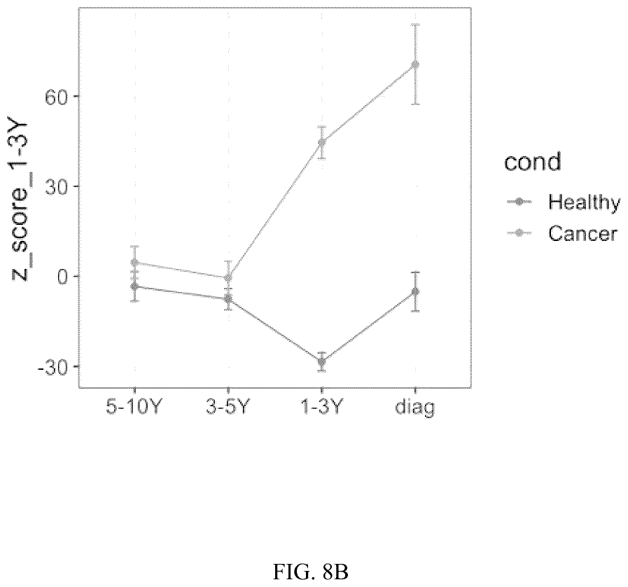

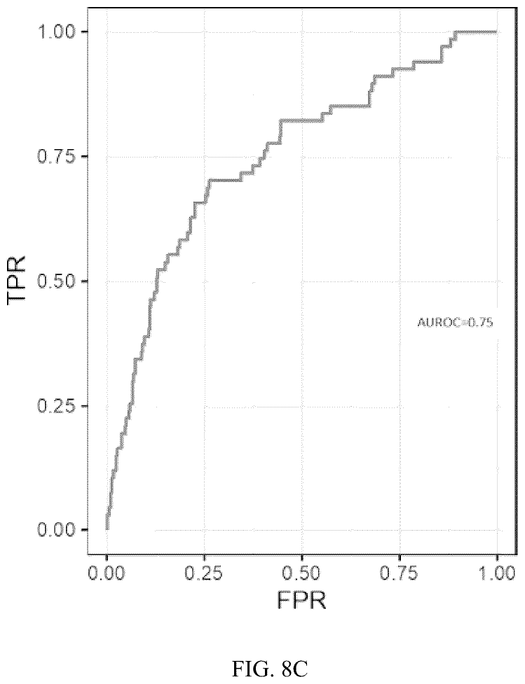

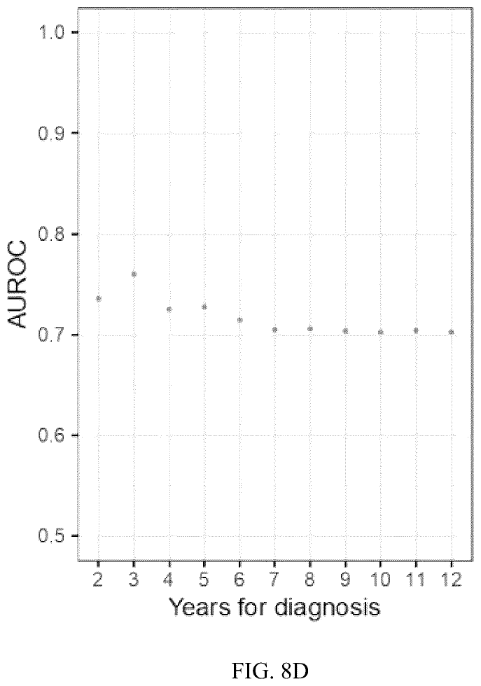

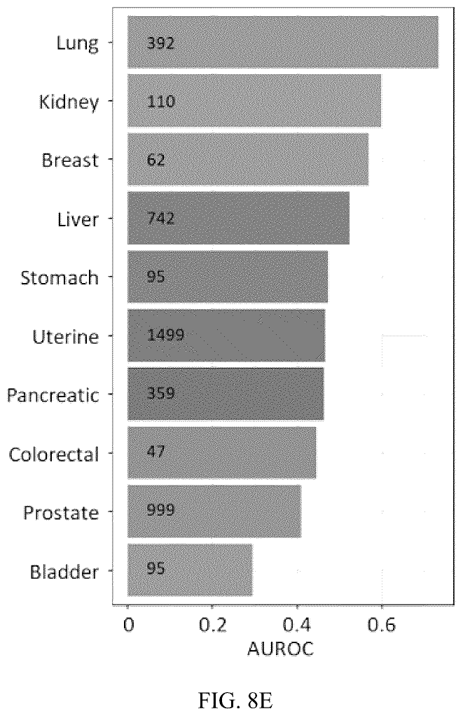

As shown in FIG. 8A, median AUCs from the cross validation ranging from 0.76 to 0.90 were generated by training four different machine learning algorithms on the LLP cohort (e.g., Elastic Net, Random Forest, Support Vector Machine (SVM), XGBoost, 5-fold cross validation repeated 5 times) using the 240 proteins in the 1-3Y cohort.

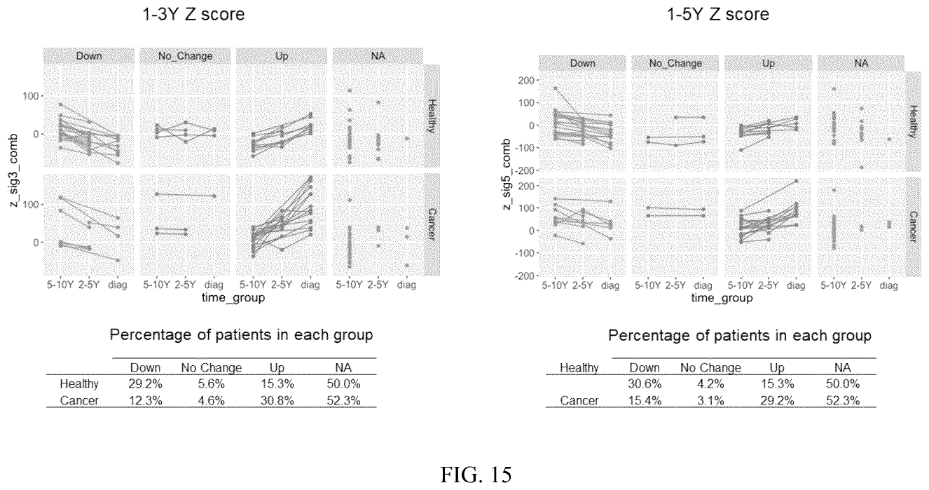

Combined z scores were generated from the differentially expressed proteins at 1-3Y before diagnosis and were plotted over time, including additional longitudinal samples (FIG. 8B). The difference between cases and controls was greater closer to diagnosis. The 1-3Y combined z score differentiated between controls and cases at 1-3 years before diagnosis, but not at 3-5 years or 5-10 years before diagnosis. Individual patient trajectories of the combined z scores indicate that patients that developed cancer were more likely to have an upward trajectory of their z score over time, as shown in FIG. 15.

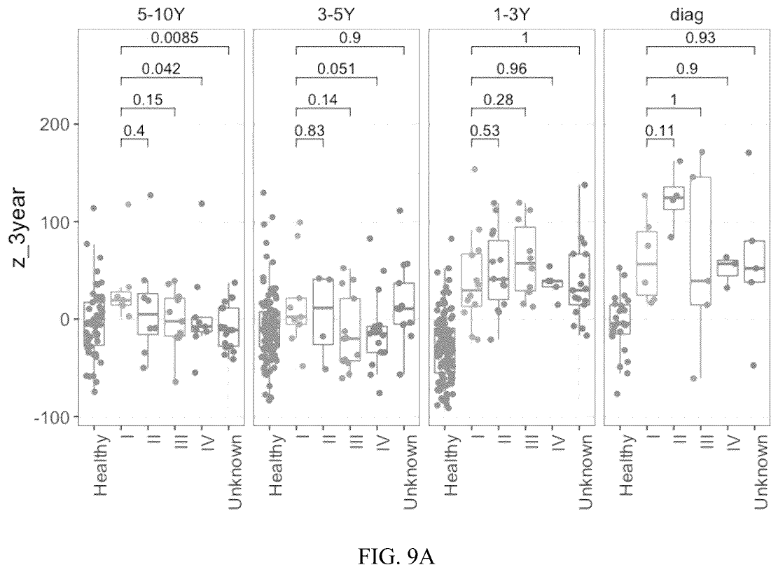

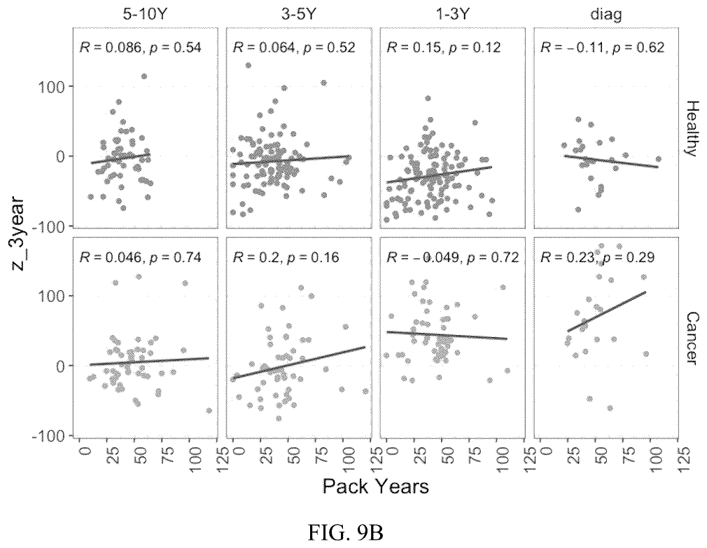

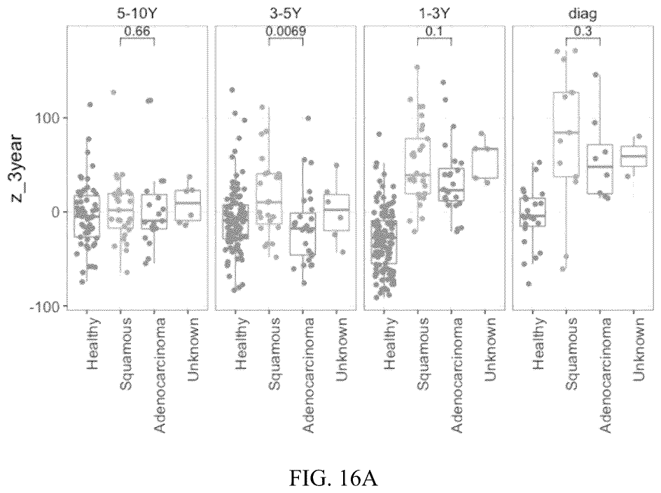

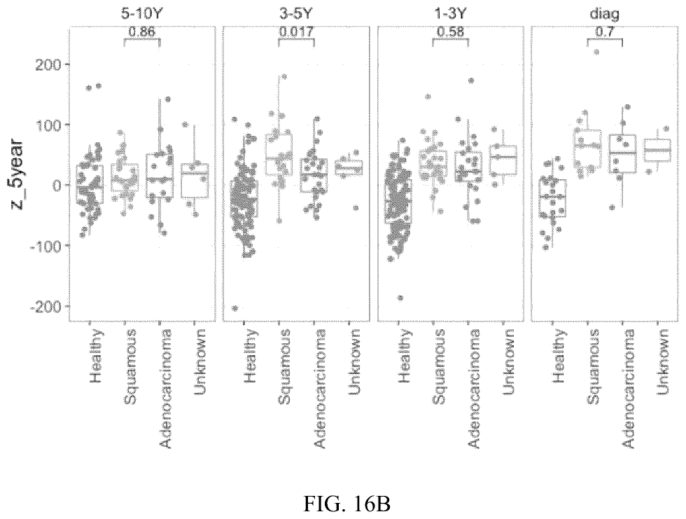

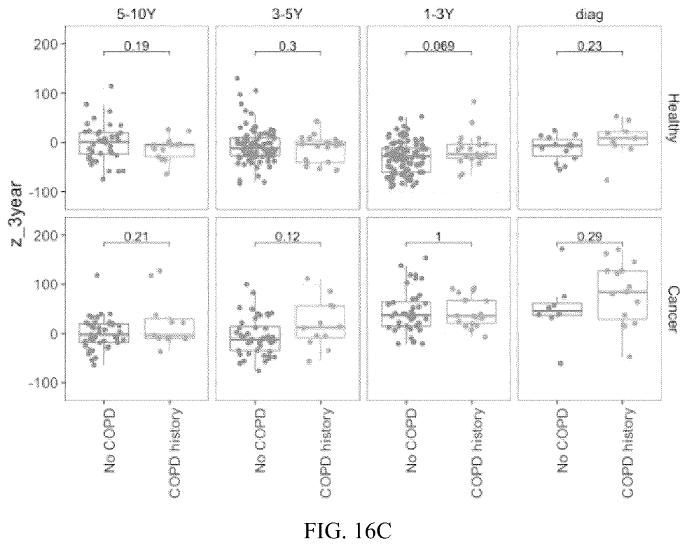

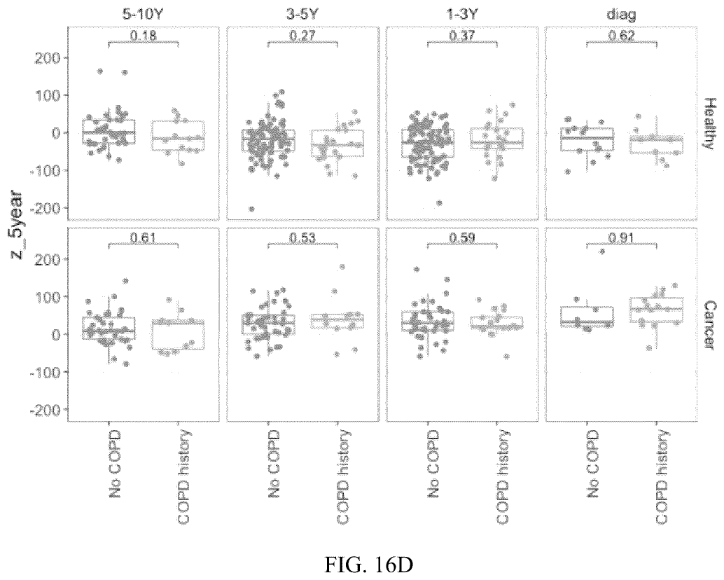

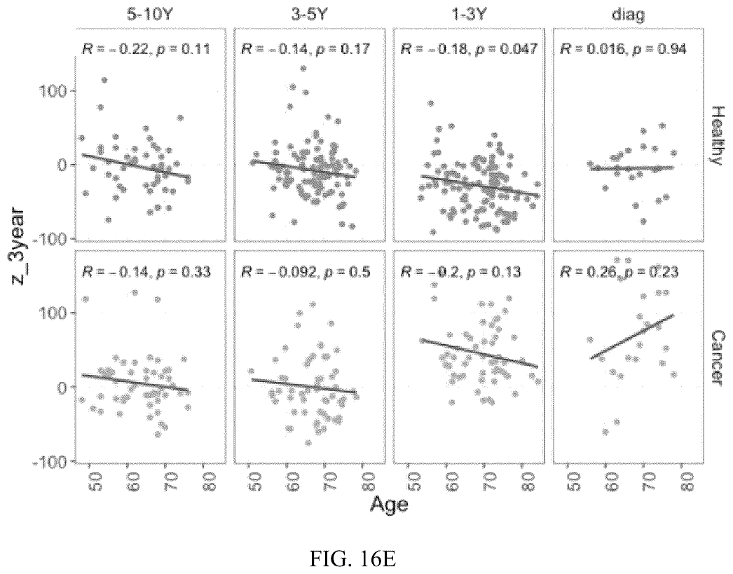

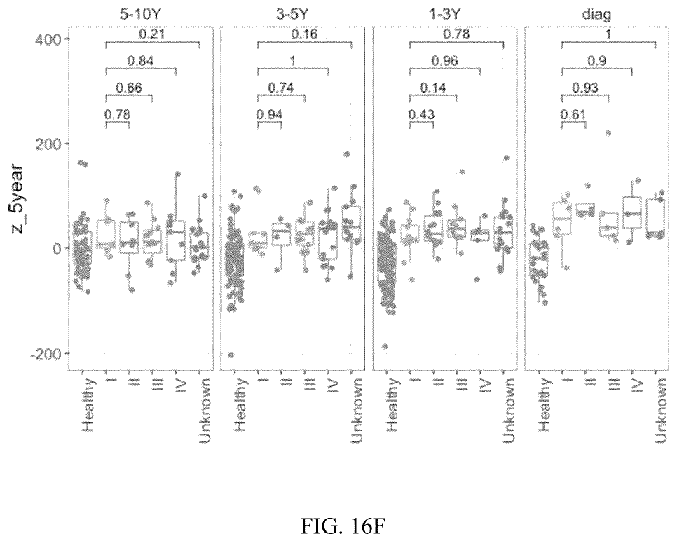

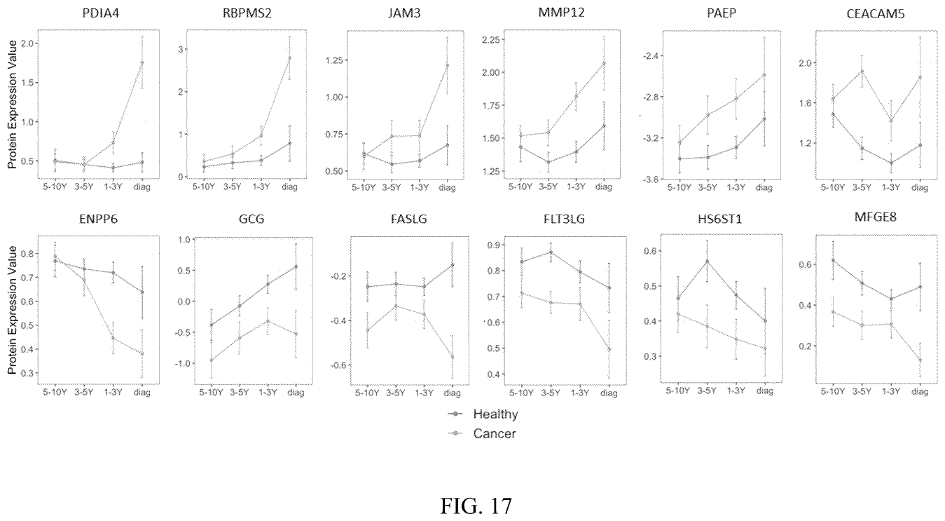

The combined z scores did not differ between stage of cancer at time of diagnosis, as shown in FIG. 9A. A difference between stages was at 5-10 years before diagnosis, where it was higher for stage I than stage IV. However, at this time point the healthy and lung cancer z-scores didn't demonstrate a difference overall. The combined z scores also did not correlate with pack years regardless of time before diagnosis, whether looking at healthy or lung cancer subject, as shown in FIG. 9B. The z score had a stronger signal in squamous cell carcinoma 3-5 years before diagnosis, had no correlation with age in pre-diagnostic samples, and had no association with diagnosis of COPD, as shown in FIG. 16.

These 1-3Y trained models were tested on samples in the UK Biobank using SVM, which was the model that had a superior performance in the training cohort. Proteins that were measured in both LLP and UKB were used in the models since the UKB cohort measured a smaller panel of proteins using the Olink Explore platform: 107/240 for the 1-3Y model. A UK biobank cohort that includes 392 future lung cancer cases and 5500 cancer-free controls was constructed. The 1-3Y model proteins gives rise to an AUC from the cross validation of 0·75 for predicting cancer 1-3Y before diagnosis, as shown in FIG. 8C. An AUC of ˜0·7 was retained for predicting cohorts that included patients 12 years prior to diagnosis, as shown in FIG. 8D. FIG. 8E demonstrates that the model in this example is highly specific to lung cancer in comparison to other types of cancer.

As shown in Table 9, sub-cohort analysis indicated that the model retained performance in non-smokers, patients younger than the age from the recommended screening guidelines and both sexes. As shown in Table 15, the model also retained performance for different histological subtypes.

### Longer Term Prediction

Further, the ability of plasma proteins to predict lung cancer were studied by repeating the analysis using sample taken 1-5 years (1-5Y) prior to diagnosis and matched controls. 489 proteins 1-5Y before diagnosis that were differentially expressed between future lung cancer and healthy subjects were identified. After filtering out proteins that were significantly associated with haemolysis, 267/493 proteins were kept for further analysis (e.g., 119 up in cases and 148 down), 117 of which were also identified for the 1-3Y analysis (e.g., 69 up in cases and 48 down in cases), as shown in Table 13. Hence, over half of those plasma proteins significantly altered in the future lung cancer cases 1-5Y before diagnosis were not identified as significantly altered 1-3Y before to diagnosis (n=150, 50 up in cases and 100 down in cases).

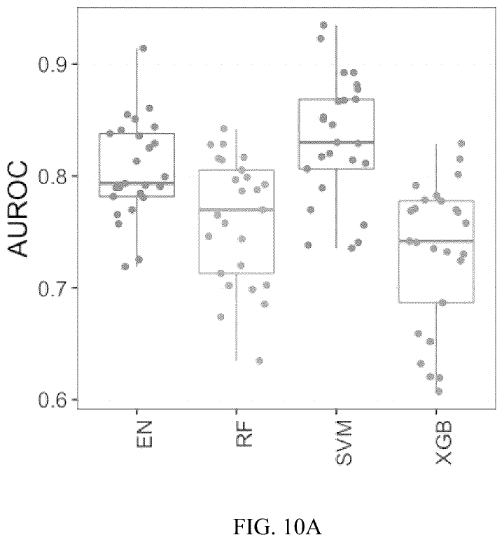

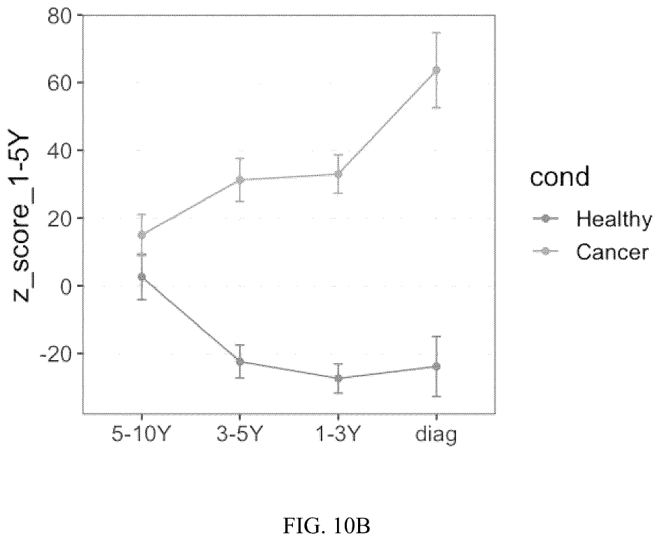

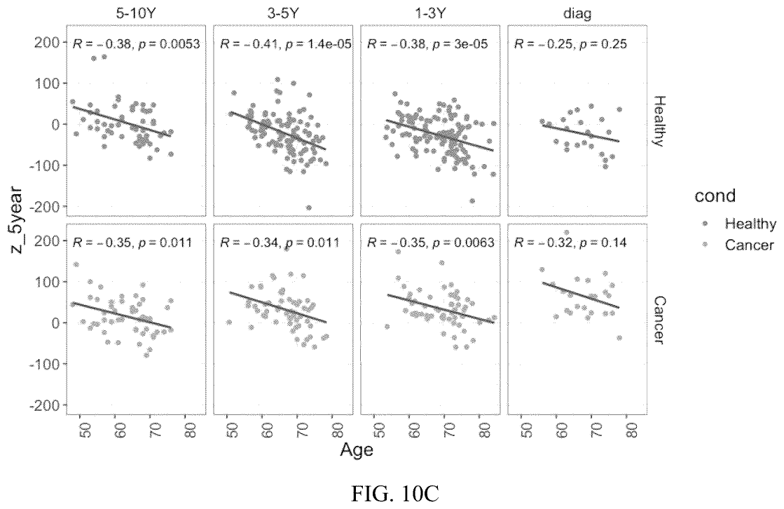

The combined z score for the 1-5Y proteins had the same relationship to histology, COPD (FIG. 16) and smoking pack year histology as the 1-3Y proteins. However, in contrast to 1-3Y proteins (FIG. 8B), the 1-5Y combined z score differentiated between controls and cases at both 1-3Y and 3-5Y before diagnosis, as shown in FIG. 10B, had no relationship to stage (FIG. 16F) and had a negative correlation with age in pre-diagnostic cancer cases and healthy controls (FIG. 10C).

Training four different machine learning algorithms (with 5-fold cross validation repeated 5 times) using the 267 1-5Y proteins (Table 13) generated median AUCs from the cross validation ranging from 0.73 to 0.83, as shown in FIG. 10A. During external validation, the model based on 129 1-5Y proteins measured in the UKB data gave an AUC of 0.69 for predicting lung cancer 1-5Y before diagnosis, which was not significantly different to the 1-3Y model. As with the 1-3Y model, AUC remained around 0.7 even for samples up to 12 years prior to diagnosis.

### Biological Pathways

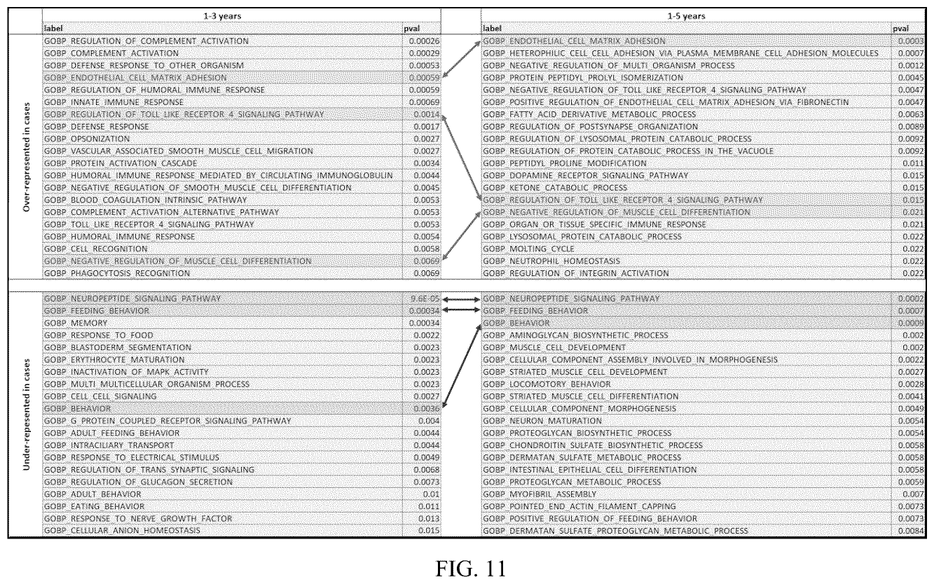

Gene enrichment analysis was performed to investigate potential biological pathways implicated in the risk of future lung cancer, being either increased in plasma (over-represented in cases) or decreased in plasma (under-represented in cases). For the top 20 pathways enriched for proteins either higher or lower in cases, there was limited overlap between 1-3Y and 1-5Y cohorts (FIG. 11); only 3 pathways over-represented in cases and 3 pathways under-represented in cases were shared between the 1-3Y and 1-5Y proteins. Of those pathways with higher plasma protein levels in cases, of the 152 pathways with P<0·05 for either cohort, 57 were significant for 1-5Y only, 83 for 1-3Y only and only 12 for both (Table 16). For proteins with lower levels in cases, of the 138 pathways with P<0·05 for either cohort, 55 were significant for 1-5Y only, 74 for 1-3Y only and only 9 for both (Table 17).

That individual proteins may be associated with different aspects of lung cancer risk and/or presence of undetected lung cancer is exemplified by looking at how levels change over time (FIG. 14) in those cases and controls with longitudinal samples (Table 10). Some increase (e.g. PDIA4, RBPMS2) or decrease (e.g. ENPP6) the closer the sample is taken to diagnosis; others are consistently higher (e.g. CEACAM5) or lower (e.g. MFGE8) varying less over time, but many exhibit a combination of both traits.

Comprehensive plasma protein discovery was performed in this example, using the Olink® Explore 3072 platform, on plasma samples from the Liverpool Lung Project (LLP) taken at various times prior to lung cancer diagnosis. The methods and results in this example provided insight into early predictive biomarkers and how they change over time. The plasma proteome provided protein biomarkers which may be used to identify those at greatest risk of lung cancer, 5 or more years prior to diagnosis. This approach may provide an opportunity to identify patients who would benefit from novel preventative approaches (for pharmaceutical or vaccination interventions) or who would be eligible for lung cancer screening despite not conforming to current smoking-related selection criteria.

Selecting proteins by bootstrapping differential expression, 425 and 493 proteins respectively in the 1-3Y and 1-5Y cohorts were identified, and many of these proteins were associated with haemolysis. As haemolysis-associated proteins would give potential false positive signals if any healthy samples were haemolysed, and it is possible that haemolysis is more often seen in lung cancer patients than healthy individuals, removal of any proteins that were associated with haemolysis was performed, leaving 240 (1-3Y) and 267 (1-5Y) proteins (as identified in Table 13) with each panel combined in a z score to investigate relationships with clinical and epidemiological factors. No association was found with smoking (pack years or duration) or with a history of COPD; a negative association with age was seen for pre-diagnostic samples and controls for the 1-5Y z score only. Hence, the plasma proteins are not directly related to known risk factors for cancer, meaning they are more likely to provide additional useful information when used in conjunction with lung cancer risk scores and be unrelated to smoking-induced inflammation. Furthermore, there was no association with stage of disease at diagnosis (apart from the 1-3Y z score association with early stage, albeit at 5-10 years pre-diagnosis, when not significantly different to control samples) and only a weak association with histological type specifically at 3-5 years before diagnosis. These results indicate that the identified proteomic signals are likely to be useful for prediction of any sub-type of non-small cell lung cancer, regardless of stage.

240 plasma proteins differentially expressed 1-3 years prior to diagnosis and 267 proteins 1-5 years prior to diagnosis were identified, and 117 of the total 390 proteins (30%) were identified in both analyses. This result has significance as the plasma proteome can reflect not just the presence of an occult, pre-diagnosis tumour (with signals most likely closer to diagnosis), but immune response to pre-malignant disease and the biological response to inflammation associated smoking and environmental factors (risk factors that are not necessarily higher at time of diagnosis). Furthermore, when mapped on to pathways by gene set enrichment analysis, there was limited overlap between the top pathways from 1-3Y and 1-5Y (only 21 pathways of 290 with significant enrichment), indicating different biological pathways drive the signal for long-term and short-term risk. Pathway analysis provides valuable insight into potential biological mechanisms underpinning the differential expression, potentially providing insights into targets for preventative treatment for those at high risk of lung cancer. The Olink panels was curated to reflect specific pathways.

The z score based on those selected based on 1-5Y samples showed a greater differential expression at 3-5 years prior to diagnosis than that based on 1-3Y protein. Nevertheless, four different machine learning algorithms demonstrated that both the 1-3Y and 1-5Y proteins were able to predict lung cancer up to 5 years prior to diagnosis (AUCs of 0.76-0.90 for the 1-3Y models and 0.73-0.83 for the 1-5Y models). Remarkably, in the UK Biobank validation it was shown that either set of proteins were able to predict lung cancer to the same extent (AUC=0.7) up to 12 years prior to diagnosis. It is important to note that this cancer prediction was exclusive to lung cancers, with other future cancers in the UK Biobank cohort not predicted, indicating that both the predisposing factors and the tumour-released proteome are likely distinctive for different tumours. Furthermore, in the UK Biobank validation, the predictive power was maintained to some extent in never smokers (AUC=0.62) compared to smokers (AUC=0.69) and was also predictive in those aged 40-55 years (AUC 0.78), who would not usually be eligible for LDCT lung cancer screening; there was also some evidence that it performed better in males (AUC 0.72) than females (AUC 0.66). It is therefore possible that plasma proteome biomarkers might help to expand lung cancer prediction risk scores for better utility within groups currently excluded from the benefit of LDCT screening. However, this would need to be tested in larger populations of younger subjects and never smokers, as these groups are under-represented in most lung cancer cohorts.

Looking at longitudinal samples, the combined z score for the 1-3Y proteins rises significantly towards diagnosis. However, for the 1-5Y protein, differences extend to earlier in disease progression and the levels of some proteins were not increased to as great an extent closer to diagnosis. This indicates that they may represent marker of risk, being indicative of either genetic predisposition or smoking-related damage, rather than being tumour-released or tumour-reactive proteins. Risk biomarkers, rather than being used for early diagnosis, may allow one to identify those who would benefit most from preventative measures, including therapeutic-prevention. For example, inflammation has been shown to be a potential target when post-hoc analysis of the CANTOS trial of Canakinumab (an anti-interleukin-10 monoclonal antibody), for prevention of recurrent vascular events in patients with a persistent pro-inflammatory response, demonstrated a protective effect on lung cancer incidence and mortality; although subsequent trails in treatment of existing cancers have so far proved inconclusive.

Plasma proteins have been shown to provide a means to predict those most at risk of future lung cancer. Similarly, the models could be considered as candidates for inclusion in risk profiling for LDCT screening, or for expedited referral of symptomatic patients.

This example demonstrated that some proteins are associated with longer-term risks, rather than increasing closer to diagnosis (and presumably either being tumour-released or indirectly associated with tumour burden).

In conclusion, the plasma proteome analysis, performed on pre-diagnostic samples from lung cancer patients and lung cancer free controls, identified two partially overlapping panels of proteins from samples 1-3 years or 1-5 years prior to cancer. These panels mapped to predominantly different pathways, but both were predictive for lung cancer on internal and external validation. That samples further from diagnosis displayed different patterns of predictive plasma proteins may indicate that they reflect biological risk, rather than tumour-associated changes. The latter are nevertheless significant in both panels, the combined z scores of which are highest at diagnosis.

The results show that for samples 1-3 years pre-diagnosis, 240 proteins were significantly different in cases; for 1-5 year samples, 117 of these and 150 further proteins were identified, mapping to significantly different pathways. Four machine learning algorithms gave median AUCs of 0.76-0.90 and 0.73-0.83 for the 1-3 year and 1-5 year proteins respectively. External validation gave AUCs of 0.75 (1-3 year) and 0.69 (1-5 year), with AUC 0.7 up to 12 years prior to diagnosis. The models were independent of age, smoking duration, cancer histology and the presence of COPD.

The findings in this example confirmed the predictive power of plasma protein profiling for prediction of future lung cancer diagnosis, identifying potential protein biomarkers for early detection. That biomarker proteins selected using longer pre-diagnostic time points partially overlap those selected using samples from later time points, and represent different molecular pathways, suggests that both biomarkers for inherent cancer risk and occult tumor detection can be identified. This is further supported by the differing longitudinal levels across multiple time points, including at diagnosis.

TABLE 1
Identification of biomarkers in Olink ® Target 96 WP2 platform

| | | Biomarker | | |
|---|---|---|---|---|
| Rank | Biomarker Category | symbol | UniProt | Biomarker Name |
| 1 | INFL_TGF.alpha | TGFA | P01135 | Protransforming growth |
| | | | | factor alpha |
| 2 | CARDIO_VAS_II_MMP12 | MMP12 | P39900 | Macrophage |
| | | | | metalloelastase |
| 3 | CARDIO_VAS_II_TNFRSF13B | TNFRSF13B | O14836 | Tumor necrosis factor |
| | | | | receptor superfamily |
| | | | | member 13B |
| 4 | INFL_TNFSF14 | TNFSF14 | O43557 | Tumor necrosis factor |
| | | | | ligand superfamily |
| | | | | member 14 |
| 5 | IMM_RES_MASP1 | MASP1 | P48740 | Mannan-binding lectin |
| | | | | serine protease 1 |
| 6 | CARDIO_VAS_II_THBS2 | THBS2 | P35442 | Thrombospondin-2 |
| 7 | INFL_GDNF | GDNF | P39905 | Glial cell line-derived |
| | | | | neurotrophic factor |
| 8 | ONCO_III_FLT1 | FLT1 | P17948 | Vascular endothelial |
| | | | | growth factor receptor 1 |
| 9 | IMM_RES_FXYD5 | FXYD5 | Q96DB9 | FXYD domain- |
| | | | | containing ion transport |
| | | | | regulator 5 |
| 10 | INFL_CST5 | CST5 | P28325 | Cystatin-D |
| 11 | IMM_RES_ARNT | ARNT | P27540 | Aryl hydrocarbon |
| | | | | receptor nuclear |
| | | | | translocator |
| 12 | INFL_CDCP1 | CDCP1 | Q9H5V8 | CUB domain-containing |
| | | | | protein 1 |
| 13 | INFL_CCL20 | CCL20 | P78556 | C-C motif chemokine |
| | | | | 20 |
| 14 | INFL_Flt3L | FLT3LG | P49771 | Fms-related tyrosine |
| | | | | kinase 3 ligand |
| 15 | IMM_RES_CLEC7A | CLEC7A | Q9BXN2 | C-type lectin domain |
| | | | | family 7 member A |
| 16 | IMM_RES_PRKCQ | PRKCQ | Q04759 | Protein kinase C theta |
| | | | | type |
| 17 | ONCO_III_SCGN | SCGN | O76038 | Secretagogin |
| 18 | INFL_IL5 | IL5 | P05113 | Interleukin-5 |
| 19 | ONCO_III_NPY | NPY | P01303 | Pro-neuropeptide Y |
| 20 | ONCO_III_S100A16 | S100A16 | Q96FQ6 | Protein S100-A16 |
| 21 | ONCO_III_IL1B | IL1B | P01584 | Interleukin-1 beta |
| 22 | CARDIO_VAS_II_CD84 | CD84 | Q9UIB8 | SLAM family member 5 |
| 23 | IMM_RES_STC1 | STC1 | P52823 | Stanniocalcin-1 |
| 24 | IMM_RES_PRDX3 | PRDX3 | P30048 | Thioredoxin-dependent |
| | | | | peroxide reductase, |
| | | | | mitochondrial |
| 25 | ONCO_III_LAP3 | LAP3 | P28838 | Cytosol aminopeptidase |
| 26 | ONCO_III_GAMT | GAMT | Q14353 | Guanidinoacetate N- |
| | | | | methyltransferase |
| 27 | ONCO_III_CASP2 | CASP2 | P42575 | Caspase-2 |
| 28 | IMM_RES_ITGA6 | ITGA6 | P23229 | Integrin alpha-6 |
| 29 | CARDIO_VAS_II_DECR1 | DECR1 | Q16698 | 2,4-dienoyl-CoA |
| | | | | reductase, mitochondrial |
| 30 | ONCO_III_YTHDF3 | YTHDF3 | Q7Z739 | YTH domain-containing |
| | | | | family protein 3 |

TABLE 2
Identification of biomarkers in “1-5 Y” prediction models in Olink ® Explore 3072 Platform

| | | Biomarker | | |
|---|---|---|---|---|
| Rank | Biomarker Category | symbol | UniProt | Biomarker Name |
| 1 | Oncology_CEACAM5 | CEACAM5 | P06731 | Carcinoembryonic antigen- |
| | | | | related cell adhesion |
| | | | | molecule 5 |
| 2 | Oncology_II_TOP1 | TOP1 | P11387 | DNA topoisomerase 1 |
| 3 | Cardiometabolic_NCAM1 | NCAM1 | P13591 | Neural cell adhesion |
| | | | | molecule 1 |
| 4 | Inflammation_SCGB3A2 | SCGB3A2 | Q96PL1 | Secretoglobin family 3A |
| | | | | member 2 |
| 5 | Cardiometabolic_II_CALY | CALY | Q9NYX4 | Neuron-specific vesicular |
| | | | | protein calcyon |
| 6 | Cardiometabolic_TGFBI | TGFBI | Q15582 | Transforming growth factor- |
| | | | | beta-induced protein ig-h3 |
| 7 | Neurology_II_CABP2 | CABP2 | Q9NPB3 | Calcium-binding protein 2 |
| 8 | Cardiometabolic_II_ENPP6 | ENPP6 | Q6UWR7 | Glycerophosphocholine |
| | | | | cholinephosphodiesterase |
| | | | | ENPP6 |
| 9 | Neurology_KRT14 | KRT14 | P02533 | Keratin, type I cytoskeletal |
| | | | | 14 |
| 10 | Neurology_II_HEPACAM2 | HEPACAM2 | A8MVW5 | HEPACAM family member |
| | | | | 2 |
| 11 | Neurology_II_TMEM25 | TMEM25 | Q86YD3 | Transmembrane protein 25 |
| 12 | Cardiometabolic_II_SGSH | SGSH | P51688 | N-sulphoglucosamine |
| | | | | sulphohydrolase |
| 13 | Neurology_II_MFAP3L | MFAP3L | O75121 | Microfibrillar-associated |
| | | | | protein 3-like |
| 14 | Neurology_TNFSF14 | TNFSF14 | O43557 | Tumor necrosis factor ligand |
| | | | | superfamily member 14 |
| 15 | Neurology_II_CD3D | CD3D | P04234 | T-cell surface glycoprotein |
| | | | | CD3 delta chain |
| 16 | Cardiometabolic_II_TMED4 | TMED4 | Q7Z7H5 | Transmembrane emp24 |
| | | | | domain-containing protein 4 |
| 17 | Cardiometabolic_II_ZP3 | ZP3 | P21754 | Zona pellucida sperm- |
| | | | | binding protein 3 |
| 18 | Oncology_MMP12 | MMP12 | P39900 | Macrophage metalloelastase |
| 19 | Oncology_GCG | GCG | P01275 | Pro-glucagon |
| 20 | Inflammation_II_AFM | AFM | P43652 | Afamin |
| 21 | Neurology_SPINT1 | SPINT1 | O43278 | Kunitz-type protease |
| | | | | inhibitor 1 |
| 22 | Cardiometabolic_II_LILRA4 | LILRA4 | P59901 | Leukocyte immunoglobulin- |
| | | | | like receptor subfamily A |
| | | | | member 4 |
| 23 | Inflammation_FLT3LG | FLT3LG | P49771 | Fms-related tyrosine kinase |
| | | | | 3 ligand |
| 24 | Neurology_II_AGBL2 | AGBL2 | Q5U5Z8 | Cytosolic carboxypeptidase |
| | | | | 2 |
| 25 | Neurology_PAEP | PAEP | P09466 | Glycodelin |
| 26 | Inflammation_II_SCGB3A1 | SCGB3A1 | Q96QR1 | Secretoglobin family 3A |
| | | | | member 1 |
| 27 | Neurology_II_LRFN2 | LRFN2 | Q9ULH4 | Leucine-rich repeat and |
| | | | | fibronectin type-III domain- |
| | | | | containing protein 2 |
| 28 | Neurology_II_TJP3 | TJP3 | O95049 | Tight junction protein ZO-3 |
| 29 | Oncology_II_FGF7 | FGF7 | P21781 | Fibroblast growth factor 7 |
| 30 | Oncology_LRIG1 | LRIG1 | Q96JA1 | Leucine-rich repeats and |
| | | | | immunoglobulin-like |
| | | | | domains protein 1 |
| 31 | Oncology_CA14 | CA14 | Q9ULX7 | Carbonic anhydrase 14 |
| 32 | Oncology_II_CEACAM18 | CEACAM18 | A8MTB9 | Carcinoembryonic antigen- |
| | | | | related cell adhesion |
| | | | | molecule 18 |
| 33 | Inflammation_II_CST1 | CST1 | P01037 | Cystatin-SN |
| 34 | Neurology_ANXA10 | ANXA10 | Q9UJ72 | Annexin A10 |
| 35 | Neurology_CDCP1 | CDCP1 | Q9H5V8 | CUB domain-containing |
| | | | | protein 1 |
| 36 | Neurology_GPC5 | GPC5 | P78333 | Glypican-5 |
| 37 | Inflammation_OSCAR | OSCAR | Q8IYS5 | Osteoclast-associated |
| | | | | immunoglobulin-like |
| | | | | receptor |
| 38 | Cardiometabolic_II_CEACAM6 | CEACAM6 | P40199 | Carcinoembryonic antigen- |
| | | | | related cell adhesion |
| | | | | molecule 6 |
| 39 | Cardiometabolic_II_CD2 | CD2 | P06729 | T-cell surface antigen CD2 |
| 40 | Neurology_SNCG | SNCG | O76070 | Gamma-synuclein |
| 41 | Cardiometabolic_GPR37 | GPR37 | O15354 | Prosaposin receptor GPR37 |
| 42 | Neurology_II_SEPTIN3 | SEPTIN3 | Q9UH03 | Neuronal-specific septin-3 |
| 43 | Cardiometabolic_II_RAB10 | RAB10 | P61026 | Ras-related protein Rab-10 |
| 44 | Neurology_DKK4 | DKK4 | Q9UBT3 | Dickkopf-related protein 4 |
| 45 | Oncology_DKKL1 | DKKL1 | Q9UK85 | Dickkopf-like protein 1 |
| 46 | Cardiometabolic_SOST | SOST | Q9BQB4 | Sclerostin |
| 47 | Inflammation_CSF3 | CSF3 | P09919 | Granulocyte colony- |
| | | | | stimulating factor |
| 48 | Oncology_II_VWA5A | VWA5A | O00534 | von Willebrand factor A |
| | | | | domain-containing protein |
| | | | | 5A |
| 49 | Neurology_II_TSPAN7 | TSPAN7 | P41732 | Tetraspanin-7 |
| 50 | Neurology_PAK4 | PAK4 | O96013 | Serine/threonine-protein |
| | | | | kinase PAK 4 |
| 51 | Cardiometabolic_BPIFB1 | BPIFB1 | Q8TDL5 | BPI fold-containing family |
| | | | | B member 1 |
| 52 | Oncology_SIGLEC9 | SIGLEC9 | Q9Y336 | Sialic acid-binding Ig-like |
| | | | | lectin 9 |
| 53 | Oncology_II_ZNRD2 | ZNRD2 | O60232 | Protein ZNRD2 |
| 54 | Cardiometabolic_PM20D1 | PM20D1 | Q6GTS8 | N-fatty-acyl-amino acid |
| | | | | synthase/hydrolase PM20D1 |
| 55 | Oncology_II_TK1 | TK1 | P04183 | Thymidine kinase, cytosolic |
| 56 | Cardiometabolic_II_RPS10 | RPS10 | P46783 | 40S ribosomal protein S10 |
| 57 | Cardiometabolic_II_PMCH | PMCH | P20382 | Pro-MCH |
| 58 | Oncology_II_RNF43 | RNF43 | Q68DV7 | E3 ubiquitin-protein ligase |
| | | | | RNF43 |
| 59 | Cardiometabolic_MEP1B | MEP1B | Q16820 | Meprin A subunit beta |
| 60 | Oncology_BGN | BGN | P21810 | Biglycan |
| 61 | Oncology_NELL1 | NELL1 | Q92832 | Protein kinase C-binding |
| | | | | protein NELL1 |
| 62 | Oncology_II_CD101 | CD101 | Q93033 | Immunoglobulin |
| | | | | superfamily member 2 |
| 63 | Neurology_II_LRP2BP | LRP2BP | Q9P2M1 | LRP2-binding protein |
| 64 | Neurology_II_PRSS53 | PRSS53 | Q2L4Q9 | Serine protease 53 |
| 65 | Neurology_MFGE8 | MFGE8 | Q08431 | Lactadherin |
| 66 | Inflammation_II_THSD1 | THSD1 | Q9NS62 | Thrombospondin type-1 |
| | | | | domain-containing protein 1 |
| 67 | Inflammation_CKMT1A_CKMT1B | CKMT1A_CKMT1B | P12532 | Creatine kinase U-type, |
| | | | | mitochondrial |
| 68 | Inflammation_MEPE | MEPE | Q9NQ76 | Matrix extracellular |
| | | | | phosphoglycoprotein |
| 69 | Inflammation_II_APOL1 | APOL1 | O14791 | Apolipoprotein L1 |
| 70 | Inflammation_II_RBPMS | RBPMS | Q93062 | RNA-binding protein with |
| | | | | multiple splicing |
| 71 | Cardiometabolic_MARCO | MARCO | Q9UEW3 | Macrophage receptor |
| | | | | MARCO |
| 72 | Neurology_II_KLRC1 | KLRC1 | P26715 | NKG2-A/NKG2-B type II |
| | | | | integral membrane protein |
| 73 | Cardiometabolic_II_FGFBP2 | FGFBP2 | Q9BYJ0 | Fibroblast growth factor- |
| | | | | binding protein 2 |
| 74 | Inflammation_II_TPSG1 | TPSG1 | Q9NRR2 | Tryptase gamma |
| 75 | Inflammation_II_SELENOP | SELENOP | P49908 | Selenoprotein P |
| 76 | Inflammation_CLEC7A | CLEC7A | Q9BXN2 | C-type lectin domain family |
| | | | | 7 member A |
| 77 | Oncology_II_UPK3BL1 | UPK3BL1 | BOFP48 | Uroplakin-3b-like protein 1 |
| 78 | Oncology_HS6ST1 | HS6ST1 | O60243 | Heparan-sulfate 6-O- |
| | | | | sulfotransferase 1 |
| 79 | Oncology_II_ENDOU | ENDOU | P21128 | Poly(U)-specific |
| | | | | endoribonuclease |
| 80 | Inflammation_II_IL12RB2 | IL12RB2 | Q99665 | Interleukin-12 receptor |
| | | | | subunit beta-2 |
| 81 | Oncology_II_CYB5A | CYB5A | P00167 | Cytochrome b5 |
| 82 | Neurology_GKN1 | GKN1 | Q9NS71 | Gastrokine-1 |
| 83 | Inflammation_NRTN | NRTN | Q99748 | Neurturin |
| 84 | Inflammation_CCL26 | CCL26 | Q9Y258 | C-C motif chemokine 26 |
| 85 | Oncology_CRNN | CRNN | Q9UBG3 | Cornulin |
| 86 | Inflammation_II_PINLYP | PINLYP | A6NC86 | phospholipase A2 inhibitor |
| | | | | and Ly6/PLAUR domain- |
| | | | | containing protein |
| 87 | Neurology_LAIR2 | LAIR2 | Q6ISS4 | Leukocyte-associated |
| | | | | immunoglobulin-like |
| | | | | receptor 2 |
| 88 | Neurology_BAG3 | BAG3 | O95817 | BAG family molecular |
| | | | | chaperone regulator 3 |
| 89 | Cardiometabolic_II_SCPEP1 | SCPEP1 | Q9HB40 | Retinoid-inducible serine |
| | | | | carboxypeptidase |
| 90 | Cardiometabolic_II_RIPK4 | RIPK4 | P57078 | Receptor-interacting |
| | | | | serine/threonine-protein |
| | | | | kinase 4 |
| 91 | Inflammation_II_CTSE | CTSE | P14091 | Cathepsin E |
| 92 | Oncology_II_TMOD4 | TMOD4 | Q9NZQ9 | Tropomodulin-4 |
| 93 | Oncology_SFTPA1 | SFTPA1 | Q8IWL2 | Pulmonary surfactant- |
| | | | | associated protein A1 |
| 94 | Neurology_SEMA4D | SEMA4D | Q92854 | Semaphorin-4D |
| 95 | Inflammation_IL17C | IL17C | Q9P0M4 | Interleukin-17C |
| 96 | Neurology_GFRA3 | GFRA3 | O60609 | GDNF family receptor |
| | | | | alpha-3 |
| 97 | Oncology_DPEP2 | DPEP2 | Q9H4A9 | Dipeptidase 2 |
| 98 | Cardiometabolic_II_EDEM2 | EDEM2 | Q9BV94 | ER degradation-enhancing |
| | | | | alpha-mannosidase-like |
| | | | | protein 2 |
| 99 | Inflammation_CD84 | CD84 | Q9UIB8 | SLAM family member 5 |
| 100 | Neurology_KIRREL2 | KIRREL2 | Q6UWL6 | Kin of IRRE-like protein 2 |
| 101 | Inflammation_II_NECTIN1 | NECTIN1 | Q15223 | Nectin-1 |
| 102 | Neurology_II_CBLN1 | CBLN1 | P23435 | Cerebellin-1 |
| 103 | Inflammation_NTF3 | NTF3 | P20783 | Neurotrophin-3 |
| 104 | Cardiometabolic_II_PYY | PYY | P10082 | Peptide YY |
| 105 | Cardiometabolic_XG | XG | P55808 | Glycoprotein Xg |
| 106 | Oncology_NPY | NPY | P01303 | Pro-neuropeptide Y |
| 107 | Inflammation_CCL20 | CCL20 | P78556 | C-C motif chemokine 20 |
| 108 | Cardiometabolic_II_SIL1 | SIL1 | Q9H173 | Nucleotide exchange factor |
| | | | | SIL1 |
| 109 | Neurology_II_PLB1 | PLB1 | Q6P1J6 | Phospholipase B1, |
| | | | | membrane-associated |
| 110 | Neurology_II_DUSP29 | DUSP29 | Q68J44 | Dual specificity phosphatase |
| | | | | 29 |
| 111 | Cardiometabolic_UMOD | UMOD | P07911 | Uromodulin |
| 112 | Neurology_II_ATXN2L | ATXN2L | Q8WWM7 | Ataxin-2-like protein |
| 113 | Neurology_II_LEO1 | LEO1 | Q8WVC0 | RNA polymerase-associated |
| | | | | protein LEO1 |
| 114 | Inflammation_II_PROS1 | PROS1 | P07225 | Vitamin K-dependent |
| | | | | protein S |
| 115 | Oncology_II_EDDM3B | EDDM3B | P56851 | Epididymal secretory protein |
| | | | | E3-beta |
| 116 | Cardiometabolic_II_ENO3 | ENO3 | P13929 | Beta-enolase |
| 117 | Oncology_DCBLD2 | DCBLD2 | Q96PD2 | Discoidin, CUB and LCCL |
| | | | | domain-containing protein 2 |
| 118 | Neurology_MMP9 | MMP9 | P14780 | Matrix metalloproteinase-9 |
| 119 | Cardiometabolic_II_KIF22 | KIF22 | Q14807 | Kinesin-like protein KIF22 |
| 120 | Cardiometabolic_II_DENND2B | DENND2B | P78524 | DENN domain-containing |
| | | | | protein 2B |
| 121 | Inflammation_II_C1RL | C1RL | Q9NZP8 | Complement C1r |
| | | | | subcomponent-like protein |
| 122 | Oncology_PVALB | PVALB | P20472 | Parvalbumin alpha |
| 123 | Inflammation_CXCL8 | CXCL8 | P10145 | Interleukin-8 |
| 124 | Oncology_PPY | PPY | P01298 | Pancreatic prohormone |
| 125 | Oncology_CCN1 | CCN1 | O00622 | CCN family member 1 |
| 126 | Oncology_KLK10 | KLK10 | O43240 | Kallikrein-10 |
| 127 | Neurology_II_RRAS | RRAS | P10301 | Ras-related protein R-Ras |
| 128 | Neurology_II_SCN3B | SCN3B | Q9NY72 | Sodium channel subunit |
| | | | | beta-3 |
| 129 | Cardiometabolic_II_BPIFB2 | BPIFB2 | Q8N4F0 | BPI fold-containing family |
| | | | | B member 2 |
| 130 | Inflammation_II_ITGAL | ITGAL | P20701 | Integrin alpha-L |
| 131 | Oncology_II_DDX1 | DDX1 | Q92499 | ATP-dependent RNA |
| | | | | helicase DDX1 |
| 132 | Cardiometabolic_II_MEGF11 | MEGF11 | A6BM72 | Multiple epidermal growth |
| | | | | factor-like domains protein |
| | | | | 11 |
| 133 | Cardiometabolic_II_NOP56 | NOP56 | O00567 | Nucleolar protein 56 |
| 134 | Oncology_NTF4 | NTF4 | P34130 | Neurotrophin-4 |
| 135 | Neurology_HNMT | HNMT | P50135 | Histamine N- |
| | | | | methyltransferase |
| 136 | Oncology_II_IL9 | IL9 | P15248 | Interleukin-9 |
| 137 | Oncology_II_SCRIB | SCRIB | Q14160 | Protein scribble homolog |
| 138 | Oncology_UXS1 | UXS1 | Q8NBZ7 | UDP-glucuronic acid |
| | | | | decarboxylase 1 |
| 139 | Oncology_II_MEP1A | MEP1A | Q16819 | Meprin A subunit alpha |
| 140 | Cardiometabolic_II_ACTN2 | ACTN2 | P35609 | Alpha-actinin-2 |
| 141 | Cardiometabolic_II_NECAP2 | NECAP2 | Q9NVZ3 | Adaptin ear-binding coat- |
| | | | | associated protein 2 |
| 142 | Neurology_CLEC10A | CLEC10A | Q8IUN9 | C-type lectin domain family |
| | | | | 10 member A |
| 143 | Neurology_II_DDX53 | DDX53 | Q86TM3 | Probable ATP-dependent |
| | | | | RNA helicase DDX53 |
| 144 | Neurology_II_SV2A | SV2A | Q7L0J3 | Synaptic vesicle |
| | | | | glycoprotein 2A |
| 145 | Neurology_ATXN10 | ATXN10 | Q9UBB4 | Ataxin-10 |
| 146 | Inflammation_II_PI16 | PI16 | Q6UXB8 | Peptidase inhibitor 16 |
| 147 | Neurology_II_KCNH2 | KCNH2 | Q12809 | Potassium voltage-gated |
| | | | | channel subfamily H |
| | | | | member 2 |
| 148 | Neurology_TNR | TNR | Q92752 | Tenascin-R |
| 149 | Cardiometabolic_PDGFRB | PDGFRB | P09619 | Platelet-derived growth |
| | | | | factor receptor beta |
| 150 | Inflammation_II_SERPINA4 | SERPINA4 | P29622 | Kallistatin |
| 151 | Oncology_CDC27 | CDC27 | P30260 | Cell division cycle protein |
| | | | | 27 homolog |
| 152 | Neurology_II_MICALL2 | MICALL2 | Q8IY33 | MICAL-like protein 2 |
| 153 | Oncology_CD28 | CD28 | P10747 | T-cell-specific surface |
| | | | | glycoprotein CD28 |
| 154 | Neurology_BRK1 | BRK1 | Q8WUW1 | Protein BRICK1 |
| 155 | Neurology_SLC16A1 | SLC16A1 | P53985 | Monocarboxylate transporter |
| | | | | 1 |
| 156 | Neurology_II_DSCAM | DSCAM | O60469 | Down syndrome cell |
| | | | | adhesion molecule |
| 157 | Oncology_II_PBXIP1 | PBXIP1 | Q96AQ6 | Pre-B-cell leukemia |
| | | | | transcription factor- |
| | | | | interacting protein 1 |
| 158 | Neurology_MATN3 | MATN3 | O15232 | Matrilin-3 |
| 159 | Oncology_SFTPA2 | SFTPA2 | Q8IWL1 | Pulmonary surfactant- |
| | | | | associated protein A2 |
| 160 | Oncology_II_PTTG1 | PTTG1 | 095997 | Securin |
| 161 | Neurology_ASAH2 | ASAH2 | Q9NR71 | Neutral ceramidase |
| 162 | Oncology_SCG2 | SCG2 | P13521 | Secretogranin-2 |
| 163 | Cardiometabolic_II_PTGR1 | PTGR1 | Q14914 | Prostaglandin reductase 1 |
| 164 | Neurology_II_GBA | GBA | P04062 | Lysosomal acid |
| | | | | glucosylceramidase |
| 165 | Cardiometabolic_II_PTPRZ1 | PTPRZ1 | P23471 | Receptor-type tyrosine- |
| | | | | protein phosphatase zeta |
| 166 | Oncology_II_ERN1 | ERN1 | O75460 | Serine/threonine-protein |
| | | | | kinase/endoribonuclease |
| | | | | IRE1 |
| 167 | Cardiometabolic_II_LECT2 | LECT2 | O14960 | Leukocyte cell-derived |
| | | | | chemotaxin-2 |
| 168 | Inflammation_SCGN | SCGN | O76038 | Secretagogin |
| 169 | Inflammation_HLA.DRA | HLA-DRA | P01903 | HLA class II |
| | | | | histocompatibility antigen, |
| | | | | DR alpha chain |
| 170 | Inflammation_IL5RA | IL5RA | Q01344 | Interleukin-5 receptor |
| | | | | subunit alpha |
| 171 | Neurology_LRPAP1 | LRPAP1 | P30533 | Alpha-2-macroglobulin |
| | | | | receptor-associated protein |
| 172 | Neurology_CXCL13 | CXCL13 | O43927 | C-X-C motif chemokine 13 |
| 173 | Inflammation_II_NEXN | NEXN | Q0ZGT2 | Nexilin |
| 174 | Cardiometabolic_II_CD248 | CD248 | Q9HCU0 | Endosialin |
| 175 | Inflammation_KYNU | KYNU | Q16719 | Kynureninase |
| 176 | Oncology_ADAMTS15 | ADAMTS15 | Q8TE58 | A disintegrin and |
| | | | | metalloproteinase with |
| | | | | thrombospondin motifs 15 |
| 177 | Inflammation_WFIKKN2 | WFIKKN2 | Q8TEU8 | WAP, Kazal, |
| | | | | immunoglobulin, Kunitz and |
| | | | | NTR domain-containing |
| | | | | protein 2 |
| 178 | Neurology_CLEC14A | CLEC14A | Q86T13 | C-type lectin domain family |
| | | | | 14 member A |
| 179 | Neurology_II_FZD10 | FZD10 | Q9ULW2 | Frizzled-10 |
| 180 | Cardiometabolic_PROC | PROC | P04070 | Vitamin K-dependent |
| | | | | protein C |
| 181 | Inflammation_LY9 | LY9 | Q9HBG7 | T-lymphocyte surface |
| | | | | antigen Ly-9 |
| 182 | Neurology_II_LRP2 | LRP2 | P98164 | Low-density lipoprotein |
| | | | | receptor-related protein 2 |
| 183 | Neurology_CX3CL1 | CX3CL1 | P78423 | Fractalkine |
| 184 | Cardiometabolic_RNASET2 | RNASET2 | O00584 | Ribonuclease T2 |
| 185 | Neurology_CTSS | CTSS | P25774 | Cathepsin S |
| 186 | Inflammation_II_MCEMP1 | MCEMP1 | Q8IX19 | Mast cell-expressed |
| | | | | membrane protein 1 |
| 187 | Cardiometabolic_COMP | COMP | P49747 | Cartilage oligomeric matrix |
| | | | | protein |
| 188 | Oncology_SIGLEC6 | SIGLEC6 | O43699 | Sialic acid-binding Ig-like |
| | | | | lectin 6 |
| 189 | Inflammation_CCL24 | CCL24 | O00175 | C-C motif chemokine 24 |
| 190 | Inflammation_AOC1 | AOC1 | P19801 | Amiloride-sensitive amine |
| | | | | oxidase [copper-containing] |
| 191 | Cardiometabolic_PLXNB3 | PLXNB3 | Q9ULL4 | Plexin-B3 |
| 192 | Oncology_TMPRSS15 | TMPRSS15 | P98073 | Enteropeptidase |
| 193 | Inflammation_FCAR | FCAR | P24071 | Immunoglobulin alpha Fc |
| | | | | receptor |
| 194 | Neurology_II_SCIN | SCIN | Q9Y6U3 | Adseverin |
| 195 | Oncology_II_IFI30 | IFI30 | P13284 | Gamma-interferon-inducible |
| | | | | lysosomal thiol reductase |
| 196 | Neurology_II_KIRREL1 | KIRREL1 | Q96J84 | Kin of IRRE-like protein 1 |
| 197 | Inflammation_FXYD5 | FXYD5 | Q96DB9 | FXYD domain-containing |
| | | | | ion transport regulator 5 |
| 198 | Neurology_S100A16 | S100A16 | Q96FQ6 | Protein S100-A16 |
| 199 | Cardiometabolic_LILRA5 | LILRA5 | A6NI73 | Leukocyte immunoglobulin- |
| | | | | like receptor subfamily A |
| | | | | member 5 |
| 200 | Neurology_CLSPN | CLSPN | Q9HAW4 | Claspin |
| 201 | Cardiometabolic_II_AHNAK2 | AHNAK2 | Q8IVF2 | Protein AHNAK2 |
| 202 | Cardiometabolic_II_CTLA4 | CTLA4 | P16410 | Cytotoxic T-lymphocyte |
| | | | | protein 4 |
| 203 | Oncology_II_INSL5 | INSL5 | Q9Y5Q6 | Insulin-like peptide INSL5 |
| 204 | Oncology_II_WDR46 | WDR46 | O15213 | WD repeat-containing |
| | | | | protein 46 |
| 205 | Neurology_CST5 | CST5 | P28325 | Cystatin-D |
| 206 | Oncology_II_PHLDB2 | PHLDB2 | Q86SQ0 | Pleckstrin homology-like |
| | | | | domain family B member 2 |
| 207 | Neurology_TREML2 | TREML2 | Q5T2D2 | Trem-like transcript 2 |
| | | | | protein |
| 208 | Neurology_GUCA2A | GUCA2A | Q02747 | Guanylin |
| 209 | Neurology_PFDN2 | PFDN2 | Q9UHV9 | Prefoldin subunit 2 |
| 210 | Cardiometabolic_II_PDIA4 | PDIA4 | P13667 | Protein disulfide-isomerase |
| | | | | A4 |
| 211 | Cardiometabolic_II_LAMA1 | LAMA1 | P25391 | Laminin subunit alpha-1 |
| 212 | Inflammation_SLAMF7 | SLAMF7 | Q9NQ25 | SLAM family member 7 |
| 213 | Inflammation_RGS8 | RGS8 | P57771 | Regulator of G-protein |
| | | | | signaling 8 |
| 214 | Inflammation_IL6 | IL6 | P05231 | Interleukin-6 |
| 215 | Neurology_PSG1 | PSG1 | P11464 | Pregnancy-specific beta-1- |
| | | | | glycoprotein 1 |
| 216 | Inflammation_II_PZP | PZP | P20742 | Pregnancy zone protein |
| 217 | Oncology_RRM2 | RRM2 | P31350 | Ribonucleoside-diphosphate |
| | | | | reductase subunit M2 |
| 218 | Neurology_II_GFRAL | GFRAL | Q6UXV0 | GDNF family receptor |
| | | | | alpha-like |
| 219 | Cardiometabolic_II_AIF1L | AIF1L | Q9BQI0 | Allograft inflammatory |
| | | | | factor 1-like |
| 220 | Inflammation_LGMN | LGMN | Q99538 | Legumain |
| 221 | Inflammation_II_C1QTNF9 | C1QTNF9 | P0C862 | Complement C1q and tumor |
| | | | | necrosis factor-related |
| | | | | protein 9A |
| 222 | Cardiometabolic_TSPAN1 | TSPAN1 | O60635 | Tetraspanin-1 |
| 223 | Cardiometabolic_II_DLL4 | DLL4 | Q9NR61 | Delta-like protein 4 |
| 224 | Inflammation_CRELD2 | CRELD2 | Q6UXH1 | Protein disulfide isomerase |
| | | | | CRELD2 |
| 225 | Cardiometabolic_SCARF1 | SCARF1 | Q14162 | Scavenger receptor class F |
| | | | | member 1 |
| 226 | Oncology_II_FGF9 | FGF9 | P31371 | Fibroblast growth factor 9 |
| 227 | Inflammation_II_JAM3 | JAM3 | Q9BX67 | Junctional adhesion |
| | | | | molecule C |
| 228 | Cardiometabolic_II_LPP | LPP | Q93052 | Lipoma-preferred partner |
| 229 | Cardiometabolic_HSPB1 | HSPB1 | P04792 | Heat shock protein beta-1 |
| 230 | Neurology_II_PPT1 | PPT1 | P50897 | Palmitoyl-protein |
| | | | | thioesterase 1 |
| 231 | Cardiometabolic_II_PPIF | PPIF | P30405 | Peptidyl-prolyl cis-trans |
| | | | | isomerase F, mitochondrial |
| 232 | Cardiometabolic_II_TRPV3 | TRPV3 | Q8NET8 | Transient receptor potential |
| | | | | cation channel subfamily V |
| | | | | member 3 |
| 233 | Inflammation_II_APOA4 | APOA4 | P06727 | Apolipoprotein A-IV |
| 234 | Neurology_II_LYSMD3 | LYSMD3 | Q7Z3D4 | LysM and putative |
| | | | | peptidoglycan-binding |
| | | | | domain-containing protein 3 |
| 235 | Inflammation_TGFA | TGFA | P01135 | Protransforming growth |
| | | | | factor alpha |
| 236 | Oncology_ATP6V1D | ATP6V1D | Q9Y5K8 | V-type proton ATPase |
| | | | | subunit D |
| 237 | Neurology_II_LRRC38 | LRRC38 | Q5VT99 | Leucine-rich repeat- |
| | | | | containing protein 38 |
| 238 | Oncology_II_CTAG1A_CTAG1B | CTAG1A | P78358 | Cancer/testis antigen 1 |
| 239 | Cardiometabolic_TINAGL1 | TINAGL1 | Q9GZM7 | Tubulointerstitial nephritis |
| | | | | antigen-like |
| 240 | Inflammation_II_POLR2A | POLR2A | P24928 | DNA-directed RNA |
| | | | | polymerase II subunit RPB1 |
| 241 | Cardiometabolic_EDIL3 | EDIL3 | O43854 | EGF-like repeat and |
| | | | | discoidin I-like domain- |
| | | | | containing protein 3 |
| 242 | Inflammation_LAP3 | LAP3 | P28838 | Cytosol aminopeptidase |
| 243 | Oncology_SORD | SORD | Q00796 | Sorbitol dehydrogenase |
| 244 | Oncology_II_ARHGAP30 | ARHGAP30 | Q7Z616 | Rho GTPase-activating |
| | | | | protein 30 |
| 245 | Cardiometabolic_II_CSPG4 | CSPG4 | Q6UVK1 | Chondroitin sulfate |
| | | | | proteoglycan 4 |
| 246 | Cardiometabolic_ART3 | ART3 | Q13508 | Ecto-ADP-ribosyltransferase |
| | | | | 3 |
| 247 | Cardiometabolic_II_GADD45GIP1 | GADD45GIP1 | Q8TAE8 | Growth arrest and DNA |
| | | | | damage-inducible proteins- |
| | | | | interacting protein 1 |
| 248 | Cardiometabolic_II_SLURP1 | SLURP1 | P55000 | Secreted Ly-6/uPAR-related |
| | | | | protein 1 |
| 249 | Neurology_LILRA2 | LILRA2 | Q8N149 | Leukocyte immunoglobulin- |
| | | | | like receptor subfamily A |
| | | | | member 2 |
| 250 | Cardiometabolic_GZMH | GZMH | P20718 | Granzyme H |
| 251 | Neurology_FKBP7 | FKBP7 | Q9Y680 | Peptidyl-prolyl cis-trans |
| | | | | isomerase FKBP7 |
| 252 | Neurology_SLC27A4 | SLC27A4 | Q6P1M0 | Long-chain fatty acid |
| | | | | transport protein 4 |
| 253 | Neurology_II_CALCB | CALCB | P10092 | Calcitonin gene-related |
| | | | | peptide 2 |
| 254 | Inflammation_II_GIT1 | GIT1 | Q9Y2X7 | ARF GTPase-activating |
| | | | | protein GIT1 |
| 255 | Inflammation_CTSO | CTSO | P43234 | Cathepsin O |
| 256 | Inflammation_II_PCBD1 | PCBD1 | P61457 | Pterin-4-alpha- |
| | | | | carbinolamine dehydratase |
| 257 | Inflammation_II_CSF3R | CSF3R | Q99062 | Granulocyte colony- |
| | | | | stimulating factor receptor |
| 258 | Neurology_II_EIF1AX | EIF1AX | P47813 | Eukaryotic translation |
| | | | | initiation factor 1A, X- |
| | | | | chromosomal |
| 259 | Neurology_II_CSPG5 | CSPG5 | O95196 | Chondroitin sulfate |
| | | | | proteoglycan 5 |
| 260 | Cardiometabolic_CD93 | CD93 | Q9NPY3 | Complement component |
| | | | | C1q receptor |
| 261 | Cardiometabolic_II_ADAMTSL5 | ADAMTSL5 | Q6ZMM2 | ADAMTS-like protein 5 |
| 262 | Cardiometabolic_II_ISM2 | ISM2 | Q6H9L7 | Isthmin-2 |
| 263 | Oncology_CPE | CPE | P16870 | Carboxypeptidase E |
| 264 | Oncology_II_WFDC1 | WFDC1 | Q9HC57 | WAP four-disulfide core |
| | | | | domain protein 1 |
| 265 | Neurology_VWC2 | VWC2 | Q2TAL6 | Brorin |
| 266 | Neurology_SPINK5 | SPINK5 | Q9NQ38 | Serine protease inhibitor |
| | | | | Kazal-type 5 |
| 267 | Oncology_II_BTN1A1 | BTN1A1 | Q13410 | Butyrophilin subfamily 1 |
| | | | | member A1 |
| 268 | Cardiometabolic_DPT | DPT | Q07507 | Dermatopontin |
| 269 | Inflammation_II_FCN1 | FCN1 | O00602 | Ficolin-1 |
| 270 | Oncology_AIF1 | AIF1 | P55008 | Allograft inflammatory |
| | | | | factor 1 |
| 271 | Oncology_GPC1 | GPC1 | P35052 | Glypican-1 |
| 272 | Cardiometabolic_FAP | FAP | Q12884 | Prolyl endopeptidase FAP |
| 273 | Neurology_II_CLNS1A | CLNS1A | P54105 | Methylosome subunit pICln |
| 274 | Oncology_CFC1 | CFC1 | P0CG37 | Cryptic protein |
| 275 | Inflammation_FASLG | FASLG | P48023 | Tumor necrosis factor ligand |
| | | | | superfamily member 6 |
| 276 | Oncology_NCS1 | NCS1 | P62166 | Neuronal calcium sensor 1 |
| 277 | Cardiometabolic_PRKAR1A | PRKAR1A | P10644 | cAMP-dependent protein |
| | | | | kinase type I-alpha |
| | | | | regulatory subunit |
| 278 | Cardiometabolic_RCOR1 | RCOR1 | Q9UKL0 | REST corepressor 1 |
| 279 | Oncology_SLITRK2 | SLITRK2 | Q9H156 | SLIT and NTRK-like protein |
| | | | | 2 |
| 280 | Cardiometabolic_SPARCL1 | SPARCL1 | Q14515 | SPARC-like protein 1 |
| 281 | Oncology_HSPB6 | HSPB6 | O14558 | Heat shock protein beta-6 |
| 282 | Oncology_TNFRSF12A | TNFRSF12A | Q9NP84 | Tumor necrosis factor |
| | | | | receptor superfamily |
| | | | | member 12A |
| 283 | Cardiometabolic_IL6 | IL6 | P05231 | Interleukin-6 |
| 284 | Inflammation_II_SERPIND1 | SERPIND1 | P05546 | Heparin cofactor 2 |
| 285 | Cardiometabolic_CEBPB | CEBPB | P17676 | CCAAT/enhancer-binding |
| | | | | protein beta |
| 286 | Neurology_II_CASC3 | CASC3 | O15234 | Protein CASC3 |
| 287 | Neurology_II_AMPD3 | AMPD3 | Q01432 | AMP deaminase 3 |
| 288 | Inflammation_YTHDF3 | YTHDF3 | Q7Z739 | YTH domain-containing |
| | | | | family protein 3 |
| 289 | Cardiometabolic_II_AAMDC | AAMDC | Q9H7C9 | Mth938 domain-containing |
| | | | | protein |
| 290 | Inflammation_II_STX7 | STX7 | O15400 | Syntaxin-7 |
| 291 | Inflammation_AGRP | AGRP | O00253 | Agouti-related protein |
| 292 | Inflammation_ICA1 | ICA1 | Q05084 | Islet cell autoantigen 1 |
| 293 | Oncology_II_CHCHD6 | CHCHD6 | Q9BRQ6 | MICOS complex subunit |
| | | | | MIC25 |
| 294 | Cardiometabolic_II_IGSF21 | IGSF21 | Q96ID5 | Immunoglobulin |
| | | | | superfamily member 21 |
| 295 | Neurology_VSTM1 | VSTM1 | Q6UX27 | V-set and transmembrane |
| | | | | domain-containing protein 1 |
| 296 | Oncology_II_PCDH7 | PCDH7 | O60245 | Protocadherin-7 |
| 297 | Oncology_VNN2 | VNN2 | O95498 | Vascular non-inflammatory |
| | | | | molecule 2 |
| 298 | Neurology_GP6 | GP6 | Q9HCN6 | Platelet glycoprotein VI |
| 299 | Oncology_ITGAV | ITGAV | P06756 | Integrin alpha-V |
| 300 | Inflammation_CD40LG | CD40LG | P29965 | CD40 ligand |
| 301 | Cardiometabolic_II_GIP | GIP | P09681 | Gastric inhibitory |
| | | | | polypeptide |
| 302 | Cardiometabolic_MB | MB | P02144 | Myoglobin |
| 303 | Inflammation_II_TPD52L2 | TPD52L2 | O43399 | Tumor protein D54 |
| 304 | Cardiometabolic_II_HPSE | HPSE | Q9Y251 | Heparanase |
| 305 | Neurology_II_GRIN2B | GRIN2B | Q13224 | Glutamate receptor |
| | | | | ionotropic, NMDA 2B |
| 306 | Inflammation_II_TREML1 | TREML1 | Q86YW5 | Trem-like transcript 1 |
| | | | | protein |
| 307 | Inflammation_II_C3 | C3 | P01024 | Complement C3 |
| 308 | Inflammation_II_TNFRSF17 | TNFRSF17 | Q02223 | Tumor necrosis factor |
| | | | | receptor superfamily |
| | | | | member 17 |
| 309 | Oncology_IL6 | IL6 | P05231 | Interleukin-6 |
| 310 | Inflammation_II_CD226 | CD226 | Q15762 | CD226 antigen |
| 311 | Oncology_II_PALM | PALM | O75781 | Paralemmin-1 |
| 312 | Neurology_II_FKBP14 | FKBP14 | Q9NWM8 | Peptidyl-prolyl cis-trans |
| | | | | isomerase FKBP14 |
| 313 | Cardiometabolic_II_RBPMS2 | RBPMS2 | Q6ZRY4 | RNA-binding protein with |
| | | | | multiple splicing 2 |
| 314 | Oncology_CLEC6A | CLEC6A | Q6EIG7 | C-type lectin domain family |
| | | | | 6 member A |
| 315 | Inflammation_II_DAAM1 | DAAM1 | Q9Y4D1 | Disheveled-associated |
| | | | | activator of morphogenesis 1 |
| 316 | Oncology_II_FAM3D | FAM3D | Q96BQ1 | Protein FAM3D |
| 317 | Cardiometabolic_WASF1 | WASF1 | Q92558 | Wiskott-Aldrich syndrome |
| | | | | protein family member 1 |
| 318 | Cardiometabolic_II_HS1BP3 | HS1BP3 | Q53T59 | HCLS1-binding protein 3 |
| 319 | Neurology_NOS3 | NOS3 | P29474 | Nitric oxide synthase, |
| | | | | endothelial |
| 320 | Inflammation_II_POF1B | POF1B | Q8WVV4 | Protein POF1B |
| 321 | Inflammation_PLXNA4 | PLXNA4 | Q9HCM2 | Plexin-A4 |
| 322 | Neurology_MITD1 | MITD1 | Q8WV92 | MIT domain-containing |
| | | | | protein 1 |
| 323 | Inflammation_II_ERMAP | ERMAP | Q96PL5 | Erythroid membrane- |
| | | | | associated protein |
| 324 | Inflammation_II_SYAP1 | SYAP1 | Q96A49 | Synapse-associated protein 1 |
| 325 | Cardiometabolic_II_LRRC59 | LRRC59 | Q96AG4 | Leucine-rich repeat- |
| | | | | containing protein 59 |
| 326 | Oncology_CNTN2 | CNTN2 | Q02246 | Contactin-2 |
| 327 | Oncology_II_RAB2B | RAB2B | Q8WUD1 | Ras-related protein Rab-2B |
| 328 | Inflammation_II_PENK | PENK | P01210 | Proenkephalin-A |
| 329 | Cardiometabolic_MCAM | MCAM | P43121 | Cell surface glycoprotein |
| | | | | MUC18 |
| 330 | Cardiometabolic_II_EIF2S2 | EIF2S2 | P20042 | Eukaryotic translation |
| | | | | initiation factor 2 subunit 2 |
| 331 | Inflammation_EGF | EGF | P01133 | Pro-epidermal growth factor |
| 332 | Inflammation_PTPN6 | PTPN6 | P29350 | Tyrosine-protein |
| | | | | phosphatase non-receptor |
| | | | | type 6 |
| 333 | Neurology_NID2 | NID2 | Q14112 | Nidogen-2 |
| 334 | Cardiometabolic_II_EHD3 | EHD3 | Q9NZN3 | EH domain-containing |
| | | | | protein 3 |
| 335 | Cardiometabolic_IGFBP6 | IGFBP6 | P24592 | Insulin-like growth factor- |
| | | | | binding protein 6 |
| 336 | Inflammation_II_LMOD1 | LMOD1 | P29536 | Leiomodin-1 |
| 337 | Cardiometabolic_II_PAGR1 | PAGR1 | Q9BTK6 | PAXIP1-associated |
| | | | | glutamate-rich protein 1 |
| 338 | Neurology_CD300C | CD300C | Q08708 | CMRF35-like molecule 6 |
| 339 | Inflammation_SKAP2 | SKAP2 | O75563 | Src kinase-associated |
| | | | | phosphoprotein 2 |
| 340 | Inflammation_II_PRKG1 | PRKG1 | Q13976 | cGMP-dependent protein |
| | | | | kinase 1 |
| 341 | Cardiometabolic_II_SYTL4 | SYTL4 | Q96C24 | Synaptotagmin-like protein 4 |
| 342 | Cardiometabolic_GYS1 | GYS1 | P13807 | Glycogen [starch] synthase, |
| | | | | muscle |
| 343 | Cardiometabolic_CASP3 | CASP3 | P42574 | Caspase-3 |
| 344 | Neurology_PILRA | PILRA | Q9UKJ1 | Paired immunoglobulin-like |
| | | | | type 2 receptor alpha |
| 345 | Cardiometabolic_CD69 | CD69 | Q07108 | Early activation antigen |
| | | | | CD69 |
| 346 | Neurology_CCN5 | CCN5 | O76076 | CCN family member 5 |
| 347 | Neurology_II_PCBP2 | PCBP2 | Q15366 | Poly(rC)-binding protein 2 |
| 348 | Oncology_II_LMOD1 | LMOD1 | P29536 | Leiomodin-1 |
| 349 | Oncology_II_PDIA5 | PDIA5 | Q14554 | Protein disulfide-isomerase |
| | | | | A5 |
| 350 | Oncology_II_PCSK7 | PCSK7 | Q16549 | Proprotein convertase |
| | | | | subtilisin/kexin type 7 |
| 351 | Neurology_SCARA5 | SCARA5 | Q6ZMJ2 | Scavenger receptor class A |
| | | | | member 5 |
| 352 | Inflammation_METAP1D | MEETAP1D | Q6UB28 | Methionine aminopeptidase |
| | | | | 1D, mitochondrial |
| 353 | Neurology_ADGRB3 | ADGRB3 | O60242 | Adhesion G protein-coupled |
| | | | | receptor B3 |
| 354 | Inflammation_MPIG6B | MPIG6B | O95866 | Megakaryocyte and platelet |
| | | | | inhibitory receptor G6b |
| 355 | Inflammation_II_NUMB | NUMB | P49757 | Protein numb homolog |
| 356 | Cardiometabolic_II_L3HYPDH | L3HYPDH | Q96EM0 | Trans-3-hydroxy-L-proline |
| | | | | dehydratase |
| 357 | Inflammation_II_DENR | DENR | O43583 | Density-regulated protein |
| 358 | Inflammation_AGRN | AGRN | O00468 | Agrin |
| 359 | Cardiometabolic_II_COX6B1 | COX6B1 | P14854 | Cytochrome c oxidase |
| | | | | subunit 6B1 |
| 360 | Neurology_JAM2 | JAM2 | P57087 | Junctional adhesion |
| | | | | molecule B |
| 361 | Cardiometabolic_TIA1 | TIA1 | P31483 | Nucleolysin TIA-1 isoform |
| | | | | p40 |
| 362 | Inflammation_II_CACYBP | CACYBP | Q9HB71 | Calcyclin-binding protein |
| 363 | Inflammation_II_SEMA6C | SEMA6C | Q9H3T2 | Semaphorin-6C |
| 364 | Oncology_VAT1 | VAT1 | Q99536 | Synaptic vesicle membrane |
| | | | | protein VAT-1 homolog |
| 365 | Cardiometabolic_SUSD1 | SUSD1 | Q6UWL2 | Sushi domain-containing |
| | | | | protein 1 |
| 366 | Oncology_RSPO3 | RSPO3 | Q9BXY4 | R-spondin-3 |
| 367 | Cardiometabolic_II_TWF2 | TWF2 | Q6IBS0 | Twinfilin-2 |
| 368 | Neurology_II_BOLA1 | BOLA1 | Q9Y3E2 | BolA-like protein 1 |
| 369 | Cardiometabolic_II_OXCT1 | OXCT1 | P55809 | Succinyl-CoA: 3-ketoacid |
| | | | | coenzyme A transferase 1, |
| | | | | mitochondrial |
| 370 | Inflammation_ITGA6 | ITGA6 | P23229 | Integrin alpha-6 |
| 371 | Neurology_BST2 | BST2 | Q10589 | Bone marrow stromal |
| | | | | antigen 2 |
| 372 | Inflammation_F2R | F2R | P25116 | Proteinase-activated receptor |
| | | | | 1 |
| 373 | Cardiometabolic_PILRB | PILRB | Q9UKJ0 | Paired immunoglobulin-like |
| | | | | type 2 receptor beta |
| 374 | Oncology_RTBDN | RTBDN | Q9BSG5 | Retbindin |
| 375 | Cardiometabolic_II_ENOX2 | ENOX2 | Q16206 | Ecto-NOX disulfide-thiol |
| | | | | exchanger 2 |
| 376 | Neurology_II_DOK1 | DOK1 | Q99704 | Docking protein 1 |
| 377 | Inflammation_VASH1 | VASH1 | Q7L8A9 | Tubulinyl-Tyr |
| | | | | carboxypeptidase 1 |
| 378 | Inflammation_II_DTD1 | DTD1 | Q8TEA8 | D-aminoacyl-tRNA |
| | | | | deacylase 1 |
| 379 | Neurology_II_DDHD2 | DDHD2 | O94830 | Phospholipase DDHD2 |
| 380 | Oncology_TBC1D23 | TBC1D23 | Q9NUY8 | TBC1 domain family |
| | | | | member 23 |
| 381 | Inflammation_II_GLRX5 | GLRX5 | Q86SX6 | Glutaredoxin-related protein |
| | | | | 5, mitochondrial |
| 382 | Oncology_CDNF | CDNF | Q49AH0 | Cerebral dopamine |
| | | | | neurotrophic factor |
| 383 | Inflammation_SIRPB1 | SIRPB1 | O00241 | Signal-regulatory protein |
| | | | | beta-1 |
| 384 | Neurology_II_NMT1 | NMT1 | P30419 | Glycylpeptide N- |
| | | | | tetradecanoyltransferase 1 |
| 385 | Cardiometabolic_STK11 | STK11 | Q15831 | Serine/threonine-protein |
| | | | | kinase STK11 |
| 386 | Cardiometabolic_II_RPL14 | RPL 14 | P50914 | 60S ribosomal protein L14 |
| 387 | Inflammation_II_PSTPIP2 | PSTPIP2 | Q9H939 | Proline-serine-threonine |
| | | | | phosphatase-interacting |
| | | | | protein 2 |
| 388 | Neurology_FHIT | FHIT | P49789 | Bis(5′-adenosyl)- |
| | | | | triphosphatase |
| 389 | Oncology_CLMP | CLMP | Q9H6B4 | CXADR-like membrane |
| | | | | protein |
| 390 | Neurology_II_LMOD1 | LMOD1 | P29536 | Leiomodin-1 |
| 391 | Inflammation_II_ERP29 | ERP29 | P30040 | Endoplasmic reticulum |
| | | | | resident protein 29 |
| 392 | Cardiometabolic_II_BECN1 | BECN1 | Q14457 | Beclin-1 |
| 393 | Oncology_CD38 | CD38 | P28907 | ADP-ribosyl cyclase/cyclic |
| | | | | ADP-ribose hydrolase 1 |
| 394 | Neurology_II_YAP1 | YAP1 | P46937 | Transcriptional coactivator |
| | | | | YAP1 |
| 395 | Cardiometabolic_CA13 | CA13 | Q8N1Q1 | Carbonic anhydrase 13 |
| 396 | Inflammation_CRKL | CRKL | P46109 | Crk-like protein |
| 397 | Inflammation_PPP1R9B | PPP1R9B | Q96SB3 | Neurabin-2 |
| 398 | Oncology_FLI1 | FLI1 | Q01543 | Friend leukemia integration |
| | | | | 1 transcription factor |
| 399 | Cardiometabolic_II_CMC1 | CMC1 | Q7Z7K0 | COX assembly |
| | | | | mitochondrial protein |
| | | | | homolog |
| 400 | Oncology_CDC37 | CDC37 | Q16543 | Hsp90 co-chaperone Cdc37 |
| 401 | Inflammation_II_ARHGAP45 | ARHGAP45 | Q92619 | Rho GTPase-activating |
| | | | | protein 45 |
| 402 | Cardiometabolic_II_PDAP1 | PDAP1 | Q13442 | 28 kDa heat- and acid-stable |
| | | | | phosphoprotein |
| 403 | Inflammation_NUDC | NUDC | Q9Y266 | Nuclear migration protein |
| | | | | nudC |
| 404 | Neurology_CLEC1B | CLEC1B | Q9P126 | C-type lectin domain family |
| | | | | 1 member B |
| 405 | Oncology_USO1 | USO1 | O60763 | General vesicular transport |
| | | | | factor p115 |
| 406 | Cardiometabolic_SNAP23 | SNAP23 | O00161 | Synaptosomal-associated |
| | | | | protein 23 |
| 407 | Oncology_HGS | HGS | O14964 | Hepatocyte growth factor- |
| | | | | regulated tyrosine kinase |
| | | | | substrate |
| 408 | Oncology_FUS | FUS | P35637 | RNA-binding protein FUS |
| 409 | Inflammation_PIK3AP1 | PIK3AP1 | Q6ZUJ8 | Phosphoinositide 3-kinase |
| | | | | adapter protein 1 |
| 410 | Neurology_F11R | F11R | Q9Y624 | Junctional adhesion |
| | | | | molecule A |
| 411 | Neurology_TBC1D17 | TBC1D17 | Q9HA65 | TBC1 domain family |
| | | | | member 17 |
| 412 | Cardiometabolic_II_ITPA | ITPA | Q9BY32 | Inosine triphosphate |
| | | | | pyrophosphatase |
| 413 | Inflammation_IL1B | IL1B | P01584 | Interleukin-1 beta |
| 414 | Neurology_ENO1 | ENO1 | P06733 | Alpha-enolase |
| 415 | Oncology_II_THTPA | THTPA | Q9BU02 | Thiamine-triphosphatase |
| 416 | Neurology_II_SAFB2 | SAFB2 | Q14151 | Scaffold attachment factor |
| | | | | B2 |
| 417 | Oncology_II_JPT2 | JPT2 | Q9H910 | Jupiter microtubule |
| | | | | associated homolog 2 |
| 418 | Inflammation_II_GIMAP7 | GIMAP7 | Q8NHV1 | GTPase IMAP family |
| | | | | member 7 |
| 419 | Cardiometabolic_II_NIT2 | NIT2 | Q9NQR4 | Omega-amidase NIT2 |
| 420 | Cardiometabolic_II_RILPL2 | RILPL2 | Q969X0 | RILP-like protein 2 |
| 421 | Neurology_PRTFDC1 | PRTFDC1 | Q9NRG1 | Phosphoribosyltransferase |
| | | | | domain-containing protein 1 |
| 422 | Oncology_II_TADA3 | TADA3 | O75528 | Transcriptional adapter 3 |
| 423 | Cardiometabolic_II_TOMM20 | TOMM20 | Q15388 | Mitochondrial import |
| | | | | receptor subunit TOM20 |
| | | | | homolog |
| 424 | Inflammation_HPCAL1 | HPCAL1 | P37235 | Hippocalcin-like protein 1 |
| 425 | Cardiometabolic_II_LONP1 | LONP1 | P36776 | Lon protease homolog, |
| | | | | mitochondrial |
| 426 | Oncology_CALCOCO1 | CALCOCO1 | Q9P1Z2 | Calcium-binding and coiled- |
| | | | | coil domain-containing |
| | | | | protein 1 |
| 427 | Oncology_II_ATRAID | ATRAID | Q6UW56 | All-trans retinoic acid- |
| | | | | induced differentiation factor |
| 428 | Cardiometabolic_TYMP | TYMP | P19971 | Thymidine phosphorylase |
| 429 | Oncology_TNFRSF19 | TNFRSF19 | Q9NS68 | Tumor necrosis factor |
| | | | | receptor superfamily |
| | | | | member 19 |
| 430 | Neurology_II_DNPEP | DNPEP | Q9ULA0 | Aspartyl aminopeptidase |
| 431 | Inflammation_II_NRGN | NRGN | Q92686 | Neurogranin |
| 432 | Cardiometabolic_STK4 | STK4 | Q13043 | Serine/threonine-protein |
| | | | | kinase 4 |
| 433 | Oncology_II_SSNA1 | SSNA1 | O43805 | Sjoegren syndrome nuclear |
| | | | | autoantigen 1 |
| 434 | Neurology_II_CRYGD | CRYGD | P07320 | Gamma-crystallin D |
| 435 | Inflammation_II_LZTFL1 | LZTFL1 | Q9NQ48 | Leucine zipper transcription |
| | | | | factor-like protein 1 |
| 436 | Oncology_SNAP29 | SNAP29 | O95721 | Synaptosomal-associated |
| | | | | protein 29 |
| 437 | Neurology_II_PDLIM5 | PDLIM5 | Q96HC4 | PDZ and LIM domain |
| | | | | protein 5 |
| 438 | Inflammation_CASP2 | CASP2 | P42575 | Caspase-2 |
| 439 | Inflammation_MANF | MANF | P55145 | Mesencephalic astrocyte- |
| | | | | derived neurotrophic factor |
| 440 | Inflammation_BACH1 | BACH1 | O14867 | Transcription regulator |
| | | | | protein BACH1 |
| 441 | Inflammation_DAPP1 | DAPP1 | Q9UN19 | Dual adapter for |
| | | | | phosphotyrosine and 3- |
| | | | | phosphotyrosine and 3- |
| | | | | phosphoinositide |
| 442 | Oncology_AKR1B1 | AKR1B1 | P15121 | Aldo-keto reductase family 1 |
| | | | | member B1 |
| 443 | Neurology_EREG | EREG | O14944 | Proepiregulin |
| 444 | Inflammation_DAG1 | DAG1 | Q14118 | Dystroglycan |
| 445 | Cardiometabolic_II_HSBP1 | HSBP1 | O75506 | Heat shock factor-binding |
| | | | | protein 1 |
| 446 | Oncology_II_DUT | DUT | P33316 | Deoxyuridine 5′-triphosphate |
| | | | | nucleotidohydrolase, |
| | | | | mitochondrial |
| 447 | Neurology_II_AKT2 | AKT2 | P31751 | RAC-beta serine/threonine- |
| | | | | protein kinase |
| 448 | Inflammation_PLA2G4A | PLA2G4A | P47712 | Cytosolic phospholipase A2 |
| 449 | Neurology_TXLNA | TXLNA | P40222 | Alpha-taxilin |
| 450 | Inflammation_II_PIKFYVE | PIKFYVE | Q9Y217 | 1-phosphatidylinositol 3- |
| | | | | phosphate 5-kinase |
| 451 | Neurology_FYB1 | FYB1 | O15117 | FYN-binding protein 1 |
| 452 | Cardiometabolic_II_CSDE1 | CSDE1 | O75534 | Cold shock domain- |
| | | | | containing protein E1 |
| 453 | Neurology_RHOC | RHOC | P08134 | Rho-related GTP-binding |
| | | | | protein RhoC |
| 454 | Cardiometabolic_HNRNPK | HNRNPK | P61978 | Heterogeneous nuclear |
| | | | | ribonucleoprotein K |
| 455 | Inflammation_II_DCTD | DCTD | P32321 | Deoxycytidylate deaminase |
| 456 | Cardiometabolic_II_SCRG1 | SCRG1 | O75711 | Scrapie-responsive protein 1 |
| 457 | Cardiometabolic_LACTB2 | LACTB2 | Q53H82 | Endoribonuclease LACTB2 |
| 458 | Neurology_II_RGCC | RGCC | Q9H4X1 | Regulator of cell cycle |
| | | | | RGCC |
| 459 | Oncology_II_GIMAP8 | GIMAP8 | Q8ND71 | GTPase IMAP family |
| | | | | member 8 |
| 460 | Cardiometabolic_II_GRHPR | GRHPR | Q9UBQ7 | Glyoxylate |
| | | | | reductase/hydroxypyruvate |
| | | | | reductase |
| 461 | Cardiometabolic_II_SNX5 | SNX5 | Q9Y5X3 | Sorting nexin-5 |
| 462 | Inflammation_NCK2 | NCK2 | O43639 | Cytoplasmic protein NCK2 |
| 463 | Inflammation_EIF4G1 | EIF4G1 | Q04637 | Eukaryotic translation |
| | | | | initiation factor 4 gamma 1 |
| 464 | Inflammation_II_BNIP3L | BNIP3L | O60238 | BCL2/adenovirus E1B 19 |
| | | | | kDa protein-interacting |
| | | | | protein 3-like |
| 465 | Oncology_II_ACOT13 | ACOT13 | Q9NPJ3 | Acyl-coenzyme A |
| | | | | thioesterase 13 |
| 466 | Cardiometabolic_II_MECR | MECR | Q9BV79 | Enoyl-[acyl-carrier-protein] |
| | | | | reductase, mitochondrial |
| 467 | Inflammation_MAP2K6 | MAP2K6 | P52564 | Dual specificity mitogen- |
| | | | | activated protein kinase |
| | | | | kinase 6 |
| 468 | Cardiometabolic_II_SEC31A | SEC31A | O94979 | Protein transport protein |
| | | | | Sec31A |
| 469 | Inflammation_MGLL | MGLL | Q99685 | Monoglyceride lipase |
| 470 | Neurology_MESD | MESD | Q14696 | LRP chaperone MESD |
| 471 | Oncology_II_NUDT16 | NUDT16 | Q96DE0 | U8 snoRNA-decapping |
| | | | | enzyme |
| 472 | Neurology_SULT1A1 | SULT1A1 | P50225 | Sulfotransferase 1A1 |
| 473 | Inflammation_GOPC | GOPC | Q9HD26 | Golgi-associated PDZ and |
| | | | | coiled-coil motif-containing |
| | | | | protein |
| 474 | Neurology_VTA1 | VTA1 | Q9NP79 | Vacuolar protein sorting- |
| | | | | associated protein VTA1 |
| | | | | homolog |
| 475 | Inflammation_PDLIM7 | PDLIM7 | Q9NR12 | PDZ and LIM domain |
| | | | | protein 7 |
| 476 | Cardiometabolic_II_ANXA2 | ANXA2 | P07355 | Annexin A2 |
| 477 | Cardiometabolic_II_GGACT | GGACT | Q9BVM4 | Gamma- |
| | | | | glutamylaminecyclotransferase |
| 478 | Neurology_PMVK | PMVK | Q15126 | Phosphomevalonate kinase |
| 479 | Cardiometabolic_USP8 | USP8 | P40818 | Ubiquitin carboxyl-terminal |
| | | | | hydrolase 8 |
| 480 | Inflammation_II_SNCA | SNCA | P37840 | Alpha-synuclein |
| 481 | Neurology_II_CAMSAP1 | CAMSAP1 | Q5T5Y3 | Calmodulin-regulated |
| | | | | spectrin-associated protein 1 |
| 482 | Inflammation_HEXIM1 | HEXIM1 | O94992 | Protein HEXIM1 |
| 483 | Inflammation_SHMT1 | SHMT1 | P34896 | Serine |
| | | | | hydroxymethyltransferase, |
| | | | | cytosolic |
| 484 | Neurology_LGALS8 | LGALS8 | O00214 | Galectin-8 |
| 485 | Inflammation_II_APPL2 | APPL2 | Q8NEU8 | DCC-interacting protein 13- |
| | | | | beta |
| 486 | Oncology_II_MAP2K1 | MAP2K1 | Q02750 | Dual specificity mitogen- |
| | | | | activated protein kinase |
| | | | | kinase 1 |
| 487 | Cardiometabolic_II_EHBP1 | EHBP1 | Q8NDI1 | EH domain-binding protein |
| | | | | 1 |
| 488 | Neurology_MAP4K5 | MAP4K5 | Q9Y4K4 | Mitogen-activated protein |
| | | | | kinase kinase kinase kinase 5 |
| 489 | Inflammation_II_PDE5A | PDE5A | O76074 | cGMP-specific 3′,5′-cyclic |
| | | | | phosphodiesterase |
| 490 | Neurology_HARS1 | HARS1 | P12081 | Histidine--tRNA ligase, |
| | | | | cytoplasmic |
| 491 | Oncology_SRC | SRC | P12931 | Proto-oncogene tyrosine- |
| | | | | protein kinase Src |
| 492 | Oncology_TACC3 | TACC3 | Q9Y6A5 | Transforming acidic coiled- |
| | | | | coil-containing protein 3 |
| 493 | Cardiometabolic_II_RAB27B | RAB27B | O00194 | Ras-related protein Rab-27B |

TABLE 3
Identification of biomarkers in “1-3 Y” prediction models in Olink ® Explore 3072 Platform

| | | Biomarker | | |
|---|---|---|---|---|
| Rank | Biomarker Category | symbol | UniProt | Biomarker Name |
| 1 | Oncology_II_VWA5A | VWA5A | O00534 | von Willebrand factor A domain- |
| | | | | containing protein 5A |
| 2 | Cardiometabolic_II_ENPP6 | ENPP6 | Q6UWR7 | Glycerophosphocholine |
| | | | | cholinephosphodiesterase ENPP6 |
| 3 | Neurology_II_TMEM25 | TMEM25 | Q86YD3 | Transmembrane protein 25 |
| 4 | Oncology_II_ALDH2 | ALDH2 | P05091 | Aldehyde dehydrogenase, |
| | | | | mitochondrial |
| 5 | Neurology_II_LEO1 | LEO1 | Q8WVC0 | RNA polymerase-associated |
| | | | | protein LEO1 |
| 6 | Cardiometabolic_II_GAMT | GAMT | Q14353 | Guanidinoacetate N- |
| | | | | methyltransferase |
| 7 | Inflammation_II_TPSG1 | TPSG1 | Q9NRR2 | Tryptase gamma |
| 8 | Cardiometabolic_II_ANK2 | ANK2 | Q01484 | Ankyrin-2 |
| 9 | Neurology_II_SCT | SCT | P09683 | Secretin |
| 10 | Neurology_II_TSPAN7 | TSPAN7 | P41732 | Tetraspanin-7 |
| 11 | Neurology_GPC5 | GPC5 | P78333 | Glypican-5 |
| 12 | Cardiometabolic_PGLYRP1 | PGLYRP1 | O75594 | Peptidoglycan recognition |
| | | | | protein 1 |
| 13 | Neurology_PAK4 | PAK4 | O96013 | Serine/threonine-protein kinase |
| | | | | PAK 4 |
| 14 | Neurology_TNFSF14 | TNFSF14 | O43557 | Tumor necrosis factor ligand |
| | | | | superfamily member 14 |
| 15 | Oncology_CLEC6A | CLEC6A | Q6EIG7 | C-type lectin domain family 6 |
| | | | | member A |
| 16 | Oncology_TMPRSS15 | TMPRSS15 | P98073 | Enteropeptidase |
| 17 | Cardiometabolic_II_PMCH | PMCH | P20382 | Pro-MCH |
| 18 | Neurology_KRT14 | KRT14 | P02533 | Keratin, type I cytoskeletal 14\″″ |
| 19 | Oncology_SFTPA1 | SFTPA1 | Q8IWL2 | Pulmonary surfactant-associated |
| | | | | protein A1 |
| 20 | Neurology_II_LRFN2 | LRFN2 | Q9ULH4 | Leucine-rich repeat and |
| | | | | fibronectin type-III domain- |
| | | | | containing protein 2 |
| 21 | Oncology_MMP12 | MMP12 | P39900 | Macrophage metalloelastase |
| 22 | Oncology_II_TNPO1 | TNPO1 | Q92973 | Transportin-1 |
| 23 | Neurology_II_GAST | GAST | P01350 | Gastrin |
| 24 | Neurology_II_CD3D | CD3D | P04234 | T-cell surface glycoprotein CD3 |
| | | | | delta chain |
| 25 | Oncology_II_TK1 | TK1 | P04183 | \Thymidine kinase, cytosolic\″″ |
| 26 | Neurology_II_DLGAP5 | DLGAP5 | Q15398 | Disks large-associated protein 5 |
| 27 | Inflammation_SCGN | SCGN | O76038 | Secretagogin |
| 28 | Inflammation_CCL24 | CCL24 | O00175 | C-C motif chemokine 24 |
| 29 | Neurology_PSG1 | PSG1 | P11464 | Pregnancy-specific beta-1- |
| | | | | glycoprotein 1 |
| 30 | Inflammation_II_CLU | CLU | P10909 | Clusterin |
| 31 | Inflammation_II_CFB | CFB | P00751 | Complement factor B |
| 32 | Cardiometabolic_LBP | LBP | P18428 | Lipopolysaccharide-binding |
| | | | | protein |
| 33 | Neurology_II_CRYM | CRYM | Q14894 | Ketimine reductase mu-crystallin |
| 34 | Neurology_LAIR2 | LAIR2 | Q6ISS4 | Leukocyte-associated |
| | | | | immunoglobulin-like receptor 2 |
| 35 | Cardiometabolic_TCN2 | TCN2 | P20062 | Transcobalamin-2 |
| 36 | Neurology_II_SV2A | SV2A | Q7L0J3 | Synaptic vesicle glycoprotein 2A |
| 37 | Inflammation_CRHBP | CRHBP | P24387 | Corticotropin-releasing factor- |
| | | | | binding protein |
| 38 | Inflammation_II_C5 | C5 | P01031 | Complement C5 |
| 39 | Inflammation_SCGB3A2 | SCGB3A2 | Q96PL1 | Secretoglobin family 3A member |
| | | | | 2 |
| 40 | Neurology_ANXA10 | ANXA10 | Q9UJ72 | Annexin A10 |
| 41 | Oncology_GCG | GCG | P01275 | Pro-glucagon |
| 42 | Neurology_II_RPGR | RPGR | Q92834 | X-linked retinitis pigmentosa |
| | | | | GTPase regulator |
| 43 | Inflammation_PAPPA | PAPPA | Q13219 | Pappalysin-1 |
| 44 | Neurology_II_FZD8 | FZD8 | Q9H461 | Frizzled-8 |
| 45 | Neurology_II_CSPG5 | CSPG5 | O95196 | Chondroitin sulfate proteoglycan |
| | | | | 5 |
| 46 | Neurology_BRK1 | BRK1 | Q8WUW1 | Protein BRICK1 |
| 47 | Neurology_OXT | OXT | P01178 | Oxytocin-neurophysin 1 |
| 48 | Cardiometabolic_II_FDX1 | FDX1 | P10109 | \Adrenodoxin, mitochondrial\″″ |
| 49 | Cardiometabolic_II_ENPEP | ENPEP | Q07075 | Glutamyl aminopeptidase |
| 50 | Inflammation_II_LRG1 | LRG1 | P02750 | Leucine-rich alpha-2- |
| | | | | glycoprotein |
| 51 | Oncology_II_PRAME | PRAME | P78395 | Melanoma antigen preferentially |
| | | | | expressed in tumors |
| 52 | Neurology_II_KIRREL1 | KIRREL1 | Q96J84 | Kin of IRRE-like protein 1 |
| 53 | Cardiometabolic_II_KIF22 | KIF22 | Q14807 | Kinesin-like protein KIF22 |
| 54 | Neurology_SPINT1 | SPINT1 | O43278 | Kunitz-type protease inhibitor 1 |
| 55 | Inflammation_II_FGA | FGA | P02671 | Fibrinogen alpha chain |
| 56 | Inflammation_II_C1QTNF9 | C1QTNF9 | P0C862 | Complement C1q and tumor |
| | | | | necrosis factor-related protein 9A |
| 57 | Oncology_II_KIR2DS4 | KIR2DS4 | P43632 | Killer cell immunoglobulin-like |
| | | | | receptor 2DS4 |
| 58 | Neurology_MMP9 | MMP9 | P14780 | Matrix metalloproteinase-9 |
| 59 | Inflammation_II_NEXN | NEXN | Q0ZGT2 | Nexilin |
| 60 | Inflammation_II_FCN1 | FCN1 | O00602 | Ficolin-1 |
| 61 | Neurology_MFGE8 | MFGE8 | Q08431 | Lactadherin |
| 62 | Oncology_II_ZNRD2 | ZNRD2 | O60232 | Protein ZNRD2 |
| 63 | Cardiometabolic_PDGFRB | PDGFRB | P09619 | Platelet-derived growth factor |
| | | | | receptor beta |
| 64 | Oncology_HS6ST1 | HS6ST1 | O60243 | Heparan-sulfate 6-O- |
| | | | | sulfotransferase 1 |
| 65 | Neurology_DUSP3 | DUSP3 | P51452 | Dual specificity protein |
| | | | | phosphatase 3 |
| 66 | Neurology_II_CABP2 | CABP2 | Q9NPB3 | Calcium-binding protein 2 |
| 67 | Neurology_II_DNM3 | DNM3 | Q9UQ16 | Dynamin-3 |
| 68 | Inflammation_II_FGL1 | FGL1 | Q08830 | Fibrinogen-like protein 1 |
| 69 | Oncology_II_TOP1 | TOP1 | P11387 | DNA topoisomerase 1 |
| 70 | Neurology_CDCP1 | CDCP1 | Q9H5V8 | CUB domain-containing protein |
| | | | | 1 |
| 71 | Cardiometabolic_II_RAB10 | RAB10 | P61026 | Ras-related protein Rab-10 |
| 72 | Inflammation_II_THSD1 | THSD1 | Q9NS62 | Thrombospondin type-1 domain- |
| | | | | containing protein 1 |
| 73 | Inflammation_FASLG | FASLG | P48023 | Tumor necrosis factor ligand |
| | | | | superfamily member 6 |
| 74 | Inflammation_II_MCEMP1 | MCEMP1 | Q8IX19 | Mast cell-expressed membrane |
| | | | | protein 1 |
| 75 | Oncology_II_COL4A4 | COL4A4 | P53420 | Collagen alpha-4(IV) chain |
| 76 | Neurology_ENO1 | ENO1 | P06733 | Alpha-enolase |
| 77 | Oncology_II_BRD1 | BRD1 | O95696 | Bromodomain-containing protein |
| | | | | 1 |
| 78 | Inflammation_II_GP5 | GP5 | P40197 | Platelet glycoprotein V |
| 79 | Cardiometabolic_II_ZP3 | ZP3 | P21754 | Zona pellucida sperm-binding |
| | | | | protein 3 |
| 80 | Inflammation_II_SERPIND1 | SERPIND1 | P05546 | Heparin cofactor 2 |
| 81 | Cardiometabolic_NCAM1 | NCAM1 | P13591 | Neural cell adhesion molecule 1 |
| 82 | Neurology_ATXN10 | ATXN10 | Q9UBB4 | Ataxin-10 |
| 83 | Oncology_MUC16 | MUC16 | Q8WXI7 | Mucin-16 |
| 84 | Neurology_II_GABRA4 | GABRA4 | P48169 | Gamma-aminobutyric acid |
| | | | | receptor subunit alpha-4 |
| 85 | Cardiometabolic_II_POSTN | POSTN | Q15063 | Periostin |
| 86 | Oncology_MAEA | MAEA | Q7L5Y9 | E3 ubiquitin-protein transferase |
| | | | | MAEA |
| 87 | Inflammation_II_SHH | SHH | Q15465 | Sonic hedgehog protein |
| 88 | Neurology_II_DDX53 | DDX53 | Q86TM3 | Probable ATP-dependent RNA |
| | | | | helicase DDX53 |
| 89 | Inflammation_II_PRKG1 | PRKG1 | Q13976 | cGMP-dependent protein kinase |
| | | | | 1 |
| 90 | Neurology_PAEP | PAEP | P09466 | Glycodelin |
| 91 | Inflammation_II_RICTOR | RICTOR | Q6R327 | Rapamycin-insensitive |
| | | | | companion of mTOR |
| 92 | Inflammation_IL6 | IL6 | P05231 | Interleukin-6 |
| 93 | Neurology_II_FKBP14 | FKBP14 | Q9NWM8 | Peptidyl-prolyl cis-trans |
| | | | | isomerase FKBP14 |
| 94 | Inflammation_CCL26 | CCL26 | Q9Y258 | C-C motif chemokine 26 |
| 95 | Neurology_II_AIDA | AIDA | Q96BJ3 | \Axin interactor, dorsalization- |
| | | | | associated protein\″″ |
| 96 | Cardiometabolic_II_GIP | GIP | P09681 | Gastric inhibitory polypeptide |
| 97 | Inflammation_TGFA | TGFA | P01135 | Protransforming growth factor |
| | | | | alpha |
| 98 | Inflammation_II_ITIH4 | ITIH4 | Q14624 | Inter-alpha-trypsin inhibitor |
| | | | | heavy chain H4 |
| 99 | Oncology_II_PCSK7 | PCSK7 | Q16549 | Proprotein convertase |
| | | | | subtilisin/kexin type 7 |
| 100 | Oncology_RARRES1 | RARRES1 | P49788 | Retinoic acid receptor responder |
| | | | | protein 1 |
| 101 | Neurology_SLC27A4 | SLC27A4 | Q6P1M0 | Long-chain fatty acid transport |
| | | | | protein 4 |
| 102 | Cardiometabolic_IL6 | IL6 | P05231 | Interleukin-6 |
| 103 | Oncology_DKKL1 | DKKL1 | Q9UK85 | Dickkopf-like protein 1 |
| 104 | Cardiometabolic_MFAP3 | MFAP3 | P55082 | Microfibril-associated |
| | | | | glycoprotein 3 |
| 105 | Inflammation_II_STX7 | STX7 | O15400 | Syntaxin-7 |
| 106 | Inflammation_II_SSBP1 | SSBP1 | Q04837 | \Single-stranded DNA-binding |
| | | | | protein, mitochondrial\″″ |
| 107 | Inflammation_II_AKR7L | AKR7L | Q8NHP1 | Aflatoxin B1 aldehyde reductase |
| | | | | member 4 |
| 108 | Cardiometabolic_II_UGDH | UGDH | O60701 | UDP-glucose 6-dehydrogenase |
| 109 | Cardiometabolic_II_IGHMBP2 | IGHMBP2 | P38935 | DNA-binding protein SMUBP-2 |
| 110 | Neurology_GBP4 | GBP4 | Q96PP9 | Guanylate-binding protein 4 |
| 111 | Inflammation_II_RBPMS | RBPMS | Q93062 | RNA-binding protein with |
| | | | | multiple splicing |
| 112 | Cardiometabolic_ST6GAL1 | ST6GAL1 | P15907 | Beta-galactoside alpha-2,6- |
| | | | | sialyltransferase 1 |
| 113 | Cardiometabolic_LILRA5 | LILRA5 | A6NI73 | Leukocyte immunoglobulin-like |
| | | | | receptor subfamily A member 5 |
| 114 | Neurology_LILRA2 | LILRA2 | Q8N149 | Leukocyte immunoglobulin-like |
| | | | | receptor subfamily A member 2 |
| 115 | Neurology_II_SOWAHA | SOWAHA | Q2M3V2 | Ankyrin repeat domain- |
| | | | | containing protein SOWAHA |
| 116 | Cardiometabolic_II_ACADSB | ACADSB | P45954 | Short/branched chain specific |
| | | | | acyl-CoA dehydrogenase, |
| | | | | mitochondrial |
| 117 | Neurology_II_CAMLG | CAMLG | P49069 | Guided entry of tail-anchored |
| | | | | proteins factor CAMLG |
| 118 | Cardiometabolic_CRTAC1 | CRTAC1 | Q9NQ79 | Cartilage acidic protein 1 |
| 119 | Cardiometabolic_SUSD1 | SUSD1 | Q6UWL2 | Sushi domain-containing protein |
| | | | | 1 |
| 120 | Neurology_IL6 | IL6 | P05231 | Interleukin-6 |
| 121 | Oncology_KLK10 | KLK10 | O43240 | Kallikrein-10 |
| 122 | Oncology_II_GRSF1 | GRSF1 | Q12849 | G-rich sequence factor 1 |
| 123 | Inflammation_II_MFAP4 | MFAP4 | P55083 | Microfibril-associated |
| | | | | glycoprotein 4 |
| 124 | Neurology_II_NMT1 | NMT1 | P30419 | Glycylpeptide N- |
| | | | | tetradecanoyltransferase 1 |
| 125 | Neurology_CNTN3 | CNTN3 | Q9P232 | Contactin-3 |
| 126 | Inflammation_II_IL36A | IL36A | Q9UHA7 | Interleukin-36 alpha |
| 127 | Cardiometabolic_II_EHD3 | EHD3 | Q9NZN3 | EH domain-containing protein 3 |
| 128 | Neurology_MAPT | MAPT | P10636 | Microtubule-associated protein |
| | | | | tau |
| 129 | Neurology_II_AGBL2 | AGBL2 | Q5U5Z8 | Cytosolic carboxypeptidase 2 |
| 130 | Oncology_II_ERN1 | ERN1 | O75460 | Serine/threonine-protein |
| | | | | kinase/endoribonuclease IRE1 |
| 131 | Cardiometabolic_II_POMC | POMC | P01189 | Pro-opiomelanocortin |
| 132 | Cardiometabolic_II_PDIA4 | PDIA4 | P13667 | Protein disulfide-isomerase A4 |
| 133 | Inflammation_LGMN | LGMN | Q99538 | Legumain |
| 134 | Neurology_EPHA10 | EPHA10 | Q5JZY3 | Ephrin type-A receptor 10 |
| 135 | Neurology_II_PCBP2 | PCBP2 | Q15366 | Poly(rC)-binding protein 2 |
| 136 | Cardiometabolic_II_PTGR1 | PTGR1 | Q14914 | Prostaglandin reductase 1 |
| 137 | Inflammation_II_GIT1 | GIT1 | Q9Y2X7 | ARF GTPase-activating protein |
| | | | | GIT1 |
| 138 | Inflammation_II_TREML1 | TREML1 | Q86YW5 | Trem-like transcript 1 protein |
| 139 | Oncology_GALNT2 | GALNT2 | Q10471 | Polypeptide N- |
| | | | | acetylgalactosaminyltransferase 2 |
| 140 | Neurology_TDGF1 | TDGF1 | P13385 | Teratocarcinoma-derived growth |
| | | | | factor 1 |
| 141 | Inflammation_II_INSR | INSR | P06213 | Insulin receptor |
| 142 | Inflammation_OSCAR | OSCAR | Q8IYS5 | Osteoclast-associated |
| | | | | immunoglobulin-like receptor |
| 143 | Inflammation_MMP10 | MMP10 | P09238 | Stromelysin-2 |
| 144 | Cardiometabolic_II_MRPL24 | MRPL24 | Q96A35 | 39S ribosomal protein L24, |
| | | | | mitochondrial |
| 145 | Neurology_II_EIF1AX | EIF1AX | P47813 | Eukaryotic translation initiation |
| | | | | factor 1A, X-chromosomal |
| 146 | Cardiometabolic_II_AHNAK2 | AHNAK2 | Q8IVF2 | Protein AHNAK2 |
| 147 | Oncology_TP53 | TP53 | P04637 | Cellular tumor antigen p53 |
| 148 | Neurology_II_GBA | GBA | P04062 | Lysosomal acid |
| | | | | glucosylceramidase |
| 149 | Neurology_II_LRRC38 | LRRC38 | Q5VT99 | Leucine-rich repeat-containing |
| | | | | protein 38 |
| 150 | Inflammation_II_CLEC12A | CLEC12A | Q5QGZ9 | C-type lectin domain family 12 |
| | | | | member A |
| 151 | Inflammation_TPT1 | TPT1 | P13693 | Translationally-controlled tumor |
| | | | | protein |
| 152 | Oncology_II_PPP1CC | PPP1CC | P36873 | Serine/threonine-protein |
| | | | | phosphatase PP1-gamma |
| | | | | catalytic subunit |
| 153 | Cardiometabolic_BPIFB1 | BPIFB1 | Q8TDL5 | BPI fold-containing family B |
| | | | | member 1 |
| 154 | Oncology_CFC1 | CFC1 | POCG37 | Cryptic protein |
| 155 | Oncology_SIGLEC9 | SIGLEC9 | Q9Y336 | Sialic acid-binding Ig-like lectin |
| | | | | 9 |
| 156 | Cardiometabolic_II_CALY | CALY | Q9NYX4 | Neuron-specific vesicular protein |
| | | | | calcyon |
| 157 | Inflammation_OSM | OSM | P13725 | Oncostatin-M |
| 158 | Inflammation_II_ADAMTS1 | ADAMTS1 | Q9UHI8 | A disintegrin and |
| | | | | metalloproteinase with |
| | | | | thrombospondin motifs 1 |
| 159 | Cardiometabolic_OSMR | OSMR | Q99650 | Oncostatin-M-specific receptor |
| | | | | subunit beta |
| 160 | Cardiometabolic_TYMP | TYMP | P19971 | Thymidine phosphorylase |
| 161 | Cardiometabolic_GPR37 | GPR37 | O15354 | Prosaposin receptor GPR37 |
| 162 | Inflammation_CLEC7A | CLEC7A | Q9BXN2 | C-type lectin domain family 7 |
| | | | | member A |
| 163 | Oncology_SMAD5 | SMAD5 | Q99717 | Mothers against decapentaplegic |
| | | | | homolog 5 |
| 164 | Oncology_SFTPA2 | SFTPA2 | Q8IWL1 | Pulmonary surfactant-associated |
| | | | | protein A2 |
| 165 | Neurology_CTSS | CTSS | P25774 | Cathepsin S |
| 166 | Neurology_HNMT | HNMT | P50135 | Histamine N-methyltransferase |
| 167 | Neurology_II_BATF | BATF | Q16520 | Basic leucine zipper |
| | | | | transcriptional factor ATF-like |
| 168 | Neurology_CCL19 | CCL19 | Q99731 | C-C motif chemokine 19 |
| 169 | Oncology_II_SHC1 | SHC1 | P29353 | SHC-transforming protein 1 |
| 170 | Inflammation_CST7 | CST7 | O76096 | Cystatin-F |
| 171 | Oncology_S100A12 | S100A12 | P80511 | Protein S100-A12 |
| 172 | Neurology_ASAH2 | ASAH2 | Q9NR71 | Neutral ceramidase |
| 173 | Cardiometabolic_PPIB | PPIB | P23284 | Peptidyl-prolyl cis-trans |
| | | | | isomerase B |
| 174 | Oncology_LYPD3 | LYPD3 | O95274 | Ly6/PLAUR domain-containing |
| | | | | protein 3 |
| 175 | Inflammation_II_APOL1 | APOL1 | O14791 | Apolipoprotein L1 |
| 176 | Inflammation_II_AFM | AFM | P43652 | Afamin |
| 177 | Cardiometabolic_SSC4D | SSC4D | Q8WTU2 | Scavenger receptor cysteine-rich |
| | | | | domain-containing group B |
| | | | | protein |
| 178 | Oncology_II_FGF7 | FGF7 | P21781 | Fibroblast growth factor 7 |
| 179 | Neurology_TDRKH | TDRKH | Q9Y2W6 | Tudor and KH domain- |
| | | | | containing protein |
| 180 | Oncology_SCG2 | SCG2 | P13521 | Secretogranin-2 |
| 181 | Cardiometabolic_ENPP2 | ENPP2 | Q13822 | Ectonucleotide |
| | | | | pyrophosphatase/phosphodiesterase |
| | | | | family member 2 |
| 182 | Cardiometabolic_PRKAR1A | PRKAR1A | P10644 | cAMP-dependent protein kinase |
| | | | | type I-alpha regulatory subunit |
| 183 | Oncology_II_FAM3D | FAM3D | Q96BQ1 | Protein FAM3D |
| 184 | Cardiometabolic_II_GADD45GIP1 | GADD45GIP1 | Q8TAE8 | Growth arrest and DNA damage- |
| | | | | inducible proteins-interacting |
| | | | | protein 1 |
| 185 | Neurology_SEMA4D | SEMA4D | Q92854 | Semaphorin-4D |
| 186 | Neurology_II_PPP1R14A | PPP1R14A | Q96A00 | Protein phosphatase 1 regulatory |
| | | | | subunit 14A |
| 187 | Inflammation_EGF | EGF | P01133 | Pro-epidermal growth factor |
| 188 | Oncology_NTF4 | NTF4 | P34130 | Neurotrophin-4 |
| 189 | Inflammation_II_SERPING1 | SERPING1 | P05155 | Plasma protease C1 inhibitor |
| 190 | Cardiometabolic_II_COX6B1 | COX6B1 | P14854 | Cytochrome c oxidase subunit |
| | | | | 6B1 |
| 191 | Cardiometabolic_II_NECAP2 | NECAP2 | Q9NVZ3 | Adaptin ear-binding coat- |
| | | | | associated protein 2 |
| 192 | Neurology_TFF1 | TFF1 | P04155 | Trefoil factor 1 |
| 193 | Neurology_IDI2 | IDI2 | Q9BXS1 | Isopentenyl-diphosphate delta- |
| | | | | isomerase 2 |
| 194 | Neurology_II_TJP3 | TJP3 | O95049 | Tight junction protein ZO-3 |
| 195 | Oncology_CA14 | CA14 | Q9ULX7 | Carbonic anhydrase 14 |
| 196 | Inflammation_II_PZP | PZP | P20742 | Pregnancy zone protein |
| 197 | Neurology_PLIN1 | PLIN1 | O60240 | Perilipin-1 |
| 198 | Oncology_ERBB4 | ERBB4 | Q15303 | Receptor tyrosine-protein kinase |
| | | | | erbB-4 |
| 199 | Oncology_TBC1D23 | TBC1D23 | Q9NUY8 | TBC1 domain family member 23 |
| 200 | Inflammation_II_CRISP3 | CRISP3 | P54108 | Cysteine-rich secretory protein 3 |
| 201 | Oncology_II_IFI30 | IFI30 | P13284 | Gamma-interferon-inducible |
| | | | | lysosomal thiol reductase |
| 202 | Inflammation_II_ITIH1 | ITIH1 | P19827 | Inter-alpha-trypsin inhibitor |
| | | | | heavy chain H1 |
| 203 | Inflammation_II_C9 | C9 | P02748 | Complement component C9 |
| 204 | Inflammation_LAP3 | LAP3 | P28838 | Cytosol aminopeptidase |
| 205 | Oncology_II_PDIA5 | PDIA5 | Q14554 | Protein disulfide-isomerase A5 |
| 206 | Oncology_II_ENDOU | ENDOU | P21128 | Poly(U)-specific |
| | | | | endoribonuclease |
| 207 | Inflammation_FLT3LG | FLT3LG | P49771 | Fms-related tyrosine kinase 3 |
| | | | | ligand |
| 208 | Oncology_VNN2 | VNN2 | O95498 | Vascular non-inflammatory |
| | | | | molecule 2 |
| 209 | Inflammation_MILR1 | MILR1 | Q7Z6M3 | Allergin-1 |
| 210 | Cardiometabolic_SDC1 | SDC1 | P18827 | Syndecan-1 |
| 211 | Oncology_II_CEACAM18 | CEACAM18 | A8MTB9 | Carcinoembryonic antigen- |
| | | | | related cell adhesion molecule 18 |
| 212 | Cardiometabolic_II_FHIP2A | FHIP2A | Q5W0V3 | FHF complex subunit HOOK |
| | | | | interacting protein 2A |
| 213 | Oncology_CEACAM5 | CEACAM5 | P06731 | Carcinoembryonic antigen- |
| | | | | related cell adhesion molecule 5 |
| 214 | Inflammation_II_F11 | F11 | P03951 | Coagulation factor XI |
| 215 | Inflammation_WFIKKN2 | WFIKKN2 | Q8TEU8 | WAP, Kazal, immunoglobulin, |
| | | | | Kunitz and NTR domain- |
| | | | | containing protein 2 |
| 216 | Oncology_USO1 | USO1 | O60763 | General vesicular transport factor |
| | | | | p115 |
| 217 | Inflammation_CD40LG | CD40LG | P29965 | CD40 ligand |
| 218 | Neurology_II_GSTT2B | GSTT2B | P0CG30 | Glutathione S-transferase theta- |
| | | | | 2B |
| 219 | Neurology_II_DUSP29 | DUSP29 | Q68J44 | Dual specificity phosphatase 29 |
| 220 | Neurology_II_ATXN2L | ATXN2L | Q8WWM7 | Ataxin-2-like protein |
| 221 | Oncology_IL6 | IL6 | P05231 | Interleukin-6 |
| 222 | Oncology_RRM2 | RRM2 | P31350 | Ribonucleoside-diphosphate |
| | | | | reductase subunit M2 |
| 223 | Oncology_FGF23 | FGF23 | Q9GZV9 | Fibroblast growth factor 23 |
| 224 | Oncology_II_ARHGAP30 | ARHGAP30 | Q7Z6I6 | Rho GTPase-activating protein |
| | | | | 30 |
| 225 | Inflammation_II_SERPINA3 | SERPINA3 | P01011 | Alpha-1-antichymotrypsin |
| 226 | Neurology_CXCL13 | CXCL13 | O43927 | C-X-C motif chemokine 13 |
| 227 | Neurology_MMP8 | MMP8 | P22894 | Neutrophil collagenase |
| 228 | Inflammation_NUDC | NUDC | Q9Y266 | Nuclear migration protein nudC |
| 229 | Oncology_II_ENOPH1 | ENOPH1 | Q9UHY7 | Enolase-phosphatase E1 |
| 230 | Oncology_II_NEK7 | NEK7 | Q8TDX7 | Serine/threonine-protein kinase |
| | | | | Nek7 |
| 231 | Cardiometabolic_II_MAN1A2 | MAN1A2 | O60476 | Mannosyl-oligosaccharide 1,2- |
| | | | | alpha-mannosidase IB |
| 232 | Cardiometabolic_II_ASAH1 | ASAH1 | Q13510 | Acid ceramidase |
| 233 | Inflammation_II_STX5 | STX5 | Q13190 | Syntaxin-5 |
| 234 | Oncology_II_IZUMO1 | IZUMO1 | Q8IYV9 | Izumo sperm-egg fusion protein |
| | | | | 1 |
| 235 | Inflammation_II_SERPINC1 | SERPINC1 | P01008 | Antithrombin-III |
| 236 | Oncology_II_IL9 | IL9 | P15248 | Interleukin-9 |
| 237 | Oncology_PVALB | PVALB | P20472 | Parvalbumin alpha |
| 238 | Cardiometabolic_GZMH | GZMH | P20718 | Granzyme H |
| 239 | Inflammation_II_FGF16 | FGF16 | O43320 | Fibroblast growth factor 16 |
| 240 | Inflammation_TFF2 | TFF2 | Q03403 | Trefoil factor 2 |
| 241 | Cardiometabolic_WASF1 | WASF1 | Q92558 | Wiskott-Aldrich syndrome |
| | | | | protein family member 1 |
| 242 | Oncology_II_TMEM106A | TMEM106A | Q96A25 | Transmembrane protein 106A |
| 243 | Cardiometabolic_GP2 | GP2 | P55259 | Pancreatic secretory granule |
| | | | | membrane major glycoprotein |
| | | | | GP2 |
| 244 | Inflammation_PLXNA4 | PLXNA4 | Q9HCM2 | Plexin-A4 |
| 245 | Oncology_GNE | GNE | Q9Y223 | Bifunctional UDP-N- |
| | | | | acetylglucosamine 2- |
| | | | | epimerase/N-acetylmannosamine |
| | | | | kinase |
| 246 | Neurology_LGALS8 | LGALS8 | O00214 | Galectin-8 |
| 247 | Inflammation_AOC1 | AOC1 | P19801 | Amiloride-sensitive amine |
| | | | | oxidase [copper-containing] |
| 248 | Neurology_FLRT2 | FLRT2 | O43155 | Leucine-rich repeat |
| | | | | transmembrane protein FLRT2 |
| 249 | Oncology_II_CHCHD6 | CHCHD6 | Q9BRQ6 | MICOS complex subunit MIC25 |
| 250 | Oncology_II_RNF43 | RNF43 | Q68DV7 | E3 ubiquitin-protein ligase |
| | | | | RNF43 |
| 251 | Inflammation_II_TPD52L2 | TPD52L2 | O43399 | Tumor protein D54 |
| 252 | Cardiometabolic_II_CSDE1 | CSDE1 | O75534 | Cold shock domain-containing |
| | | | | protein E1 |
| 253 | Oncology_II_GPD1 | GPD1 | P21695 | Glycerol-3-phosphate |
| | | | | dehydrogenase [NAD(+)], |
| | | | | cytoplasmic |
| 254 | Inflammation_PLA2G4A | PLA2G4A | P47712 | Cytosolic phospholipase A2 |
| 255 | Oncology_LRIG1 | LRIG1 | Q96JA1 | Leucine-rich repeats and |
| | | | | immunoglobulin-like domains |
| | | | | protein 1 |
| 256 | Neurology_NGF | NGF | P01138 | Beta-nerve growth factor |
| 257 | Cardiometabolic_II_RAB27B | RAB27B | O00194 | Ras-related protein Rab-27B |
| 258 | Oncology_VAT1 | VAT1 | Q99536 | Synaptic vesicle membrane |
| | | | | protein VAT-1 homolog |
| 259 | Oncology_II_NUDT16 | NUDT16 | Q96DE0 | U8 snoRNA-decapping enzyme |
| 260 | Cardiometabolic_II_TRAF3IP2 | TRAF3IP2 | O43734 | E3 ubiquitin ligase TRAF3IP2 |
| 261 | Cardiometabolic_MARCO | MARCO | Q9UEW3 | Macrophage receptor MARCO |
| 262 | Cardiometabolic_UMOD | UMOD | P07911 | Uromodulin |
| 263 | Inflammation_PIK3AP1 | PIK3AP1 | Q6ZUJ8 | Phosphoinositide 3-kinase |
| | | | | adapter protein 1 |
| 264 | Cardiometabolic_II_MEGF11 | MEGF11 | A6BM72 | Multiple epidermal growth |
| | | | | factor-like domains protein 11 |
| 265 | Inflammation_II_NEDD4L | NEDD4L | Q96PU5 | E3 ubiquitin-protein ligase |
| | | | | NEDD4-like |
| 266 | Cardiometabolic_II_PKD2 | PKD2 | Q13563 | Polycystin-2 |
| 267 | Cardiometabolic_CEBPB | CEBPB | P17676 | CCAAT/enhancer-binding |
| | | | | protein beta |
| 268 | Cardiometabolic_II_RILPL2 | RILPL2 | Q969X0 | RILP-like protein 2 |
| 269 | Oncology_II_IL3 | IL3 | P08700 | Interleukin-3 |
| 270 | Neurology_II_RGCC | RGCC | Q9H4X1 | Regulator of cell cycle RGCC |
| 271 | Cardiometabolic_II_SARG | SARG | Q9BW04 | Specifically androgen-regulated |
| | | | | gene protein |
| 272 | Oncology_II_SMAD2 | SMAD2 | Q15796 | Mothers against decapentaplegic |
| | | | | homolog 2 |
| 273 | Cardiometabolic_CTSH | CTSH | P09668 | Pro-cathepsin H |
| 274 | Inflammation_II_KLKB1 | KLKB1 | P03952 | Plasma kallikrein |
| 275 | Oncology_ERP44 | ERP44 | Q9BS26 | Endoplasmic reticulum resident |
| | | | | protein 44 |
| 276 | Inflammation_SULT2A1 | SULT2A1 | Q06520 | Bile salt sulfotransferase |
| 277 | Oncology_SORD | SORD | Q00796 | Sorbitol dehydrogenase |
| 278 | Oncology_II_IFNAR1 | IFNAR1 | P17181 | Interferon alpha/beta receptor 1 |
| 279 | Oncology_KLK11 | KLK11 | Q9UBX7 | Kallikrein-11 |
| 280 | Cardiometabolic_II_TOMM20 | TOMM20 | Q15388 | Mitochondrial import receptor |
| | | | | subunit TOM20 homolog |
| 281 | Inflammation_II_C3 | C3 | P01024 | Complement C3 |
| 282 | Cardiometabolic_II_ADRA2A | ADRA2A | P08913 | Alpha-2A adrenergic receptor |
| 283 | Inflammation_NCK2 | NCK2 | O43639 | Cytoplasmic protein NCK2 |
| 284 | Neurology_KIRREL2 | KIRREL2 | Q6UWL6 | Kin of IRRE-like protein 2 |
| 285 | Neurology_II_CACNB3 | CACNB3 | P54284 | Voltage-dependent L-type |
| | | | | calcium channel subunit beta-3 |
| 286 | Inflammation_SKAP2 | SKAP2 | O75563 | Src kinase-associated |
| | | | | phosphoprotein 2 |
| 287 | Cardiometabolic_II_CEACAM6 | CEACAM6 | P40199 | Carcinoembryonic antigen- |
| | | | | related cell adhesion molecule 6 |
| 288 | Neurology_II_DNAJC21 | DNAJC21 | Q5F1R6 | DnaJ homolog subfamily C |
| | | | | member 21 |
| 289 | Inflammation_II_PROS1 | PROS1 | P07225 | Vitamin K-dependent protein S |
| 290 | Cardiometabolic_NRCAM | NRCAM | Q92823 | Neuronal cell adhesion molecule |
| 291 | Oncology_NPY | NPY | P01303 | Pro-neuropeptide Y |
| 292 | Neurology_FYB1 | FYB1 | O15117 | FYN-binding protein 1 |
| 293 | Oncology_II_RAB2B | RAB2B | Q8WUD1 | Ras-related protein Rab-2B |
| 294 | Inflammation_MANF | MANF | P55145 | Mesencephalic astrocyte-derived |
| | | | | neurotrophic factor |
| 295 | Cardiometabolic_II_MECR | MECR | Q9BV79 | Enoyl-[acyl-carrier-protein] |
| | | | | reductase, mitochondrial |
| 296 | Inflammation_II_LPA | LPA | P08519 | Apolipoprotein(a) |
| 297 | Inflammation_II_DAAM1 | DAAM1 | Q9Y4D1 | Disheveled-associated activator |
| | | | | of morphogenesis 1 |
| 298 | Inflammation_II_DCTD | DCTD | P32321 | Deoxycytidylate deaminase |
| 299 | Inflammation_FXYD5 | FXYD5 | Q96DB9 | FXYD domain-containing ion |
| | | | | transport regulator 5 |
| 300 | Inflammation_II_CRELD1 | CRELD1 | Q96HD1 | Protein disulfide isomerase |
| | | | | CRELD1 |
| 301 | Neurology_II_PLEKHO1 | PLEKHO1 | Q53GL0 | Pleckstrin homology domain- |
| | | | | containing family O member 1 |
| 302 | Cardiometabolic_TINAGL1 | TINAGL1 | Q9GZM7 | Tubulointerstitial nephritis |
| | | | | antigen-like |
| 303 | Oncology_ZBTB16 | ZBTB16 | Q05516 | Zinc finger and BTB domain- |
| | | | | containing protein 16 |
| 304 | Inflammation_PROK1 | PROK1 | P58294 | Prokineticin-1 |
| 305 | Oncology_II_MAP2K1 | MAP2K1 | Q02750 | Dual specificity mitogen- |
| | | | | activated protein kinase kinase 1 |
| 306 | Inflammation_DAPP1 | DAPP1 | Q9UN19 | Dual adapter for phosphotyrosine |
| | | | | and 3-phosphotyrosine and 3- |
| | | | | phosphoinositide |
| 307 | Oncology_DSG4 | DSG4 | Q86SJ6 | Desmoglein-4 |
| 308 | Inflammation_PPP1R9B | PPP1R9B | Q96SB3 | Neurabin-2 |
| 309 | Oncology_RILP | RILP | Q96NA2 | Rab-interacting lysosomal |
| | | | | protein |
| 310 | Inflammation_EIF4G1 | EIF4G1 | Q04637 | Eukaryotic translation initiation |
| | | | | factor 4 gamma 1 |
| 311 | Neurology_SESTD1 | SESTD1 | Q86VW0 | SEC14 domain and spectrin |
| | | | | repeat-containing protein 1 |
| 312 | Oncology_KIFBP | KIFBP | Q96EK5 | KIF-binding protein |
| 313 | Oncology_HGS | HGS | O14964 | Hepatocyte growth factor- |
| | | | | regulated tyrosine kinase |
| | | | | substrate |
| 314 | Cardiometabolic_CD14 | CD14 | P08571 | Monocyte differentiation antigen |
| | | | | CD14 |
| 315 | Inflammation_II_ANKMY2 | ANKMY2 | Q8IV38 | Ankyrin repeat and MYND |
| | | | | domain-containing protein 2 |
| 316 | Inflammation_WNT9A | WNT9A | O14904 | Protein Wnt-9a |
| 317 | Cardiometabolic_CA13 | CA13 | Q8N1Q1 | Carbonic anhydrase 13 |
| 318 | Cardiometabolic_II_GP1BB | GP1BB | P13224 | Platelet glycoprotein Ib beta |
| | | | | chain |
| 319 | Inflammation_CLIP2 | CLIP2 | Q9UDT6 | CAP-Gly domain-containing |
| | | | | linker protein 2 |
| 320 | Inflammation_BANK1 | BANK1 | Q8NDB2 | B-cell scaffold protein with |
| | | | | ankyrin repeats |
| 321 | Oncology_II_WDR46 | WDR46 | O15213 | WD repeat-containing protein 46 |
| 322 | Cardiometabolic_HSPB1 | HSPB1 | P04792 | Heat shock protein beta-1 |
| 323 | Cardiometabolic_II_CSF2 | CSF2 | P04141 | Granulocyte-macrophage colony- |
| | | | | stimulating factor |
| 324 | Inflammation_II_SNCA | SNCA | P37840 | Alpha-synuclein |
| 325 | Neurology_II_RRAS | RRAS | P10301 | Ras-related protein R-Ras |
| 326 | Neurology_PRTFDC1 | PRTFDC1 | Q9NRG1 | Phosphoribosyltransferase |
| | | | | domain-containing protein 1 |
| 327 | Cardiometabolic_II_RBPMS2 | RBPMS2 | Q6ZRY4 | RNA-binding protein with |
| | | | | multiple splicing 2 |
| 328 | Oncology_II_LARP1 | LARP1 | Q6PKG0 | La-related protein 1 |
| 329 | Oncology_II_KAZN | KAZN | Q674X7 | Kazrin |
| 330 | Neurology_CLSPN | CLSPN | Q9HAW4 | Claspin |
| 331 | Neurology_RHOC | RHOC | P08134 | Rho-related GTP-binding protein |
| | | | | RhoC |
| 332 | Neurology_II_PPT1 | PPT1 | P50897 | Palmitoyl-protein thioesterase 1 |
| 333 | Oncology_DPEP2 | DPEP2 | Q9H4A9 | Dipeptidase 2 |
| 334 | Inflammation_METAP1D | METAP1D | Q6UB28 | Methionine aminopeptidase 1D, |
| | | | | mitochondrial |
| 335 | Cardiometabolic_STK11 | STK11 | Q15831 | Serine/threonine-protein kinase |
| | | | | STK11 |
| 336 | Inflammation_II_CFH | CFH | P08603 | Complement factor H |
| 337 | Inflammation_II_PDE5A | PDE5A | O76074 | cGMP-specific 3′,5′-cyclic |
| | | | | phosphodiesterase |
| 338 | Inflammation_II_MRC1 | MRC1 | P22897 | Macrophage mannose receptor 1 |
| 339 | Neurology_BIN2 | BIN2 | Q9UBW5 | Bridging integrator 2 |
| 340 | Inflammation_IL17A | IL17A | Q16552 | Interleukin-17A |
| 341 | Oncology_II_PXDNL | PXDNL | A1KZ92 | Peroxidasin-like protein |
| 342 | Neurology_GP6 | GP6 | Q9HCN6 | Platelet glycoprotein VI |
| 343 | Inflammation_EPO | EPO | P01588 | Erythropoietin |
| 344 | Oncology_MAP3K5 | MAP3K5 | Q99683 | Mitogen-activated protein kinase |
| | | | | kinase kinase 5 |
| 345 | Neurology_II_MCEE | MCEE | Q96PE7 | Methylmalonyl-CoA epimerase, |
| | | | | mitochondrial |
| 346 | Neurology_II_DDHD2 | DDHD2 | O94830 | Phospholipase DDHD2 |
| 347 | Oncology_II_PHLDB2 | PHLDB2 | Q86SQ0 | Pleckstrin homology-like domain |
| | | | | family B member 2 |
| 348 | Inflammation_II_NECTIN1 | NECTIN1 | Q15223 | Nectin-1 |
| 349 | Neurology_II_CCDC50 | CCDC50 | Q8IVM0 | Coiled-coil domain-containing |
| | | | | protein 50 |
| 350 | Neurology_GKN1 | GKN1 | Q9NS71 | Gastrokine-1 |
| 351 | Inflammation_MPIG6B | MPIG6B | O95866 | Megakaryocyte and platelet |
| | | | | inhibitory receptor G6b |
| 352 | Cardiometabolic_CBLIF | CBLIF | P27352 | Cobalamin binding intrinsic |
| | | | | factor |
| 353 | Cardiometabolic_II_SYTL4 | SYTL4 | Q96C24 | Synaptotagmin-like protein 4 |
| 354 | Oncology_II_SSH3 | SSH3 | Q8TE77 | Protein phosphatase Slingshot |
| | | | | homolog 3 |
| 355 | Cardiometabolic_II_PDZD2 | PDZD2 | O15018 | PDZ domain-containing protein 2 |
| 356 | Neurology_SULT1A1 | SULT1A1 | P50225 | Sulfotransferase 1A1 |
| 357 | Neurology_II_DLG4 | DLG4 | P78352 | Disks large homolog 4 |
| 358 | Inflammation_HPCAL1 | HPCAL1 | P37235 | Hippocalcin-like protein 1 |
| 359 | Inflammation_ICA1 | ICA1 | Q05084 | Islet cell autoantigen 1 |
| 360 | Cardiometabolic_GDF15 | GDF15 | Q99988 | Growth/differentiation factor 15 |
| 361 | Inflammation_CD160 | CD160 | O95971 | CD 160 antigen |
| 362 | Inflammation_II_APPL2 | APPL2 | Q8NEU8 | DCC-interacting protein 13-beta |
| 363 | Neurology_GRN | GRN | P28799 | Progranulin |
| 364 | Neurology_IL17RA | IL17RA | Q96F46 | Interleukin-17 receptor A |
| 365 | Oncology_II_CDC42BPB | CDC42BPB | Q9Y5S2 | Serine/threonine-protein kinase |
| | | | | MRCK beta |
| 366 | Oncology_C4BPB | C4BPB | P20851 | C4b-binding protein beta chain |
| 367 | Inflammation_DAG1 | DAG1 | Q14118 | Dystroglycan |
| 368 | Oncology_II_CMIP | CMIP | Q8IY22 | C-Maf-inducing protein |
| 369 | Inflammation_KYNU | KYNU | Q16719 | Kynureninase |
| 370 | Inflammation_II_NUMB | NUMB | P49757 | Protein numb homolog |
| 371 | Oncology_PPY | PPY | P01298 | Pancreatic prohormone |
| 372 | Cardiometabolic_II_PPIF | PPIF | P30405 | Peptidyl-prolyl cis-trans |
| | | | | isomerase F, mitochondrial |
| 373 | Inflammation_II_CFI | CFI | P05156 | Complement factor I |
| 374 | Inflammation_II_DTD1 | DTD1 | Q8TEA8 | D-aminoacyl-tRNA deacylase 1 |
| 375 | Neurology_II_LDLRAP1 | LDLRAP1 | Q5SW96 | Low density lipoprotein receptor |
| | | | | adapter protein 1 |
| 376 | Oncology_II_FGF9 | FGF9 | P31371 | Fibroblast growth factor 9 |
| 377 | Neurology_II_STXBP1 | STXBP1 | P61764 | Syntaxin-binding protein 1 |
| 378 | Cardiometabolic_II_CMC1 | CMC1 | Q7Z7K0 | COX assembly mitochondrial |
| | | | | protein homolog |
| 379 | Inflammation_GOPC | GOPC | Q9HD26 | Golgi-associated PDZ and coiled- |
| | | | | coil motif-containing protein |
| 380 | Neurology_II_SMTN | SMTN | P53814 | Smoothelin |
| 381 | Inflammation_PTPN6 | PTPN6 | P29350 | Tyrosine-protein phosphatase |
| | | | | non-receptor type 6 |
| 382 | Cardiometabolic_II_L3HYPDH | L3HYPDH | Q96EM0 | Trans-3-hydroxy-L-proline |
| | | | | dehydratase |
| 383 | Cardiometabolic_II_PDAP1 | PDAP1 | Q13442 | 28 kDa heat- and acid-stable |
| | | | | phosphoprotein |
| 384 | Cardiometabolic_II_LPP | LPP | Q93052 | Lipoma-preferred partner |
| 385 | Oncology_II_THTPA | THTPA | Q9BU02 | Thiamine-triphosphatase |
| 386 | Cardiometabolic_XG | XG | P55808 | Glycoprotein Xg |
| 387 | Inflammation_AGRP | AGRP | O00253 | Agouti-related protein |
| 388 | Cardiometabolic_II_RAB11FIP3 | RAB11FIP3 | O75154 | Rab11 family-interacting protein |
| | | | | 3 |
| 389 | Neurology_F11R | F11R | Q9Y624 | Junctional adhesion molecule A |
| 390 | Inflammation_BCR | BCR | P11274 | Breakpoint cluster region protein |
| 391 | Cardiometabolic_II_LONP1 | LONP1 | P36776 | Lon protease homolog, |
| | | | | mitochondrial |
| 392 | Inflammation_II_BNIP3L | BNIP3L | O60238 | BCL2/adenovirus E1B 19 kDa |
| | | | | protein-interacting protein 3-like |
| 393 | Cardiometabolic_SELP | SELP | P16109 | P-selectin |
| 394 | Cardiometabolic_GYS1 | GYS1 | P13807 | Glycogen [starch] synthase, |
| | | | | muscle |
| 395 | Inflammation_MGLL | MGLL | Q99685 | Monoglyceride lipase |
| 396 | Neurology_II_PDLIM5 | PDLIM5 | Q96HC4 | PDZ and LIM domain protein 5 |
| 397 | Neurology_MESD | MESD | Q14696 | LRP chaperone MESD |
| 398 | Neurology_II_DNPEP | DNPEP | Q9ULA0 | Aspartyl aminopeptidase |
| 399 | Oncology_SRC | SRC | P12931 | Proto-oncogene tyrosine-protein |
| | | | | kinase Src |
| 400 | Neurology_PMVK | PMVK | Q15126 | Phosphomevalonate kinase |
| 401 | Neurology_II_ITPRIP | ITPRIP | Q8IWB1 | Inositol 1,4,5-trisphosphate |
| | | | | receptor-interacting protein |
| 402 | Cardiometabolic_CD69 | CD69 | Q07108 | Early activation antigen CD69 |
| 403 | Oncology_CALCOCO1 | CALCOCO1 | Q9P1Z2 | Calcium-binding and coiled-coil |
| | | | | domain-containing protein 1 |
| 404 | Oncology_II_PAFAH2 | PAFAH2 | Q99487 | Platelet-activating factor |
| | | | | acetylhydrolase 2, cytoplasmic |
| 405 | Oncology_II_GIPC3 | GIPC3 | Q8TF64 | PDZ domain-containing protein |
| | | | | GIPC3 |
| 406 | Cardiometabolic_SNAP23 | SNAP23 | O00161 | Synaptosomal-associated protein |
| | | | | 23 |
| 407 | Oncology_STAT5B | STAT5B | P51692 | Signal transducer and activator of |
| | | | | transcription 5B |
| 408 | Oncology_RSPO3 | RSPO3 | Q9BXY4 | R-spondin-3 |
| 409 | Neurology_AKT1S1 | AKT1S1 | Q96B36 | Proline-rich AKT1 substrate 1 |
| 410 | Oncology_SNAP29 | SNAP29 | O95721 | Synaptosomal-associated protein |
| | | | | 29 |
| 411 | Inflammation_CASP2 | CASP2 | P42575 | Caspase-2 |
| 412 | Neurology_II_AKT2 | AKT2 | P31751 | RAC-beta serine/threonine- |
| | | | | protein kinase |
| 413 | Oncology_NELL1 | NELL1 | Q92832 | Protein kinase C-binding protein |
| | | | | NELL1 |
| 414 | Oncology_II_MCTS1 | MCTS1 | Q9ULC4 | Malignant T-cell-amplified |
| | | | | sequence 1 |
| 415 | Cardiometabolic_TIA1 | TIA1 | P31483 | Nucleolysin TIA-1 isoform p40 |
| 416 | Cardiometabolic_II_SCRG1 | SCRG1 | O75711 | Scrapie-responsive protein 1 |
| 417 | Oncology_II_CIRBP | CIRBP | Q14011 | Cold-inducible RNA-binding |
| | | | | protein |
| 418 | Cardiometabolic_SEMA3F | SEMA3F | Q13275 | Semaphorin-3F |
| 419 | Neurology_II_SOX2 | SOX2 | P48431 | Transcription factor SOX-2 |
| 420 | Inflammation_II_NRGN | NRGN | Q92686 | Neurogranin |
| 421 | Inflammation_II_PSTPIP2 | PSTPIP2 | Q9H939 | Proline-serine-threonine |
| | | | | phosphatase-interacting protein 2 |
| 422 | Cardiometabolic_II_ISM2 | ISM2 | Q6H9L7 | Isthmin-2 |
| 423 | Cardiometabolic_II_EHBP1 | EHBP1 | Q8NDI1 | EH domain-binding protein 1 |
| 424 | Neurology_VTA1 | VTA1 | Q9NP79 | Vacuolar protein sorting- |
| | | | | associated protein VTA1 |
| | | | | homolog |
| 425 | Oncology_II_DUT | DUT | P33316 | Deoxyuridine 5′-triphosphate |
| | | | | nucleotidohydrolase, |
| | | | | mitochondrial |

TABLE 4
Model performance using Olink ® Target 96 platform

| Model | test AUC |
|---|---|
| Elastic Net (EN) | 0.6777 |
| Support Vector Machie (SVM) | 0.7118 |
| Random Forest (RF) | 0.6978 |
| XGBoost (XGB) | 0.7033 |

TABLE 5
Model performance for “1-5 Y” prediction models in Olink ® Explore 3072 platform

| Model | Min. | 1st. Qu. | Median | Mean | 3rd. Qu. | Max. |
|---|---|---|---|---|---|---|
| Elastic | 0.70971074 | 0.78099174 | 0.80731225 | 0.81637442 | 0.85177866 | 0.92786561 |
| Net (EN) | | | | | | |
| Support | 0.74011858 | 0.79841897 | 0.84288538 | 0.83101868 | 0.86466942 | 0.91304348 |
| Vector | | | | | | |
| Machine | | | | | | |
| (SVM) | | | | | | |
| Random | 0.62796443 | 0.6947314 | 0.72875494 | 0.73175799 | 0.77816206 | 0.82756917 |
| Forest (RF) | | | | | | |
| XGBoost | 0.60968379 | 0.68478261 | 0.72529644 | 0.71994071 | 0.75494071 | 0.88735178 |
| (XGB) | | | | | | |

TABLE 6
Model performance for “1-3 Y” prediction models in Olink ® Explore 3072 platform

| Model | Min. | 1st. Qu. | Median | Mean | 3rd. Qu. | Max. |
|---|---|---|---|---|---|---|
| Elastic | 0.74305556 | 0.83333333 | 0.86561265 | 0.87041118 | 0.89492754 | 0.98913043 |
| Net (EN) | | | | | | |
| Support | 0.73106061 | 0.81944444 | 0.85375494 | 0.86073342 | 0.90513834 | 0.97348485 |
| Vector | | | | | | |
| Machine | | | | | | |
| (SVM) | | | | | | |
| Random | 0.58333333 | 0.68576389 | 0.73517787 | 0.74461435 | 0.78472222 | 0.91847826 |
| Forest (RF) | | | | | | |
| XGBoost | 0.61594203 | 0.69927536 | 0.75889328 | 0.75169412 | 0.81818182 | 0.87747036 |
| (XGB) | | | | | | |

TABLE 7
LLP Cohorts used for 1-3 year and 1-5 year discovery

| | Cases 1-3 years prior to diagnosis | | | | Cases 1-5 years prior to diagnosis | | | |
|---|---|---|---|---|---|---|---|---|
| | Cancer | Control | Total | P value (test)* | Cancer | Control | Total | P value (test)* |
| Sex n (%) Female | 14 (35.0) | 39 (38.2) | 53 (37.3) | X² 0.13 | 27 (36.0) | 77 (41.4) | 104 (39.8) | X² 0.65 |
| Male | 26 (65.0) | 63 (61.8) | 89 (62.7) | P = 0.72 | 48 (64.0) | 109 (58.6) | 157 (60.2) | 0.42 (CS) |
| | | | | (CS) | | | | |
| Age (years) | 69.5 | 70.1 | 69.8 | 0.96 | 68.3 | 68.2 | 68.1 | 0.88 |
| Median (IQR) | (62.3-74.2) | (62.0-74.3) | (62.0-74.2) | (MW) | (62.0-73.3) | (61.9-73.2) | (62.0-73.2) | (MW) |
| Smoking status n (%) current | 11 (27.5) | 38 (37.3) | 49 (34.5) | X² 1.08 | 27 (36.0) | 74 (39.8) | 101 (38.7) | X² 0.51 |
| former | 27 (67.5) | 61 (59.8) | 88 (62.0) | P = 0.58 | 43 (57.3) | 104 (55.9) | 147 (56.3) | P = 0.77 |
| never | 1 (2.5) | 3 (2.9) | 4 (2.8) | (CS) | 2 (2.7) | 8 (4.3) | 10 (3.8) | (CS) |
| unknown | 1 (2.5) | 0 (0) | 1 (0.7) | | 3 (4.0) | 0 (0) | 3 (1.1) | |
| Smoking duration (years) | 44 | 43 | 43 | 0.47 | 44 | 44 | 44 | 0.76 |
| Median (IQR) | (33-48) | (35-50) | (34-49) | (MW) | (34-49) | (35-49) | (35-49) | (MW) |
| Smoking pack years | 43.5 | 39.8 | 39.9 | 0.68 | 41.3 | 37.5 | 38.4 | 0.19 |
| Median (IQR) | (25.0-51.5) | (22.7-53.8) | (24.6-52.8) | (MW) | (25.5-51.8) | (21.8-49.2) | (23.3-50.4) | (MW) |
| Smoking quit years | 0 | 2 | 0 | 0.75 | 0 | 0 | 0 | 0.59 |
| Median (IQR) | (0-10) | (0-12.3) | (1-11.5) | (MW) | (0-10) | (0-9) | (0-8) | (MW) |
| COPD n (%) Yes | 9 (22.5) | 18 (17.6) | 27 (19.0) | X² 0.44 | 16 (21.3) | 33 (17.7) | 49 (18.8) | X² 0.45 |
| No | 31 (77.5) | 84 (82.4) | 115 (81.0) | P = 0.51 | 59 (78.7) | 153 (82.3) | 212 (81.2) | P = 0.50 |
| | | | | (CS) | | | | (CS) |
| Body Mass Index | 26.6 | 26.5 | 26.6 | 0.47 | 26.6 | 26.6 | 26.6 | 0.86 |
| Median (IQR) | (26.2-29.3) | (24.3-28.1) | (24.6-28.2) | (MW) | (24.8-27.4) | (24.5-28.1) | (24.5-28.1) | (MW) |
| Total subjects | 40 | 102 | 142 | | 75 | 186 | 261 | |
| Plasma samples | 58 | 117 | 175 | | 114 | 220 | 334 | |

IQR = Inter-quartile range;
*CS = Chi-square; MW = Mann-Whitney (tests only performed for known values)

TABLE 8
Validation of 1-5 Y lung cancer prediction model in UK Biobank data

| | PPV at sensitivity of: | | | enrichment | | Population | | Prevalence |
|---|---|---|---|---|---|---|---|---|
| | 0.05 | 0.10 | 0.25 | at 0.05 | AUC | Size | Cases | in subgroup |
| Smoker | 47.4 | 37.1 | 21.7 | 5.6 | 0.693 | 4235 | 356 | 8.41 |
| Non-smoker | 7.7 | 8.1 | 6.6 | 3.9 | 0.615 | 1654 | 33 | 2 |
| Age 40-55 y | 100 | 62.5 | 27.9 | 39 | 0.775 | 1913 | 49 | 2.56 |
| Age 55-70 y | 30.4 | 31.5 | 21.3 | 3.5 | 0.683 | 3979 | 343 | 8.62 |
| Male | 55.6 | 29.9 | 20.2 | 7.8 | 0.721 | 2878 | 204 | 7.09 |
| Female | 31 | 31.7 | 17.6 | 5.0 | 0.663 | 3014 | 188 | 6.24 |
| Total | 40.8 | 30 | 19.1 | 6.1 | 0.694 | 5892 | 392 | 6.65 |

PPP = positive predictive value;
AUC = Area under Curve ROC value

TABLE 9
Stage and histology distribution of discovery cohort
and all lung cancer cases (including longitudinal)

| | | | NSCLC | | | Early/ |
|---|---|---|---|---|---|---|
| | z | AdC | NOS | SqC | Total | Late |
| Discovery | IA | 8 | 0 | 4 | 13 | 33 (46%) |
| Cohort | IB | 3 | 0 | 5 | 6 | |
| | IIA | 4 | 0 | 3 | 7 | |
| | IIB | 1 | 0 | 3 | 3 | |
| | Early NOS | 0 | 0 | 4 | 4 | |
| | IIIA | 4 | 1 | 4 | 9 | 39 (54%) |
| | IIIB | 3 | 0 | 1 | 4 | |
| | IV | 8 | 4 | 4 | 17 | |
| | Late NOS | 5 | 1 | 3 | 9 | |
| | no stage | 2 | 0 | 1 | 3 | |
| | Total | 39 | 6 | 30 | 75 | |
| Full | IA | 10 | 0 | 7 | 17 | 51 (42%) |
| Cohort | IB | 5 | 0 | 5 | 10 | |
| | IIA | 6 | 0 | 7 | 13 | |
| | IIB | 1 | 0 | 4 | 5 | |
| | Early NOS | 0 | 1 | 5 | 6 | |
| | IIIA | 8 | 2 | 6 | 16 | 71 (58%) |
| | IIIB | 3 | 1 | 4 | 8 | |
| | IV | 16 | 5 | 7 | 28 | |
| | Late NOS | 7 | 2 | 10 | 19 | |
| | no stage | 2 | 1 | 2 | 5 | |
| | Total | 58 | 12 | 57 | 127 | |

TABLE 10
Longitudinal sample distribution, by number of samples analysed
for cases and by stage at diagnosis; matched sample at each
time point from 1 control per case were also analysed.

| | Time of sample | | | | |
|---|---|---|---|---|---|
| | relative to diagnosis | | | | |
| | 5-10 | 3-5 | 1-3 | At | |
| | years | years | years | diagnosis | Total |
| 4 samples | 4 | 4 | 7 | 5 | 20 |
| 3 samples | 10 | 12 | 10 | 7 | 39 |
| 2 samples | 19 | 14 | 4 | 11 | 48 |
| Total samples | 33 | 30 | 21 | 23 | 107 |
| Early stage cases | 16 | 8 | 8 | 13 | 19 |
| Late stage cases | 16 | 15 | 7 | 10 | 22 |
| Unknown stage cases | 1 | 0 | 1 | 0 | 1 |
| Total cases | 33 | 23 | 16 | 23 | 42 |

| Biomarker | Estimate | P value | EDR |
|---|---|---|---|
| Inilammation_II_PRDX2 | 0.622 | 4.5E−57 | 1.33E−53 |
| Neurology_BL_VRB | 0.800 | 3.3E−56 | 4.79E−53 |
| Inflammation_II_PSMG4 | 0.813 | 2.9E−55 | 2.82E−52 |
| Neurology_CA2 | 1.046 | 1.2E−51 | 8.49E−49 |
| Inflammation_II_CAT | 0.656 | 1.7E−51 | 9.77E−48 |
| Oncology_HAGH | 1.114 | 2.2E−50 | 1.09E−47 |
| Inflammation_II_DDI2 | 0.831 | 2.1E−49 | 8.78E−47 |
| Cardiometabolic_CA13 | 0.762 | 2.0E−48 | 7.25E−46 |
| Oncology_II_C90rf40 | 0.940 | 7.0E−48 | 2.30E−45 |
| Neurology_AHSP | 0.967 | 1.1E−47 | 3.23E−45 |
| Inflammation_PSMG3 | 0.684 | 3.1E−46 | 8.35E−44 |
| Cardiometabolic_EIF4EBP1 | 0.872 | 4.5E−46 | 1.10E−43 |
| Cardiometabolic_AK1 | 0.908 | 9.2E−46 | 2.07E−43 |
| Inflammation_DNPH1 | 0.756 | 2.1E−45 | 4.39E−43 |
| Neurology_II_DNAJA4 | 0.767 | 2.3E−45 | 4.58E−43 |
| Oncology_PSMD9 | 0.902 | 2.5E−45 | 4.58E−43 |
| Inflammation_II_DNAJB2 | 0.782 | 3.8E−45 | 6.50E−43 |
| Cardiometabolic_II_YOD1 | 0.937 | 7.4E−45 | 1.21E−42 |
| Oncology_ATG4A | 0.886 | 1.4E−44 | 2.23E−42 |
| Neurology_LXN | 0.823 | 2.4E−44 | 3.54E−42 |
| Cardiometabolic_SOD1 | 0.597 | 4.4E−44 | 6.20E−42 |
| Oncology_UBAC1 | 0.465 | 5.5E−44 | 7.35E−42 |
| Oncology_II_CENPF | 0.618 | 6.1E−44 | 7.75E−42 |
| Oncology_HBQ1 | 0.622 | 4.1E−43 | 5.01E−41 |
| Neurology_NSFL1C | 0.872 | 2.2E−42 | 2.61E−40 |
| Cardiometabolic_TGM2 | 0.781 | 2.5E−42 | 2.79E−40 |
| Neurology_II_AMPD3 | 0.671 | 3.7E−42 | 3.97E−40 |
| Inflammation_II_MDH1 | 0.520 | 3.8E−42 | 3.97E−40 |
| Neurology_II_ATXN3 | 0.881 | 1.9E−41 | 1.94E−39 |
| Inflammation_LHPP | 0.729 | 2.0E−41 | 1.96E−39 |
| Neuology_PEBP1 | 0.790 | 2.5E−41 | 2.39E−39 |
| Neurolology_CCS | 0.595 | 4.6E−41 | 4.18E−39 |
| Oncology_AARSD1 | 0.821 | 6.5E−41 | 5.76E−39 |
| Neurology_II_IMPACT | 0.775 | 7.0E−41 | 6.03E−39 |
| Inflammation_PKLR | 0.676 | 8.2E−41 | 6.88E−39 |
| Oncology_PPME1 | 0.924 | 1.0E−40 | 8.28E−39 |
| Oncology_II_DNAJC9 | 1.115 | 1.6E−41 | 2.50E−39 |
| Neurology_II_IGBP1 | 0.915 | 1.7E−40 | 1.30E−38 |
| Inflammation_PIK3AP1 | 0.881 | 2.2E−40 | 1.68E−38 |
| Oncology_PRDX6 | 0.620 | 3.8E−40 | 2.79E−38 |
| Neurology_CARHSP1 | 0.660 | 6.5E−40 | 4.69E−38 |
| Cardiometabolic_II_BOLA2_BOLA2B | 0.723 | 7.4E−40 | 5.20E−38 |
| Inflammation_II_TXN | 0.552 | 8.5E−40 | 5.83E−38 |
| Neurology_PSME2 | 0.504 | 8.9E−40 | 5.96E−38 |
| Cardiometabolic_CD2AP | 0.712 | 1.1E−39 | 7.29E−38 |
| Inflammation_II_ACYP1 | 0.826 | 1.2E−39 | 7.71E−38 |
| Neurology_RBKS | 0.602 | 1.4E−39 | 8.49E−38 |
| Neurology_STIP1 | 0.805 | 2.3E−39 | 1.43E−37 |
| Oncology_RILP | 0.774 | 4.5E−39 | 2.70E−37 |
| Inflammation_II_ST13 | 0.716 | 5.7E−39 | 3.36E−37 |
| Neurology_PARK7 | 0.718 | 7.4E−39 | 4.24E−37 |
| Neurology_PSME1 | 0.530 | 1.1E−38 | 6.24E−37 |
| Cardiometabolic_GLRX | 0.762 | 4.2E−38 | 2.33E−36 |
| Inflammation_II_UROD | 0.718 | 1.7E−37 | 9.26E−36 |
| Neurology_PPCDC | 0.540 | 1.8E−37 | 9.74E−36 |
| Cardiometabolic_II_MYL4 | 0.641 | 2.1E−37 | 1.12E−35 |
| Oncology_HMBS | 0.547 | 3.3E−37 | 1.69E−35 |
| Inflammation_II_SNX15 | 0.648 | 5.0E−37 | 2.53E−35 |
| Oncology_ARG1 | 0.702 | 5.5E−37 | 2.73E−35 |
| Inflammation_GLOD4 | 0.489 | 9.0E−37 | 4.39E−35 |
| Cardiometabolic_II_DTYMK | 0.903 | 1.3E−36 | 6.22E−35 |
| Oncology_S100A4 | 0.632 | 1.8E−36 | 8.57E−35 |
| Neurology_II_SH3GLB2 | 0.775 | 3.2E−36 | 1.48E−34 |
| Oncology_II_HDDC2 | 0.488 | 4.3E−36 | 1.96E−34 |
| Inflammation_II_ACP1 | 0.362 | 6.6E−36 | 2.97E−34 |
| Neurology_CPPED1 | 0.820 | 7.9E−36 | 3.50E−34 |
| Inflammation_RABGAP1L | 0.719 | 8.8E−36 | 3.88E−34 |
| Neurology_TBC1D17 | 0.566 | 1.7E−35 | 7.18E−34 |
| Cardiometabolic_II_TSNAX | 0.584 | 2.5E−35 | 1.08E−33 |
| Cardiometabolic_II_GGCT | 0.604 | 7.4E−35 | 3.11E−33 |
| Cardiometabolic_CA3 | 0.592 | 1.1E−34 | 4.74E−33 |
| Neurology_STAMBP | 0.648 | 1.3E−34 | 5.22E−33 |
| Oncology_II_NAP1L4 | 0.670 | 1.3E−34 | 5.24E−33 |
| Neurology_II_CIT | 0.542 | 2.0E−34 | 8.02E−33 |
| Inflammation_II_TBCA | 1.065 | 2.5E−34 | 9.82E−33 |
| Neurology_AKT1S1 | 0.741 | 2.9E−34 | 1.12E−32 |
| Oncology_II_UBE2B | 0.481 | 4.0E−34 | 1.53E−32 |
| Cardiometabolic_II_CNP | 0.918 | 4.9E−34 | 1.84E−32 |
| Neurology_PRDX1 | 0.784 | 5.7E−34 | 2.11E−32 |
| Inflammation_II_UBXN1 | 0.685 | 6.4E−34 | 2.34E−32 |
| Cardiometabolic_PLPBP | 0.883 | 7.2E−34 | 2.61E−32 |
| Oncology_DNAJB1 | 0.918 | 9.9E−34 | 3.56E−32 |
| Inflammation_II_GMPR2 | 0.866 | 1.3E−33 | 4.65E−32 |
| Neurology_II_PSMD1 | 0.790 | 1.4E−33 | 4.80E−32 |
| Oncology_II_SSNA1 | 0.704 | 1.6E−33 | 5.65E−32 |
| Inflammation_II_NEDD4L | 0.429 | 1.0E−32 | 3.53E−31 |
| Cardiometabolic_II_DDT | 0.517 | 1.1E−32 | 3.73E−31 |
| Neurology_PDCD5 | 0.707 | 1.2E−32 | 4.13E−31 |
| Inflammation_II_TP53I3 | 0.536 | 1.3E−32 | 4.15E−31 |
| Neurology_RWDD1 | 0.763 | 2.5E−32 | 8.04E−31 |
| Cardiometabolic_II_RANBP1 | 0.536 | 3.8E−32 | 1.23E−30 |
| Cardiometabolic_II_TALDO1 | 0.599 | 5.0E−32 | 1.61E−30 |
| Neurology_MIF | 0.913 | 5.3E−32 | 1.67E−30 |
| Cardiometabolic_II_BECN1 | 0.709 | 5.8E−32 | 1.80E−30 |
| Neurology_EIF4B | 0.728 | 6.8E−32 | 2.10E−30 |
| Neurology_ALDH1A1 | 0.518 | 1.2E−31 | 3.71E−30 |
| Cardiometabolic_GLO1 | 0.561 | 1.3E−31 | 3.88E−30 |
| Inflammation_II_PTRHD1 | 0.727 | 1.8E−31 | 5.27E−30 |
| Inflammation_II_TRAF3 | 0.561 | 2.4E−31 | 7.20E−30 |
| Neurology_NUDT5 | 0.536 | 3.2E−31 | 9.33E−30 |
| Inflammation_II_ADD1 | 0.654 | 4.4E−31 | 1.29E−29 |
| Inflammation_TRAF2 | 0.693 | 5.7E−31 | 1.65E−29 |
| Oncology_II_FKBPL | 0.556 | 6.1E−31 | 1.75E−29 |
| Inflammation_GMPR | 0.591 | 7.4E−31 | 2.09E−29 |
| Cardiometabolic_QDPR | 0.429 | 8.8E−31 | 2.46E−29 |
| Oncology_II_RPE | 0.689 | 1.2E−30 | 3.20E−29 |
| Neurology_FHIT | 0.907 | 1.0E−29 | 2.87E−28 |
| Neurology_II_NAPRT | 0.350 | 1.1E−29 | 3.06E−28 |
| Neurology_II_DXO | 0.639 | 1.3E−29 | 3.55E−28 |
| Cardiometabolic_II_INPP5D | 0.780 | 3.0E−29 | 8.12E−28 |
| Cardiometabolic_II_PAGR1 | 0.498 | 4.3E−29 | 1.13E−27 |
| Oncology_SIRT2 | 0.867 | 4.3E−29 | 1.13E−27 |
| Neurology_CRADD | 0.831 | 4.5E−29 | 1.16E−27 |
| Inflammation_DFFA | 0.708 | 5.2E−29 | 1.35E−27 |
| Cardiometabolic_II_PGD | 0.659 | 5.5E−29 | 1.42E−27 |
| Neurology_II_HNRNPUL1 | 0.850 | 8.1E−29 | 2.06E−27 |
| Cardiometabolic_II_NIT1 | 0.582 | 1.0E−28 | 2.61E−27 |
| Cardiometabolic_KYAT1 | 0.584 | 1.6E−28 | 4.00E−27 |
| Oncology_II_USP25 | 0.711 | 2.5E−28 | 6.13E−27 |
| Neurology_II_DNPEP | 0.474 | 2.7E−28 | 6.56E−27 |
| Inflammation_II_LZTFL1 | 0.661 | 3.4E−28 | 8.34E−27 |
| Neurology_II_MRI1 | 0.510 | 4.1E−28 | 9.80E−27 |
| Neurology_II_ASPSCR1 | 0.602 | 4.4E−28 | 1.06E−26 |
| Oncology_HGS | 0.715 | 6.9E−28 | 1.65E−26 |
| Inflammation_II_DGKA | 0.578 | 9.4E−28 | 2.22E−26 |
| Oncology_II_ZFYVE19 | 0.776 | 1.3E−27 | 2.95E−26 |
| Neurology_TXNRD1 | 0.374 | 1.5E−27 | 3.54E−26 |
| Oncology_CIAPIN1 | 0.657 | 1.7E−27 | 3.88E−26 |
| Cardiometabolic_II_GCLM | 0.348 | 1.7E−27 | 3.90E−26 |
| Oncology_CASP8 | 0.929 | 2.3E−27 | 5.17E−26 |
| Oncology_METAP2 | 0.596 | 2.5E−27 | 5.64E−26 |
| Inflammation_HSPA1A | 0.713 | 2.9E−27 | 6.46E−26 |
| Neurology_II_CRYGD | 0.885 | 4.0E−27 | 8.82E−26 |
| Cardiometabolic_II_DNAJC6 | 0.759 | 5.1E−27 | 1.12E−25 |
| Neurology_CC2D1A | 0.779 | 5.5E−27 | 1.19E−25 |
| Inflammation_II_SNCA | 1.226 | 5.8E−27 | 1.25E−25 |
| Oncology_DCTN1 | 0.700 | 6.5E−27 | 1.39E−25 |
| Cardiometabolic_MNDA | 1.335 | 7.6E−27 | 1.61E−25 |
| Oncology_II_MAP2K1 | 0.692 | 7.8E−27 | 1.65E−25 |
| Neurology_II_PCBP2 | 0.575 | 9.7E−27 | 2.03E−25 |
| Inflammation_II_ACHE | 0.516 | 1.4E−26 | 2.93E−25 |
| Neurology_II_SPTBN2 | 0.320 | 1.9E−26 | 3.97E−25 |
| Oncology_II_THTPA | 0.686 | 2.9E−26 | 6.00E−25 |
| Inflammation_NT5C3A | 0.938 | 3.9E−26 | 8.03E−25 |
| Neurology_APRT | 0.645 | 4.0E−26 | 8.03E−25 |
| Oncology_SF3B4 | 0.801 | 5.2E−26 | 1.05E−24 |
| Neurology_DARS1 | 0.787 | 5.5E−26 | 1.10E−24 |
| Inflammation_11_EIF4E | 0.803 | 7.8E−26 | 1.56E−24 |
| Oncology_TPMT | 0.698 | 1.1E−25 | 2.24E−24 |
| Cardiometabolic_THOP1 | 0.281 | 1.4E−25 | 2.66E−24 |
| Neurology_ABHD14B | 0.562 | 1.4E−25 | 2.77E−24 |
| Oncology_HDGF | 0.773 | 1.6E−25 | 3.13E−24 |
| Oncology_SUGT1 | 0.701 | 1.8E−25 | 3.39E−24 |
| Cardiometabolic_SNX9 | 0.508 | 2.0E−25 | 3.77E−24 |
| Neurology_II_CLNS1A | 0.293 | 2.8E−25 | 5.38E−24 |
| Inflammation_II_RABEP1 | 0.695 | 2.9E−25 | 5.42E−24 |
| Oncology_II_LARP1 | 0.611 | 3.0E−25 | 5.61E−24 |
| Cardiometabolic_II_RPL14 | 0.519 | 3.0E−25 | 5.64E−24 |
| Inflammation_BID | 0.814 | 3.1E−25 | 5.64E−24 |
| Cardiometabolic_II_SLC4A1 | 0.697 | 3.7E−25 | 6.88E−24 |
| Inflammation_EGLN1 | 0.977 | 4.4E−25 | 8.09E−24 |
| Cardiometabolic_HNRNPK | 1.208 | 4.5E−25 | 8.17E−24 |
| Neurology_VTA1 | 0.689 | 4.6E−25 | 8.37E−24 |
| Inflammation_TRIM21 | 0.712 | 7.9E−25 | 1.42E−23 |
| Inflammation_NBN | 0.989 | 8.1E−25 | 1.44E−23 |
| Inflammation_PARP1 | 0.947 | 1.1E−24 | 1.93E−23 |
| Oncology_II_OTUD6B | 0.570 | 1.3E−24 | 2.24E−23 |
| Neurology_FKBP4 | 0.405 | 1.4E−24 | 2.48E−23 |
| Cardiometabolic_II_CRYZL1 | 0.823 | 1.5E−24 | 2.53E−23 |
| Cardiometabolic_ANXA4 | 0.797 | 1.9E−24 | 3.20E−23 |
| Cardiometabolic_OLR1 | 0.635 | 1.9E−24 | 3.20E−23 |
| Cardiometabolic_COMT | 0.810 | 4.7E−24 | 7.98E−23 |
| Cardiometabolic_II_AAMDC | 0.372 | 6.0E−24 | 1.02E−22 |
| Inflammation_II_TOP2B | 0.927 | 6.2E−24 | 1.05E−22 |
| Oncology_II_YJU2 | 0.420 | 6.8E−24 | 1.14E−22 |
| Cardiometabolic_II_ATP6V1G1 | 0.697 | 8.7E−24 | 1.45E−22 |
| Neurology_II_CSNK2A1 | 0.274 | 1.0E−23 | 1.70E−22 |
| Oncology_II_OGA | 0.609 | 1.0E−23 | 1.70E−22 |
| Cardiometabolic_II_NAGK | 0.631 | 1.4E−23 | 2.37E−22 |
| Neurology_WWP2 | 0.581 | 1.5E−23 | 2.52E−22 |
| Oncology_APBB1IP | 0.661 | 1.6E−23 | 2.53E−22 |
| Oncology_II_IST1 | 0.775 | 1.7E−23 | 2.70E−22 |
| Cardiometabolic_CEP43 | 0.683 | 1.7E−23 | 2.74E−22 |
| Inflammation_SCRN1 | 0.512 | 2.0E−23 | 3.17E−22 |
| Oncology_II_PFDN4 | 0.373 | 2.7E−23 | 4.30E−22 |
| Cardiometabolic_II_GRHPR | 0.571 | 2.8E−23 | 4.38E−22 |
| Inflammation_II_YWHAQ | 0.675 | 3.5E−23 | 5.50E−22 |
| Cardiometabolic_FADD | 0.828 | 3.6E−23 | 5.67E−22 |
| Oncology_II_SMNDC1 | 1.084 | 3.8E−23 | 5.90E−22 |
| Cardiometabolic_II_SART1 | 0.797 | 4.1E−23 | 6.39E−22 |
| Inflammation_NCF2 | 1.136 | 4.2E−23 | 6.48E−22 |
| Oncology_NAMPT | 1.018 | 4.3E−23 | 6.54E−22 |
| Inflammation_II_MK167 | 0.827 | 4.5E−23 | 6.92E−22 |
| Inflammation_II_DENR | 0.460 | 4.8E−23 | 7.34E−22 |
| Neurology_EZR | 0.259 | 5.2E−23 | 7.78E−22 |
| Cardiometabolic_NADK | 0.730 | 6.6E−23 | 9.93E−22 |
| Neurology_II_UROS | 0.494 | 7.8E−23 | 1.16E−21 |
| Oncology_OGFR | 0.328 | 8.8E−23 | 1.31E−21 |
| Inflammation_NUB1 | 0.866 | 9.0E−23 | 1.34E−21 |
| Inflammation_II_PAXX | 0.488 | 1.0E−22 | 1.50E−21 |
| Cardiometabolic_II_LRCH4 | 0.767 | 1.0E−22 | 1.52E−21 |
| Cardiometabolic_STK11 | 0.608 | 1.2E−22 | 1.71E−21 |
| Oncology_II_RAB44 | 1.003 | 1.2E−22 | 1.74E−21 |
| Oncology_RNF41 | 0.753 | 1.5E−22 | 2.12E−21 |
| Neurology_ATP6V1F | 0.732 | 1.5E−22 | 2.14E−21 |
| Inflammation_ADA | 0.343 | 1.5E−22 | 2.14E−21 |
| Inflammation_IRAK4 | 0.867 | 1.6E−22 | 2.24E−21 |
| Cardiometabolic_II_NFE2 | 0.719 | 1.7E−22 | 2.37E−21 |
| Oncology_PFKFB2 | 1.001 | 1.8E−22 | 2.48E−21 |
| Inflammation_II_ANXA1 | 0.707 | 1.8E−22 | 2.54E−21 |
| Oncology_NFKBIE | 0.659 | 2.7E−22 | 3.75E−21 |
| Oncology_ELOA | 0.930 | 3.2E−22 | 4.37E−21 |
| Neurology_NMNAT1 | 1.063 | 3.3E−22 | 4.50E−21 |
| Cardiometabolic_S100A11 | 0.651 | 3.4E−22 | 4.70E−21 |
| Oncology_II_ERI1 | 0.522 | 4.0E−22 | 5.53E−21 |
| Inflammation_II_BCL2L15 | 0.724 | 4.8E−22 | 6.53E−21 |
| Oncology_FEN1 | 1.207 | 5.5E−22 | 7.47E−21 |
| Neurology_II_STX3 | 0.268 | 5.8E−22 | 7.81E−21 |
| Oncology_CCT5 | 0.363 | 6.0E−22 | 8.11E−21 |
| Oncology_II_TDP1 | 0.824 | 6.1E−22 | 8.11E−21 |
| Inflammation_II_GPI | 0.593 | 6.6E−22 | 8.79E−21 |
| Neurology_TBCC | 0.727 | 8.7E−22 | 1.15E−20 |
| Neurology_II_SNRPB2 | 1.023 | 9.0E−22 | 1.19E−20 |
| Oncology_STAT5B | 1.037 | 1.1E−21 | 1.49E−20 |
| Oncology_DCTN2 | 0.905 | 1.2E−21 | 1.58E−20 |
| Inflammation_II_TSPYL1 | 0.271 | 1.2E−21 | 1.59E−20 |
| Oncology_DDX58 | 0.944 | 1.3E−21 | 1.72E−20 |
| Neurology_MPO | 0.520 | 1.5E−21 | 1.91E−20 |
| Neurology_II_ZHX2 | 0.595 | 2.0E−21 | 2.61E−20 |
| Cardiometabolic_LACTB2 | 0.476 | 2.2E−21 | 2.75E−20 |
| Neurology_PADI4 | 1.180 | 2.2E−21 | 2.85E−20 |
| Oncology_II_DUT | 0.735 | 2.4E−21 | 3.02E−20 |
| Neurology_II_PRKAR2A | 0.826 | 2.4E−21 | 3.05E−20 |
| Oncology_II_GLYR1 | 0.714 | 2.9E−21 | 3.62E−20 |
| Oncology_ANKRD54 | 0.590 | 2.9E−21 | 3.67E−20 |
| Oncology_II_LRRFIP1 | 0.529 | 3.0E−21 | 3.74E−20 |
| Cardiometabolic_USP8 | 0.704 | 3.4E−21 | 4.16E−20 |
| Oncology_SRP14 | 0.786 | 3.9E−21 | 4.84E−20 |
| Cardiometabolic_BAG6 | 0.314 | 5.1E−21 | 6.34E−20 |
| Inflammation_II_BNIP3L | 0.449 | 5.4E−21 | 6.59E−20 |
| Neurology_HARS1 | 0.592 | 5.8E−21 | 7.02E−20 |
| Oncology_II_CWC15 | 0.784 | 8.1E−21 | 9.82E−20 |
| Neurology_LBR | 0.979 | 8.4E−21 | 1.02E−19 |
| Inflammation_HCLS1 | 0.677 | 8.7E−21 | 1.05E−19 |
| Cardiometabolic_II_ASRGL1 | 0.828 | 9.7E−21 | 1.16E−19 |
| Neurology_II_HDGFL2 | 0.817 | 1.4E−20 | 1.66E−19 |
| Neurology_FMNL1 | 1.055 | 1.4E−20 | 1.70E−19 |
| Neurology_CHMP1A | 0.587 | 1.4E−20 | 1.70E−19 |
| Neurology_ANXA3 | 0.929 | 1.6E−20 | 1.88E−19 |
| Neurology_II_BAP18 | 0.969 | 1.8E−20 | 2.09E−19 |
| Neurology_II_C7orf50 | 0.461 | 1.8E−20 | 2.09E−19 |
| Oncology_II_JPT2 | 0.626 | 1.8E−20 | 2.12E−19 |
| Oncology_RASSF2 | 0.967 | 1.9E−20 | 2.16E−19 |
| Neurology_PXN | 0.699 | 2.3E−20 | 2.64E−19 |
| Inflammation_II_DAPK2 | 0.975 | 2.6E−20 | 3.02E−19 |
| Neurology_II_CASC3 | 0.321 | 2.7E−20 | 3.09E−19 |
| Oncology_FUS | 0.511 | 3.2E−20 | 3.64E−19 |
| Inflammation_PSIP1 | 0.878 | 3.3E−20 | 3.76E−19 |
| Cardiometabolic_II_TPR | 0.852 | 3.3E−20 | 3.77E−19 |
| Oncology_POLR2F | 0.529 | 3.4E−20 | 3.81E−19 |
| Cardiometabolic_AZU1 | 0.813 | 3.4E−20 | 3.81E−19 |
| Oncology_APEX1 | 0.821 | 3.5E−20 | 3.92E−19 |
| Inflammation_SAMD9L | 0.750 | 3.7E−20 | 4.11E−19 |
| Oncology_CDC37 | 0.669 | 3.9E−20 | 4.32E−19 |
| Neurology_SERPINB1 | 1.014 | 4.6E−20 | 5.14E−19 |
| Cardiometabolic_MPHOSPH8 | 0.721 | 4.8E−20 | 5.32E−19 |
| Oncology_II_YARS1 | 1.107 | 5.0E−20 | 5.51E−19 |
| Oncology_II_LMNB1 | 0.817 | 5.4E−20 | 5.93E−19 |
| Cardiometabolic_II_GGACT | 0.490 | 5.6E−20 | 6.15E−19 |
| Inflammation_LSP1 | 0.435 | 5.9E−20 | 6.43E−19 |
| Cardiometabolic_II_TOR1AIP1 | 0.915 | 6.0E−20 | 6.54E−19 |
| Neurology_ENO2 | 0.418 | 6.2E−20 | 6.69E−19 |
| Neurology_II_MORC3 | 0.494 | 6.8E−20 | 7.32E−19 |
| Neurology_II_INPP5J | 0.305 | 7.2E−20 | 7.74E−19 |
| Cardiometabolic_II_PACS2 | 0.461 | 7.5E−20 | 8.04E−19 |
| Cardiometabolic_AHCY | 0.606 | 8.0E−20 | 8.48E−19 |
| Cardiometabolic_CSTB | 0.359 | 8.7E−20 | 9.27E−19 |
| Inflammation_DNAJA2 | 0.719 | 8.8E−20 | 9.27E−19 |
| Cardiometabolic_RNASE3 | 1.110 | 9.1E−20 | 9.63E−19 |
| Inflammation_BACH1 | 0.533 | 9.8E−20 | 1.03E−18 |
| Inflammation_IRAK1 | 0.510 | 1.1E−19 | 1.11E−18 |
| Inflammation_DBNL | 0.823 | 1.2E−19 | 1.24E−18 |
| Neurology_II_NARS1 | 0.401 | 1.3E−19 | 1.35E−18 |
| Neurology_II_DYNLT1 | 0.719 | 1.6E−19 | 1.70E−18 |
| Inflammation_PRDX5 | 0.676 | 1.9E−19 | 1.95E−18 |
| Neurology_NPM1 | 0.958 | 2.0E−19 | 2.04E−18 |
| Neurology_TNFSF14 | 0.427 | 2.3E−19 | 2.34E−18 |
| Neurology_CASP10 | 0.979 | 2.3E−19 | 2.34E−18 |
| Cardiometabolic_CEBPB | 0.449 | 2.3E−19 | 2.34E−18 |
| Cardiometabolic_II_NIT2 | 0.600 | 2.7E−19 | 2.73E−18 |
| Oncology_II_TNFAIP2 | 0.647 | 2.7E−19 | 2.75E−18 |
| Cardiometabolic_ZBTB17 | 0.468 | 2.8E−19 | 2.83E−18 |
| Cardiometabolic_II_RNF5 | 0.517 | 2.9E−19 | 2.91E−18 |
| Oncology_II_CDC26 | 0.420 | 2.9E−19 | 2.91E−18 |
| Neurology_FGR | 1.048 | 3.1E−19 | 3.04E−18 |
| Oncology_II_TRIM25 | 0.870 | 3.2E−19 | 3.19E−18 |
| Neurology_TBCB | 0.873 | 3.6E−19 | 3.55E−18 |
| Oncology_RP2 | 0.370 | 4.2E−19 | 4.19E−18 |
| Inflammation_II_GCHFR | 0.397 | 5.4E−19 | 5.34E−18 |
| Oncology_MSRA | 0.660 | 5.9E−19 | 5.83E−18 |
| Cardiometabolic_II_NFKB1 | 0.683 | 6.0E−19 | 5.88E−18 |
| Inflammation_HEXIM1 | 0.590 | 6.2E−19 | 6.05E−18 |
| Inflammation_CRKL | 0.737 | 6.3E−19 | 6.13E−18 |
| Inflammation_II_ZBP1 | 0.480 | 6.8E−19 | 6.58E−18 |
| Oncology_II_EIF2AK2 | 1.035 | 7.2E−19 | 6.90E−18 |
| Oncology_CHAC2 | 0.584 | 7.4E−19 | 7.11E−18 |
| Oncology_II_FAM13A | 0.566 | 7.8E−19 | 7.43E−18 |
| Oncology_II_RBP7 | 0.664 | 8.4E−19 | 8.01E−18 |
| Cardiometabolic_CHEK2 | 0.764 | 8.8E−19 | 8.39E−18 |
| Neurology_II_GOLGA3 | 0.548 | 8.9E−19 | 8.41E−18 |
| Inflammation_IKBKG | 0.764 | 9.7E−19 | 9.13E−18 |
| Inflammation_II_FOXJ3 | 0.550 | 1.0E−18 | 9.46E−18 |
| Oncology_PQBP1 | 0.718 | 1.0E−18 | 9.56E−18 |
| Oncology_RAD23B | 0.386 | 1.1E−18 | 9.87E−18 |
| Cardiometabolic_II_GMFG | 0.685 | 1.1E−18 | 9.87E−18 |
| Oncology_II_ARF6 | 0.842 | 1.2E−18 | 1.10E−17 |
| Oncology_PRKRA | 0.633 | 1.4E−18 | 1.33E−17 |
| Neurology_II_ARHGEF1 | 0.684 | 1.8E−18 | 1.66E−17 |
| Neurology_FABP5 | 0.554 | 1.9E−18 | 1.71E−17 |
| Neurology_II_KCTD5 | 0.517 | 1.9E−18 | 1.71E−17 |
| Neurology_II_FGD3 | 0.737 | 2.0E−18 | 1.80E−17 |
| Inflammation_SRPK2 | 0.535 | 2.0E−18 | 1.83E−17 |
| Neurology_IPCEF1 | 0.794 | 2.0E−18 | 1.84E−17 |
| Neurology_II_RNASEH2A | 0.465 | 2.1E−18 | 1.92E−17 |
| Neurology_II_BOLA1 | 0.348 | 2.2E−18 | 2.03E−17 |
| Neurology_II_TNIP1 | 0.906 | 2.3E−18 | 2.05E−17 |
| Oncology_II_DHPS | 0.341 | 2.3E−18 | 2.07E−17 |
| Oncology_SORD | 0.473 | 2.7E−18 | 2.41E−17 |
| Neurology_II_SAFB2 | 0.392 | 2.8E−18 | 2.50E−17 |
| Neurology_II_OMP | 0.283 | 3.0E−18 | 2.66E−17 |
| Inflammation_II_BAG4 | 0.526 | 3.4E−18 | 2.99E−17 |
| Neurology_ENO1 | 0.693 | 3.7E−18 | 3.26E−17 |
| Cardiometabolic_PPP1R2 | 0.526 | 3.8E−18 | 3.38E−17 |
| Cardiometabolic_II_PDAP1 | 0.550 | 3.9E−18 | 3.40E−17 |
| Oncology_II_TRIM26 | 0.685 | 4.2E−18 | 3.67E−17 |
| Oncology_II_SWAP70 | 0.361 | 4.2E−18 | 3.70E−17 |
| Cardiometabolic_II_ITPA | 0.469 | 4.6E−18 | 3.99E−17 |
| Inflammation_II_NEDD9 | 0.413 | 4.6E−18 | 3.99E−17 |
| Oncology_II_RALY | 0.620 | 4.9E−18 | 4.23E−17 |
| Inflammation_II_SPART | 0.657 | 5.2E−18 | 4.49E−17 |
| Inflammation_EIF4G1 | 0.808 | 5.3E−18 | 4.60E−17 |
| Oncology_II_NMI | 0.576 | 8.4E−18 | 7.21E−17 |
| Neurology_GPKOW | 0.399 | 1.0E−17 | 8.58E−17 |
| Oncology_II_NUDT16 | 0.643 | 1.1E−17 | 9.00E−17 |
| Cardiometabolic_PLIN3 | 0.404 | 1.2E−17 | 1.00E−16 |
| Oncology_II_FNTA | 0.239 | 1.5E−17 | 1.25E−16 |
| Neurology_ARID4B | 0.629 | 1.5E−17 | 1.25E−16 |
| Neurology_TARBP2 | 0.584 | 1.5E−17 | 1.26E−16 |
| Neurology_ING1 | 0.782 | 1.6E−17 | 1.36E−16 |
| Inflammation_II_VTI1A | 0.483 | 1.7E−17 | 1.42E−16 |
| Neurology_SETMAR | 0.323 | 2.0E−17 | 1.67E−16 |
| Neurology_II_ELAC1 | 0.623 | 2.0E−17 | 1.68E−16 |
| Neurology_II_KLF4 | 0.445 | 2.1E−17 | 1.76E−16 |
| Inflammation_CD40LG | 0.607 | 2.1E−17 | 1.77E−16 |
| Cardiometabolic_II_GNPDA1 | 0.342 | 2.1E−17 | 1.77E−16 |
| Cardiometabolic_II_ENTR1 | 0.432 | 2.4E−17 | 1.96E−16 |
| Inflammation_ANXA11 | 0.941 | 2.8E−17 | 2.32E−16 |
| Neurology_II_GBP1 | 0.719 | 3.0E−17 | 2.43E−16 |
| Neurology_ILKAP | 0.693 | 3.2E−17 | 2.59E−16 |
| Neurology_FKBP5 | 0.759 | 3.5E−17 | 2.84E−16 |
| Cardiometabolic_II_EIF5 | 0.391 | 3.8E−17 | 3.07E−16 |
| Cardiometabolic_II_NFYA | 0.416 | 4.3E−17 | 3.47E−16 |
| Neurology_II_AZI2 | 0.529 | 4.7E−17 | 3.78E−16 |
| Neurology_CASP1 | 0.861 | 5.1E−17 | 4.14E−16 |
| Cardiometabolic_II_HSBP1 | 0.632 | 5.4E−17 | 4.31E−16 |
| Inflammation_SHMT1 | 0.577 | 6.0E−17 | 4.80E−16 |
| Neurology_II_PIBF1 | 0.763 | 6.1E−17 | 4.87E−16 |
| Oncology_II_SH3BP1 | 0.498 | 6.7E−17 | 5.33E−16 |
| Inflammation_SERPINB8 | 0.691 | 7.4E−17 | 5.91E−16 |
| Cardiometabolic_II_ANXA2 | 0.685 | 7.5E−17 | 5.96E−16 |
| Oncology_STX4 | 0.570 | 8.8E−17 | 6.98E−16 |
| Neurology_MAD1L1 | 0.670 | 9.0E−17 | 7.10E−16 |
| Neurology_II_AP3S2 | 0.379 | 9.3E−17 | 7.30E−16 |
| Neurology_II_MYCBP2 | 0.499 | 9.5E−17 | 7.45E−16 |
| Oncology_II_SUGP1 | 0.415 | 9.8E−17 | 7.63E−16 |
| Oncology_MAEA | 0.425 | 9.8E−17 | 7.63E−16 |
| Oncology_DRG2 | 0.352 | 1.0E−16 | 7.83E−16 |
| Cardiometabolic_PAG1 | 0.701 | 1.1E−16 | 8.17E−16 |
| Cardiometabolic_II_CALCOCO2 | 0.601 | 1.3E−16 | 9.97E−16 |
| Cardiometabolic_BLMH | 0.206 | 1.6E−16 | 1.21E−15 |
| Neurology_TXLNA | 0.626 | 1.8E−16 | 1.35E−15 |
| Oncology_II_GIMAP8 | 0.478 | 1.8E−16 | 1.40E−15 |
| Oncology_II_WDR46 | 0.529 | 1.9E−16 | 1.44E−15 |
| Inflammation_II_CEBPA | 0.289 | 1.9E−16 | 1.46E−15 |
| Oncology_II_DNAJB14 | 0.666 | 1.9E−16 | 1.46E−15 |
| Oncology_II_PPP2R5A | 0.896 | 2.1E−16 | 1.58E−15 |
| Oncology_II_MTHFSD | 0.624 | 2.9E−16 | 2.18E−15 |
| Neurology_PTS | 0.359 | 2.9E−16 | 2.19E−15 |
| Oncology_II_ATG16L1 | 0.498 | 2.9E−16 | 2.21E−15 |
| Inflammation_II_TNFAIP8L2 | 0.510 | 3.1E−16 | 2.33E−15 |
| Oncology_LPCAT2 | 0.558 | 3.1E−16 | 2.34E−15 |
| Cardiometabolic_II_ENOX2 | 0.286 | 3.1E−16 | 2.34E−15 |
| Neurology_II_DNAJC21 | 0.218 | 3.1E−16 | 2.34E−15 |
| Neurology_II_TAX1BP1 | 0.500 | 3.2E−16 | 2.38E−15 |
| Neurology_II_SATB1 | 0.410 | 3.8E−16 | 2.82E−15 |
| Cardiometabolic_II_EEF1D | 0.770 | 4.6E−16 | 3.42E−15 |
| Inflammation_II_EP300 | 0.435 | 4.7E−16 | 3.46E−15 |
| Neurology_II_EDF1 | 0.657 | 4.8E−16 | 3.51E−15 |
| Oncology_II_PPP1R12B | 0.497 | 5.4E−16 | 3.98E−15 |
| Neurology_PTPN1 | 0.681 | 5.4E−16 | 4.00E−15 |
| Neurology_II_WASHC3 | 0.709 | 5.5E−16 | 4.05E−15 |
| Oncology_II_VPS4B | 0.584 | 7.1E−16 | 5.22E−15 |
| Neurology_II_SEPTIN8 | 0.228 | 7.4E−16 | 5.41E−15 |
| Neurology_MMP8 | 0.593 | 7.5E−16 | 5.47E−15 |
| Oncology_II_BRAP | 0.752 | 7.8E−16 | 5.66E−15 |
| Inflammation_II_MARS1 | 0.631 | 8.3E−16 | 6.00E−15 |
| Neurology_SSB | 0.555 | 8.9E−16 | 6.39E−15 |
| Inflammation_II_RIDA | 0.306 | 9.3E−16 | 6.70E−15 |
| Neurology_XRCC4 | 0.302 | 9.5E−16 | 6.79E−15 |
| Oncology_CRACR2A | 0.915 | 1.2E−15 | 8.25E−15 |
| Oncology_II_TRIM58 | 0.545 | 1.3E−15 | 9.03E−15 |
| Neurology_TIGAR | 0.494 | 1.3E−15 | 9.37E−15 |
| Cardiometabolic_II_CDA | 0.416 | 1.6E−15 | 1.14E−14 |
| Cardiometabolic_II_NT5C | 0.467 | 1.8E−15 | 1.30E−14 |
| Cardiometabolic_II_OPLAH | 0.410 | 1.9E−15 | 1.33E−14 |
| Neurology_SERPINB9 | 0.295 | 2.0E−15 | 1.38E−14 |
| Inflammation_IL16 | 0.451 | 2.0E−15 | 1.43E−14 |
| Inflammation_II_TERF1 | 0.387 | 2.1E−15 | 1.47E−14 |
| Inflammation_FOXO1 | 0.648 | 2.2E−15 | 1.51E−14 |
| Cardiometabolic_II_FAM172A | 0.426 | 2.6E−15 | 1.84E−14 |
| Cardiometabolic_II_ARL2BP | 0.460 | 2.8E−15 | 1.93E−14 |
| Cardiometabolic_II_UBE2L6 | 0.397 | 2.8E−15 | 1.96E−14 |
| Oncology_DCXR | 0.317 | 2.9E−15 | 1.98E−14 |
| Oncology_II_CEP152 | 0.584 | 3.0E−15 | 2.09E−14 |
| Oncology_II_STAU1 | 0.401 | 3.1E−15 | 2.14E−14 |
| Cardiometabolic_II_COMMD1 | 0.497 | 3.1E−15 | 2.16E−14 |
| Oncology_FXN | 0.406 | 3.1E−15 | 2.16E−14 |
| Inflammation_II_TREML1 | 0.451 | 3.2E−15 | 2.17E−14 |
| Oncology_AKR1B1 | 0.574 | 3.4E−15 | 2.35E−14 |
| Neurology_WARS | 0.236 | 3.6E−15 | 2.47E−14 |
| Oncology_LYAR | 0.672 | 3.7E−15 | 2.49E−14 |
| Oncology_ATOX1 | 0.489 | 3.7E−15 | 2.51E−14 |
| Cardiometabolic_CORO1A | 0.871 | 3.8E−15 | 2.61E−14 |
| Oncology_II_AP1G2 | 0.580 | 4.0E−15 | 2.69E−14 |
| Cardiometabolic_II_TCOF1 | 0.356 | 4.1E−15 | 2.76E−14 |
| Inflammation_II_PIKFYVE | 0.350 | 4.2E−15 | 2.81E−14 |
| Neurology_II_SNAPIN | 0.413 | 4.5E−15 | 3.03E−14 |
| Inflammation_II_EVI5 | 0.596 | 4.6E−15 | 3.05E−14 |
| Oncology_II_THAP12 | 0.552 | 4.9E−15 | 3.26E−14 |
| Oncology_INPPL1 | 0.629 | 5.2E−15 | 3.45E−14 |
| Cardiometabolic_II_EIF2S2 | 0.275 | 5.7E−15 | 3.77E−14 |
| Inflammation_IL1B | 0.626 | 5.8E−15 | 3.86E−14 |
| Cardiometabolic_II_GOT1 | 0.274 | 6.3E−15 | 4.19E−14 |
| Cardiometabolic_VIM | 0.637 | 6.3E−15 | 4.19E−14 |
| Neurology_IMPA1 | 0.288 | 6.5E−15 | 4.29E−14 |
| Cardiometabolic_RCOR1 | 0.459 | 6.5E−15 | 4.29E−14 |
| Oncology_TJAP1 | 0.599 | 6.6E−15 | 4.30E−14 |
| Inflammation_ICA1 | 0.498 | 7.5E−15 | 4.88E−14 |
| Neurology_EBAG9 | 0.553 | 7.7E−15 | 5.06E−14 |
| Oncology_MPI | 0.512 | 8.0E−15 | 5.19E−14 |
| Neurology_II_PLCB2 | 0.749 | 9.0E−15 | 5.87E−14 |
| Cardiometabolic_II_NUP50 | 0.272 | 9.0E−15 | 5.87E−14 |
| Neurology_DBI | 0.463 | 1.1E−14 | 6.94E−14 |
| Neurology_II_HIP1R | 0.499 | 1.1E−14 | 7.04E−14 |
| Oncology_II_AP3B1 | 0.461 | 1.2E−14 | 7.45E−14 |
| Inflammation_II_TP53BP1 | 0.392 | 1.2E−14 | 7.83E−14 |
| Neurology_II_CCDC50 | 0.418 | 1.3E−14 | 8.19E−14 |
| Cardiometabolic_II_NUDT10 | 0.224 | 1.3E−14 | 8.45E−14 |
| Inflammation_II_DNAJB6 | 0.499 | 1.3E−14 | 8.47E−14 |
| Oncology_PPP1R12A | 0.622 | 1.4E−14 | 8.95E−14 |
| Inflammation_II_NUMB | 0.477 | 1.5E−14 | 9.38E−14 |
| Oncology_II_CIRBP | 0.690 | 1.6E−14 | 1.00E−13 |
| Inflammation_II_SIRT1 | 0.302 | 1.6E−14 | 1.01E−13 |
| Oncology_II_RFC4 | 0.236 | 1.7E−14 | 1.08E−13 |
| Inflammation_SH2D1A | 0.637 | 1.8E−14 | 1.11E−13 |
| Neurology_HMOX2 | 0.416 | 2.0E−14 | 1.26E−13 |
| Oncology_FOXO3 | 0.764 | 2.0E−14 | 1.29E−13 |
| Neurology_FYB1 | 0.692 | 2.1E−14 | 1.31E−13 |
| Cardiometabolic_CLC | 0.423 | 2.1E−14 | 1.33E−13 |
| Neurology_II_FARSA | 0.358 | 2.2E−14 | 1.40E−13 |
| Cardiometabolic_EDIL3 | −0.287 | 2.3E−14 | 1.44E−13 |
| Neurology_II_CCAR2 | 0.414 | 2.4E−14 | 1.47E−13 |
| Inflammation_MAPK9 | 0.300 | 2.7E−14 | 1.65E−13 |
| Oncology_FLI1 | 0.602 | 2.8E−14 | 1.76E−13 |
| Oncology_II_COMMD9 | 0.335 | 3.9E−14 | 2.42E−13 |
| Cardiometabolic_II_CEP112 | 0.303 | 4.1E−14 | 2.50E−13 |
| Neurology_II_ARFIP1 | 0.342 | 4.3E−14 | 2.65E−13 |
| Cardiometabolic_II_GET3 | 0.357 | 5.0E−14 | 3.04E−13 |
| Neurology_TMSB10 | 0.589 | 5.0E−14 | 3.04E−13 |
| Oncology_ARSB | 0.322 | 5.4E−14 | 3.28E−13 |
| Oncology_USO1 | 0.835 | 5.4E−14 | 3.32E−13 |
| Neurology_II_DDHD2 | 0.323 | 6.1E−14 | 3.73E−13 |
| Oncology_S100A12 | 0.581 | 7.0E−14 | 4.25E−13 |
| Oncology_CEP85 | 0.593 | 7.6E−14 | 4.59E−13 |
| Cardiometabolic_II_BRD3 | 0.360 | 9.0E−14 | 5.43E−13 |
| Oncology_II_MAPKAPK2 | 0.393 | 9.0E−14 | 5.46E−13 |
| Neurology_II_ESYT2 | 0.533 | 9.1E−14 | 5.46E−13 |
| Inflammation_II_BLNK | 0.342 | 9.1E−14 | 5.49E−13 |
| Neurology_II_GCC1 | 0.671 | 9.6E−14 | 5.78E−13 |
| Neurology_PFDN2 | 0.361 | 1.0E−13 | 6.23E−13 |
| Oncology_II_SDCCAG8 | 0.735 | 1.0E−13 | 6.24E−13 |
| Neurology_CERT | 0.656 | 1.2E−13 | 7.27E−13 |
| Inflammation_II_GIMAP7 | 0.346 | 1.3E−13 | 7.85E−13 |
| Cardiometabolic_II_ABRAXAS2 | 0.280 | 1.4E−13 | 8.21E−13 |
| Neurology_SKAP1 | 0.822 | 1.4E−13 | 8.33E−13 |
| Oncology_II_STAM | 0.293 | 1.5E−13 | 8.89E−13 |
| Oncology_II_AHSA1 | 0.286 | 1.5E−13 | 8.97E−13 |
| Neurology_II_DOC2B | 0.328 | 1.6E−13 | 9.70E−13 |
| Neurology_DCTN6 | 0.504 | 1.9E−13 | 1.14E−12 |
| Oncology_II_RAPGEF2 | 0.355 | 2.0E−13 | 1.19E−12 |
| Inflammation_TANK | 0.618 | 2.1E−13 | 1.25E−12 |
| Oncology_II_IFIT3 | 0.429 | 2.3E−13 | 1.32E−12 |
| Inflammation_II_XIAP | 0.465 | 2.6E−13 | 1.51E−12 |
| Inflammation_FIS1 | 0.400 | 2.7E−13 | 1.55E−12 |
| Oncology_II_TARS1 | 0.347 | 2.7E−13 | 1.56E−12 |
| Cardiometabolic_II_CEP170 | 0.538 | 2.8E−13 | 1.64E−12 |
| Oncology_II_MNAT1 | 0.370 | 3.2E−13 | 1.87E−12 |
| Oncology_VAT1 | 0.147 | 3.6E−13 | 2.07E−12 |
| Oncology_VPS37A | 0.564 | 3.8E−13 | 2.21E−12 |
| Inflammation_MAP2K6 | 0.557 | 4.2E−13 | 2.41E−12 |
| Oncology_II_SMAD3 | 0.299 | 4.6E−13 | 2.65E−12 |
| Inflammation_II_ZNF174 | 0.342 | 4.7E−13 | 2.71E−12 |
| Cardiometabolic_II_SNX5 | 0.188 | 4.9E−13 | 2.82E−12 |
| Oncology_CAMKK1 | 0.358 | 4.9E−13 | 2.82E−12 |
| Inflammation_II_VAMP8 | 0.598 | 5.3E−13 | 3.01E−12 |
| Inflammation_NUDC | 0.371 | 5.6E−13 | 3.16E−12 |
| Neurology_II_GIGYF2 | 0.511 | 5.8E−13 | 3.29E−12 |
| Inflammation_EGF | 0.635 | 5.8E−13 | 3.30E−12 |
| Inflammation_MYO9B | 0.493 | 6.2E−13 | 3.53E−12 |
| Inflammation_TBC1D5 | 0.565 | 6.7E−13 | 3.77E−12 |
| Cardiometabolic_SNAP23 | 0.661 | 7.1E−13 | 4.02E−12 |
| Inflammation_II_SYAP1 | 0.245 | 7.6E−13 | 4.30E−12 |
| Cardiometabolic_II_CHMP6 | 0.292 | 7.8E−13 | 4.40E−12 |
| Oncology_II_UFD1 | 0.777 | 8.0E−13 | 4.49E−12 |
| Inflammation_STX8 | 0.389 | 8.1E−13 | 4.54E−12 |
| Neurology_EREG | 0.370 | 1.1E−12 | 5.88E−12 |
| Neurology_II_PLEKHO1 | 0.311 | 1.1E−12 | 5.91E−12 |
| Cardiometabolic_CEACAM8 | 0.358 | 1.1E−12 | 6.37E−12 |
| Cardiometabolic_II_EPPK1 | 0.466 | 1.2E−12 | 6.53E−12 |
| Oncology_DDAH1 | 0.332 | 1.2E−12 | 6.69E−12 |
| Oncology_CALCOCO1 | 0.591 | 1.2E−12 | 6.85E−12 |
| Cardiometabolic_II_SEC31A | 0.345 | 1.3E−12 | 7.27E−12 |
| Inflammation_II_MCEMP1 | 0.560 | 1.4E−12 | 7.46E−12 |
| Cardiometabolic_PRTN3 | 0.362 | 1.4E−12 | 7.51E−12 |
| Neurology_II_CAMSAP1 | 0.714 | 1.5E−12 | 8.10E−12 |
| Neurology_II_VAV3 | 0.619 | 1.5E−12 | 8.47E−12 |
| Neurology_MAX | 0.716 | 1.6E−12 | 8.79E−12 |
| Inflammation_PTPN6 | 0.611 | 1.9E−12 | 1.01E−11 |
| Cardiometabolic_II_TWF2 | 0.518 | 2.0E−12 | 1.08E−11 |
| Inflammation_II_CACYBP | 0.647 | 2.0E−12 | 1.11E−11 |
| Oncology_ABL1 | 0.411 | 2.1E−12 | 1.12E−11 |
| Inflammation_MGMT | 0.712 | 2.2E−12 | 1.17E−11 |
| Neurology_DNMBP | 0.458 | 2.2E−12 | 1.18E−11 |
| Neurology_II_TIMM8A | 0.476 | 2.3E−12 | 1.22E−11 |
| Inflammation_PPP1R9B | 0.505 | 2.6E−12 | 1.42E−11 |
| Oncology_VPS53 | 0.424 | 2.7E−12 | 1.47E−11 |
| Oncology_DPY30 | 0.513 | 2.8E−12 | 1.50E−11 |
| Inflammation_II_STX7 | 0.289 | 3.2E−12 | 1.72E−11 |
| Cardiometabolic_II_SNU13 | 0.441 | 3.3E−12 | 1.75E−11 |
| Oncology_II_MORF4L2 | 0.261 | 3.3E−12 | 1.78E−11 |
| Inflammation_CCL13 | 0.325 | 3.5E−12 | 1.89E−11 |
| Oncology_SNAP29 | 0.531 | 3.7E−12 | 1.95E−11 |
| Oncology_II_NACC1 | 0.361 | 4.2E−12 | 2.22E−11 |
| Oncology_SEPTIN9 | 0.250 | 4.4E−12 | 2.34E−11 |
| Neurology_II_RANBP2 | 0.268 | 4.4E−12 | 2.34E−11 |
| Neurology_II_DGCR6 | 0.294 | 4.9E−12 | 2.59E−11 |
| Inflammation_II_ARHGAP45 | 0.490 | 4.9E−12 | 2.60E−11 |
| Oncology_CAPG | 0.455 | 5.2E−12 | 2.76E−11 |
| Oncology_ARHGAP1 | 0.240 | 5.5E−12 | 2.86E−11 |
| Inflammation_II_SLC9A3R1 | 0.387 | 5.8E−12 | 3.06E−11 |
| Oncology_TACC3 | 0.697 | 5.9E−12 | 3.11E−11 |
| Inflammation_II_TPD52L2 | 0.617 | 7.2E−12 | 3.76E−11 |
| Neurology_MAP4K5 | 0.538 | 8.0E−12 | 4.17E−11 |
| Cardiometabolic_IRAG2 | 0.676 | 8.4E−12 | 4.36E−11 |
| Cardiometabolic_II_HS1BP3 | 0.402 | 9.2E−12 | 4.76E−11 |
| Cardiometabolic_GYS1 | 0.617 | 9.2E−12 | 4.77E−11 |
| Neurology_KEL | 0.187 | 9.2E−12 | 4.77E−11 |
| Oncology_STX6 | 0.469 | 9.3E−12 | 4.81E−11 |
| Oncology_LAT2 | 0.519 | 9.9E−12 | 5.11E−11 |
| Inflammation_BCR | 0.459 | 1.0E−11 | 5.26E−11 |
| Oncology_ERBIN | 0.548 | 1.1E−11 | 5.54E−11 |
| Oncology_II_OFD1 | 0.285 | 1.1E−11 | 5.76E−11 |
| Neurology_CD63 | 0.302 | 1.1E−11 | 5.88E−11 |
| Neurology_MITD1 | 0.589 | 1.2E−11 | 5.89E−11 |
| Cardiometabolic_S100P | 0.407 | 1.2E−11 | 6.27E−11 |
| Cardiometabolic_II_PPM1F | 0.268 | 1.3E−11 | 6.42E−11 |
| Neurology_II_MINK1 | 0.528 | 1.3E−11 | 6.42E−11 |
| Inflammation_DGKZ | 0.305 | 1.3E−11 | 6.54E−11 |
| Oncology_II_CYB5R2 | 0.334 | 1.6E−11 | 7.89E−11 |
| Inflammation_II_STAT2 | 0.344 | 1.6E−11 | 8.06E−11 |
| Inflammation_IL1RN | 0.381 | 1.8E−11 | 9.32E−11 |
| Cardiometabolic_II_NFX1 | 0.318 | 1.9E−11 | 9.71E−11 |
| Cardiometabolic_TIA1 | 0.483 | 2.2E−11 | 1.09E−10 |
| Oncology_CEP20 | 0.500 | 2.2E−11 | 1.09E−10 |
| Oncology_II_MORF4L1 | 0.263 | 2.2E−11 | 1.12E−10 |
| Inflammation_TRIM5 | 0.480 | 2.5E−11 | 1.26E−10 |
| Inflammation_SKAP2 | 0.674 | 2.6E−11 | 1.30E−10 |
| Inflammation_II_GAPDH | 0.218 | 2.6E−11 | 1.30E−10 |
| Inflammation_TGFA | 0.219 | 2.8E−11 | 1.37E−10 |
| Neurology_II_C2orf69 | 0.313 | 3.2E−11 | 1.59E−10 |
| Neurology_II_USP28 | 0.212 | 3.4E−11 | 1.69E−10 |
| Inflammation_II_GIT1 | 0.498 | 3.7E−11 | 1.85E−10 |
| Inflammation_RAB6A | 0.301 | 3.9E−11 | 1.91E−10 |
| Inflammation_ITGA6 | 0.216 | 3.9E−11 | 1.92E−10 |
| Neurology_II_NAA80 | 0.477 | 4.0E−11 | 1.95E−10 |
| Inflammation_II_GSR | 0.129 | 4.2E−11 | 2.08E−10 |
| Inflammation_II_RPA2 | 0.227 | 4.3E−11 | 2.12E−10 |
| Inflammation_II_DDX39A | 0.179 | 4.5E−11 | 2.20E−10 |
| Inflammation_II_MTDH | 0.505 | 4.9E−11 | 2.40E−10 |
| Oncology_II_MAPK13 | 0.398 | 5.2E−11 | 2.57E−10 |
| Oncology_II_BCL2 | 0.348 | 5.6E−11 | 2.76E−10 |
| Inflammation_CXCL17 | −0.274 | 6.2E−11 | 3.02E−10 |
| Neurology_II_REEP4 | 0.306 | 7.1E−11 | 3.45E−10 |
| Oncology_II_PBK | 0.181 | 7.6E−11 | 3.69E−10 |
| Neurology_TDRKH | 0.459 | 7.9E−11 | 3.85E−10 |
| Oncology_MAP3K5 | 0.549 | 8.0E−11 | 3.87E−10 |
| Cardiometabolic_II_HBZ | 0.473 | 8.2E−11 | 3.98E−10 |
| Inflammation_SIT1 | 0.453 | 8.9E−11 | 4.28E−10 |
| Neurology_II_AP2B1 | 0.195 | 8.9E−11 | 4.31E−10 |
| Neurology_II_CASP7 | 0.324 | 9.0E−11 | 4.33E−10 |
| Inflammation_AXIN1 | 0.441 | 9.0E−11 | 4.35E−10 |
| Oncology_MZT1 | 0.420 | 9.7E−11 | 4.68E−10 |
| Inflammation_NFATC1 | 0.421 | 1.1E−10 | 5.19E−10 |
| Oncology_II_VPS28 | 0.254 | 1.1E−10 | 5.34E−10 |
| Neurology_II_BCL2L1 | 0.438 | 1.1E−10 | 5.36E−10 |
| Inflammation_II_PTP4A3 | 0.204 | 1.2E−10 | 5.59E−10 |
| Oncology_NUDT2 | 0.397 | 1.2E−10 | 5.65E−10 |
| Oncology_CDC27 | 0.444 | 1.2E−10 | 5.91E−10 |
| Cardiometabolic_II_DDA1 | 0.306 | 1.3E−10 | 6.05E−10 |
| Neurology_II_HHEX | 0.390 | 1.3E−10 | 6.36E−10 |
| Inflammation_PRKAB1 | 0.294 | 1.3E−10 | 6.36E−10 |
| Oncology_SCAMP3 | 0.399 | 1.4E−10 | 6.45E−10 |
| Inflammation_OSM | 0.385 | 1.5E−10 | 6.92E−10 |
| Cardiometabolic_II_NECAP2 | 0.295 | 1.5E−10 | 7.11E−10 |
| Neurology_ITGAM | 0.172 | 1.5E−10 | 7.19E−10 |
| Cardiometabolic_SEMA7A | 0.148 | 1.7E−10 | 7.80E−10 |
| Oncology_II_CETN3 | 0.335 | 1.7E−10 | 8.18E−10 |
| Neurology_II_BLOC1S3 | 0.211 | 1.9E−10 | 9.06E−10 |
| Oncology_INPP1 | 0.365 | 1.9E−10 | 9.06E−10 |
| Neurology_GSTP1 | 0.413 | 2.0E−10 | 9.10E−10 |
| Neurology_II_GNAS | 0.177 | 2.0E−10 | 9.49E−10 |
| Inflammation_CEP164 | 0.447 | 2.1E−10 | 9.88E−10 |
| Oncology_MED18 | 0.397 | 2.2E−10 | 1.02E−09 |
| Inflammation_II_CSNK1D | 0.217 | 2.3E−10 | 1.08E−09 |
| Neurology_MMP9 | 0.314 | 2.4E−10 | 1.12E−09 |
| Cardiometabolic_II_RILPL2 | 0.506 | 2.9E−10 | 1.32E−09 |
| Oncology_KIFBP | 0.558 | 3.1E−10 | 1.44E−09 |
| Neurology_II_AK2 | 0.556 | 3.2E−10 | 1.49E−09 |
| Neurology_II_IDO1 | 0.310 | 3.4E−10 | 1.54E−09 |
| Oncology_DPEP2 | 0.152 | 3.4E−10 | 1.55E−09 |
| Neurology_II_NMT1 | 0.286 | 3.5E−10 | 1.58E−09 |
| Cardiometabolic_II_LRRC59 | 0.316 | 3.5E−10 | 1.61E−09 |
| Neurology_SERPINB6 | 0.276 | 3.8E−10 | 1.74E−09 |
| Oncology_CDKN2D | 0.595 | 4.8E−10 | 2.19E−09 |
| Neurology_C2CD2L | 0.201 | 5.5E−10 | 2.51E−09 |
| Oncology_II_ZNF830 | 0.384 | 5.6E−10 | 2.55E−09 |
| Neurology_II_DOK1 | 0.699 | 5.7E−10 | 2.58E−09 |
| Inflammation_TNFAIP8 | 0.552 | 5.8E−10 | 2.63E−09 |
| Neurology_APP | 0.286 | 6.6E−10 | 2.98E−09 |
| Oncology_II_IDO1 | 0.305 | 6.9E−10 | 3.12E−09 |
| Inflammation_CD6 | 0.321 | 7.1E−10 | 3.21E−09 |
| Cardiometabolic_STK4 | 0.364 | 9.0E−10 | 4.04E−09 |
| Oncology_II_PCYT2 | 0.364 | 9.2E−10 | 4.12E−09 |
| Oncology_II_GORASP2 | 0.229 | 9.2E−10 | 4.14E−09 |
| Oncology_MAVS | 0.433 | 9.4E−10 | 4.21E−09 |
| Inflammation_CSF3 | −0.287 | 1.0E−09 | 4.64E−09 |
| Oncology_II_TMED8 | 0.421 | 1.1E−09 | 4.74E−09 |
| Inflammation_II_GNPDA2 | 0.163 | 1.1E−09 | 4.82E−09 |
| Neurology_CCL2 | 0.170 | 1.1E−09 | 5.07E−09 |
| Cardiometabolic_GRAP2 | 0.567 | 1.2E−09 | 5.17E−09 |
| Inflammation_II_DAAM1 | 0.386 | 1.2E−09 | 5.52E−09 |
| Inflammation_ANGPT1 | 0.342 | 1.4E−09 | 6.14E−09 |
| Oncology_LYN | 0.337 | 1.6E−09 | 6.95E−09 |
| Neurology_II_OSBPL2 | 0.179 | 1.6E−09 | 7.06E−09 |
| Neurology_II_BRD2 | 0.186 | 1.6E−09 | 7.09E−09 |
| Cardiometabolic_II_CRYBB1 | 0.245 | 1.6E−09 | 7.09E−09 |
| Oncology_II_HSPA2 | 0.177 | 1.8E−09 | 7.74E−09 |
| Oncology_TBL1X | 0.431 | 1.8E−09 | 8.11E−09 |
| Neurology_II_RGS10 | 0.286 | 1.9E−09 | 8.13E−09 |
| Neurology_II_SPAG1 | 0.328 | 2.1E−09 | 9.26E−09 |
| Cardiometabolic_LGALS3 | 0.145 | 2.2E−09 | 9.44E−09 |
| Neurology_STK24 | 0.403 | 2.2E−09 | 9.81E−09 |
| Neurology_II_NGRN | 0.105 | 2.3E−09 | 9.98E−09 |
| Neurology_II_CHM | 0.259 | 2.6E−09 | 1.11E−08 |
| Inflammation_GOPC | 0.504 | 2.8E−09 | 1.22E−08 |
| Neurology_II_SMS | 0.242 | 2.9E−09 | 1.25E−08 |
| Cardiometabolic_HEBP1 | 0.271 | 2.9E−09 | 1.25E−08 |
| Oncology_II_SMAD2 | 0.106 | 2.9E−09 | 1.27E−08 |
| Oncology_HBEGF | 0.337 | 3.1E−09 | 1.33E−08 |
| Cardiometabolic_SUSD1 | 0.348 | 3.1E−09 | 1.35E−08 |
| Neurology_II_ARHGEF5 | 0.377 | 3.2E−09 | 1.37E−08 |
| Cardiometabolic_II_NAA10 | 0.370 | 3.4E−09 | 1.45E−08 |
| Neurology_GPC5 | 0.213 | 3.5E−09 | 1.49E−08 |
| Neurology_LGALS8 | 0.219 | 3.5E−09 | 1.49E−08 |
| Inflammation_II_YY1 | 0.202 | 3.8E−09 | 1.61E−08 |
| Oncology_II_MLLT1 | 0.263 | 3.9E−09 | 1.67E−08 |
| Neurology_BIN2 | 0.540 | 4.1E−09 | 1.74E−08 |
| Cardiometabolic_SDC4 | 0.310 | 4.3E−09 | 1.85E−08 |
| Neurology_II_SPTLC1 | 0.272 | 4.4E−09 | 1.86E−08 |
| Oncology_AIF1 | 0.697 | 4.4E−09 | 1.87E−08 |
| Cardiometabolic_II_ZCCHC8 | 0.378 | 4.5E−09 | 1.91E−08 |
| Cardiometabolic_II_AHNAK | 0.200 | 5.4E−09 | 2.29E−08 |
| Cardiometabolic_CD59 | 0.127 | 5.7E−09 | 2.41E−08 |
| Cardiometabolic_II_SERPINE2 | 0.331 | 5.9E−09 | 2.50E−08 |
| Oncology_II_ARHGAP30 | 0.173 | 5.9E−09 | 2.50E−08 |
| Inflammation_II_OLFM4 | 0.481 | 6.4E−09 | 2.69E−08 |
| Oncology_II_TRIM24 | 0.236 | 6.7E−09 | 2.82E−08 |
| Neurology_PPP3R1 | 0.223 | 7.1E−09 | 2.99E−08 |
| Inflammation_PLXNA4 | 0.377 | 7.5E−09 | 3.15E−08 |
| Inflammation_CCL26 | 0.428 | 7.6E−09 | 3.20E−08 |
| Cardiometabolic_II_PKD2 | 0.318 | 8.7E−09 | 3.63E−08 |
| Oncology_RRM2B | 0.318 | 8.7E−09 | 3.66E−08 |
| Neurology_II_AKT2 | 0.560 | 8.8E−09 | 3.69E−08 |
| Neurology_SULT1A1 | 0.616 | 9.2E−09 | 3.82E−08 |
| Neurology_PMVK | 0.729 | 9.3E−09 | 3.86E−08 |
| Inflammation_HLA-E | −0.125 | 9.6E−09 | 3.98E−08 |
| Cardiometabolic_PRKAR1A | 0.448 | 9.8E−09 | 4.06E−08 |
| Inflammation_PDGFB | 0.387 | 9.9E−09 | 4.11E−08 |
| Inflammation_HPCAL1 | 0.341 | 1.0E−08 | 4.16E−08 |
| Neurology_II_LMNB2 | 0.197 | 1.0E−08 | 4.28E−08 |
| Oncology_II_SLK | 0.316 | 1.1E−08 | 4.36E−08 |
| Neurology_II_ATXN2L | 0.140 | 1.2E−08 | 4.82E−08 |
| Neurology_II_RBM17 | 0.259 | 1.2E−08 | 5.02E−08 |
| Cardiometabolic_PDGFA | 0.339 | 1.2E−08 | 5.07E−08 |
| Oncology_VEGFC | 0.229 | 1.2E−08 | 5.07E−08 |
| Neurology_NID2 | 0.275 | 1.3E−08 | 5.24E−08 |
| Cardiometabolic_DIABLO | 0.589 | 1.3E−08 | 5.37E−08 |
| Cardiometabolic_NID1 | 0.151 | 1.3E−08 | 5.38E−08 |
| Neurology_II_NFIC | 0.186 | 1.4E−08 | 5.52E−08 |
| Neurology_II_DLGAP5 | 0.192 | 1.6E−08 | 6.68E−08 |
| Neurology_F11R | 0.207 | 1.7E−08 | 7.03E−08 |
| Oncology_GNE | 0.288 | 2.1E−08 | 8.44E−08 |
| Inflammation_PLA2G4A | 0.357 | 2.1E−08 | 8.71E−08 |
| Oncology_II_TMEM106A | 0.254 | 2.3E−08 | 9.29E−08 |
| Neurology_DKK1 | 0.241 | 2.4E−08 | 9.91E−08 |
| Neurology_DRAXIN | −0.171 | 2.5E−08 | 1.02E−07 |
| Neurology_BAX | 0.526 | 2.5E−08 | 1.02E−07 |
| Neurology_II_GID8 | 0.135 | 2.6E−08 | 1.03E−07 |
| Oncology_IQGAP2 | 0.334 | 2.7E−08 | 1.07E−07 |
| Oncology_II_RCC1 | 0.314 | 2.9E−08 | 1.16E−07 |
| Inflammation_II_VASP | 0.254 | 3.0E−08 | 1.19E−07 |
| Cardiometabolic_CXCL8 | 0.234 | 3.3E−08 | 1.31E−07 |
| Oncology_CPXM1 | 0.261 | 3.9E−08 | 1.58E−07 |
| Inflammation_II_DTD1 | 0.444 | 4.2E−08 | 1.68E−07 |
| Inflammation_DAPP1 | 0.597 | 4.4E−08 | 1.74E−07 |
| Oncology_II_LAMTOR5 | 0.209 | 4.8E−08 | 1.90E−07 |
| Neurology_GP6 | 0.333 | 5.0E−08 | 2.00E−07 |
| Inflammation_LAT | 0.350 | 5.3E−08 | 2.09E−07 |
| Oncology_BIRC2 | 0.274 | 5.5E−08 | 2.19E−07 |
| Cardiometabolic_II_GP1BB | 0.267 | 5.7E−08 | 2.28E−07 |
| Neurology_II_ARID3A | 0.185 | 5.9E−08 | 2.33E−07 |
| Oncology_FES | 0.265 | 5.9E−08 | 2.35E−07 |
| Inflammation_MPIG6B | 0.398 | 6.0E−08 | 2.37E−07 |
| Oncology_STX16 | 0.533 | 6.2E−08 | 2.43E−07 |
| Cardiometabolic_II_MYH9 | 0.491 | 6.8E−08 | 2.67E−07 |
| Neurology_II_GTPBP2 | 0.279 | 6.9E−08 | 2.72E−07 |
| Neurology_II_PHACTR2 | 0.407 | 7.2E−08 | 2.82E−07 |
| Inflammation_II_PSTPIP2 | 0.490 | 7.2E−08 | 2.82E−07 |
| Cardiometabolic_II_RAB10 | 0.205 | 8.1E−08 | 3.16E−07 |
| Inflammation_II_ERP29 | 0.448 | 8.5E−08 | 3.33E−07 |
| Neurology_II_GIPC2 | 0.130 | 9.0E−08 | 3.53E−07 |
| Cardiometabolic_II_PRKD2 | 0.158 | 1.0E−07 | 4.04E−07 |
| Oncology_TRIAP1 | 0.243 | 1.1E−07 | 4.14E−07 |
| Cardiometabolic_SERPINE1 | 0.270 | 1.1E−07 | 4.21E−07 |
| Cardiometabolic_TYMP | 0.230 | 1.1E−07 | 4.35E−07 |
| Oncology_II_PPP1CC | 0.221 | 1.1E−07 | 4.35E−07 |
| Cardiometabolic_II_IDO1 | 0.261 | 1.2E−07 | 4.48E−07 |
| Inflammation_GZMB | 0.330 | 1.3E−07 | 5.04E−07 |
| Neurology_AMFR | 0.244 | 1.4E−07 | 5.24E−07 |
| Cardiometabolic_II_ADGRF5 | 0.098 | 1.4E−07 | 5.44E−07 |
| Inflammation_II_IDO1 | 0.245 | 1.6E−07 | 6.02E−07 |
| Oncology_CXCL8 | 0.231 | 1.6E−07 | 6.18E−07 |
| Neurology_II_OTUD7B | 0.197 | 1.7E−07 | 6.45E−07 |
| Inflammation_ARHGEF12 | 0.361 | 1.7E−07 | 6.47E−07 |
| Oncology_STXBP3 | 0.260 | 1.7E−07 | 6.61E−07 |
| Oncology_ANGPT2 | −0.140 | 1.7E−07 | 6.63E−07 |
| Neurology_II_EIF4G3 | 0.274 | 1.9E−07 | 7.40E−07 |
| Neurology_CXCL8 | 0.229 | 1.9E−07 | 7.40E−07 |
| Cardiometabolic_II_CBX2 | 0.173 | 2.4E−07 | 9.33E−07 |
| Cardiometabolic_II_PMM2 | 0.261 | 2.5E−07 | 9.45E−07 |
| Oncology_II_UNC79 | 0.232 | 2.5E−07 | 9.71E−07 |
| Inflammation_BANK1 | 0.385 | 2.6E−07 | 9.88E−07 |
| Inflammation_II_GP5 | 0.165 | 2.8E−07 | 1.06E−06 |
| Neurology_II_PMS1 | 0.217 | 2.9E−07 | 1.09E−06 |
| Inflammation_CCN2 | 0.209 | 3.1E−07 | 1.19E−06 |
| Oncology_RABEPK | 0.248 | 3.3E−07 | 1.27E−06 |
| Inflammation_HGF | 0.149 | 3.4E−07 | 1.28E−06 |
| Cardiometabolic_II_HIP1 | 0.230 | 3.5E−07 | 1.31E−06 |
| Inflammation_CXCL8 | 0.217 | 3.6E−07 | 1.34E−06 |
| Oncology_KLK13 | −0.189 | 3.6E−07 | 1.37E−06 |
| Cardiometabolic_CD69 | 0.385 | 3.7E−07 | 1.39E−06 |
| Neurology_CXCL11 | 0.272 | 3.8E−07 | 1.41E−06 |
| Neurology_PTEN | 0.561 | 3.8E−07 | 1.42E−06 |
| Neurology_II_TXNDC9 | 0.176 | 3.9E−07 | 1.45E−06 |
| Oncology_ZBTB16 | 0.280 | 3.9E−07 | 1.45E−06 |
| Neurology_SLC27A4 | 0.334 | 3.9E−07 | 1.47E−06 |
| Inflammation_II_STX5 | 0.134 | 4.0E−07 | 1.48E−06 |
| Cardiometabolic_CLTA | 0.271 | 4.2E−07 | 1.56E−06 |
| Neurology_CETN2 | 0.538 | 4.4E−07 | 1.63E−06 |
| Oncology_II_SNX18 | 0.219 | 4.7E−07 | 1.75E−06 |
| Inflammation_CCL11 | 0.128 | 4.8E−07 | 1.78E−06 |
| Oncology_II_SAT2 | 0.208 | 5.3E−07 | 1.95E−06 |
| Inflammation_NCK2 | 0.307 | 5.3E−07 | 1.97E−06 |
| Oncology_ADAMTS15 | −0.195 | 5.8E−07 | 2.16E−06 |
| Inflammation_PDLIM7 | 0.442 | 6.0E−07 | 2.23E−06 |
| Oncology_II_TRDMT1 | 0.336 | 6.3E−07 | 2.32E−06 |
| Inflammation_II_PCBD1 | 0.162 | 6.5E−07 | 2.39E−06 |
| Neurology_II_EIF1AX | 0.242 | 6.7E−07 | 2.45E−06 |
| Cardiometabolic_DOK2 | 0.402 | 7.3E−07 | 2.70E−06 |
| Neurology_II_PDRG1 | 0.140 | 7.6E−07 | 2.79E−06 |
| Oncology_II_DYNC1H1 | 0.186 | 8.0E−07 | 2.94E−06 |
| Cardiometabolic_II_USP47 | 0.210 | 8.2E−07 | 3.02E−06 |
| Cardiometabolic_DEFA1_DEFA1B | 0.263 | 8.4E−07 | 3.07E−06 |
| Neurology_ATXN10 | 0.407 | 1.0E−06 | 3.76E−06 |
| Cardiometabolic_II_EDN1 | −0.121 | 1.2E−06 | 4.38E−06 |
| Cardiometabolic_II_COL2A1 | 0.263 | 1.2E−06 | 4.41E−06 |
| Oncology_II_TAB2 | 0.341 | 1.2E−06 | 4.49E−06 |
| Cardiometabolic_II_ADAMTSL4 | −0.101 | 1.3E−06 | 4.81E−06 |
| Neurology_SMARCA2 | 0.293 | 1.4E−06 | 4.92E−06 |
| Inflammation_II_ERMAP | 0.166 | 1.4E−06 | 4.96E−06 |
| Cardiometabolic_II_RAB33A | 0.224 | 1.4E−06 | 5.17E−06 |
| Cardiometabolic_II_TPK1 | 0.108 | 1.4E−06 | 5.19E−06 |
| Cardiometabolic_II_EHD3 | 0.419 | 1.6E−06 | 5.60E−06 |
| Inflammation_IL18 | 0.173 | 1.6E−06 | 5.83E−06 |
| Inflammation_II_PPBP | 0.300 | 1.6E−06 | 5.85E−06 |
| Cardiometabolic_II_RBM19 | 0.279 | 1.7E−06 | 6.05E−06 |
| Neurology_CLEC1B | 0.286 | 1.7E−06 | 6.29E−06 |
| Cardiometabolic_II_RAB27B | 0.425 | 1.8E−06 | 6.33E−06 |
| Cardiometabolic_II_ELOB | 0.144 | 1.8E−06 | 6.38E−06 |
| Oncology_II_KAZN | 0.362 | 1.8E−06 | 6.49E−06 |
| Oncology_BAIAP2 | 0.281 | 1.8E−06 | 6.58E−06 |
| Oncology_II_MCTS1 | 0.078 | 1.9E−06 | 6.92E−06 |
| Cardiometabolic_SORT1 | 0.116 | 1.9E−06 | 6.94E−06 |
| Oncology_LTA4H | 0.150 | 2.1E−06 | 7.59E−06 |
| Inflammation_YTHDF3 | 0.434 | 2.2E−06 | 7.85E−06 |
| Neurology_II_PPP1R14A | 0.254 | 2.3E−06 | 8.20E−06 |
| Inflammation_GBP2 | 0.383 | 2.3E−06 | 8.31E−06 |
| Oncology_II_DTNB | 0.168 | 2.5E−06 | 8.84E−06 |
| Cardiometabolic_HSPB1 | 0.304 | 2.6E−06 | 9.23E−06 |
| Chcology_II_DDX1 | 0.226 | 2.6E−06 | 9.36E−06 |
| Inflammation_II_DCTD | 0.322 | 2.7E−06 | 9.42E−06 |
| Neurology_II_NT5C1A | 0.136 | 2.9E−06 | 1.03E−05 |
| Oncology_ATP6V1D | 0.074 | 2.9E−06 | 1.04E−05 |
| Neurology_II_LEO1 | 0.116 | 3 2E−06 | 1.11E−05 |
| Neurology_ADAM8 | 0.119 | 3.2E−06 | 1.14E−05 |
| Cardiometabolic_II_IGHMBP2 | 0.240 | 3.9E−06 | 1.36E−05 |
| Inflammation_MGLL | 0.326 | 4.0E−06 | 1.40E−05 |
| Oncology_II_RAD51 | 0.161 | 4.4E−06 | 1.55E−05 |
| Inflammation_FGF2 | 0.157 | 4.6E−06 | 1.59E−05 |
| Oncology_RRM2 | 0.186 | 5.0E−06 | 1.75E−05 |
| Inflammation_II_GLRX5 | 0.248 | 5.1E−06 | 1.77E−05 |
| Oncology_TP53 | 0.319 | 5.2E−06 | 1.81E−05 |
| Inflammation_CCL7 | 0.204 | 5.5E−06 | 1.92E−05 |
| Neurology_II_OPHN1 | 0.295 | 5.6E−06 | 1.95E−05 |
| Oncology_NINJ1 | 0.197 | 5.6E−06 | 1.95E−05 |
| Oncology_II_CYTH3 | 0.199 | 5.8E−06 | 2.02E−05 |
| Inflammation_II_BABAM1 | 0.091 | 5.9E−06 | 2.05E−05 |
| Inflammation_CCL21 | −0.131 | 6.3E−06 | 2.17E−05 |
| Cardiometabolic_II_EHBP1 | 0.212 | 6.5E−06 | 2.25E−05 |
| Cardiometabolic_II_BDNF | 0.297 | 6.5E−06 | 2.26E−05 |
| Oncology_II_MTIF3 | 0.305 | 6.9E−06 | 2.40E−05 |
| Cardiometabolic_APLP1 | −0.182 | 7.2E−06 | 2.50E−05 |
| Neurology_TCL1A | 0.396 | 7.3E−06 | 2.51E−05 |
| Neurology_II_RTN4IP1 | 0.292 | 8.9E−06 | 3.06E−05 |
| Neurology_II_IFT20 | 0.154 | 9.2E−06 | 3.18E−05 |
| Oncology_SPARC | 0.252 | 9.5E−06 | 3.25E−05 |
| Inflammation_II_PPM1B | 0.122 | 1.0E−05 | 3.49E−05 |
| Oncology_HTRA2 | 0.187 | 1.0E−05 | 3.59E−05 |
| Cardiometabolic_II_EXOSC10 | 0.166 | 1.1E−05 | 3.60E−05 |
| Inflammation_TIMP3 | 0.376 | 1.2E−05 | 3.95E−05 |
| Cardiometabolic_CNST | 0.324 | 1.2E−05 | 4.03E−05 |
| Cardiometabolic_CTF1 | 0.342 | 1.2E−05 | 4.07E−05 |
| Inflammation_FXYD5 | 0.250 | 1.2E−05 | 4.25E−05 |
| Inflammation_ATP51F1 | 0.328 | 1.3E−05 | 4.45E−05 |
| Oncology_11_CD101 | 0.120 | 1.4E−05 | 4.62E−05 |
| Cardiometabolic_ITGB2 | 0.096 | 1.4E−05 | 4.66E−05 |
| Oncology_DTX3 | 0.115 | 1.4E−05 | 4.77E−05 |
| Oncology_MAGED1 | 0.203 | 1.5E−05 | 4.96E−05 |
| Oncology_II_SCRIB | 0.283 | 1.5E−05 | 5.20E−05 |
| Inflammation_IL4 | 0.627 | 1.6E−05 | 5.27E−05 |
| Cardiometabolic_HK2 | 0.373 | 1.6E−05 | 5.43E−05 |
| Inflammation_EDAR | 0.211 | 1.6E−05 | 5.48E−05 |
| Cardiometabolic_II_KIF1C | 0.128 | 1.7E−05 | 5.60E−05 |
| Cardiometabolic_II_TIMM10 | 0.121 | 1.7E−05 | 5.71E−05 |
| Inflammation_II_C1UTNF9 | −0.138 | 1.8E−05 | 5.89E−05 |
| Inflammation_CXCL6 | 0.239 | 1.8E−05 | 6.04E−05 |
| Oncology_RUVBL1 | 0.322 | 1.9E−05 | 6.29E−05 |
| Inflammation_II_TSC1 | 0.130 | 1.9E−05 | 6.34E−05 |
| Inflammation_II_ANKMY2 | 0.227 | 1.9E−05 | 6.44E−05 |
| Neurology_II_PGM2 | 0.188 | 1.9E−05 | 6.47E−05 |
| Inflammation_JUN | 0.206 | 2.1E−05 | 6.85E−05 |
| Inflammation_II_NFAT5 | 0.260 | 2.1E−05 | 6.92E−05 |
| Oncology_SH2B3 | 0.367 | 2.1E−05 | 7.14E−05 |
| Neurology_II_BATF | 0.232 | 2.3E−05 | 7.77E−05 |
| Inflammation_II_NRGN | 0.247 | 2.4E−05 | 7.86E−05 |
| Neurology_II_CACNB3 | 0.246 | 2.4E−05 | 7.86E−05 |
| Neurology_II_LYSMD3 | 0.219 | 2.4E−05 | 7.89E−05 |
| Oncology_II_TADA3 | 0.158 | 2.4E−05 | 7.98E−05 |
| Oncology_II_PDIA5 | 0.131 | 2.5E−05 | 8.29E−05 |
| Inflammation_II_C3 | 0.123 | 2.5E−05 | 8.30E−05 |
| Oncology_II_RAB2B | 0.203 | 2.8E−05 | 9.13E−05 |
| Oncology_II_CEP290 | 0.176 | 2.9E−05 | 9.64E−05 |
| Inflammation_CASP2 | 0.176 | 3.2E−05 | 0.0001 |
| Inflammation_DECR1 | 0.278 | 3.2E−05 | 0.0001 |
| Oncology_II_ZNRD2 | 0.226 | 3.4E−05 | 0.0001 |
| Cardiometabolic_GZMH | 0.313 | 3.5E−05 | 0.0001 |
| Cardiometabolic_II_ITPR1 | 0.186 | 3.6E−05 | 0.0001 |
| Inflammation_PTX3 | 0.144 | 3.6E−05 | 0.0001 |
| Cardiometabolic_PLXNB3 | 0.113 | 3.8E−05 | 0.0001 |
| Oncology_FMR1 | 0.445 | 4.1E−05 | 0.0001 |
| Cardiometabolic_II_EIF2AK3 | 0.215 | 4.1E−05 | 0.0001 |
| Oncology_II_EFCAB2 | 0.112 | 4.1E−05 | 0.0001 |
| Neurology_II_STXBP1 | 0.231 | 4.2E−05 | 0.0001 |
| Cardiometabolic_II_SARG | 0.264 | 4.3E−05 | 0.0001 |
| Oncology_GALNT2 | 0.081 | 4.4E−05 | 0.0001 |
| Cardiometabolic_II_HPSE | 0.279 | 4.6E−05 | 0.0001 |
| Inflammation_II_APPL2 | 0.377 | 4.6E−05 | 0.0002 |
| Neurology_FUT8 | 0.153 | 4.9E−05 | 0.0002 |
| Cardiometabolic_GAS6 | −0.068 | 5.2E−05 | 0.0002 |
| Neurology_CD164 | 0.073 | 5.2E−05 | 0.0002 |
| Inflammation_MVK | 0.287 | 5.3E−05 | 0.0002 |
| Oncology_II_IFI30 | 0.140 | 5.7E−05 | 0.0002 |
| Cardiometabolic_DCTPP1 | 0.101 | 5.7E−05 | 0.0002 |
| Oncology_II_NFU1 | 0.293 | 5.8E−05 | 0.0002 |
| Neurology_II_LDLRAP1 | 0.222 | 6.0E−05 | 0.0002 |
| Inflammation_F2R | 0.140 | 6.2E−05 | 0.0002 |
| Neurology_CTSS | 0.050 | 6.3E−05 | 0.0002 |
| Oncology_II_ARAF | 0.180 | 6.6E−05 | 0.0002 |
| Inflammation_II_ASGR2 | −0.090 | 7.0E−05 | 0.0002 |
| Neurology_OGN | −0.133 | 7.1E−05 | 0.0002 |
| Cardiometabolic_LPL | −0.135 | 7.2E−05 | 0.0002 |
| Cardiometabolic_CD55 | 0.076 | 7.5E−05 | 0.0002 |
| Inflammation_PADI2 | 0.292 | 7.7E−05 | 0.0002 |
| Oncology_II_MTSS2 | 0.266 | 7.8E−05 | 0.0002 |
| Neurology_WFIKKN1 | 0.131 | 8.3E−05 | 0.0003 |
| Neurology_II_SCRIB | 0.243 | 8.6E−05 | 0.0003 |
| Cardiometabolic_II_SCRIB | 0.256 | 8.7E−05 | 0.0003 |
| Inflammation_METAP1D | 0.241 | 8.7E−05 | 0.0003 |
| Inflammation_II_PF4 | 0.265 | 9.0E−05 | 0.0003 |
| Inflammation_WAS | 0.296 | 9.4E−05 | 0.0003 |
| Oncology_II_SSH3 | 0.067 | 9.8E−05 | 0.0003 |
| Inflammation_SPINT2 | 0.111 | 9.9E−05 | 0.0003 |
| Inflammation_II_SCRIB | 0.251 | 0.0001 | 0.0003 |
| Neurology_LAYN | −0.114 | 0.0001 | 0.0003 |
| Oncology_ERP44 | 0.066 | 0.0001 | 0.0003 |
| Oncology_II_ACOT13 | 0.345 | 0.0001 | 0.0003 |
| Oncology_II_BTLA | 0.234 | 0.0001 | 0.0004 |
| Inflammation_C1QA | −0.062 | 0.0001 | 0.0004 |
| Cardiometabolic_GP1BA | 0.090 | 0.0001 | 0.0004 |
| Inflammation_ACTN4 | 0.096 | 0.0001 | 0.0004 |
| Inflammation_CD276 | −0.091 | 0.0001 | 0.0004 |
| Cardiometabolic_II_CSDE1 | 0.295 | 0.0001 | 0.0004 |
| Neurology_II_RGCC | 0.174 | 0.0001 | 0.0004 |
| Inflammation_II_ITGAL | 0.220 | 0.0001 | 0.0004 |
| Cardiometabolic_EFEMP1 | −0.092 | 0.0001 | 0.0004 |
| Inflammation_PROK1 | 0.195 | 0.0001 | 0.0004 |
| Neurology_II_CAMLG | 0.160 | 0.0001 | 0.0004 |
| Inflammation_II_S100A13 | 0.089 | 0.0002 | 0.0005 |
| Inflammation_LGALS9 | 0.088 | 0.0002 | 0.0005 |
| Oncology_GFER | 0.176 | 0.0002 | 0.0005 |
| Oncology_II_SNX2 | 0.188 | 0.0002 | 0.0005 |
| Inflammation_CLIP2 | 0.339 | 0.0002 | 0.0005 |
| Neurology_GGA1 | 0.172 | 0.0002 | 0.0005 |
| Inflammation_MANF | 0.377 | 0.0002 | 0.0005 |
| Inflammation_CD84 | 0.083 | 0.0002 | 0.0005 |
| Oncology_II_CEP350 | 0.144 | 0.0002 | 0.0006 |
| Inflammation_II_EPHA4 | −0.091 | 0.0002 | 0.0006 |
| Cardiometabolic_PGLYRP1 | 0.119 | 0.0002 | 0.0006 |
| Inflammation_II_RNF168 | 0.086 | 0.0002 | 0.0006 |
| Inflammation_II_HIF1A | 0.161 | 0.0002 | 0.0006 |
| Cardiometabolic_VSIR | 0.147 | 0.0002 | 0.0006 |
| Oncology_TBC1D23 | 0.233 | 0.0002 | 0.0006 |
| Neurology_II_SLA2 | 0.227 | 0.0002 | 0.0006 |
| Oncology_II_GIPC3 | 0.315 | 0.0002 | 0.0007 |
| Cardiometabolic_II_KIF22 | 0.317 | 0.0002 | 0.0007 |
| Inflammation_II_LATS1 | 0.206 | 0.0002 | 0.0007 |
| Inflammation_II_CD226 | 0.115 | 0.0002 | 0.0007 |
| Neurology_CGA | −0.230 | 0.0003 | 0.0008 |
| Oncology_EPHA2 | −0.085 | 0.0003 | 0.0008 |
| Neurology_HNMT | 0.141 | 0.0003 | 0.0008 |
| Inflammation_REG4 | −0.125 | 0.0003 | 0.0008 |
| Cardiometabolic_PPIB | 0.228 | 0.0003 | 0.0008 |
| Oncology_II_VSIG2 | −0.184 | 0.0003 | 0.0009 |
| Cardiometabolic_CA13 | 0.315 | 0.0003 | 0.0009 |
| Oncology_II_FOS | 0.109 | 0.0003 | 0.0009 |
| Inflammation_II_NXPH3 | −0.085 | 0.0003 | 0.0009 |
| Inflammation_LAP3 | 0.248 | 0.0003 | 0.0009 |
| Oncology_BTC | 0.228 | 0.0003 | 0.0009 |
| Neurology_II_MTHFD2 | 0.061 | 0.0003 | 0.0010 |
| Neurology_II_MICALL2 | 0.178 | 0.0003 | 0.0010 |
| Oncology_NCS1 | 0.082 | 0.0003 | 0.0010 |
| Inflammation_II_BMPER | −0.060 | 0.0003 | 0.0010 |
| Inflammation_SPINK4 | −0.170 | 0.0003 | 0.0010 |
| Inflammation_LAMA4 | 0.077 | 0.0003 | 0.0010 |
| Inflammation_II_MOCS2 | 0.130 | 0.0004 | 0.0011 |
| Oncology_II_GPD1 | 0.124 | 0.0004 | 0.0011 |
| Cardiometabolic_II_GUK1 | 0.102 | 0.0004 | 0.0011 |
| Cardiometabolic_SELP | 0.114 | 0.0004 | 0.0011 |
| Cardiometabolic_II_ATP6V1G2 | 0.158 | 0.0004 | 0.0011 |
| Oncology_II_CDC42BPB | 0.246 | 0.0004 | 0.0012 |
| Neurology_II_CRYM | 0.142 | 0.0004 | 0.0012 |
| Cardiometabolic_II_RAB39B | 0.184 | 0.0004 | 0.0012 |
| Inflammation_II_A1BG | −0.045 | 0.0004 | 0.0012 |
| Inflammation_BSG | −0.050 | 0.0004 | 0.0013 |
| Cardiometabolic_II_COCH | 0.100 | 0.0005 | 0.0013 |
| Cardiometabolic_II_BNIP2 | 0.102 | 0.0005 | 0.0013 |
| Cardiometabolic_ITGB1BP2 | 0.333 | 0.0005 | 0.0014 |
| Neurology_II_TTF2 | 0.164 | 0.0005 | 0.0014 |
| Neurology_II_CDK5RAP3 | 0.127 | 0.0005 | 0.0014 |
| Oncology_SRC | 0.301 | 0.0005 | 0.0014 |
| Inflammation_CXCL1 | 0.190 | 0.0005 | 0.0014 |
| Inflammation_II_CD36 | 0.103 | 0.0005 | 0.0014 |
| Neurology_CLEC10A | −0.080 | 0.0005 | 0.0015 |
| Cardiometabolic_LCN2 | 0.115 | 0.0005 | 0.0015 |
| Neurology_II_FGFBP3 | 0.097 | 0.0005 | 0.0015 |
| Oncology_LRP1 | −0.084 | 0.0005 | 0.0016 |
| Inflammation_II_CD7 | 0.122 | 0.0005 | 0.0016 |
| Inflammation_IL15 | −0.074 | 0.0005 | 0.0016 |
| Neurology_MESD | 0.278 | 0.0006 | 0.0017 |
| Inflammation_TNFSF13 | 0.073 | 0.0006 | 0.0017 |
| Neurology_PSG1 | −0.284 | 0.0006 | 0.0017 |
| Inflammation_LGMN | 0.084 | 0.0006 | 0.0018 |
| Neurology_CLPP | 0.234 | 0.0006 | 0.0018 |
| Neurology_ISLR2 | −0.096 | 0.0006 | 0.0018 |
| Inflammation_II_AKAP12 | −0.057 | 0.0006 | 0.0019 |
| Neurology_ACVRL1 | −0.066 | 0.0007 | 0.0019 |
| Oncology_SIAE | 0.091 | 0.0007 | 0.0019 |
| Oncology_AIFM1 | 0.259 | 0.0007 | 0.0021 |
| Oncology_DCBLD2 | −0.089 | 0.0008 | 0.0022 |
| Neurology_II_PLSCR3 | 0.109 | 0.0008 | 0.0022 |
| Inflammation_TFF2 | −0.161 | 0.0008 | 0.0023 |
| Inflammation_LGALS4 | −0.128 | 0.0008 | 0.0023 |
| Cardiometabolic_II_RAB11FIP3 | 0.284 | 0.0008 | 0.0024 |
| Inflammation_II_CLEC12A | 0.060 | 0.0008 | 0.0024 |
| Cardiometabolic_COL1A1 | −0.111 | 0.0008 | 0.0024 |
| Cardiometabolic_GH1 | −0.406 | 0.0009 | 0.0025 |
| Cardiometabolic_II_CMC1 | 0.180 | 0.0009 | 0.0026 |
| Cardiometabolic_TFRC | 0.090 | 0.0009 | 0.0027 |
| Inflammation_CCL17 | 0.204 | 0.0009 | 0.0027 |
| Neurology_SLC16A1 | 0.162 | 0.0010 | 0.0028 |
| Oncology_ITGB1BP1 | 0.222 | 0.0010 | 0.0028 |
| Neurology_PRTFDC1 | 0.389 | 0.0010 | 0.0029 |
| Neurology_PLA2G7 | 0.073 | 0.0010 | 0.0029 |
| Inflammation_II_FGL1 | −0.149 | 0.0010 | 0.0029 |
| Oncology_II_PAFAH2 | 0.105 | 0.0010 | 0.0030 |
| Inflammation_II_CTSE | 0.112 | 0.0011 | 0.0031 |
| Cardiometabolic_THPO | 0.098 | 0.0011 | 0.0031 |
| Oncology_CD5 | 0.103 | 0.0011 | 0.0031 |
| Cardiometabolic_CLEC5A | 0.091 | 0.0011 | 0.0031 |
| Oncology_MSLN | −0.158 | 0.0011 | 0.0032 |
| Oncology_II_SLMAP | 0.209 | 0.0011 | 0.0032 |
| Neurology_II_TEX101 | 0.168 | 0.0011 | 0.0032 |
| Inflammation_II_CCNE1 | 0.103 | 0.0012 | 0.0033 |
| Cardiometabolic_NPPB | −0.397 | 0.0012 | 0.0033 |
| Cardiometabolic_SCARF1 | 0.094 | 0.0012 | 0.0034 |
| Neurology_CLEC14A | −0.072 | 0.0013 | 0.0036 |
| Neurology_KIRREL2 | −0.077 | 0.0013 | 0.0037 |
| Oncology_GFRA1 | −0.070 | 0.0013 | 0.0037 |
| Cardiometabolic_II_SGSH | 0.141 | 0.0014 | 0.0039 |
| Cardiometabolic_CGREF1 | −0.087 | 0.0014 | 0.0039 |
| Inflammation_LIFR | −0.052 | 0.0014 | 0.0040 |
| Cardiometabolic_II_DMP1 | −0.119 | 0.0014 | 0.0040 |
| Cardiometabolic_II_HADH | −0.126 | 0.0015 | 0.0041 |
| Inflammation_II_APOA2 | −0.116 | 0.0015 | 0.0041 |
| Cardiometabolic_ST6GAL1 | 0.068 | 0.0015 | 0.0042 |
| Neurology_II_CABP2 | 0.149 | 0.0015 | 0.0042 |
| Inflammation_II_NHLRC3 | −0.073 | 0.0015 | 0.0043 |
| Inflammation_II_MXRA8 | −0.076 | 0.0016 | 0.0045 |
| Oncology_II_VCPKMT | 0.165 | 0.0016 | 0.0046 |
| Oncology_CCL8 | 0.135 | 0.0017 | 0.0046 |
| Oncology_PVALB | 0.213 | 0.0017 | 0.0046 |
| Neurology_RHOC | 0.257 | 0.0017 | 0.0048 |
| Neurology_TNFRSF10A | −0.073 | 0.0018 | 0.0048 |
| Oncology_CEACAM3 | 0.164 | 0.0018 | 0.0049 |
| Cardiometabolic_II_KLK3 | 0.381 | 0.0018 | 0.0049 |
| Oncology_CNPY4 | 0.136 | 0.0018 | 0.0050 |
| Cardiometabolic_BMP6 | 0.086 | 0.0019 | 0.0052 |
| Inflammation_DAG1 | 0.087 | 0.0019 | 0.0053 |
| Inflammation_TNFSF12 | 0.059 | 0.0019 | 0.0053 |
| Oncology_SCG2 | −0.082 | 0.0020 | 0.0054 |
| Oncology_II_SUSD4 | −0.100 | 0.0020 | 0.0054 |
| Cardiometabolic_WASF1 | 0.175 | 0.0020 | 0.0054 |
| Cardiometabolic_II_BCAT1 | 0.076 | 0.0020 | 0.0055 |
| Inflammation_II_ACE | −0.067 | 0.0020 | 0.0055 |
| Cardiometabolic_II_BGLAP | −0.194 | 0.0020 | 0.0055 |
| Cardiometabolic_CD93 | −0.064 | 0.0021 | 0.0056 |
| Cardiometabolic_REG1A | −0.124 | 0.0021 | 0.0057 |
| Oncology_VNN2 | 0.099 | 0.0021 | 0.0057 |
| Oncology_II_RGL2 | 0.172 | 0.0021 | 0.0057 |
| Oncology_CDKN1A | 0.204 | 0.0022 | 0.0059 |
| Cardiometabolic_TFPI | 0.059 | 0.0022 | 0.0059 |
| Inflammation_TNFSF10 | −0.052 | 0.0022 | 0.0060 |
| Inflammation_CLEC4D | 0.144 | 0.0024 | 0.0064 |
| Neurology_DSG2 | −0.055 | 0.0024 | 0.0065 |
| Oncology_II_ACRBP | 0.055 | 0.0025 | 0.0067 |
| Inflammation_II_INSR | −0.035 | 0.0025 | 0.0067 |
| Oncology_SCLY | 0.099 | 0.0025 | 0.0068 |
| Neurology_II_INSL3 | 0.390 | 0.0025 | 0.0068 |
| Inflammation_II_SCGB3A1 | −0.075 | 0.0026 | 0.0069 |
| Cardiometabolic_LGALS1 | 0.083 | 0.0026 | 0.0069 |
| Neurology_TNFRSF9 | −0.090 | 0.0026 | 0.0070 |
| Inflammation_II_PENK | −0.077 | 0.0026 | 0.0070 |
| Oncology_DAB2 | 0.193 | 0.0026 | 0.0071 |
| Neurology_SEMA4D | 0.068 | 0.0026 | 0.0071 |
| Inflammation_CCL25 | −0.097 | 0.0027 | 0.0072 |
| Inflammation_II_ACRV1 | 0.242 | 0.0027 | 0.0073 |
| Cardiometabolic_II_MECR | 0.196 | 0.0028 | 0.0074 |
| Oncology_II_CENPJ | 0.151 | 0.0028 | 0.0075 |
| Inflammation_II_PRSS22 | −0.075 | 0.0029 | 0.0077 |
| Cardiometabolic_II_SYTL4 | 0.150 | 0.0029 | 0.0077 |
| Oncology_II_MINDY1 | 0.257 | 0.0029 | 0.0078 |
| Inflammation_CD79B | −0.093 | 0.0030 | 0.0079 |
| Oncology_II_GATA3 | 0.094 | 0.0030 | 0.0081 |
| Inflammation_II_TCN1 | 0.074 | 0.0030 | 0.0081 |
| Neurology_AGR2 | −0.219 | 0.0033 | 0.0087 |
| Oncology_II_CDK1 | 0.116 | 0.0033 | 0.0087 |
| Oncology_II_PAIP2B | 0.095 | 0.0033 | 0.0088 |
| Oncology_COX5B | 0.156 | 0.0033 | 0.0088 |
| Inflammation_BCL2L11 | 0.079 | 0.0035 | 0.0092 |
| Oncology_CLEC6A | 0.109 | 0.0035 | 0.0093 |
| Inflammation_II_RNASE1 | −0.075 | 0.0035 | 0.0094 |

TABLE 12
UK Biobank demographics for lung cancer cases and selected cancer-free controls

| | Cancer | | Controls | | Overall | | P value (test)* |
|---|---|---|---|---|---|---|---|
| Sex n (%) | | | | | | | X2 1.3 |
| Female | 188 | (48.0) | 2826 | (51.4) | 3014 | (51.2) | 0.25 |
| Male | 204 | (52.0) | 2674 | (48.6) | 2878 | (48.8) | (CS) |
| Age (years) | | | | | | | |
| Mean (SD) | 62.2 | (6.09) | 57.6 | (7.80) | 57.9 | (7.78) | <0.00001 |
| Median [IQR] | 64.0 | [59-67] | 58 | [52-64] | 59 | [52-65] | (MW) |
| Smoking Status n (%) | | | | | | | |
| Never | 33 | (8.4) | 1621 | (29.5) | 1654 | (28.1) | X2 76.1 |
| Current or Former | 356 | (90.8) | 3879 | (70.5) | 4235 | (71.9) | <0.00001 |
| Missing | 3 | (0.8) | 0 | (0) | 3 | (0.1) | (CS) |
| Smoking pack years* | | | | | | | |
| Mean (SD) | 38.9 | (25.7) | 22.3 | (17.9) | 24.3 | (19.8) | <0.00001 |
| Median [IQR] | 34.5 | [21.0-48.6] | 18.0 | [9.4-30.5] | 19.5 | [10, 64] | (MW) |

| | | | |
|---|---|---|---|
| Total | 392 | 5500 | 5892 |

*Pack-year data only given for known non-zero values

TABLE 13
Plasma proteins differentially expressed in 1-3 Y and
1-5 Y samples, with direction of change P value and FDR

| | Gene | Up or | | 1-5 Y | 1-5 Y | 1-5 Y | 1-3 Y | 1-3 Y | 1-3 Y |
|---|---|---|---|---|---|---|---|---|---|
| UniProt | Name | Down | Cohort | Estimate | P Value | FDR | Estimate | P Value | FDR |
| P01350 | GAST | Down | 1-3 Y only | | | | −0.807 | 0.0014 | 0.879 |
| Q13822 | ENPP2 | Down | 1-3 Y only | | | | −0.131 | 0.003 | 0.933 |
| Q9H461 | FZD8 | Down | 1-3 Y only | | | | −0.207 | 0.009 | 0.933 |
| Q9GZV9 | FGF23 | Down | 1-3 Y only | | | | −0.422 | 0.010 | 0.933 |
| P04155 | TFF1 | Down | 1-3 Y only | | | | −0.389 | 0.026 | 0.933 |
| P10636 | MAPT | Down | 1-3 Y only | | | | −1.048 | 0.037 | 0.933 |
| O43320 | FGF16 | Down | 1-3 Y only | | | | −0.264 | 0.038 | 0.933 |
| P01178 | OXT | Down | 1-3 Y only | | | | −0.561 | 0.040 | 0.933 |
| O95696 | BRD1 | Down | 1-3 Y only | | | | −0.181 | 0.042 | 0.933 |
| P55083 | MFAP4 | Down | 1-3 Y only | | | | −0.145 | 0.042 | 0.933 |
| O14904 | WNT9A | Down | 1-3 Y only | | | | −0.129 | 0.049 | 0.933 |
| O43155 | FLRT2 | Down | 1-3 Y only | | | | −0.104 | 0.049 | 0.933 |
| Q9NQ79 | CRTAC1 | Down | 1-3 Y only | | | | −0.105 | 0.053 | 0.933 |
| Q13219 | PAPPA | Down | 1-3 Y only | | | | −0.252 | 0.053 | 0.933 |
| P01189 | POMC | Down | 1-3 Y only | | | | −0.310 | 0.063 | 0.933 |
| P01138 | NGF | Down | 1-3 Y only | | | | −0.040 | 0.065 | 0.933 |
| Q9BXS1 | IDI2 | Down | 1-3 Y only | | | | −0.235 | 0.065 | 0.933 |
| P13693 | TPT1 | Down | 1-3 Y only | | | | −0.468 | 0.066 | 0.933 |
| Q5JZY3 | EPHA10 | Down | 1-3 Y only | | | | −0.338 | 0.068 | 0.933 |
| P55082 | MFAP3 | Down | 1-3 Y only | | | | −0.218 | 0.072 | 0.933 |
| Q2M3V2 | SOWAHA | Down | 1-3 Y only | | | | −0.141 | 0.074 | 0.933 |
| P49788 | RARRES1 | Down | 1-3 Y only | | | | −0.112 | 0.082 | 0.933 |
| P51452 | DUSP3 | Down | 1-3 Y only | | | | −0.563 | 0.091 | 0.933 |
| Q13275 | SEMA3F | Down | 1-3 Y only | | | | −0.094 | 0.095 | 0.933 |
| Q9P232 | CNTN3 | Down | 1-3 Y only | | | | −0.109 | 0.102 | 0.933 |
| P08519 | LPA | Down | 1-3 Y only | | | | −0.484 | 0.110 | 0.933 |
| Q9UBX7 | KLK11 | Down | 1-3 Y only | | | | −0.097 | 0.111 | 0.933 |
| Q92834 | RPGR | Down | 1-3 Y only | | | | −0.163 | 0.112 | 0.933 |
| P01588 | EPO | Down | 1-3 Y only | | | | −0.295 | 0.114 | 0.933 |
| P13385 | TDGF1 | Down | 1-3 Y only | | | | −0.570 | 0.114 | 0.933 |
| Q16552 | IL17A | Down | 1-3 Y only | | | | −0.323 | 0.115 | 0.933 |
| O95971 | CD160 | Down | 1-3 Y only | | | | −0.154 | 0.121 | 0.933 |
| Q92973 | TNPO1 | Down | 1-3 Y only | | | | −0.117 | 0.125 | 0.933 |
| Q14353 | GAMT | Down | 1-3 Y only | | | | −0.108 | 0.275 | 0.933 |
| O60635 | TSPAN1 | Down | 1-5 Y only | −0.206 | 0.088 | 0.933 | | | |
| P10747 | CD28 | Down | 1-5 Y only | −0.141 | 0.109 | 0.933 | | | |
| Q9NY72 | SCN3B | Down | 1-5 Y only | −0.169 | 0.110 | 0.933 | | | |
| O60242 | ADGRB3 | Down | 1-5 Y only | −0.093 | 0.125 | 0.933 | | | |
| P24592 | IGFBP6 | Down | 1-5 Y only | −0.092 | 0.130 | 0.933 | | | |
| Q99748 | NRTN | Down | 1-5 Y only | −0.213 | 0.133 | 0.933 | | | |
| Q9BQI0 | AIF1L | Down | 1-5 Y only | −0.153 | 0.136 | 0.933 | | | |
| O14558 | HSPB6 | Down | 1-5 Y only | −0.132 | 0.137 | 0.933 | | | |
| P02144 | MB | Down | 1-5 Y only | −0.156 | 0.141 | 0.933 | | | |
| Q9NS68 | TNFRSF19 | Down | 1-5 Y only | −0.114 | 0.144 | 0.933 | | | |
| Q01344 | IL5RA | Down | 1-5 Y only | −0.173 | 0.144 | 0.933 | | | |
| Q92752 | TNR | Down | 1-5 Y only | −0.116 | 0.146 | 0.933 | | | |
| Q49AH0 | CDNF | Down | 1-5 Y only | −0.110 | 0.148 | 0.933 | | | |
| P01037 | CST1 | Down | 1-5 Y only | −0.275 | 0.148 | 0.933 | | | |
| Q9BYJ0 | FGFBP2 | Down | 1-5 Y only | −0.156 | 0.150 | 0.933 | | | |
| Q96FQ6 | S100A16 | Down | 1-5 Y only | −0.170 | 0.152 | 0.933 | | | |
| Q9HCU0 | CD248 | Down | 1-5 Y only | −0.138 | 0.157 | 0.933 | | | |
| O60609 | GFRA3 | Down | 1-5 Y only | −0.088 | 0.157 | 0.933 | | | |
| P29536 | LMOD1 | Down | 1-5 Y only | −0.127 | 0.158 | 0.933 | | | |
| Q8WVV4 | POF1B | Down | 1-5 Y only | −0.123 | 0.159 | 0.933 | | | |
| P78524 | DENND2B | Down | 1-5 Y only | −0.166 | 0.160 | 0.933 | | | |
| P49747 | COMP | Down | 1-5 Y only | −0.093 | 0.165 | 0.933 | | | |
| Q02246 | CNTN2 | Down | 1-5 Y only | −0.130 | 0.165 | 0.933 | | | |
| Q6ZMJ2 | SCARA5 | Down | 1-5 Y only | −0.094 | 0.165 | 0.933 | | | |
| Q6UVK1 | CSPG4 | Down | 1-5 Y only | −0.092 | 0.169 | 0.933 | | | |
| P06756 | ITGAV | Down | 1-5 Y only | −0.049 | 0.171 | 0.933 | | | |
| Q9BQB4 | SOST | Down | 1-5 Y only | −0.103 | 0.176 | 0.933 | | | |
| P29622 | SERPINA4 | Down | 1-5 Y only | −0.071 | 0.177 | 0.933 | | | |
| P59901 | LILRA4 | Down | 1-5 Y only | −0.153 | 0.178 | 0.933 | | | |
| Q9NQ38 | SPINK5 | Down | 1-5 Y only | −0.080 | 0.182 | 0.933 | | | |
| A6NC86 | PINLYP | Down | 1-5 Y only | −0.141 | 0.182 | 0.933 | | | |
| P35609 | ACTN2 | Down | 1-5 Y only | −0.186 | 0.182 | 0.933 | | | |
| P57087 | JAM2 | Down | 1-5 Y only | −0.070 | 0.184 | 0.933 | | | |
| Q12884 | FAP | Down | 1-5 Y only | −0.067 | 0.186 | 0.933 | | | |
| Q9NZQ9 | TMOD4 | Down | 1-5 Y only | −0.134 | 0.187 | 0.933 | | | |
| Q02747 | GUCA2A | Down | 1-5 Y only | −0.099 | 0.188 | 0.933 | | | |
| O75121 | MFAP3L | Down | 1-5 Y only | −0.124 | 0.195 | 0.933 | | | |
| Q9UBT3 | DKK4 | Down | 1-5 Y only | −0.115 | 0.197 | 0.933 | | | |
| P25391 | LAMA1 | Down | 1-5 Y only | −0.143 | 0.197 | 0.933 | | | |
| O95817 | BAG3 | Down | 1-5 Y only | −0.094 | 0.198 | 0.933 | | | |
| O76070 | SNCG | Down | 1-5 Y only | −0.200 | 0.202 | 0.933 | | | |
| Q9UH03 | SEPTIN3 | Down | 1-5 Y only | −0.169 | 0.203 | 0.933 | | | |
| Q2TAL6 | VWC2 | Down | 1-5 Y only | −0.100 | 0.208 | 0.933 | | | |
| P26715 | KLRC1 | Down | 1-5 Y only | −0.147 | 0.210 | 0.933 | | | |
| Q6UW56 | ATRAID | Down | 1-5 Y only | −0.070 | 0.217 | 0.933 | | | |
| Q13508 | ART3 | Down | 1-5 Y only | −0.082 | 0.224 | 0.933 | | | |
| Q9H156 | SLITRK2 | Down | 1-5 Y only | −0.092 | 0.240 | 0.933 | | | |
| O43699 | SIGLEC6 | Down | 1-5 Y only | −0.068 | 0.245 | 0.933 | | | |
| Q7Z7H5 | TMED4 | Down | 1-5 Y only | −0.118 | 0.268 | 0.933 | | | |
| Q9NQ25 | SLAMF7 | Down | 1-5 Y only | −0.128 | 0.283 | 0.933 | | | |
| P12532 | CKMT1A | Down | 1-5 Y only | −0.131 | 0.283 | 0.933 | | | |
| Q9H3T2 | SEMA6C | Down | 1-5 Y only | −0.049 | 0.298 | 0.933 | | | |
| P06729 | CD2 | Down | 1-5 Y only | −0.071 | 0.299 | 0.933 | | | |
| P28325 | CST5 | Down | 1-5 Y only | −0.108 | 0.300 | 0.933 | | | |
| Q96AQ6 | PBXIP1 | Down | 1-5 Y only | −0.051 | 0.311 | 0.933 | | | |
| O14960 | LECT2 | Down | 1-5 Y only | −0.163 | 0.317 | 0.933 | | | |
| P10082 | PYY | Down | 1-5 Y only | −0.164 | 0.323 | 0.933 | | | |
| O00468 | AGRN | Down | 1-5 Y only | −0.070 | 0.327 | 0.933 | | | |
| Q9Y5Q6 | INSL5 | Down | 1-5 Y only | −0.173 | 0.334 | 0.933 | | | |
| P28907 | CD38 | Down | 1-5 Y only | −0.065 | 0.344 | 0.933 | | | |
| Q6UXB8 | PI16 | Down | 1-5 Y only | −0.047 | 0.351 | 0.933 | | | |
| O76076 | CCN5 | Down | 1-5 Y only | −0.065 | 0.368 | 0.933 | | | |
| Q02223 | TNFRSF17 | Down | 1-5 Y only | −0.077 | 0.379 | 0.933 | | | |
| Q9HBG7 | LY9 | Down | 1-5 Y only | −0.049 | 0.394 | 0.933 | | | |
| P35052 | GPC1 | Down | 1-5 Y only | −0.050 | 0.396 | 0.933 | | | |
| Q9H6B4 | CLMP | Down | 1-5 Y only | −0.034 | 0.408 | 0.933 | | | |
| Q16820 | MEP1B | Down | 1-5 Y only | −0.202 | 0.410 | 0.933 | | | |
| O00622 | CCN1 | Down | 1-5 Y only | −0.159 | 0.413 | 0.933 | | | |
| O60245 | PCDH7 | Down | 1-5 Y only | −0.049 | 0.416 | 0.933 | | | |
| Q14515 | SPARCL1 | Down | 1-5 Y only | −0.042 | 0.419 | 0.933 | | | |
| Q9UBG3 | CRNN | Down | 1-5 Y only | −0.113 | 0.420 | 0.933 | | | |
| Q6GTS8 | PM20D1 | Down | 1-5 Y only | −0.340 | 0.428 | 0.933 | | | |
| Q9NP84 | TNFRSF12A | Down | 1-5 Y only | −0.060 | 0.436 | 0.933 | | | |
| O60469 | DSCAM | Down | 1-5 Y only | −0.060 | 0.464 | 0.933 | | | |
| O75781 | PALM | Down | 1-5 Y only | −0.048 | 0.483 | 0.933 | | | |
| P78423 | CX3CL1 | Down | 1-5 Y only | −0.046 | 0.484 | 0.933 | | | |
| Q16819 | MEP1A | Down | 1-5 Y only | −0.084 | 0.487 | 0.933 | | | |
| P55000 | SLURP1 | Down | 1-5 Y only | −0.049 | 0.524 | 0.933 | | | |
| P06727 | APOA4 | Down | 1-5 Y only | −0.052 | 0.526 | 0.933 | | | |
| Q6ZMM2 | ADAMTSL5 | Down | 1-5 Y only | −0.058 | 0.535 | 0.933 | | | |
| Q9NQ76 | MEPE | Down | 1-5 Y only | −0.031 | 0.536 | 0.933 | | | |
| Q9HC57 | WFDC1 | Down | 1-5 Y only | −0.036 | 0.550 | 0.935 | | | |
| P46783 | RPS10 | Down | 1-5 Y only | −0.041 | 0.551 | 0.935 | | | |
| Q08708 | CD300C | Down | 1-5 Y only | −0.041 | 0.561 | 0.936 | | | |
| P57078 | RIPK4 | Down | 1-5 Y only | −0.098 | 0.581 | 0.937 | | | |
| P10092 | CALCB | Down | 1-5 Y only | −0.038 | 0.583 | 0.937 | | | |
| Q9BSG5 | RTBDN | Down | 1-5 Y only | −0.033 | 0.624 | 0.942 | | | |
| P13929 | ENO3 | Down | 1-5 Y only | −0.055 | 0.649 | 0.947 | | | |
| P20783 | NTF3 | Down | 1-5 Y only | −0.032 | 0.668 | 0.950 | | | |
| P23471 | PTPRZ1 | Down | 1-5 Y only | −0.032 | 0.680 | 0.952 | | | |
| Q9P2M1 | LRP2BP | Down | 1-5 Y only | −0.072 | 0.722 | 0.955 | | | |
| P16870 | CPE | Down | 1-5 Y only | −0.022 | 0.730 | 0.958 | | | |
| P43121 | MCAM | Down | 1-5 Y only | −0.020 | 0.743 | 0.960 | | | |
| P21810 | BGN | Down | 1-5 Y only | −0.026 | 0.763 | 0.964 | | | |
| Q6P1J6 | PLB1 | Down | 1-5 Y only | −0.035 | 0.767 | 0.967 | | | |
| P46937 | YAP1 | Down | 1-5 Y only | −0.015 | 0.789 | 0.968 | | | |
| Q15582 | TGFBI | Down | 1-5 Y only | −0.010 | 0.845 | 0.969 | | | |
| P00167 | CYB5A | Down | 1-5 Y only | −0.012 | 0.921 | 0.987 | | | |
| P56851 | EDDM3B | Down | 1-5 Y only | −0.008 | 0.947 | 0.992 | | | |
| P49908 | SELENOP | Down | 1-5 Y only | −0.003 | 0.963 | 0.994 | | | |
| Q6UWR7 | ENPP6 | Down | Both | −0.265 | 0.0006 | 0.879 | −0.265 | 0.0006 | 0.879 |
| Q86YD3 | TMEM25 | Down | Both | −0.288 | 0.002 | 0.879 | −0.288 | 0.002 | 0.879 |
| P09681 | GIP | Down | Both | −0.414 | 0.002 | 0.879 | −0.414 | 0.002 | 0.879 |
| O95196 | CSPG5 | Down | Both | −0.311 | 0.005 | 0.933 | −0.311 | 0.005 | 0.933 |
| O76038 | SCGN | Down | Both | −0.271 | 0.009 | 0.933 | −0.271 | 0.009 | 0.933 |
| P98073 | TMPRSS15 | Down | Both | −0.403 | 0.014 | 0.933 | −0.403 | 0.014 | 0.933 |
| Q6ISS4 | LAIR2 | Down | Both | −0.406 | 0.017 | 0.933 | −0.406 | 0.017 | 0.933 |
| Q96J84 | KIRREL1 | Down | Both | −0.302 | 0.023 | 0.933 | −0.302 | 0.023 | 0.933 |
| P34130 | NTF4 | Down | Both | −0.217 | 0.025 | 0.933 | −0.217 | 0.025 | 0.933 |
| P41732 | TSPAN7 | Down | Both | −0.245 | 0.030 | 0.933 | −0.245 | 0.030 | 0.933 |
| P21128 | ENDOU | Down | Both | −0.205 | 0.034 | 0.933 | −0.205 | 0.034 | 0.933 |
| O43240 | KLK10 | Down | Both | −0.159 | 0.037 | 0.933 | −0.159 | 0.037 | 0.933 |
| O00175 | CCL24 | Down | Both | −0.320 | 0.037 | 0.933 | −0.320 | 0.037 | 0.933 |
| O15354 | GPR37 | Down | Both | −0.280 | 0.038 | 0.933 | −0.280 | 0.038 | 0.933 |
| P04234 | CD3D | Down | Both | −0.131 | 0.039 | 0.933 | −0.131 | 0.039 | 0.933 |
| O95049 | TJP3 | Down | Both | −0.238 | 0.041 | 0.933 | −0.238 | 0.041 | 0.933 |
| Q9UK85 | DKKL1 | Down | Both | −0.326 | 0.042 | 0.933 | −0.326 | 0.042 | 0.933 |
| POCG37 | CFC1 | Down | Both | −0.187 | 0.045 | 0.933 | −0.187 | 0.045 | 0.933 |
| Q5VT99 | LRRC38 | Down | Both | −0.169 | 0.048 | 0.933 | −0.169 | 0.048 | 0.933 |
| P01275 | GCG | Down | Both | −0.524 | 0.051 | 0.933 | −0.524 | 0.051 | 0.933 |
| Q5U5Z8 | AGBL2 | Down | Both | −0.408 | 0.057 | 0.933 | −0.408 | 0.057 | 0.933 |
| P48023 | FASLG | Down | Both | −0.133 | 0.060 | 0.933 | −0.133 | 0.060 | 0.933 |
| Q8IVF2 | AHNAK2 | Down | Both | −0.117 | 0.065 | 0.933 | −0.117 | 0.065 | 0.933 |
| Q8TEU8 | WFIKKN2 | Down | Both | −0.132 | 0.068 | 0.933 | −0.132 | 0.068 | 0.933 |
| Q9UJ72 | ANXA10 | Down | Both | −0.352 | 0.068 | 0.933 | −0.352 | 0.068 | 0.933 |
| O60243 | HS6ST1 | Down | Both | −0.122 | 0.071 | 0.933 | −0.122 | 0.071 | 0.933 |
| Q68J44 | DUSP29 | Down | Both | −0.266 | 0.072 | 0.933 | −0.266 | 0.072 | 0.933 |
| Q9ULX7 | CA14 | Down | Both | −0.127 | 0.074 | 0.933 | −0.127 | 0.074 | 0.933 |
| Q9BXN2 | CLEC7A | Down | Both | −0.200 | 0.078 | 0.933 | −0.200 | 0.078 | 0.933 |
| Q86SQ0 | PHLDB2 | Down | Both | −0.270 | 0.080 | 0.933 | −0.270 | 0.080 | 0.933 |
| O75711 | SCRG1 | Down | Both | −0.103 | 0.084 | 0.933 | −0.103 | 0.084 | 0.933 |
| Q9BXY4 | RSPO3 | Down | Both | −0.119 | 0.091 | 0.933 | −0.119 | 0.091 | 0.933 |
| P11387 | TOP1 | Down | Both | −0.395 | 0.094 | 0.933 | −0.395 | 0.094 | 0.933 |
| Q9GZM7 | TINAGL1 | Down | Both | −0.086 | 0.099 | 0.933 | −0.086 | 0.099 | 0.933 |
| P13591 | NCAM1 | Down | Both | −0.091 | 0.102 | 0.933 | −0.091 | 0.102 | 0.933 |
| Q96BQ1 | FAM3D | Down | Both | −0.210 | 0.107 | 0.933 | −0.210 | 0.107 | 0.933 |
| P49771 | FLT3LG | Down | Both | −0.128 | 0.109 | 0.933 | −0.128 | 0.109 | 0.933 |
| P21754 | ZP3 | Down | Both | −0.743 | 0.116 | 0.933 | −0.743 | 0.116 | 0.933 |
| O00253 | AGRP | Down | Both | −0.202 | 0.116 | 0.933 | −0.202 | 0.116 | 0.933 |
| Q9NR71 | ASAH2 | Down | Both | −0.179 | 0.124 | 0.933 | −0.179 | 0.124 | 0.933 |
| P09619 | PDGFRB | Down | Both | −0.102 | 0.126 | 0.933 | −0.102 | 0.126 | 0.933 |
| P43652 | AFM | Down | Both | −0.065 | 0.128 | 0.933 | −0.065 | 0.128 | 0.933 |
| P01303 | NPY | Down | Both | −0.227 | 0.130 | 0.933 | −0.227 | 0.130 | 0.933 |
| P01298 | PPY | Down | Both | −0.297 | 0.131 | 0.933 | −0.297 | 0.131 | 0.933 |
| P55808 | XG | Down | Both | −0.099 | 0.146 | 0.933 | −0.099 | 0.146 | 0.933 |
| Q08431 | MFGE8 | Down | Both | −0.112 | 0.183 | 0.933 | −0.112 | 0.183 | 0.933 |
| P07225 | PROS1 | Down | Both | −0.070 | 0.224 | 0.933 | −0.070 | 0.224 | 0.933 |
| A6BM72 | MEGF11 | Down | Both | −0.068 | 0.295 | 0.933 | −0.068 | 0.295 | 0.933 |
| P09683 | SCT | Up | 1-3 Y only | | | | 0.344 | 0.003 | 0.933 |
| P00751 | CFB | Up | 1-3 Y only | | | | 0.167 | 0.007 | 0.933 |
| P03951 | F11 | Up | 1-3 Y only | | | | 0.143 | 0.009 | 0.933 |
| Q01484 | ANK2 | Up | 1-3 Y only | | | | 0.338 | 0.010 | 0.933 |
| Q9UHY7 | ENOPH1 | Up | 1-3 Y only | | | | 0.160 | 0.020 | 0.933 |
| O60701 | UGDH | Up | 1-3 Y only | | | | 0.253 | 0.024 | 0.933 |
| Q13510 | ASAH1 | Up | 1-3 Y only | | | | 0.166 | 0.025 | 0.933 |
| Q15303 | ERBB4 | Up | 1-3 Y only | | | | 0.117 | 0.027 | 0.933 |
| Q9UHA7 | IL36A | Up | 1-3 Y only | | | | 0.270 | 0.028 | 0.933 |
| P02671 | FGA | Up | 1-3 Y only | | | | 0.164 | 0.031 | 0.933 |
| P01031 | C5 | Up | 1-3 Y only | | | | 0.087 | 0.032 | 0.933 |
| Q99650 | OSMR | Up | 1-3 Y only | | | | 0.084 | 0.038 | 0.933 |
| Q04837 | SSBP1 | Up | 1-3 Y only | | | | 0.155 | 0.039 | 0.933 |
| Q6R327 | RICTOR | Up | 1-3 Y only | | | | 0.177 | 0.039 | 0.933 |
| P02750 | LRG1 | Up | 1-3 Y only | | | | 0.137 | 0.040 | 0.933 |
| P20851 | C4BPB | Up | 1-3 Y only | | | | 0.167 | 0.040 | 0.933 |
| Q96BJ3 | AIDA | Up | 1-3 Y only | | | | 0.239 | 0.041 | 0.933 |
| Q8WTU2 | SSC4D | Up | 1-3 Y only | | | | 0.508 | 0.043 | 0.933 |
| P28799 | GRN | Up | 1-3 Y only | | | | 0.097 | 0.045 | 0.933 |
| P17181 | IFNAR1 | Up | 1-3 Y only | | | | 0.088 | 0.048 | 0.933 |
| Q07075 | ENPEP | Up | 1-3 Y only | | | | 0.160 | 0.049 | 0.933 |
| P45954 | ACADSB | Up | 1-3 Y only | | | | 0.282 | 0.051 | 0.933 |
| O60476 | MAN1A2 | Up | 1-3 Y only | | | | 0.104 | 0.053 | 0.933 |
| Q96PP9 | GBP4 | Up | 1-3 Y only | | | | 0.123 | 0.056 | 0.933 |
| P05155 | SERPING1 | Up | 1-3 Y only | | | | 0.063 | 0.056 | 0.933 |
| P53420 | COL4A4 | Up | 1-3 Y only | | | | 0.399 | 0.057 | 0.933 |
| P48431 | SOX2 | Up | 1-3 Y only | | | | 0.075 | 0.060 | 0.933 |
| Q12849 | GRSF1 | Up | 1-3 Y only | | | | 0.205 | 0.064 | 0.933 |
| P78395 | PRAME | Up | 1-3 Y only | | | | 0.128 | 0.065 | 0.933 |
| P43632 | KIR2DS4 | Up | 1-3 Y only | | | | 0.570 | 0.067 | 0.933 |
| Q9UHI8 | ADAMTS1 | Up | 1-3 Y only | | | | 0.131 | 0.068 | 0.933 |
| Q8IWB1 | ITPRIP | Up | 1-3 Y only | | | | 0.168 | 0.071 | 0.933 |
| P54108 | CRISP3 | Up | 1-3 Y only | | | | 0.083 | 0.072 | 0.933 |
| Q86SJ6 | DSG4 | Up | 1-3 Y only | | | | 0.168 | 0.073 | 0.933 |
| Q14624 | ITIH4 | Up | 1-3 Y only | | | | 0.111 | 0.073 | 0.933 |
| P22897 | MRC1 | Up | 1-3 Y only | | | | 0.113 | 0.073 | 0.933 |
| P48169 | GABRA4 | Up | 1-3 Y only | | | | 0.240 | 0.075 | 0.933 |
| P01011 | SERPINA3 | Up | 1-3 Y only | | | | 0.059 | 0.076 | 0.933 |
| Q7Z6M3 | MILR1 | Up | 1-3 Y only | | | | 0.160 | 0.076 | 0.933 |
| O60240 | PLIN1 | Up | 1-3 Y only | | | | 0.147 | 0.078 | 0.933 |
| Q15465 | SHH | Up | 1-3 Y only | | | | 0.162 | 0.078 | 0.933 |
| P03952 | KLKB1 | Up | 1-3 Y only | | | | 0.085 | 0.080 | 0.933 |
| Q96F46 | IL17RA | Up | 1-3 Y only | | | | 0.135 | 0.084 | 0.933 |
| P09238 | MMP10 | Up | 1-3 Y only | | | | 0.217 | 0.087 | 0.933 |
| P18428 | LBP | Up | 1-3 Y only | | | | 0.219 | 0.087 | 0.933 |
| Q99717 | SMAD5 | Up | 1-3 Y only | | | | 0.052 | 0.088 | 0.933 |
| P08913 | ADRA2A | Up | 1-3 Y only | | | | 0.155 | 0.089 | 0.933 |
| Q86VW0 | SESTD1 | Up | 1-3 Y only | | | | 0.244 | 0.090 | 0.933 |
| P05156 | CFI | Up | 1-3 Y only | | | | 0.076 | 0.091 | 0.933 |
| Q8NHP1 | AKR7L | Up | 1-3 Y only | | | | 0.201 | 0.092 | 0.933 |
| P09668 | CTSH | Up | 1-3 Y only | | | | 0.268 | 0.092 | 0.933 |
| O95274 | LYPD3 | Up | 1-3 Y only | | | | 0.121 | 0.094 | 0.933 |
| P27352 | CBLIF | Up | 1-3 Y only | | | | 0.311 | 0.094 | 0.933 |
| P53814 | SMTN | Up | 1-3 Y only | | | | 0.301 | 0.095 | 0.933 |
| P08603 | CFH | Up | 1-3 Y only | | | | 0.086 | 0.095 | 0.933 |
| P01008 | SERPINC1 | Up | 1-3 Y only | | | | 0.050 | 0.096 | 0.933 |
| Q99988 | GDF15 | Up | 1-3 Y only | | | | 0.153 | 0.100 | 0.933 |
| O15018 | PDZD2 | Up | 1-3 Y only | | | | 0.207 | 0.101 | 0.933 |
| P05091 | ALDH2 | Up | 1-3 Y only | | | | 0.099 | 0.102 | 0.933 |
| Q8IYV9 | IZUMO1 | Up | 1-3 Y only | | | | 0.097 | 0.103 | 0.933 |
| Q9UQ16 | DNM3 | Up | 1-3 Y only | | | | 0.136 | 0.104 | 0.933 |
| Q99731 | CCL19 | Up | 1-3 Y only | | | | 0.400 | 0.105 | 0.933 |
| P04141 | CSF2 | Up | 1-3 Y only | | | | 0.110 | 0.107 | 0.933 |
| Q96PE7 | MCEE | Up | 1-3 Y only | | | | 0.087 | 0.110 | 0.933 |
| P10109 | FDX1 | Up | 1-3 Y only | | | | 0.142 | 0.114 | 0.933 |
| P18827 | SDC1 | Up | 1-3 Y only | | | | 0.138 | 0.118 | 0.933 |
| Q15063 | POSTN | Up | 1-3 Y only | | | | 0.167 | 0.118 | 0.933 |
| P55259 | GP2 | Up | 1-3 Y only | | | | 0.246 | 0.119 | 0.933 |
| O76096 | CST7 | Up | 1-3 Y only | | | | 0.256 | 0.119 | 0.933 |
| P08571 | CD14 | Up | 1-3 Y only | | | | 0.135 | 0.119 | 0.933 |
| Q8TDX7 | NEK7 | Up | 1-3 Y only | | | | 0.143 | 0.122 | 0.933 |
| P29353 | SHC1 | Up | 1-3 Y only | | | | 0.237 | 0.125 | 0.933 |
| Q96HD1 | CRELD1 | Up | 1-3 Y only | | | | 0.129 | 0.125 | 0.933 |
| P20062 | TCN2 | Up | 1-3 Y only | | | | 0.110 | 0.128 | 0.933 |
| Q8IY22 | CMIP | Up | 1-3 Y only | | | | 0.169 | 0.130 | 0.933 |
| P24387 | CRHBP | Up | 1-3 Y only | | | | 0.079 | 0.131 | 0.933 |
| P02748 | C9 | Up | 1-3 Y only | | | | 0.125 | 0.135 | 0.933 |
| A1KZ92 | PXDNL | Up | 1-3 Y only | | | | 0.127 | 0.139 | 0.933 |
| Q92823 | NRCAM | Up | 1-3 Y only | | | | 0.096 | 0.140 | 0.933 |
| P78352 | DLG4 | Up | 1-3 Y only | | | | 0.161 | 0.140 | 0.933 |
| O43734 | TRAF3IP2 | Up | 1-3 Y only | | | | 0.114 | 0.149 | 0.933 |
| Q06520 | SULT2A1 | Up | 1-3 Y only | | | | 0.156 | 0.155 | 0.933 |
| P0CG30 | GSTT2B | Up | 1-3 Y only | | | | 0.443 | 0.167 | 0.933 |
| P19827 | ITIH1 | Up | 1-3 Y only | | | | 0.049 | 0.172 | 0.933 |
| Q96A35 | MRPL24 | Up | 1-3 Y only | | | | 0.099 | 0.194 | 0.933 |
| Q8WXI7 | MUC16 | Up | 1-3 Y only | | | | 0.268 | 0.195 | 0.933 |
| P08700 | IL3 | Up | 1-3 Y only | | | | 0.234 | 0.216 | 0.933 |
| P10909 | CLU | Up | 1-3 Y only | | | | 0.079 | 0.272 | 0.933 |
| Q5W0V3 | FHIP2A | Up | 1-3 Y only | | | | 0.098 | 0.468 | 0.933 |
| P43234 | CTSO | Up | 1-5 Y only | 0.092 | 0.116 | 0.933 | | | |
| P16410 | CTLA4 | Up | 1-5 Y only | 0.180 | 0.143 | 0.933 | | | |
| Q99062 | CSF3R | Up | 1-5 Y only | 0.080 | 0.147 | 0.933 | | | |
| P24071 | FCAR | Up | 1-5 Y only | 0.161 | 0.154 | 0.933 | | | |
| P78358 | CTAG1A | Up | 1-5 Y only | 0.107 | 0.157 | 0.933 | | | |
| Q9HB40 | SCPEP1 | Up | 1-5 Y only | 0.122 | 0.170 | 0.933 | | | |
| Q2L4Q9 | PRSS53 | Up | 1-5 Y only | 0.095 | 0.181 | 0.933 | | | |
| Q6UXH1 | CRELD2 | Up | 1-5 Y only | 0.123 | 0.186 | 0.933 | | | |
| Q9UKJ1 | PILRA | Up | 1-5 Y only | 0.110 | 0.194 | 0.933 | | | |
| P04070 | PROC | Up | 1-5 Y only | 0.066 | 0.206 | 0.933 | | | |
| Q7L8A9 | VASH1 | Up | 1-5 Y only | 0.132 | 0.210 | 0.933 | | | |
| P29474 | NOS3 | Up | 1-5 Y only | 0.126 | 0.212 | 0.933 | | | |
| Q8N4F0 | BPIFB2 | Up | 1-5 Y only | 0.163 | 0.214 | 0.933 | | | |
| BOFP48 | UPK3BL1 | Up | 1-5 Y only | 0.115 | 0.222 | 0.933 | | | |
| O00567 | NOP56 | Up | 1-5 Y only | 0.211 | 0.251 | 0.933 | | | |
| Q9BX67 | JAM3 | Up | 1-5 Y only | 0.101 | 0.267 | 0.933 | | | |
| P01903 | HLA-DRA | Up | 1-5 Y only | 0.123 | 0.270 | 0.933 | | | |
| Q9H173 | SIL1 | Up | 1-5 Y only | 0.073 | 0.273 | 0.933 | | | |
| Q8NET8 | TRPV3 | Up | 1-5 Y only | 0.098 | 0.277 | 0.933 | | | |
| Q9BV94 | EDEM2 | Up | 1-5 Y only | 0.107 | 0.283 | 0.933 | | | |
| P24928 | POLR2A | Up | 1-5 Y only | 0.062 | 0.289 | 0.933 | | | |
| P23435 | CBLN1 | Up | 1-5 Y only | 0.120 | 0.309 | 0.933 | | | |
| Q9Y680 | FKBP7 | Up | 1-5 Y only | 0.134 | 0.312 | 0.933 | | | |
| P78556 | CCL20 | Up | 1-5 Y only | 0.191 | 0.318 | 0.933 | | | |
| Q9UKJ0 | PILRB | Up | 1-5 Y only | 0.110 | 0.329 | 0.933 | | | |
| O00241 | SIRPB1 | Up | 1-5 Y only | 0.083 | 0.340 | 0.933 | | | |
| Q6UX27 | VSTM1 | Up | 1-5 Y only | 0.120 | 0.395 | 0.933 | | | |
| Q10589 | BST2 | Up | 1-5 Y only | 0.067 | 0.449 | 0.933 | | | |
| Q9NR61 | DLL4 | Up | 1-5 Y only | 0.114 | 0.467 | 0.933 | | | |
| Q9NZP8 | C1RL | Up | 1-5 Y only | 0.027 | 0.471 | 0.933 | | | |
| O00584 | RNASET2 | Up | 1-5 Y only | 0.037 | 0.473 | 0.933 | | | |
| Q12809 | KCNH2 | Up | 1-5 Y only | 0.116 | 0.478 | 0.933 | | | |
| Q99665 | IL12RB2 | Up | 1-5 Y only | 0.053 | 0.493 | 0.933 | | | |
| Q9ULW2 | FZD10 | Up | 1-5 Y only | 0.063 | 0.553 | 0.936 | | | |
| P55809 | OXCT1 | Up | 1-5 Y only | 0.069 | 0.605 | 0.942 | | | |
| Q5T2D2 | TREML2 | Up | 1-5 Y only | 0.031 | 0.654 | 0.947 | | | |
| Q13224 | GRIN2B | Up | 1-5 Y only | 0.023 | 0.763 | 0.964 | | | |
| Q6UXV0 | GFRAL | Up | 1-5 Y only | 0.030 | 0.779 | 0.968 | | | |
| P57771 | RGS8 | Up | 1-5 Y only | 0.024 | 0.831 | 0.969 | | | |
| P30533 | LRPAP1 | Up | 1-5 Y only | 0.027 | 0.833 | 0.969 | | | |
| P98164 | LRP2 | Up | 1-5 Y only | 0.018 | 0.834 | 0.969 | | | |
| Q96ID5 | IGSF21 | Up | 1-5 Y only | 0.014 | 0.838 | 0.969 | | | |
| Q07507 | DPT | Up | 1-5 Y only | 0.009 | 0.847 | 0.969 | | | |
| A8MVW5 | HEPACAM2 | Up | 1-5 Y only | 0.014 | 0.853 | 0.970 | | | |
| O15232 | MATN3 | Up | 1-5 Y only | 0.008 | 0.902 | 0.984 | | | |
| Q8NBZ7 | UXS1 | Up | 1-5 Y only | 0.013 | 0.905 | 0.984 | | | |
| O95997 | PTTG1 | Up | 1-5 Y only | 0.007 | 0.913 | 0.985 | | | |
| Q13410 | BTN1A1 | Up | 1-5 Y only | 0.005 | 0.921 | 0.987 | | | |
| Q9P0M4 | IL17C | Up | 1-5 Y only | 0.011 | 0.941 | 0.992 | | | |
| Q9Y6U3 | SCIN | Up | 1-5 Y only | 0.000 | 0.997 | 0.999 | | | |
| P04183 | TK1 | Up | Both | 0.203 | 0.003 | 0.933 | 0.203 | 0.003 | 0.933 |
| Q9NWM8 | FKBP14 | Up | Both | 0.425 | 0.004 | 0.933 | 0.425 | 0.004 | 0.933 |
| O00534 | VWA5A | Up | Both | 0.343 | 0.004 | 0.933 | 0.343 | 0.004 | 0.933 |
| Q13976 | PRKG1 | Up | Both | 0.659 | 0.006 | 0.933 | 0.659 | 0.006 | 0.933 |
| Q7LOJ3 | SV2A | Up | Both | 0.388 | 0.007 | 0.933 | 0.388 | 0.007 | 0.933 |
| P20382 | PMCH | Up | Both | 0.493 | 0.008 | 0.933 | 0.493 | 0.008 | 0.933 |
| Q0ZGT2 | NEXN | Up | Both | 0.318 | 0.009 | 0.933 | 0.318 | 0.009 | 0.933 |
| Q9H5V8 | CDCP1 | Up | Both | 0.264 | 0.009 | 0.933 | 0.264 | 0.009 | 0.933 |
| Q86TM3 | DDX53 | Up | Both | 0.159 | 0.011 | 0.933 | 0.159 | 0.011 | 0.933 |
| Q9NS62 | THSD1 | Up | Both | 0.148 | 0.012 | 0.933 | 0.148 | 0.012 | 0.933 |
| O96013 | PAK4 | Up | Both | 0.453 | 0.013 | 0.933 | 0.453 | 0.013 | 0.933 |
| P39900 | MMP12 | Up | Both | 0.321 | 0.013 | 0.933 | 0.321 | 0.013 | 0.933 |
| O00602 | FCN1 | Up | Both | 0.265 | 0.013 | 0.933 | 0.265 | 0.013 | 0.933 |
| P07911 | UMOD | Up | Both | 0.258 | 0.014 | 0.933 | 0.258 | 0.014 | 0.933 |
| P13667 | PDIA4 | Up | Both | 0.306 | 0.014 | 0.933 | 0.306 | 0.014 | 0.933 |
| P05231 | IL6 | Up | Both | 0.444 | 0.018 | 0.933 | 0.444 | 0.018 | 0.933 |
| Q8WUW1 | BRK1 | Up | Both | 0.158 | 0.024 | 0.933 | 0.158 | 0.024 | 0.933 |
| Q8N149 | LILRA2 | Up | Both | 0.166 | 0.027 | 0.933 | 0.166 | 0.027 | 0.933 |
| Q6ZRY4 | RBPMS2 | Up | Both | 0.511 | 0.028 | 0.933 | 0.511 | 0.028 | 0.933 |
| P05546 | SERPIND1 | Up | Both | 0.145 | 0.029 | 0.933 | 0.145 | 0.029 | 0.933 |
| Q9NRR2 | TPSG1 | Up | Both | 0.271 | 0.030 | 0.933 | 0.271 | 0.030 | 0.933 |
| P06731 | CEACAM5 | Up | Both | 0.433 | 0.030 | 0.933 | 0.433 | 0.030 | 0.933 |
| P31371 | FGF9 | Up | Both | 0.238 | 0.030 | 0.933 | 0.238 | 0.030 | 0.933 |
| P30405 | PPIF | Up | Both | 0.261 | 0.031 | 0.933 | 0.261 | 0.031 | 0.933 |
| Q68DV7 | RNF43 | Up | Both | 0.274 | 0.035 | 0.933 | 0.274 | 0.035 | 0.933 |
| Q9Y336 | SIGLEC9 | Up | Both | 0.111 | 0.037 | 0.933 | 0.111 | 0.037 | 0.933 |
| Q15388 | TOMM20 | Up | Both | 0.301 | 0.042 | 0.933 | 0.301 | 0.042 | 0.933 |
| O76074 | PDE5A | Up | Both | 0.409 | 0.043 | 0.933 | 0.409 | 0.043 | 0.933 |
| Q92832 | NELL1 | Up | Both | 0.193 | 0.045 | 0.933 | 0.193 | 0.045 | 0.933 |
| P04062 | GBA | Up | Both | 0.187 | 0.047 | 0.933 | 0.187 | 0.047 | 0.933 |
| P09466 | PAEP | Up | Both | 0.383 | 0.049 | 0.933 | 0.383 | 0.049 | 0.933 |
| O75460 | ERN1 | Up | Both | 0.180 | 0.055 | 0.933 | 0.180 | 0.055 | 0.933 |
| Q16549 | PCSK7 | Up | Both | 0.176 | 0.058 | 0.933 | 0.176 | 0.058 | 0.933 |
| Q9BRQ6 | CHCHD6 | Up | Both | 0.104 | 0.058 | 0.933 | 0.104 | 0.058 | 0.933 |
| Q9UEW3 | MARCO | Up | Both | 0.095 | 0.062 | 0.933 | 0.095 | 0.062 | 0.933 |
| Q8IWL2 | SFTPA1 | Up | Both | 0.239 | 0.067 | 0.933 | 0.239 | 0.067 | 0.933 |
| P15248 | IL9 | Up | Both | 0.202 | 0.069 | 0.933 | 0.202 | 0.069 | 0.933 |
| Q16719 | KYNU | Up | Both | 0.149 | 0.071 | 0.933 | 0.149 | 0.071 | 0.933 |
| O43278 | SPINT1 | Up | Both | 0.107 | 0.073 | 0.933 | 0.107 | 0.073 | 0.933 |
| Q9ULH4 | LRFN2 | Up | Both | 0.178 | 0.075 | 0.933 | 0.178 | 0.075 | 0.933 |
| Q15223 | NECTIN1 | Up | Both | 0.095 | 0.079 | 0.933 | 0.095 | 0.079 | 0.933 |
| Q8IYS5 | OSCAR | Up | Both | 0.121 | 0.080 | 0.933 | 0.121 | 0.080 | 0.933 |
| P20742 | PZP | Up | Both | 0.164 | 0.080 | 0.933 | 0.164 | 0.080 | 0.933 |
| Q8TDL5 | BPIFB1 | Up | Both | 0.241 | 0.084 | 0.933 | 0.241 | 0.084 | 0.933 |
| A6NI73 | LILRA5 | Up | Both | 0.114 | 0.088 | 0.933 | 0.114 | 0.088 | 0.933 |
| Q9NYX4 | CALY | Up | Both | 0.121 | 0.093 | 0.933 | 0.121 | 0.093 | 0.933 |
| P10301 | RRAS | Up | Both | 0.232 | 0.095 | 0.933 | 0.232 | 0.095 | 0.933 |
| Q8TAE8 | GADD45GIP1 | Up | Both | 0.157 | 0.097 | 0.933 | 0.157 | 0.097 | 0.933 |
| Q6H9L7 | ISM2 | Up | Both | 0.169 | 0.102 | 0.933 | 0.169 | 0.102 | 0.933 |
| Q96PL1 | SCGB3A2 | Up | Both | 0.345 | 0.107 | 0.933 | 0.345 | 0.107 | 0.933 |
| P40199 | CEACAM6 | Up | Both | 0.238 | 0.108 | 0.933 | 0.238 | 0.108 | 0.933 |
| Q93052 | LPP | Up | Both | 0.175 | 0.112 | 0.933 | 0.175 | 0.112 | 0.933 |
| Q9NS71 | GKN1 | Up | Both | 0.076 | 0.118 | 0.933 | 0.076 | 0.118 | 0.933 |
| Q96JA1 | LRIG1 | Up | Both | 0.120 | 0.120 | 0.933 | 0.120 | 0.120 | 0.933 |
| Q9HAW4 | CLSPN | Up | Both | 0.139 | 0.120 | 0.933 | 0.139 | 0.120 | 0.933 |
| O43927 | CXCL13 | Up | Both | 0.183 | 0.123 | 0.933 | 0.183 | 0.123 | 0.933 |
| Q8IWL1 | SFTPA2 | Up | Both | 0.244 | 0.124 | 0.933 | 0.244 | 0.124 | 0.933 |
| P14854 | COX6B1 | Up | Both | 0.149 | 0.125 | 0.933 | 0.149 | 0.125 | 0.933 |
| Q14914 | PTGR1 | Up | Both | 0.279 | 0.131 | 0.933 | 0.279 | 0.131 | 0.933 |
| Q93062 | RBPMS | Up | Both | 0.156 | 0.132 | 0.933 | 0.156 | 0.132 | 0.933 |
| P50897 | PPT1 | Up | Both | 0.215 | 0.133 | 0.933 | 0.215 | 0.133 | 0.933 |
| P19801 | AOC1 | Up | Both | 0.333 | 0.135 | 0.933 | 0.333 | 0.135 | 0.933 |
| Q96HC4 | PDLIM5 | Up | Both | 0.265 | 0.139 | 0.933 | 0.265 | 0.139 | 0.933 |
| Q96EM0 | L3HYPDH | Up | Both | 0.234 | 0.139 | 0.933 | 0.234 | 0.139 | 0.933 |
| P36776 | LONP1 | Up | Both | 0.205 | 0.145 | 0.933 | 0.205 | 0.145 | 0.933 |
| O14791 | APOL1 | Up | Both | 0.166 | 0.149 | 0.933 | 0.166 | 0.149 | 0.933 |
| A8MTB9 | CEACAM18 | Up | Both | 0.216 | 0.152 | 0.933 | 0.216 | 0.152 | 0.933 |
| P21781 | FGF7 | Up | Both | 0.171 | 0.182 | 0.933 | 0.171 | 0.182 | 0.933 |
| P02533 | KRT14 | Up | Both | 0.198 | 0.205 | 0.933 | 0.198 | 0.205 | 0.933 |

TABLE 14
Correlations between protein relative levels from Olink
Target 96 platform and the Olink Explore platform

| | Pearson Correlation | | |
|---|---|---|---|
| Gene | Coefficient | P value | FDR |
| CXCL5 | 0.963 | 8.4E−263 | 2.2E−260 |
| ANGPT1 | 0.959 | 1.1E−253 | 1.4E−251 |
| CXCL9 | 0.958 | 4.0E−251 | 3.5E−249 |
| CDHR2 | 0.956 | 1.2E−247 | 8.1E−246 |
| REN | 0.953 | 5.2E−240 | 2.7E−238 |
| DFFA | 0.952 | 4.6E−239 | 2.0E−237 |
| CCL17 | 0.951 | 4.1E−237 | 1.6E−235 |
| CALCOCO1 | 0.951 | 4.9E−237 | 1.6E−235 |
| ALPP | 0.950 | 1.5E−235 | 4.4E−234 |
| SH2D1A | 0.950 | 7.5E−234 | 2.0E−232 |
| KLK4 | 0.949 | 5.9E−233 | 1.4E−231 |
| ELOA | 0.948 | 4.4E−231 | 9.7E−230 |
| PSIP1 | 0.947 | 1.9E−228 | 3.9E−227 |
| HEXIM1 | 0.945 | 5.1E−226 | 9.7E−225 |
| CXCL11 | 0.945 | 5.7E−225 | 1.0E−223 |
| CTRC | 0.945 | 3.3E−224 | 5.4E−223 |
| TPSAB1 | 0.944 | 3.1E−223 | 4.8E−222 |
| SERPINA12 | 0.944 | 3.3E−223 | 4.8E−222 |
| ADA | 0.940 | 1.1E−217 | 1.6E−216 |
| LACTB2 | 0.940 | 2.5E−217 | 3.4E−216 |
| HCLS1 | 0.940 | 5.0E−217 | 6.3E−216 |
| MGMT | 0.940 | 7.4E−217 | 8.9E−216 |
| VPS53 | 0.940 | 1.1E−216 | 1.3E−215 |
| PRDX5 | 0.938 | 6.3E−214 | 7.0E−213 |
| CXCL6 | 0.937 | 3.7E−212 | 3.8E−211 |
| HMBS | 0.937 | 3.6E−212 | 3.8E−211 |
| SIT1 | 0.937 | 1.2E−211 | 1.2E−210 |
| PIK3AP1 | 0.937 | 1.4E−211 | 1.3E−210 |
| HBQ1 | 0.936 | 2.3E−211 | 2.1E−210 |
| TRAF2 | 0.936 | 4.4E−210 | 3.9E−209 |
| CLIP2 | 0.936 | 5.7E−210 | 4.9E−209 |
| SIRT2 | 0.935 | 4.5E−209 | 3.7E−208 |
| EGLN1 | 0.934 | 4.2E−208 | 3.3E−207 |
| PLXNA4 | 0.934 | 1.2E−207 | 9.3E−207 |
| IL16 | 0.934 | 2.3E−207 | 1.8E−206 |
| NT5C3A | 0.933 | 1.8E−206 | 1.3E−205 |
| LSP1 | 0.932 | 1.0E−204 | 7.4E−204 |
| CLEC7A | 0.931 | 4.7E−204 | 3.3E−203 |
| IFNGR2 | 0.931 | 9.1E−203 | 6.2E−202 |
| OSM | 0.930 | 1.7E−201 | 1.1E−200 |
| DAPP1 | 0.929 | 2.8E−200 | 1.8E−199 |
| PSMD9 | 0.928 | 2.5E−199 | 1.6E−198 |
| NAMPT | 0.927 | 1.2E−198 | 7.7E−198 |
| TNFSF14 | 0.927 | 1.4E−198 | 8.2E−198 |
| FGF2 | 0.927 | 3.5E−198 | 2.1E−197 |
| EDAR | 0.926 | 2.7E−197 | 1.6E−196 |
| LAMP3 | 0.925 | 6.4E−196 | 3.6E−195 |
| CDCP1 | 0.925 | 1.9E−195 | 1.1E−194 |
| SCLY | 0.924 | 9.1E−195 | 4.9E−194 |
| SRPK2 | 0.924 | 1.4E−194 | 7.5E−194 |
| FUS | 0.924 | 4.5E−194 | 2.3E−193 |
| MMP12 | 0.924 | 1.1E−193 | 5.4E−193 |
| CCL20 | 0.923 | 1.6E−193 | 8.2E−193 |
| CXCL10 | 0.923 | 5.8E−193 | 2.8E−192 |
| DCTN1 | 0.923 | 1.1E−192 | 5.2E−192 |
| KLRD1 | 0.922 | 1.6E−191 | 7.5E−191 |
| TRIM21 | 0.922 | 1.7E−191 | 7.9E−191 |
| CLEC4D | 0.921 | 4.9E−191 | 2.2E−190 |
| DDX58 | 0.921 | 5.2E−190 | 2.4E−189 |
| CEACAM8 | 0.920 | 1.4E−188 | 6.4E−188 |
| CCL4 | 0.918 | 2.0E−187 | 8.7E−187 |
| EIF4G1 | 0.916 | 1.2E−184 | 5.3E−184 |
| CCL25 | 0.914 | 1.7E−182 | 7.0E−182 |
| CD6 | 0.912 | 7.7E−180 | 3.2E−179 |
| IL6 | 0.912 | 8.1E−180 | 3.3E−179 |
| AFP | 0.911 | 6.6E−179 | 2.7E−178 |
| HSPB6 | 0.909 | 4.2E−177 | 1.7E−176 |
| IL18 | 0.908 | 1.4E−175 | 5.5E−175 |
| HNMT | 0.908 | 1.6E−175 | 6.2E−175 |
| ICAM4 | 0.907 | 4.4E−175 | 1.7E−174 |
| TNFRSF9 | 0.906 | 9.1E−174 | 3.4E−173 |
| STAMBP | 0.906 | 1.7E−173 | 6.1E−173 |
| DECR1 | 0.905 | 4.7E−172 | 1.7E−171 |
| ITGB1BP2 | 0.904 | 5.5E−172 | 2.0E−171 |
| UBAC1 | 0.903 | 9.6E−171 | 3.4E−170 |
| GOPC | 0.903 | 1.9E−170 | 6.5E−170 |
| IL7 | 0.901 | 2.6E−169 | 9.1E−169 |
| AXIN1 | 0.901 | 6.9E−169 | 2.3E−168 |
| VMO1 | 0.900 | 3.7E−168 | 1.3E−167 |
| CASP2 | 0.900 | 1.1E−167 | 3.7E−167 |
| IRAK4 | 0.898 | 8.7E−166 | 2.9E−165 |
| CASA | 0.898 | 2.4E−165 | 7.6E−165 |
| IRAK1 | 0.896 | 1.0E−164 | 3.2E−164 |
| AREG | 0.896 | 6.8E−164 | 2.2E−163 |
| FCRL6 | 0.895 | 1.3E−163 | 3.9E−163 |
| HSD11B1 | 0.895 | 1.3E−163 | 4.1E−163 |
| CCL19 | 0.894 | 1.5E−162 | 4.5E−162 |
| MLN | 0.893 | 6.5E−162 | 2.0E−161 |
| PRSS27 | 0.893 | 2.9E−161 | 8.7E−161 |
| GLO1 | 0.890 | 5.5E−159 | 1.6E−158 |
| GPA33 | 0.890 | 9.7E−159 | 2.8E−158 |
| VEGFA | 0.888 | 1.3E−157 | 3.7E−157 |
| TRIM5 | 0.887 | 1.9E−156 | 5.5E−156 |
| ACE2 | 0.887 | 2.7E−156 | 7.5E−156 |
| HGF | 0.887 | 2.7E−156 | 7.6E−156 |
| NELL1 | 0.884 | 3.2E−154 | 8.7E−154 |
| SLAMF7 | 0.884 | 1.2E−153 | 3.2E−153 |
| KRT19 | 0.881 | 5.3E−152 | 1.4E−151 |
| VSIG2 | 0.882 | 6.0E−152 | 1.6E−151 |
| MYO9B | 0.880 | 4.9E−151 | 1.3E−150 |
| CD300E | 0.880 | 7.7E−151 | 2.0E−150 |
| ZBTB16 | 0.875 | 7.3E−147 | 1.9E−146 |
| AKR1B1 | 0.874 | 1.4E−146 | 3.7E−146 |
| CST5 | 0.874 | 2.1E−146 | 5.3E−146 |
| BACH1 | 0.874 | 2.4E−146 | 6.0E−146 |
| CCL11 | 0.874 | 4.1E−146 | 1.0E−145 |
| RP2 | 0.873 | 1.1E−145 | 2.6E−145 |
| IL1B | 0.873 | 2.6E−145 | 6.5E−145 |
| VAT1 | 0.871 | 2.1E−144 | 5.1E−144 |
| SCG2 | 0.871 | 6.8E−144 | 1.6E−143 |
| DRG2 | 0.870 | 3.8E−143 | 9.0E−143 |
| INPP1 | 0.870 | 4.6E−143 | 1.1E−142 |
| CD22 | 0.869 | 1.4E−142 | 3.2E−142 |
| PPP1R9B | 0.867 | 3.2E−141 | 7.4E−141 |
| CD40 | 0.867 | 3.5E−141 | 8.1E−141 |
| DPP10 | 0.866 | 7.0E−141 | 1.6E−140 |
| SORT1 | 0.867 | 1.1E−140 | 2.4E−140 |
| SRC | 0.865 | 2.3E−139 | 5.1E−139 |
| IDUA | 0.863 | 3.6E−138 | 7.9E−138 |
| CDSN | 0.862 | 8.7E−138 | 1.9E−137 |
| TNFRSF11A | 0.862 | 1.0E−137 | 2.3E−137 |
| TBL1X | 0.861 | 3.9E−137 | 8.4E−137 |
| TNFRSF13B | 0.861 | 4.0E−137 | 8.6E−137 |
| SH2B3 | 0.860 | 1.3E−136 | 2.7E−136 |
| FABP2 | 0.861 | 1.5E−136 | 3.1E−136 |
| VWA1 | 0.858 | 2.7E−135 | 5.7E−135 |
| PRSS8 | 0.858 | 1.2E−134 | 2.6E−134 |
| FAM3B | 0.857 | 2.4E−134 | 4.9E−134 |
| NCR1 | 0.856 | 5.4E−134 | 1.1E−133 |
| CNTNAP2 | 0.856 | 6.9E−134 | 1.4E−133 |
| COL9A1 | 0.853 | 6.3E−132 | 1.3E−131 |
| LAP3 | 0.851 | 6.4E−131 | 1.3E−130 |
| ADM | 0.848 | 7.1E−129 | 1.4E−128 |
| CD84 | 0.847 | 4.1E−128 | 8.2E−128 |
| STK4 | 0.846 | 1.3E−127 | 2.6E−127 |
| C1QA | 0.845 | 2.4E−127 | 4.6E−127 |
| CLEC4C | 0.845 | 5.8E−127 | 1.1E−126 |
| PSPN | 0.844 | 9.0E−127 | 1.7E−126 |
| TGM2 | 0.844 | 2.5E−126 | 4.9E−126 |
| LPL | 0.842 | 2.5E−125 | 4.8E−125 |
| ITGA6 | 0.840 | 3.5E−124 | 6.5E−124 |
| CD5 | 0.839 | 1.2E−123 | 2.2E−123 |
| PTH1R | 0.839 | 1.4E−123 | 2.7E−123 |
| CCL23 | 0.838 | 2.7E−123 | 5.0E−123 |
| SCGN | 0.837 | 1.1E−122 | 2.0E−122 |
| VEGFD | 0.836 | 1.4E−121 | 2.6E−121 |
| LEP | 0.834 | 9.1E−121 | 1.6E−120 |
| CXADR | 0.832 | 1.1E−119 | 2.0E−119 |
| IL1RL2 | 0.831 | 7.5E−119 | 1.3E−118 |
| CBLN4 | 0.829 | 4.5E−118 | 7.9E−118 |
| VPS37A | 0.827 | 3.1E−117 | 5.4E−117 |
| XCL1 | 0.827 | 7.1E−117 | 1.2E−116 |
| GFER | 0.822 | 1.3E−114 | 2.3E−114 |
| DCTPP1 | 0.819 | 6.6E−113 | 1.1E−112 |
| NCS1 | 0.817 | 4.8E−112 | 8.2E−112 |
| THPO | 0.817 | 7.1E−112 | 1.2E−111 |
| ACAA1 | 0.816 | 1.6E−111 | 2.7E−111 |
| CCT5 | 0.814 | 1.1E−110 | 1.8E−110 |
| CD8A | 0.814 | 1.7E−110 | 2.9E−110 |
| USO1 | 0.813 | 3.3E−110 | 5.4E−110 |
| CD83 | 0.813 | 6.0E−110 | 9.8E−110 |
| IL17F | 0.813 | 6.4E−110 | 1.1E−109 |
| SPRY2 | 0.811 | 4.1E−109 | 6.7E−109 |
| SPINK4 | 0.809 | 4.9E−108 | 7.9E−108 |
| LILRB4 | 0.809 | 5.1E−108 | 8.3E−108 |
| GLB1 | 0.808 | 8.7E−108 | 1.4E−107 |
| CPVL | 0.804 | 7.6E−106 | 1.2E−105 |
| BTN3A2 | 0.801 | 2.0E−104 | 3.1E−104 |
| CLEC6A | 0.796 | 2.9E−102 | 4.6E−102 |
| LY75 | 0.794 | 1.2E−101 | 1.9E−101 |
| DCBLD2 | 0.794 | 1.6E−101 | 2.5E−101 |
| CD4 | 0.792 | 1.3E−100 | 2.0E−100 |
| IFNLR1 | 0.791 | 2.8E−100 | 4.2E−100 |
| MILR1 | 0.787 | 9.7E−99 | 1.5E−98 |
| CXCL1 | 0.786 | 7.5E−98 | 1.1E−97 |
| ICA1 | 0.781 | 3.7E−96 | 5.5E−96 |
| TNFRSF10A | 0.780 | 2.4E−95 | 3.7E−95 |
| ITM2A | 0.776 | 4.7E−94 | 7.0E−94 |
| ITGA11 | 0.773 | 5.0E−93 | 7.4E−93 |
| CCL3 | 0.772 | 3.3E−92 | 4.9E−92 |
| CDC27 | 0.768 | 5.3E−91 | 7.8E−91 |
| CX3CL1 | 0.765 | 6.9E−90 | 1.0E−89 |
| TXNDC15 | 0.764 | 9.5E−90 | 1.4E−89 |
| CLEC4A | 0.764 | 1.2E−89 | 1.7E−89 |
| BRK1 | 0.764 | 1.5E−89 | 2.2E−89 |
| SPON2 | 0.764 | 3.0E−89 | 4.3E−89 |
| CKAP4 | 0.760 | 3.2E−88 | 4.6E−88 |
| NFATC3 | 0.756 | 1.4E−86 | 1.9E−86 |
| ITGB6 | 0.754 | 4.7E−86 | 6.6E−86 |
| IL10 | 0.754 | 8.0E−86 | 1.1E−85 |
| YTHDF3 | 0.753 | 1.1E−85 | 1.6E−85 |
| DNER | 0.751 | 5.1E−85 | 7.0E−85 |
| CLEC4G | 0.751 | 6.5E−85 | 8.9E−85 |
| CCL28 | 0.751 | 7.4E−85 | 1.0E−84 |
| ERP44 | 0.751 | 8.2E−85 | 1.1E−84 |
| CD244 | 0.750 | 1.9E−84 | 2.6E−84 |
| TPMT | 0.739 | 5.4E−81 | 7.3E−81 |
| MARCO | 0.738 | 1.5E−80 | 2.0E−80 |
| PRDX1 | 0.737 | 2.2E−80 | 3.0E−80 |
| PTX3 | 0.737 | 3.8E−80 | 5.1E−80 |
| PTPRM | 0.736 | 4.1E−80 | 5.4E−80 |
| MANSC1 | 0.729 | 8.8E−78 | 1.2E−77 |
| AMBP | 0.727 | 8.2E−77 | 1.1E−76 |
| GDNF | 0.725 | 1.2E−76 | 1.6E−76 |
| PGF | 0.721 | 5.4E−75 | 6.9E−75 |
| LICAM | 0.719 | 1.4E−74 | 1.8E−74 |
| PRDX3 | 0.718 | 2.2E−74 | 2.8E−74 |
| PADI2 | 0.716 | 1.1E−73 | 1.3E−73 |
| SFTPA1 | 0.712 | 1.3E−72 | 1.7E−72 |
| THBS2 | 0.709 | 1.3E−71 | 1.7E−71 |
| MASP1 | 0.708 | 1.6E−71 | 2.0E−71 |
| FCRL3 | 0.700 | 2.2E−69 | 2.8E−69 |
| S100A16 | 0.697 | 1.7E−68 | 2.1E−68 |
| LAG3 | 0.695 | 4.9E−68 | 6.1E−68 |
| BOC | 0.695 | 8.2E−68 | 1.0E−67 |
| NPY | 0.692 | 4.8E−67 | 5.9E−67 |
| AGRP | 0.692 | 5.9E−67 | 7.2E−67 |
| MMP7 | 0.689 | 4.7E−66 | 5.8E−66 |
| SLAMF1 | 0.687 | 7.0E−66 | 8.5E−66 |
| MERTK | 0.688 | 7.1E−66 | 8.5E−66 |
| CXCL14 | 0.679 | 1.1E−63 | 1.3E−63 |
| PRELP | 0.672 | 6.0E−62 | 7.1E−62 |
| DCN | 0.672 | 9.2E−62 | 1.1E−61 |
| LIF | 0.667 | 1.2E−60 | 1.4E−60 |
| STC1 | 0.648 | 1.9E−56 | 2.3E−56 |
| BIRC2 | 0.645 | 8.8E−56 | 1.0E−55 |
| NTF4 | 0.643 | 2.4E−55 | 2.8E−55 |
| TANK | 0.641 | 8.8E−55 | 1.0E−54 |
| GALNT7 | 0.624 | 2.5E−51 | 2.9E−51 |
| CD28 | 0.614 | 3.2E−49 | 3.7E−49 |
| FOXO3 | 0.614 | 3.2E−49 | 3.7E−49 |
| FXYD5 | 0.613 | 5.2E−49 | 6.0E−49 |
| TNFAIP8 | 0.610 | 2.4E−48 | 2.7E−48 |
| RAB6A | 0.609 | 3.8E−48 | 4.3E−48 |
| PAPPA | 0.605 | 2.1E−47 | 2.4E−47 |
| CNPY2 | 0.587 | 3.3E−44 | 3.7E−44 |
| IL12RB1 | 0.584 | 1.4E−43 | 1.6E−43 |
| IL5 | 0.567 | 1.4E−40 | 1.6E−40 |
| GALNT3 | 0.550 | 6.1E−38 | 6.8E−38 |
| TSLP | 0.532 | 4.7E−35 | 5.2E−35 |
| FLT1 | 0.526 | 2.7E−34 | 3.0E−34 |
| TPT1 | 0.504 | 3.6E−31 | 4.0E−31 |
| DGKZ | 0.504 | 4.0E−31 | 4.4E−31 |
| FLT3 | 0.501 | 9.3E−31 | 1.0E−30 |
| AIF1 | 0.498 | 2.3E−30 | 2.4E−30 |
| ACTN4 | 0.494 | 8.3E−30 | 9.0E−30 |
| NRTN | 0.481 | 4.4E−28 | 4.7E−28 |
| CXCL12 | 0.472 | 5.4E−27 | 5.8E−27 |
| PCDH1 | 0.465 | 3.3E−26 | 3.5E−26 |
| IL13 | 0.463 | 6.1E−26 | 6.5E−26 |
| SOD2 | 0.450 | 2.3E−24 | 2.5E−24 |
| ARTN | 0.441 | 2.0E−23 | 2.1E−23 |
| GAMT | 0.431 | 2.5E−22 | 2.6E−22 |
| ATP6V1D | 0.391 | 2.4E−18 | 2.5E−18 |
| PRKCQ | 0.386 | 7.1E−18 | 7.4E−18 |
| GGA1 | 0.373 | 1.2E−16 | 1.2E−16 |
| JUN | 0.359 | 1.8E−15 | 1.8E−15 |
| EIF5A | 0.352 | 6.3E−15 | 6.5E−15 |
| IL33 | 0.279 | 1.1E−09 | 1.1E−09 |
| TNF | 0.261 | 1.3E−08 | 1.3E−08 |
| ARNT | 0.258 | 1.8E−08 | 1.8E−08 |
| JCHAIN | −0.131 | 0.005 | 0.005 |
| IL4 | 0.085 | 0.068 | 0.068 |
| TF | −0.049 | 0.290 | 0.291 |
| IL2 | 0.046 | 0.326 | 0.326 |

TABLE 15
Validation of 1-5 Y lung cancer prediction model in UK Biobank data

| | PPV (%) at | | | enrichment | | | | |
|---|---|---|---|---|---|---|---|---|
| | sensitivity of: | | | at 0.05 | | Population | | Prevalence |
| Histological subtype | 0.05 | 0.10 | 0.25 | sensitivity | AUC | Size | Cases | in subgroup |
| Adenocarcinoma | 18.6 | 9.6 | 4.6 | 7.7 | 0.652 | 6440 | 157 | 2.43 |
| Non-small cell carcinoma | 12.5 | 4 | 1.4 | 19.8 | 0.713 | 6323 | 40 | 0.63 |
| Small cell carcinoma | 14.3 | 10.3 | 4 | 13.8 | 0.692 | 6349 | 66 | 1.04 |
| Squamous cell carcinoma | 11.6 | 8.7 | 4.2 | 7.5 | 0.697 | 6382 | 99 | 1.55 |
| Carcinoid | 6.7 | 2.2 | 0.7 | 18.6 | 0.621 | 6306 | 23 | 0.36 |
| Unspecified | 17.6 | 5.5 | 3.3 | 17.8 | 0.718 | 6346 | 63 | 0.99 |
| Large cell carcinoma | 6.7 | 6.7 | 1 | 30.5 | 0.683 | 6297 | 14 | 0.22 |

TABLE 16
Pathway enrichment for 1-3 Y and 1-5 Y proteins - upregulated proteins

| | P value | P value | | |
|---|---|---|---|---|
| Pathway label | 1-5 Y up | 1-3 Y up | Hits 1-5 Y | Hits 1-3 Y |
| GOBP_ENDOTHELIAL_CELL_MATRIX_ADHESION | 0.0003 | 0.0006 | CEACAM6, MMP12, RRAS | CEACAM6, MMP12, RRAS |
| GOBP_REGULATION_OF_COMPLEMENT_ACTIVATION | 1 | 0.0003 | | C4BPB, C5, C9, CFB, CFH, CFI, CLU, |
| | | | | SERPING1 |
| GOBP_COMPLEMENT_ACTIVATION | 0.51 | 0.0003 | C1RL, FCN1 | C4BPB, C5, C9, CFB, CFH, CFI, CLU, |
| | | | | FCN1, SERPING1 |
| GOBP_DEFENSE_RESPONSE_TO_OTHER_ORGANISM | 0.29 | 0.0005 | APOL1, BPIFB1, BST2, C1RL, | APOL1, BPIFB1, C4BPB, C5, C9, CCL19, |
| | | | CCL20, CXCL13, FCN1, HLA-DRA, | CD14, CFB, CFH, CFI, CLU, CRISP3, |
| | | | IL6, KYNU, LILRA2, LILRA5, | CXCL13, FCN1, FGA, GBP4, GRN, |
| | | | MARCO, MMP12, RNASET2, SIRPB1, | IFNAR1, IL17RA, IL36A, IL6, |
| | | | UMOD | KIR2DS4, KYNU, LBP, LILRA2, LILRA5, |
| | | | | MARCO, MMP12, MRC1, MUC16, |
| | | | | SERPING1, SHC1, TRAF3IP2, UMOD |
| GOBP_REGULATION_OF_HUMORAL_IMMUNE_RESPONSE | 0.85 | 0.0006 | CXCL13 | C4BPB, C5, C9, CFB, CFH, CFI, CLU, |
| | | | | CXCL13, SERPING1 |
| GOBP_INNATE_IMMUNE_RESPONSE | 0.3 | 0.0007 | APOL1, BPIFB1, BST2, C1RL, CCL20, | APOL1, BPIFB1, C4BPB, C5, C9, CCL19, |
| | | | FCN1, HLA-DRA, KYNU, LILRA2, | CD14, CFB, CFH, CFI, CLU, CRISP3, |
| | | | LILRA5, MARCO, MMP12, RNASET2, | FCN1, FGA, GBP4, GRN, IFNAR1, |
| | | | SIRPB1 | IL17RA, IL36A, KIR2DS4, KYNU, |
| | | | | LBP, LILRA2, LILRA5, MARCO, |
| | | | | MMP12, MRC1, MUC16, SERPING1 |
| GOBP_HETEROPHILIC_CELL_CELL_ADHESION_VIA_PLASMA_MEMBRANE_{—} | 0.0007 | 0.06 | CBLN1, CEACAM5, CEACAM6, | CEACAM5, CEACAM6, NECTIN1, |
| CELL_ADHESION_MOLECULES | | | IGSF21, NECTIN1, UMOD | UMOD |
| GOBP_NEGATIVE_REGULATION_OF_MULTI_ORGANISM_PROCESS | 0.0012 | 0.037 | BST2, LILRA2, PAEP | LILRA2, PAEP |
| GOBP_REGULATION_OF_TOLL_LIKE_RECEPTOR_4_SIGNALING_PATHWAY | 0.015 | 0.0014 | BPIFB1, LILRA2 | BPIFB1, LBP, LILRA2 |
| GOBP_DEFENSE_RESPONSE | 0.49 | 0.0017 | APOL1, BPIFB1, BST2, C1RL, CCL20, | APOL1, BPIFB1, C4BPB, C5, C9, |
| | | | CSF3R, CXCL13, FCN1, GBA, HLA- | CCL19, CD14, CFB, CFH, CFI, CLU, |
| | | | DRA, IL17C, IL6, IL9, JAM3, KYNU, | CRHBP, CRISP3, CST7, CXCL13, FCN1, |
| | | | LILRA2, LILRA5, MARCO, MMP12, | FGA, GBA, GBP4, GRN, IFNAR1, |
| | | | PROC, RNASET2, SIRPB1, UMOD | IL17RA, IL36A, IL6, IL9, ITIH4, |
| | | | | KIR2DS4, KLKB1, KYNU, LBP, LILRA2, |
| | | | | LILRA5, MARCO, MMP12, MRC1, MUC16, |
| | | | | OSMR, RICTOR, SDC1, SERPINA3, |
| | | | | SERPINC1, SERPING1, SHC1, TRAF3IP2, |
| | | | | UMOD |
| GOBP_OPSONIZATION | 0.22 | 0.0027 | SFTPA1 | C4BPB, LBP, SFTPA1 |
| GOBP_VASCULAR_ASSOCIATED_SMOOTH_MUSCLE_CELL_MIGRATION | 0.022 | 0.0027 | FGF9, PRKG1 | ADAMTS1, FGF9, PRKG1 |
| GOBP_PROTEIN_ACTIVATION_CASCADE | 1 | 0.0034 | | F11, FGA, KLKB1, SERPINC1, SERPING1 |
| GOBP_HUMORAL_IMMUNE_RESPONSE_MEDIATED_BY_CIRCULATING_{—} | 0.71 | 0.0044 | C1RL | C4BPB, C5, C9, CFI, CLU, SERPING1 |
| IMMUNOGLOBULIN | | | | |
| GOBP_NEGATIVE_REGULATION_OF_SMOOTH_MUSCLE_CELL_{—} | 0.03 | 0.0045 | FGF9, RBPMS2 | FGF9, RBPMS2, SHH |
| DIFFERENTIATION | | | | |
| GOBP_PROTEIN_PEPTIDYL_PROLYL_ISOMERIZATION | 0.0045 | 0.081 | FKBP14, FKBP7, PPIF | FKBP14, PPIF |
| GOBP_NEGATIVE_REGULATION_OF_TOLL_LIKE_RECEPTOR_4_SIGNALING_{—} | 0.0047 | 0.0083 | BPIFB1, LILRA2 | BPIFB1, LILRA2 |
| PATHWAY | | | | |
| GOBP_POSITIVE_REGULATION_OF_ENDOTHELIAL_CELL_MATRIX_{—} | 0.0047 | 0.0083 | CEACAM6, RRAS | CEACAM6, RRAS |
| ADHESION_VIA_FIBRONECTIN | | | | |
| GOBP_BLOOD_COAGULATION_INTRINSIC_PATHWAY | 1 | 0.0053 | | F11, KLKB1, SERPINC1, SERPING1 |
| GOBP_COMPLEMENT_ACTIVATION_ALTERNATIVE_PATHWAY | 1 | 0.0053 | | C5, C9, CFB, CFH |
| GOBP_TOLL_LIKE_RECEPTOR_4_SIGNALING_PATHWAY | 0.11 | 0.0053 | BPIFB1, LILRA2 | BPIFB1, CD14, LBP, LILRA2 |
| GOBP_HUMORAL_IMMUNE_RESPONSE | 0.38 | 0.0054 | BPIFB1, BPIFB2, C1RL, CXCL13, | BPIFB1, C4BPB, C5, C9, CFB, CFH, |
| | | | FCN1, IL6 | CFI, CLU, CXCL13, FCN1, FGA, IL6, |
| | | | | SERPING1, TRAF3IP2 |
| GOBP_CELL_RECOGNITION | 0.25 | 0.0058 | FCN1, NEXN, PAEP, SFTPA1 | C4BPB, CCL19, FCN1, IZUMO1, LBP, |
| | | | | NEXN, NRCAM, PAEP, SFTPA1 |
| GOBP_FATTY_ACID_DERIVATIVE_METABOLIC_PROCESS | 0.0063 | 0.097 | OXCT1, PPT1, PTGR1 | PPT1, PTGR1 |
| GOBP_NEGATIVE_REGULATION_OF_MUSCLE_CELL_DIFFERENTIATION | 0.021 | 0.0069 | CEACAM5, FGF9, RBPMS2 | CEACAM5, FGF9, RBPMS2, SHH |
| GOBP_PHAGOCYTOSIS_RECOGNITION | 0.12 | 0.0069 | FCN1, SFTPA1 | C4BPB, FCN1, LBP, SFTPA1 |
| GOBP_ENDOCYTOSIS | 0.39 | 0.0072 | APOL1, CALY, FCN1, LRP2, LRPAP1, | ANK2, APOL1, C4BPB, CALY, CCL19, |
| | | | MARCO, PPT1, SCGB3A2, SFTPA1 | CD14, CFI, CLU, DLG4, DNM3, FCN1, |
| | | | | LBP, MARCO, MRC1, PPT1, SCGB3A2, |
| | | | | SFTPA1, SHH, SSC4D |
| GOBP_POSITIVE_REGULATION_OF_TUMOR_NECROSIS_FACTOR_{—} | 0.26 | 0.0081 | IL6, LILRA2, LILRA5 | CCL19, CD14, CLU, IL6, LBP, LILRA2, |
| SUPERFAMILY_CYTOKINE_PRODUCTION | | | | LILRA5 |
| GOBP_COBALT_ION_TRANSPORT | 1 | 0.0083 | | CBL1F, TCN2 |
| GOBP_ETHANOL_CATABOLIC_PROCESS | 1 | 0.0083 | | ALDH2, SULT2A1 |
| GOBP_NUCLEOSIDE_BISPHOSPHATE_METABOLIC_PROCESS | 0.13 | 0.0088 | KYNU, PPT1 | KYNU, MCEE, PPT1, SULT2A1 |
| GOBP_REGULATION_OF_POSTSYNAPSE_ORGANIZATION | 0.0089 | 0.1 | CBLN1, GRIN2B, LRFN2, PDLIM5 | DNM3, LRFN2, PDLIM5 |
| GOBP_REGULATION_OF_LYSOSOMAL_PROTEIN_CATABOLIC_PROCESS | 0.0092 | 0.2 | GBA, LRP2 | GBA |
| GOBP_REGULATION_OF_PROTEIN_CATABOLIC_PROCESS_IN_THE_{—} | 0.0092 | 0.2 | GBA, LRP2 | GBA |
| VACUOLE | | | | |
| GOBP_WATER_HOMEOSTASIS | 0.049 | 0.01 | GBA, UMOD | GBA, SCT, UMOD |
| GOBP_CYTOLYSIS | 0.51 | 0.011 | APOL1 | APOL1, C5, C9, LBP |
| GOBP_PEPTIDYL_PROLINE_MODIFICATION | 0.011 | 0.13 | FKBP14, FKBP7, PPIF | FKBP14, PPIF |
| GOBP_PATTERN_RECOGNITION_RECEPTOR_SIGNALING_PATHWAY | 0.088 | 0.012 | BPIFB1, FCN1, LILRA2, SFTPA1, | BPIFB1, CD14, FCN1, FGA, LBP, |
| | | | SFTPA2 | LILRA2, SFTPA1, SFTPA2 |
| GOBP_REGULATION_OF_BODY_FLUID_LEVELS | 0.39 | 0.012 | GBA, IL6, NOS3, PRKG1, PROC, | ADRA2A, C4BPB, ERBB4, F11, FGA, |
| | | | SERPIND1, UMOD | GBA, IL6, KLKB1, PRKG1, SCT, |
| | | | | SERPINC1, SERPIND1, SERPING1, |
| | | | | SHH, UMOD |
| GOBP_MOLTING_CYCLE | 0.022 | 0.013 | FGF7, KRT14, LRIG1, TRPV3 | DSG4, FGF7, KRT14, LRIG1, SHH |
| GOBP_DENDRITIC_SPINE_DEVELOPMENT | 0.16 | 0.014 | PAK4, PDLIM5 | DLG4, DNM3, PAK4, PDLIM5 |
| GOBP_DENDRITIC_SPINE_MORPHOGENESIS | 0.34 | 0.014 | PDLIM5 | DLG4, DNM3, PDLIM5 |
| GOBP_REGULATION_OF_MICROGLIAL_CELL_ACTIVATION | 0.34 | 0.014 | IL6 | CST7, GRN, IL6 |
| GOBP_KETONE_CATABOLIC_PROCESS | 0.015 | 0.24 | KYNU, OXCT1 | KYNU |
| GOBP_DOPAMINE_RECEPTOR_SIGNALING_PATHWAY | 0.015 | 0.24 | CALY, RGS8 | CALY |
| GOBP_COBALAMIN_TRANSPORT | 1 | 0.016 | | CBLIF, TCN2 |
| GOBP_EMBRYONIC_DIGESTIVE_TRACT_MORPHOGENESIS | 0.15 | 0.016 | RBPMS2 | RBPMS2, SHH |
| GOBP_LYSOSOMAL_LUMEN_ACIDIFICATION | 0.15 | 0.016 | PPT1 | GRN, PPT1 |
| GOBP_POSITIVE_REGULATION_OF_VASCULAR_ASSOCIATED_SMOOTH_{—} | 0.15 | 0.016 | FGF9 | ADAMTS1, FGF9 |
| MUSCLE_CELL_MIGRATION | | | | |
| GOBP_REGULATION_OF_LYSOSOMAL_LUMEN_PH | 0.15 | 0.016 | PPT1 | GRN, PPT1 |
| GOBP_RESPIRATORY_BURST_INVOLVED_IN_INFLAMMATORY_RESPONSE | 1 | 0.016 | | GRN, LBP |
| GOBP_VACUOLAR_ACIDIFICATION | 0.15 | 0.016 | PPT1 | GRN, PPT1 |
| GOBP_FIBRINOLYSIS | 1 | 0.016 | | F11, FGA, KLKB1, SERPING1 |
| GOBP_POSITIVE_REGULATION_OF_INTERLEUKIN_23_PRODUCTION | 1 | 0.016 | | CSF2, IL17RA |
| GOBP_EMBRYONIC_DIGESTIVE_TRACT_DEVELOPMENT | 0.37 | 0.018 | RBPMS2 | RBPMS2, SCT, SHH |
| GOBP_TOLL_LIKE_RECEPTOR_SIGNALING_PATHWAY | 0.15 | 0.018 | BPIFB1, LILRA2, SFTPA1, SFTPA2 | BPIFB1, CD14, FGA, LBP, LILRA2, |
| | | | | SFTPA1, SFTPA2 |
| GOBP_ACUTE_INFLAMMATORY_RESPONSE | 0.88 | 0.018 | IL6 | IL6, ITIH4, KLKB1, LBP, OSMR, |
| | | | | SERPINA3, SERPINC1 |
| GOBP_ACID_SECRETION | 0.07 | 0.018 | SV2A, UMOD | SCT, SV2A, UMOD |
| GOBP_RESPONSE_TO_BIOTIC_STIMULUS | 0.59 | 0.019 | APOL1, BPIFB1, BPIFB2, BST2, | APOL1, BPIFB1, C4BPB, C5, C9, |
| | | | C1RL, CCL20, CXCL13, FCN1, HLA- | CCL19, CD14, CFB, CFH, CFI, CLU, |
| | | | DRA, IL6, KYNU, LILRA2, LILRA5, | CRISP3, CSF2, CXCL13, FCN1, FGA, |
| | | | MARCO, MMP12, NOS3, RNASET2, | GBP4, GRN, IFNAR1, IL17RA, IL36A, |
| | | | SIRPB1, UMOD | IL6, KIR2DS4, KYNU, LBP, LILRA2, |
| | | | | LILRA5, LRG1, MARCO, MMP12, |
| | | | | MRC1, MUC16, SERPING1, SHC1, |
| | | | | TRAF3IP2, UMOD |
| GOBP_REGULATION_OF_VASCULAR_ASSOCIATED_SMOOTH_MUSCLE_{—} | 0.045 | 0.02 | ERN1, FGF9, PRKG1 | ADAMTS1, ERN1, FGF9, PRKG1 |
| CELL_PROLIFERATION | | | | |
| GOBP_ORGAN_OR_TISSUE_SPECIFIC_IMMUNE_RESPONSE | 0.021 | 0.043 | BPIFB1, IL6, UMOD | BPIFB1, IL6, UMOD |
| GOBP_LYSOSOMAL_PROTEIN_CATABOLIC_PROCESS | 0.022 | 0.28 | GBA, LRP2 | GBA |
| GOBP_NEUTROPHIL_HOMEOSTASIS | 0.022 | 0.28 | IL6, JAM3 | IL6 |
| GOBP_REGULATION_OF_INTEGRIN_ACTIVATION | 0.022 | 0.28 | CXCL13, JAM3 | CXCL13 |
| GOBP_MIDBRAIN_DEVELOPMENT | 0.082 | 0.023 | COX6B1, FGF9 | COX6B1, FGF9, SHH |
| GOBP_THIOESTER_METABOLIC_PROCESS | 0.082 | 0.023 | KYNU, PPT1 | KYNU, MCEE, PPT1 |
| GOBP_REGULATION_OF_TOLL_LIKE_RECEPTOR_SIGNALING_PATHWAY | 0.21 | 0.023 | BPIFB1, LILRA2 | BPIFB1, CD14, LBP, LILRA2 |
| GOBP_EMBRYONIC_PATTERN_SPECIFICATION | 1 | 0.023 | | ERBB4, SHH, SMAD5 |
| GOBP_CELLULAR_RESPONSE_TO_POTASSIUM_ION | 1 | 0.026 | | CRHBP, DLG4 |
| GOBP_PRIMARY_ALCOHOL_CATABOLIC_PROCESS | 1 | 0.026 | | ALDH2, SULT2A1 |
| GOBP_RESPIRATORY_BURST_INVOLVED_IN_DEFENSE_RESPONSE | 1 | 0.026 | | GRN, LBP |
| GOBP_REGULATION_OF_COAGULATION | 0.25 | 0.026 | NOS3, PRKG1, PROC | F11, FGA, KLKB1, PRKG1, SERPINC1, |
| | | | | SERPING1 |
| GOBP_NEGATIVE_REGULATION_OF_MICROGLIAL_CELL_ACTIVATION | 1 | 0.026 | | CST7, GRN |
| GOBP_INTERLEUKIN_23_PRODUCTION | 1 | 0.026 | | CSF2, IL17RA |
| GOBP_SPHINGOSINE_BIOSYNTHETIC_PROCESS | 0.19 | 0.026 | GBA | ASAH1, GBA |
| GOBP_CELLULAR_RESPONSE_TO_VIRUS | 0.095 | 0.029 | IL6, MMP12 | CCL19, IL6, MMP12 |
| GOBP_POSITIVE_REGULATION_OF_VASCULAR_ASSOCIATED_SMOOTH_{—} | 0.095 | 0.029 | ERN1, FGF9 | ADAMTS1, ERN1, FGF9 |
| MUSCLE_CELL_PROLIFERATION | | | | |
| GOBP_REGULATION_OF_SMOOTH_MUSCLE_CELL_DIFFERENTIATION | 0.095 | 0.029 | FGF9, RBPMS2 | FGF9, RBPMS2, SHH |
| GOBP_NEGATIVE_REGULATION_OF_TOLL_LIKE_RECEPTOR_SIGNALING_{—} | 0.095 | 0.029 | BPIFB1, LILRA2 | BPIFB1, CD14, LILRA2 |
| PATHWAY | | | | |
| GOBP_CEREBELLAR_CORTEX_FORMATION | 0.03 | 0.32 | CBLN1, GBA | GBA |
| GOBP_POSITIVE_REGULATION_OF_ACTIN_NUCLEATION | 0.03 | 0.32 | BRK1, SCIN | BRK1 |
| GOBP_PROTEIN_CATABOLIC_PROCESS_IN_THE_VACUOLE | 0.03 | 0.32 | GBA, LRP2 | GBA |
| GOBP_SUBSTANTIA_NIGRA_DEVELOPMENT | 0.03 | 0.05 | COX6B1, FGF9 | COX6B1, FGF9 |
| GOBP_POSITIVE_REGULATION_OF_CYTOKINE_PRODUCTION | 0.45 | 0.03 | FCN1, IL12RB2, IL6, IL9, LILRA2, | ADRA2A, C5, CCL19, CD14, CLU, |
| | | | LILRA5, MMP12, PAEP | CSF2, FCN1, IL17RA, IL6, IL9, LBP, |
| | | | | LILRA2, LILRA5, MMP12, PAEP, |
| | | | | POSTN |
| GOBP_RESPONSE_TO_INORGANIC_SUBSTANCE | 0.36 | 0.03 | AOC1, ERN1, IL6, KRT14, LONP1, | AOC1, CCL19, CD14, CRHBP, CSF2, |
| | | | NOS3, PPIF, UMOD | DLG4, ERN1, FGA, IL6, KRT14, |
| | | | | LONP1, PPIF, SDC1, SHH, UMOD |
| GOBP_PLACENTA_BLOOD_VESSEL_DEVELOPMENT | 0.03 | 0.32 | SPINT1, VASH1 | SPINT1 |
| GOBP_RECEPTOR_MEDIATED_ENDOCYTOSIS | 0.15 | 0.032 | APOL1, CALY, LRP2, LRPAP1, | APOL1, CALY, CCL19, CD14, CLU, |
| | | | MARCO, PPT1, SCGB3A2 | DLG4, DNM3, MARCO, MRC1, PPT1, |
| | | | | SCGB3A2 |
| GOBP_PROTEIN_LOCALIZATION_TO_CELL_SURFACE | 0.063 | 0.032 | FCN1, FGF7, JAM3 | ANK2, ERBB4, FCN1, FGF7 |
| GOBP_MUSCLE_CELL_PROLIFERATION | 0.15 | 0.032 | ERN1, FGF9, IL6, PRKG1, RBPMS2 | ADAMTS1, ERBB4, ERN1, FGF9, IL6, |
| | | | | PRKG1, RBPMS2, SHH |
| GOBP_POSITIVE_REGULATION_OF_CHEMOKINE_PRODUCTION | 0.76 | 0.033 | IL6 | C5, IL17RA, IL6, LBP, POSTN |
| GOBP_REGULATION_OF_MULTI_ORGANISM_PROCESS | 0.034 | 0.25 | BST2, LILRA2, PAEP | LILRA2, PAEP |
| GOBP_REGULATION_OF_SYNAPSE_STRUCTURE_OR_ACTIVITY | 0.035 | 0.23 | CBLN1, GRIN2B, LRFN2, NECTIN1, | DNM3, LRFN2, NECTIN1, PDLIM5, |
| | | | PDLIM5, PPT1 | PPT1 |
| GOBP_LYTIC_VACUOLE_ORGANIZATION | 0.11 | 0.036 | GBA, PPT1 | GBA, GRN, PPT1 |
| GOBP_CHAPERONE_MEDIATED_PROTEIN_COMPLEX_ASSEMBLY | 0.22 | 0.037 | LONP1 | CLU, LONP1 |
| GOBP_ETHANOL_METABOLIC_PROCESS | 1 | 0.037 | | ALDH2, SULT2A1 |
| GOBP_NEGATIVE_REGULATION_OF_RELEASE_OF_CYTOCHROME_C_FROM_{—} | 0.22 | 0.037 | PPIF | CLU, PPIF |
| MITOCHONDRIA | | | | |
| GOBP_POSTSYNAPTIC_NEUROTRANSMITTER_RECEPTOR_{—} | 0.22 | 0.037 | CALY | CALY, DNM3 |
| INTERNALIZATION | | | | |
| GOBP_RESPONSE_TO_LIPOTEICHOIC_ACID | 1 | 0.037 | | CD14, LBP |
| GOBP_RESPONSE_TO_POTASSIUM_ION | 1 | 0.037 | | CRHBP, DLG4 |
| GOBP_CERAMIDE_CATABOLIC_PROCESS | 0.22 | 0.037 | GBA | ASAH1, GBA |
| GOBP_COMPLEMENT_ACTIVATION_LECTIN_PATHWAY | 0.22 | 0.037 | FCN1 | FCN1, SERPING1 |
| GOBP_REGULATION_OF_RESPIRATORY_BURST | 1 | 0.037 | | GRN, LBP |
| GOBP_SPHINGOID_METABOLIC_PROCESS | 0.22 | 0.037 | GBA | ASAH1, GBA |
| GOBP_POSITIVE_REGULATION_OF_INTERLEUKIN_1_PRODUCTION | 0.07 | 0.037 | IL6, LILRA2, LILRA5 | CCL19, IL6, LILRA2, LILRA5 |
| GOBP_NEGATIVE_REGULATION_OF_ANOIKIS | 0.039 | 0.065 | CEACAM5, CEACAM6 | CEACAM5, CEACAM6 |
| GOBP_NEGATIVE_REGULATION_OF_VIRAL_LIFE_CYCLE | 0.039 | 0.36 | BST2, FCN1 | FCN1 |
| GOBP_NEURAL_NUCLEUS_DEVELOPMENT | 0.039 | 0.065 | COX6B1, FGF9 | COX6B1, FGF9 |
| GOBP_VASODILATION | 0.039 | 0.36 | NOS3, PRKG1 | PRKG1 |
| GOBP_GLYCOPROTEIN_CATABOLIC_PROCESS | 0.039 | 0.36 | EDEM2, MMP12 | MMP12 |
| GOBP_ADHERENS_JUNCTION_ASSEMBLY | 0.04 | 1 | JAM3 | |
| GOBP_CELLULAR_RESPONSE_TO_HISTAMINE | 0.04 | 0.054 | AOC1 | AOC1 |
| GOBP_CGMP_CATABOLIC_PROCESS | 0.04 | 0.054 | PDE5A | PDE5A |
| GOBP_CITRATE_TRANSPORT | 0.04 | 0.054 | UMOD | UMOD |
| GOBP_EXCITATORY_CHEMICAL_SYNAPTIC_TRANSMISSION | 0.04 | 1 | GRIN2B | |
| GOBP_GLUCOSYLCERAMIDE_METABOLIC_PROCESS | 0.04 | 0.054 | GBA | GBA |
| GOBP_INTERLEUKIN_21_PRODUCTION | 0.04 | 0.054 | IL6 | IL6 |
| GOBP_LEUKOTRIENE_B4_METABOLIC_PROCESS | 0.04 | 0.054 | PTGR1 | PTGR1 |
| GOBP_LIPOXIN_METABOLIC_PROCESS | 0.04 | 0.054 | PTGR1 | PTGR1 |
| GOBP_MEMBRANE_REPOLARIZATION_DURING_VENTRICULAR_CARDIAC_{—} | 0.04 | 1 | KCNH2 | |
| MUSCLE_CELL_ACTION_POTENTIAL | | | | |
| GOBP_MHC_PROTEIN_COMPLEX_ASSEMBLY | 0.04 | 1 | HLA-DRA | |
| GOBP_MRNA_CLEAVAGE_INVOLVED_IN_MRNA_PROCESSING | 0.04 | 0.054 | ERN1 | ERN1 |
| GOBP_NEGATIVE_REGULATION_OF_CELL_CHEMOTAXIS_TO_FIBROBLAST_{—} | 0.04 | 0.054 | CXCL13 | CXCL13 |
| GROWTH_FACTOR | | | | |
| GOBP_POSITIVE_REGULATION_OF_ACTION_POTENTIAL | 0.04 | 0.054 | GBA | GBA |
| GOBP_POTASSIUM_ION_EXPORT_ACROSS_PLASMA_MEMBRANE | 0.04 | 1 | KCNH2 | |
| GOBP_QUINOLINATE_METABOLIC_PROCESS | 0.04 | 0.054 | KYNU | KYNU |
| GOBP_REGULATION_OF_PHENOTYPIC_SWITCHING | 0.04 | 0.054 | FGF9 | FGF9 |
| GOBP_REGULATION_OF_POTASSIUM_ION_EXPORT_ACROSS_PLASMA_{—} | 0.04 | 1 | KCNH2 | |
| MEMBRANE | | | | |
| GOBP_RENAL_SODIUM_ION_ABSORPTION | 0.04 | 0.054 | UMOD | UMOD |
| GOBP_RESPONSE_TO_HISTAMINE | 0.04 | 0.054 | AOC1 | AOC1 |
| GOBP_T_FOLLICULAR_HELPER_CELL_DIFFERENTIATION | 0.04 | 0.054 | IL6 | IL6 |
| GOBP_TYPE_B_PANCREATIC_CELL_APOPTOTIC_PROCESS | 0.04 | 0.054 | IL6 | IL6 |
| GOBP_UBIQUITIN_DEPENDENT_GLYCOPROTEIN_ERAD_PATHWAY | 0.04 | 1 | EDEM2 | |
| GOBP_URATE_METABOLIC_PROCESS | 0.04 | 0.054 | UMOD | UMOD |
| GOBP_NEGATIVE_REGULATION_OF_COAGULATION | 0.18 | 0.041 | NOS3, PRKG1, PROC | F11, FGA, KLKB1, PRKG1, SERPING1 |
| GOBP_PLASMINOGEN_ACTIVATION | 1 | 0.043 | | F11, FGA, KLKB1 |
| GOBP_POSITIVE_REGULATION_OF_SMOOTH_MUSCLE_CELL_MIGRATION | 0.46 | 0.043 | FGF9 | ADAMTS1, FGF9, POSTN |
| GOBP_MEMBRANE_LIPID_CATABOLIC_PROCESS | 0.12 | 0.043 | GBA, PPT1 | ASAH1, GBA, PPT1 |
| GOBP_RESPONSE_TO_FIBROBLAST_GROWTH_FACTOR | 0.043 | 0.26 | CXCL13, DLL4, FGF7, FGF9, | CXCL13, FGF7, FGF9, POSTN |
| | | | POLR2A | |
| GOBP_CELLULAR_MACROMOLECULE_CATABOLIC_PROCESS | 0.044 | 0.33 | CTSO, EDEM2, ERN1, GBA, IL6, | CLU, CTSH, ERN1, GBA, GRSF1, |
| | | | LONP1, LRP2, MMP12, NELL1, | IL6, LONP1, MMP12, NELL1, RNF43, |
| | | | PTTG1, RNASET2, RNF43, UMOD | SHH, UMOD |
| GOBP_INFLAMMATORY_RESPONSE | 0.78 | 0.044 | CCL20, CXCL13, GBA, IL17C, IL6, | C5, CCL19, CD14, CRHBP, CST7, |
| | | | IL9, JAM3, LILRA5, PROC, UMOD | CXCL13, GBA, GRN, IL17RA, IL36A, |
| | | | | IL6, IL9, ITIH4, KLKB1, LBP, |
| | | | | LILRA5, OSMR, RICTOR, SDC1, |
| | | | | SERPINA3, SERPINC1, TRAF3IP2, |
| | | | | UMOD |
| GOBP_AMYLOID_BETA_CLEARANCE | 0.045 | 0.29 | LRP2, LRPAP1, MARCO | CLU, MARCO |
| GOBP_IMPORT_ACROSS_PLASMA_MEMBRANE | 0.045 | 1 | KCNH2, LRP2, TRPV3 | |
| GOBP_COAGULATION | 0.51 | 0.045 | IL6, NOS3, PRKG1, PROC, | ADRA2A, C4BPB, F11, FGA, IL6, |
| | | | SERPIND1 | KLKB1, PRKG1, SERPINC1, SERPIND1, |
| | | | | SERPING1, SHH |
| GOBP_POSITIVE_REGULATION_OF_INTERLEUKIN_6_PRODUCTION | 0.19 | 0.046 | IL6, LILRA2, LILRA5 | IL17RA, IL6, LBP, LILRA2, LILRA5 |
| GOBP_HEAD_DEVELOPMENT | 0.048 | 0.2 | CBLN1, COX6B1, FGF9, GBA, | COX6B1, ERBB4, FGF9, GBA, PPT1, |
| | | | GRIN2B, LRP2, OXCT1, PPT1, | PRKG1, RRAS, SCT, SHH, SOX2 |
| | | | PRKG1, RRAS | |
| GOBP_REGULATION_OF_PATTERN_RECOGNITION_RECEPTOR_SIGNALING_{—} | 0.28 | 0.048 | BPIFB1, LILRA2 | BPIFB1, CD14, LBP, LILRA2 |
| PATHWAY | | | | |
| GOBP_AMELOGENESIS | 0.049 | 0.39 | CSF3R, NECTIN1 | NECTIN1 |
| GOBP_POSITIVE_REGULATION_OF_RNA_SPLICING | 0.049 | 0.39 | ERN1, POLR2A | ERN1 |
| GOBP_REGULATION_OF_ANOIKIS | 0.049 | 0.081 | CEACAM5, CEACAM6 | CEACAM5, CEACAM6 |
| GOBP_TRANSCYTOSIS | 0.049 | 1 | LRP2, LRPAP1 | |
| GOBP_MACROPHAGE_ACTIVATION | 0.88 | 0.049 | IL6 | CLU, CSF2, CST7, GRN, IL6, LBP |
| GOBP_EMBRYO_DEVELOPMENT | 0.67 | 0.049 | BRK1, DLL4, LRIG1, LRP2, | BRK1, C5, CMIP, CSF2, ERBB4, |
| | | | RBPMS2, SPINT1,_VASH1 | GRSF1, IL3, LRIG1, RBPMS2, RICTOR, |
| | | | | SCT, SHH, SMAD5, SOX2, SPINT1, |
| | | | | UGDH |

TABLE 17
Pathway enrichment for 1-3 Y and 1-5 Y proteins - downregulated proteins

| | P value | P value | | |
|---|---|---|---|---|
| Pathway label | 1-5 Y down | 1-3 Y down | Hits 1-5 Y | Hits 1-3 Y |
| GOBP_NEUROPEPTIDE_SIGNALING_PATHWAY | 0.00016 | 0.000096 | AGRP, CPE, GPR37, NPY, PPY, PYY | AGRP, GPR37, NPY, POMC, PPY |
| GOBP_FEEDING_BEHAVIOR | 0.00073 | 0.00034 | AGRP, GCG, INSL5, NPY, PPY, PYY | AGRP, GCG, NPY, OXT, PPY |
| GOBP_MEMORY | 0.11 | 0.00034 | GIP, NTF3, NTF4 | GIP, MAPT, NGF, NTF4, OXT |
| GOBP_BEHAVIOR | 0.00089 | 0.0036 | ADGRB3, AGRP, DSCAM, GCG, GIP, | AGRP, GCG, GIP, GPR37, MAPT, |
| | | | GPR37, INSL5, NPY, NTF3, NTF4, PPY, | NGF, NPY, NTF4, OXT, PPY |
| | | | PTPRZ1, PYY, SLURP1, SNCG, TNR | |
| GOBP_MUSCLE_CELL_DEVELOPMENT | 0.002 | 0.28 | ACTN2, COMP, LMOD1, PDGFRB, | PDGFRB, WFIKKN2 |
| | | | PI16, TMOD4, WFIKKN2 | |
| GOBP_AMINOGLYCAN_BIOSYNTHETIC_PROCESS | 0.002 | 0.082 | AGRN, BGN, CSPG4, CSPG5, GPC1, | CSPG5, HS6ST1, PDGFRB |
| | | | HS6ST1, PDGFRB | |
| GOBP_CELLULAR_COMPONENT_ASSEMBLY_INVOLVED_IN_MORPHOGENESIS | 0.0022 | 0.18 | ACTN2, GPC1, LMOD1, PDGFRB, | PDGFRB, PHLDB2 |
| | | | PHLDB2, TMOD4 | |
| GOBP_RESPONSE_TO_FOOD | 0.4 | 0.0022 | NPY | GAST, NPY, OXT |
| GOBP_MULTI_MULTICELLULAR_ORGANISM_PROCESS | 0.25 | 0.0023 | AGRP, CD38, DKKL1, ENDOU, GIP | AGRP, DKKL1, ENDOU, EPO, GIP, |
| | | | | OXT, PAPPA |
| GOBP_BLASTODERM_SEGMENTATION | 1 | 0.0023 | | SEMA3F, TDGF1 |
| GOBP_ERYTHROCYTE_MATURATION | 1 | 0.0023 | | BRD1, EPO |
| GOBP_INACTIVATION_OF_MAPK_ACTIVITY | 0.14 | 0.0023 | DUSP29 | DUSP29, DUSP3 |
| GOBP_CELL_CELL_SIGNALING | 0.069 | 0.0027 | AGRN, CCL24, CCN5, CD38, CPE, | CCL24, CSPG5, DKKL1, FAM3D, |
| | | | CSPG5, CX3CL1, DKK4, DKKL1, FAM3D, | FASLG, FGF16, FGF23, FZD8, GCG, |
| | | | FASLG, FGFBP2, GCG, GIP, IGFBP6, | GIP, IL17A, MAPT, NGF, NPY, NTF4, |
| | | | NPY, NTF3, NTF4, RSPO3, SCGN, | OXT, POMC, RSPO3, SCGN, TMEM25, |
| | | | SCN3B, SIGLEC6, SNCG, SOST, | WNT9A |
| | | | TMEM25, TNR, YAP1 | |
| GOBP_STRIATED_MUSCLE_CELL_DEVELOPMENT | 0.0027 | 0.19 | ACTN2, COMP, LMOD1, PDGFRB, | PDGFRB, WFIKKN2 |
| | | | TMOD4, WFIKKN2 | |
| GOBP_LOCOMOTORY_BEHAVIOR | 0.0028 | 0.093 | DSCAM, GIP, GPR37, NTF4, SLURP1, | GIP, GPR37, NTF4 |
| | | | SNCG, TNR | |
| GOBP_G_PROTEIN_COUPLED_RECEPTOR_SIGNALING_PATHWAY | 0.0087 | 0.004 | ACTN2, ADGRB3, AGRN, AGRP, | AGRP, CCL24, FZD8, GAST, GCG, |
| | | | CALCB, CCL24, CPE, CX3CL1, GCG, | GIP, GPR37, NPY, OXT, PDGFRB, |
| | | | GIP, GPR37, INSL5, NPY, PALM, PDGFRB, | POMC, PPY |
| | | | PPY, PYY | |
| GOBP_STRIATED_MUSCLE_CELL_DIFFERENTIATION | 0.0041 | 0.54 | ACTN2, ADGRB3, COMP, JAM2, | PDGFRB, WFIKKN2 |
| | | | LMOD1, PDGFRB, PI16, TMOD4, | |
| | | | WFIKKN2 | |
| GOBP_ADULT_FEEDING_BEHAVIOR | 0.014 | 0.0044 | AGRP, NPY | AGRP, NPY |
| GOBP_INTRACILIARY_TRANSPORT | 1 | 0.0044 | | RPGR, TNPO1 |
| GOBP_RESPONSE_TO_ELECTRICAL_STIMULUS | 0.49 | 0.0049 | PALM | BRD1, EPO, OXT |
| GOBP_CELLULAR_COMPONENT_MORPHOGENESIS | 0.0049 | 0.022 | ACTN2, ADGRB3, CNTN2, CSPG5, | CSPG5, ENPP2, EPHA10, FLRT2, |
| | | | DSCAM, GFRA3, GPC1, LAMA1, | MAPT, NCAM1, NGF, NTF4, PDGFRB, |
| | | | LMOD1, NCAM1, NRTN, NTF3, NTF4, | PHLDB2, SEMA3F |
| | | | PDGFRB, PHLDB2, SEMA6C, SLITRK2, | |
| | | | TMOD4, TNR | |
| GOBP_NEURON_MATURATION | 0.0054 | 1 | ADGRB3, AGRN, CNTN2, CX3CL1 | |
| GOBP_PROTEOGLYCAN_BIOSYNTHETIC_PROCESS | 0.0054 | 0.064 | BGN, CSPG4, CSPG5, HS6ST1 | CSPG5, HS6ST1 |
| GOBP_CHONDROITIN_SULFATE_BIOSYNTHETIC_PROCESS | 0.0058 | 0.2 | BGN, CSPG4, CSPG5 | CSPG5 |
| GOBP_DERMATAN_SULFATE_METABOLIC_PROCESS | 0.0058 | 0.2 | BGN, CSPG4, CSPG5 | CSPG5 |
| GOBP_INTESTINAL_EPITHELIAL_CELL_DIFFERENTIATION | 0.0058 | 0.2 | NPY, PYY, YAP1 | NPY |
| GOBP_PROTEOGLYCAN_METABOLIC_PROCESS | 0.0059 | 0.14 | BGN, CSPG4, CSPG5, GPC1, HS6ST1 | CSPG5, HS6ST1 |
| GOBP_REGULATION_OF_TRANS_SYNAPTIC_SIGNALING | 0.03 | 0.0068 | CD38, CSPG5, CX3CL1, GIP, NTF3, | CSPG5, GIP, MAPT, NGF, NTF4, OXT, |
| | | | NTF4, SCGN, SNCG, TMEM25, TNR | SCGN, TMEM25 |
| GOBP_MYOFIBRIL_ASSEMBLY | 0.007 | 0.36 | ACTN2, LMOD1, PDGFRB, TMOD4 | PDGFRB |
| GOBP_REGULATION_OF_GLUCAGON_SECRETION | 0.023 | 0.0073 | FAM3D, GIP | FAM3D, GIP |
| GOBP_POINTED_END_ACTIN_FILAMENT_CAPPING | 0.0073 | 1 | LMOD1, TMOD4 | |
| GOBP_POSITIVE_REGULATION_OF_FEEDING_BEHAVIOR | 0.0073 | 0.081 | AGRP, INSL5 | AGRP |
| GOBP_DERMATAN_SULFATE_PROTEOGLYCAN_METABOLIC_PROCESS | 0.0084 | 0.22 | BGN, CSPG4, CSPG5 | CSPG5 |
| GOBP_ANATOMICAL_STRUCTURE_FORMATION_INVOLVED_IN_{—} | 0.0086 | 0.23 | ACTN2, ADGRB3, CCL24, CCN1, | CCL24, CD160, ENPP2, FASLG, |
| MORPHOGENESIS | | | CSPG4, DKK4, DSCAM, FAP, FASLG, | MEGF11, MFGE8, NTF4, PDGFRB, |
| | | | GPC1, HSPB6, ITGAV, JAM2, LMOD1, | PHLDB2, RSPO3, TDGF1, ZP3 |
| | | | MCAM, MEGF11, MFGE8, NTF4, | |
| | | | PDGFRB, PHLDB2, RSPO3, SPINK5, | |
| | | | TGFBI, TMOD4, TNFRSF12A, YAP1, | |
| | | | ZP3 | |
| GOBP_ADULT_BEHAVIOR | 0.018 | 0.01 | AGRP, GIP, NPY, NTF4, SNCG | AGRP, GIP, NPY, NTF4 |
| GOBP_EATING_BEHAVIOR | 0.27 | 0.011 | AGRP | AGRP, OXT |
| GOBP_MUSCLE_CELL_DIFFERENTIATION | 0.012 | 0.45 | ACTN2, ADGRB3, COMP, DUSP29, | DUSP29, PDGFRB, WFIKKN2 |
| | | | JAM2, LMOD1, PDGFRB, PI16, | |
| | | | TMOD4, WFIKKN2 | |
| GOBP_CHONDROITIN_SULFATE_PROTEOGLYCAN_BIOSYNTHETIC_PROCESS | 0.012 | 0.25 | BGN, CSPG4, CSPG5 | CSPG5 |
| GOBP_POSITIVE_REGULATION_OF_LIPASE_ACTIVITY | 0.013 | 0.19 | APOA4, CCN1, NTF3, NTF4, | NTF4, PDGFRB |
| | | | PDGFRB | |
| GOBP_RESPONSE_TO_NERVE_GROWTH_FACTOR | 0.23 | 0.013 | NTF3, NTF4 | MAPT, NGF, NTF4 |
| GOBP_DETECTION_OF_CELL_DENSITY | 0.014 | 1 | FAP, YAP1 | |
| GOBP_NEGATIVE_REGULATION_OF_STRIATED_MUSCLE_CELL_APOPTOTIC_{—} | 0.014 | 1 | BAG3, HSPB6 | |
| PROCESS | | | | |
| GOBP_RESPONSE_TO_HYDROPEROXIDE | 0.014 | 1 | APOA4, CD38 | |
| GOBP_CELLULAR_ANION_HOMEOSTASIS | 0.3 | 0.015 | FASLG | FASLG, FGF23 |
| GOBP_FIBROBLAST_ACTIVATION | 0.3 | 0.015 | PDGFRB | IL17A, PDGFRB |
| GOBP_NODAL_SIGNALING_PATHWAY | 0.3 | 0.015 | CFC1 | CFC1, TDGF1 |
| GOBP_REGULATION_OF_APPETITE | 0.3 | 0.015 | NPY | NPY, POMC |
| GOBP_NERVE_DEVELOPMENT | 0.067 | 0.015 | NRTN, NTF3, NTF4 | NGF, NTF4, SEMA3F |
| GOBP_CHONDROITIN_SULFATE_CATABOLIC_PROCESS | 0.015 | 0.27 | BGN, CSPG4, CSPG5 | CSPG5 |
| GOBP_ERYTHROCYTE_DEVELOPMENT | 1 | 0.015 | | BRD1, EPO |
| GOBP_PROTEIN_TRANSPORT_ALONG_MICROTUBULE | 0.3 | 0.015 | BAG3 | RPGR, TNPO1 |
| GOBP_RESPONSE_TO_HYPEROXIA | 0.3 | 0.015 | PDGFRB | EPO, PDGFRB |
| GOBP_SYNAPTIC_SIGNALING | 0.058 | 0.018 | AGRN, CD38, CSPG5, CX3CL1, | CSPG5, GIP, MAPT, NGF, NPY, |
| | | | GIP, NPY, NTF3, NTF4, SCGN, SNCG, | NTF4, OXT, SCGN, TMEM25 |
| | | | TMEM25, TNR | |
| GOBP_POSITIVE_REGULATION_OF_CYTOKINE_PRODUCTION_INVOLVED_IN_{—} | 0.34 | 0.019 | CLEC7A | CLEC7A, IL17A |
| INFLAMMATORY_RESPONSE | | | | |
| GOBP_NERVE_GROWTH_FACTOR_SIGNALING_PATHWAY | 0.058 | 0.019 | NTF3, NTF4 | NGF, NTF4 |
| GOBP_POSITIVE_REGULATION_OF_ENDOTHELIAL_CELL_APOPTOTIC_PROCESS | 0.058 | 0.019 | CD248, FASLG | CD160, FASLG |
| GOBP_RESPONSE_TO_INCREASED_OXYGEN_LEVELS | 0.34 | 0.019 | PDGFRB | EPO, PDGFRB |
| GOBP_NEURONAL_ION_CHANNEL_CLUSTERING | 0.023 | 1 | AGRN, CNTN2 | |
| GOBP_BLASTOCYST_FORMATION | 0.023 | 0.13 | YAP1, ZP3 | ZP3 |
| GOBP_POSITIVE_REGULATION_OF_OSTEOBLAST_PROLIFERATION | 0.023 | 1 | CCN1, ITGAV | |
| GOBP_REGULATION_OF_FEEDING_BEHAVIOR | 0.023 | 0.13 | AGRP, INSL5 | AGRP |
| GOBP_VASCULAR_ASSOCIATED_SMOOTH_MUSCLE_CONTRACTION | 0.023 | 1 | CD38, COMP | |
| GOBP_HYPEROSMOTIC_RESPONSE | 1 | 0.024 | | EPO, OXT |
| GOBP_RESPONSE_TO_SALT_STRESS | 1 | 0.024 | | EPO, OXT |
| GOBP_EMBRYONIC_CLEAVAGE | 0.05 | 0.028 | TOP1 | TOP1 |
| GOBP_FAT_CELL_PROLIFERATION | 1 | 0.028 | | FGF16 |
| GOBP_HISTONE_H3_K23_ACETYLATION | 1 | 0.028 | | BRD1 |
| GOBP_INTRACELLULAR_DISTRIBUTION_OF_MITOCHONDRIA | 1 | 0.028 | | MAPT |
| GOBP_LOCOMOTION_INVOLVED_IN_LOCOMOTORY_BEHAVIOR | 0.05 | 0.028 | GPR37 | GPR37 |
| GOBP_MITOCHONDRION_DISTRIBUTION | 1 | 0.028 | | MAPT |
| GOBP_NEGATIVE_REGULATION_OF_TUBULIN_DEACETYLATION | 1 | 0.028 | | MAPT |
| GOBP_PHOSPHATIDYLSERINE_EXPOSURE_ON_APOPTOTIC_CELL_SURFACE | 0.05 | 0.028 | FASLG | FASLG |
| GOBP_PLUS_END_DIRECTED_ORGANELLE_TRANSPORT_ALONG_MICROTUBULE | 1 | 0.028 | | MAPT |
| GOBP_POSITIVE_REGULATION_OF_HISTONE_H3_K4_METHYLATION | 0.05 | 0.028 | GCG | GCG |
| GOBP_POSITIVE_REGULATION_OF_PHOSPHOLIPID_TRANSLOCATION | 0.05 | 0.028 | FASLG | FASLG |
| GOBP_POSITIVE_REGULATION_OF_UTERINE_SMOOTH_MUSCLE_CONTRACTION | 1 | 0.028 | | OXT |
| GOBP_PROTEIN_SIDE_CHAIN_DEGLUTAMYLATION | 0.05 | 0.028 | AGBL2 | AGBL2 |
| GOBP_REGULATION_OF_FEMALE_RECEPTIVITY | 1 | 0.028 | | OXT |
| GOBP_REGULATION_OF_INTRINSIC_APOPTOTIC_SIGNALING_PATHWAY_IN_{—} | 1 | 0.028 | | EPO |
| RESPONSE_TO_OSMOTIC_STRESS | | | | |
| GOBP_REGULATION_OF_PHOSPHOLIPID_TRANSLOCATION | 0.05 | 0.028 | FASLG | FASLG |
| GOBP_RESPONSE_TO_MOLECULE_OF_FUNGAL_ORIGIN | 0.05 | 0.028 | CLEC7A | CLEC7A |
| GOBP_SPERM_EJACULATION | 1 | 0.028 | | OXT |
| GOBP_TRIPARTITE_REGIONAL_SUBDIVISION | 1 | 0.028 | | TDGF |
| GOBP_UTERINE_SMOOTH_MUSCLE_CONTRACTION | 1 | 0.028 | | OXT |
| GOBP_DIGESTION | 0.37 | 0.03 | APOA4, ASAH2, GUCA2A | ASAH2, IL17A, OXT, TFF1 |
| GOBP_POSITIVE_REGULATION_OF_CALCIUM_ION_IMPORT | 0.087 | 0.03 | GCG, PDGFRB | GCG, PDGFRB |
| GOBP_ACTIN_FILAMENT_DEPOLYMERIZATION | 0.03 | 1 | ACTN2, LMOD1, TMOD4 | |
| GOBP_OSTEOBLAST_PROLIFERATION | 0.03 | 1 | ATRAID, CCN1, ITGAV | |
| GOBP_REGULATION_OF_OSTEOBLAST_PROLIFERATION | 0.03 | 1 | ATRAID, CCN1, ITGAV | |
| GOBP_REGULATION_OF_SUPEROXIDE_ANION_GENERATION | 0.4 | 0.03 | CLEC7A | CLEC7A, MAPT |
| GOBP_RESPONSE_TO_RETINOIC_ACID | 0.13 | 0.031 | CD38, PDGFRB, YAP1 | OXT, PDGFRB, WNT9A |
| GOBP_TRANSPORT_ALONG_MICROTUBULE | 0.73 | 0.031 | BAG3 | MAPT, RPGR, TNPO1 |
| GOBP_POSITIVE_REGULATION_OF_SMALL_GTPASE_MEDIATED_SIGNAL_{—} | 0.73 | 0.031 | PDGFRB | EPO, NGF, PDGFRB |
| TRANSDUCTION | | | | |
| GOBP_REGIONALIZATION | 0.66 | 0.033 | CFC1, NTF4 | CFC1, NTF4, SEMA3F, TDGF1 |
| GOBP_COGNITION | 0.11 | 0.033 | ADGRB3, GIP, NTF3, NTF4, PTPRZ1, | GIP, MAPT, NGF, NTF4, OXT |
| | | | TNR | |
| GOBP_MULTICELLULAR_ORGANISMAL_MOVEMENT | 0.033 | 1 | COMP, MB | |
| GOBP_NEURON_REMODELING | 0.033 | 1 | ADGRB3, CX3CL1 | |
| GOBP_POSITIVE_REGULATION_OF_ACROSOME_REACTION | 0.033 | 0.16 | PLB1, ZP3 | ZP3 |
| GOBP_REGULATION_OF_EXECUTION_PHASE_OF_APOPTOSIS | 0.033 | 0.16 | AP, GCG | GCG |
| GOBP_RETINA_LAYER_FORMATION | 0.033 | 0.16 | DSCAM, MEGF11 | MEGF11 |
| GOBP_PEPTIDYL_SERINE_MODIFICATION | 0.46 | 0.034 | BGN, GCG, NTF3, NTF4, TOP1 | EPO, GCG, NGF, NTF4, TDGF1, TOP1 |
| GOBP_PERIPHERAL_NERVOUS_SYSTEM_DEVELOPMENT | 0.034 | 0.15 | GFRA3, GPC1, NTF3, NTF4 | NGF, NTF4 |
| GOBP_ANION_HOMEOSTASIS | 0.43 | 0.036 | FASLG | FASLG, FGF23 |
| GOBP_MAINTENANCE_OF_GASTROINTESTINAL_EPITHELIUM | 1 | 0.036 | | IL17A, TFF1 |
| GOBP_REGULATION_OF_KERATINOCYTE_PROLIFERATION | 0.036 | 1 | CRNN, SLURP1, YAP1 | |
| GOBP_REGULATION_OF_PROTEIN_DEPOLYMERIZATION | 0.036 | 1 | ACTN2, LMOD1, TMOD4 | |
| GOBP_CHONDROITIN_SULFATE_PROTEOGLYCAN_METABOLIC_PROCESS | 0.036 | 0.35 | BGN, CSPG4, CSPG5 | CSPG5 |
| GOBP_GLYCEROPHOSPHOLIPID_CATABOLIC_PROCESS | 0.43 | 0.036 | ENPP6 | ENPP2, ENPP6 |
| GOBP_SEGMENTATION | 1 | 0.036 | | SEMA3F, TDGF1 |
| GOBP_REGULATION_OF_CELL_JUNCTION_ASSEMBLY | 0.44 | 0.037 | ADGRB3, AGRN, PHLDB2, SLITRK2 | DUSP3, FLRT2, IL17A, OXT, |
| | | | | PHLDB2 |
| GOBP_MICROTUBULE_BASED_TRANSPORT | 0.75 | 0.038 | BAG3 | MAPT, RPGR, TNPO1 |
| GOBP_AMINOGLYCAN_METABOLIC_PROCESS | 0.04 | 0.26 | AGRN, BGN, CSPG4, CSPG5, GPC1, | CSPG5, HS6ST1, PDGFRB |
| | | | HS6ST1, PDGFRB | |
| GOBP_CYTOSKELETON_DEPENDENT_INTRACELLULAR_TRANSPORT | 0.77 | 0.041 | BAG3 | MAPT, RPGR, TNPO1 |
| GOBP_EMBRYONIC_PATTERN_SPECIFICATION | 1 | 0.042 | | SEMA3F, TDGF1 |
| GOBP_RESPONSE_TO_INTERLEUKIN_6 | 0.46 | 0.042 | YAP1 | FGF23, TDGF1 |
| GOBP_NEURON_PROJECTION_GUIDANCE | 0.042 | 0.26 | CNTN2, DSCAM, GFRA3, GPC1, | EPHA10, FLRT2, NCAM1, SEMA3F |
| | | | LAMA1, NCAM1, NRTN, SEMA6C, TNR | |
| GOBP_BASEMENT_MEMBRANE_ORGANIZATION | 0.46 | 0.042 | PHLDB2 | FLRT2, PHLDB2 |
| GOBP_NEGATIVE_REGULATION_OF_T_CELL_RECEPTOR_SIGNALING_PATHWAY | 1 | 0.042 | | CD160, DUSP3 |
| GOBP_REGULATION_OF_SUPEROXIDE_METABOLIC_PROCESS | 0.46 | 0.042 | CLEC7A | CLEC7A, MAPT |
| GOBP_RESPONSE_TO_IMMOBILIZATION_STRESS | 1 | 0.042 | | BRD1, TFF1 |
| GOBP_RESPONSE_TO_FIBROBLAST_GROWTH_FACTOR | 0.92 | 0.043 | GPC1 | FGF16, FGF23, FLRT2, TDGF1 |
| GOBP_CARDIAC_CELL_DEVELOPMENT | 0.043 | 0.36 | ACTN2, PDGFRB, PI16 | PDGFRB |
| GOBP_BLOOD_VESSEL_MORPHOGENESIS | 0.043 | 0.33 | ADGRB3, CCL24, CCN1, COMP, | CCL24, CD160, ENPP2, FASLG, |
| | | | CSPG4, FAP, FASLG, HSPB6, ITGAV, | MFGE8, PDGFRB, RSPO3, TDGF1 |
| | | | LAMA1, MCAM, MFGE8, PDGFRB, | |
| | | | RSPO3, SPINK5, TGFBI, TNFRSF12A, | |
| | | | YAP1 | |
| GOBP_POSITIVE_REGULATION_OF_CYSTEINE_TYPE_ENDOPEPTIDASE_{—} | 0.44 | 0.043 | CCN1, CLEC7A, FASLG | CLEC7A, FASLG, MAPT, NGF |
| ACTIVITY | | | | |
| GOBP_AMINOGLYCAN_CATABOLIC_PROCESS | 0.044 | 0.67 | AGRN, BGN, CSPG4, CSPG5, GPC1 | CSPG5 |
| GOBP_EMBRYONIC_PLACENTA_MORPHOGENESIS | 0.045 | 0.18 | CCN1, RSPO3 | RSPO3 |
| GOBP_LABYRINTHINE_LAYER_MORPHOGENESIS | 0.045 | 0.18 | CCN1, RSPO3 | RSPO3 |
| GOBP_NEGATIVE_REGULATION_OF_MUSCLE_CELL_APOPTOTIC_PROCESS | 0.045 | 1 | BAG3, HSPB6 | |
| GOBP_POSITIVE_REGULATION_OF_BEHAVIOR | 0.045 | 0.18 | AGRP, INSL5 | AGRP |
| GOBP_POSITIVE_REGULATION_OF_INFLAMMATORY_RESPONSE_TO_{—} | 0.045 | 0.18 | CD28, ZP3 | ZP3 |
| ANTIGENIC_STIMULUS | | | | |
| GOBP_RETINA_VASCULATURE_DEVELOPMENT_IN_CAMERA_TYPE_EYE | 0.045 | 0.18 | LAMA1, PDGFRB | PDGFRB |
| GOBP_MUSCLE_CONTRACTION | 0.046 | 0.66 | ACTN2, CD38, COMP, HSPB6, | GAMT, OXT |
| | | | LMOD1, MB, SCN3B, TMOD4 | |
| GOBP_POSITIVE_REGULATION_OF_CELLULAR_COMPONENT_ORGANIZATION | 0.51 | 0.047 | ACTN2, ADGRB3, AGRN, CCL24, | CCL24, CLEC7A, DUSP3, ENPP2, |
| | | | CD28, CLEC7A, CX3CL1, DSCAM, | EPO, FASLG, FLRT2, GCG, IL17A, |
| | | | FASLG, GCG, LMOD1, NTF3, PALM, | MAPT, NGF, OXT, PDGFRB, PHLDB2 |
| | | | PDGFRB, PHLDB2, SLITRK2 | |
| GOBP_DIGESTIVE_SYSTEM_DEVELOPMENT | 0.048 | 0.31 | CLMP, GIP, NPY, PYY, YAP1 | GIP, NPY |
| GOBP_SIGNAL_RELEASE | 0.57 | 0.048 | CD38, CSPG5, FAM3D, GCG, GIP, | CSPG5, FAM3D, FGF23, GCG, GIP, |
| | | | SNCG | OXT, POMC |
| GOBP_EPITHELIAL_STRUCTURE_MAINTENANCE | 1 | 0.049 | | IL17A, TFF1 |
| GOBP_POSITIVE_REGULATION_OF_HUMORAL_IMMUNE_RESPONSE | 0.49 | 0.049 | ZP3 | IL17A, ZP3 |
| GOBP_REGULATION_OF_PHOSPHOLIPASE_ACTIVITY | 0.049 | 0.18 | CCN1, NTF3, NTF4, PDGFRB | NTF4, PDGFRB |

1. A method for predicting risk of cancer in a subject, the method comprising:
obtaining or having obtained a dataset derived from the subject comprising quantitative levels of a plurality of biomarkers, wherein the plurality of biomarkers comprises protein biomarkers comprising two or more of TSPAN1, CD28, SCN3B, ADGRB3, and IGFBP6, and
generating a prediction of risk of cancer for the subject by applying a predictive model to the quantitative values of the plurality of biomarkers.

2-4. (canceled)

5. The method of claim 1, wherein the protein biomarkers further comprise one or more of NRTN, AIF1L, HSPB6, MB, TNFRSF19, IL5RA, TNR, CDNF, CST1, FGFBP2, S100A16, CD248, GFRA3, LMOD1, and POF1B.

6-8. (canceled)

9. The method of claim 1, wherein the protein biomarkers further comprise one or more of DENND2B, COMP, CNTN2, SCARA5, CSPG4, ITGAV, SOST, SERPINA4, LILRA4, SPINK5, PINLYP, ACTN2, JAM2, FAP, TMOD4, GUCA2A, MFAP3L, DKK4, LAMA1, BAG3, SNCG, SEPTIN3, VWC2, KLRC1, ATRAID, ART3, SLITRK2, SIGLEC6, TMED4, and SLAMF7.

10-13. (canceled)

14. The method of claim 1, wherein the protein biomarkers further comprise one or more of CKMT1A, SEMA6C, CD2, CST5, PBXIP1, LECT2, PYY, AGRN, INSL5, CD38, PI16, CCN5, TNFRSF17, LY9, GPC1, CLMP, MEP1B, CCN1, PCDH7, SPARCL1, CRNN, PM20D1, TNFRSF12A, DSCAM, PALM, CX3CL1, MEP1A, SLURP1, APOA4, ADAMTSL5, MEPE, WFDC1, RPS10, CD300C, RIPK4, CALCB, RTBDN, ENO3, NTF3, PTPRZ1, LRP2BP, CPE, MCAM, BGN, PLB1, YAP1, TGFBI, CYB5A, EDDM3B, and SELENOP.

15-20. (canceled)

21. The method of claim 1, wherein the protein biomarkers further comprise one or more of ENPP6, TMEM25, GIP, CSPG5, SCGN, TMPRSS15, LAIR2, KIRREL1, NTF4, TSPAN7, ENDOU, KLK10, CCL24, GPR37, CD3D, TJP3, DKKL1, CFC1, LRRC38, GCG, AGBL2, FASLG, AHNAK2, WFIKKN2, ANXA10, HS6ST1, DUSP29, CA14, CLEC7A, PHLDB2, SCRG1, RSPO3, TOP1, TINAGL1, NCAM1, FAM3D, FLT3LG, ZP3, AGRP, ASAH2, PDGFRB, AFM, NPY, PPY, XG, MFGE8, PROS1, MEGF11, CTSO, CTLA4, CSF3R, FCAR, CTAG1A, SCPEP1, PRSS53, CRELD2, PILRA, PROC, VASH1, NOS3, BPIFB2, UPK3BL1, NOP56, JAM3, HLA-DRA, SIL1, TRPV3, EDEM2, POLR2A, CBLN1, FKBP7, CCL20, PILRB, SIRPB1, VSTM1, BST2, DLL4, C1RL, RNASET2, KCNH2, IL12RB2, FZD10, OXCT1, TREML2, GRIN2B, GFRAL, RGS8, LRPAP1, LRP2, IGSF21, DPT, HEPACAM2, MATN3, UXS1, PTTG1, BTN1A1, IL17C, SCIN, TK1, FKBP14, VWA5A, PRKG1, SV2A, PMCH, NEXN, CDCP1, DDX53, THSD1, PAK4, MMP12, FCN1, UMOD, PDIA4, IL6, BRK1, LILRA2, RBPMS2, SERPIND1, TPSG1, CEACAM5, FGF9, PPIF, RNF43, SIGLEC9, TOMM20, PDE5A, NELL1, GBA, PAEP, ERN1, PCSK7, CHCHD6, MARCO, SFTPA1, IL9, KYNU, SPINT1, LRFN2, NECTIN1, OSCAR, PZP, BPIFB1, LILRA5, CALY, RRAS, GADD45GIP1, ISM2, SCGB3A2, CEACAM6, LPP, GKN1, LRIG1, CLSPN, CXCL13, SFTPA2, COX6B1, PTGR1, RBPMS, PPT1, AOC1, PDLIM5, L3HYPDH, LONP1, APOL1, CEACAM18, FGF7, and KRT14.

22. The method of claim 1, wherein the predictive model comprises a elastic net regression model, and wherein the predictive model achieves an area under a curve (AUC) value of at least 0.85.

23. The method of claim 1, wherein the predictive model comprises a support vector machine, and wherein the predictive model achieves an area under a curve (AUC) value of at least 0.84.

24. The method of claim 1, wherein the predictive model comprises a random forest model, and wherein the predictive model achieves an area under a curve (AUC) value of at least 0.72.

25. The method of claim 1, wherein the predictive model comprises a XGBoost model, and wherein the predictive model achieves an area under a curve (AUC) value of at least 0.73.

26-65. (canceled)

66. The method of claim 1, wherein the cancer is lung cancer.

67-75. (canceled)

76. A non-transitory computer readable medium comprising instructions that, when executed by a processor, cause the processor to:
obtain or have obtained a dataset derived from the subject comprising quantitative levels of a plurality of biomarkers, wherein the plurality of biomarkers comprises protein biomarkers comprising two or more of TSPAN1, CD28, SCN3B, ADGRB3, and IGFBP6, and
generate a prediction of risk of cancer for the subject by applying a predictive model to the quantitative values of the plurality of biomarkers.

77-79. (canceled)

80. The non-transitory computer readable medium of claim 76, wherein the protein biomarkers further comprise one or more of NRTN, AIF1L, HSPB6, MB, TNFRSF19, IL5RA, TNR, CDNF, CST1, FGFBP2, S100A16, CD248, GFRA3, LMOD1, and POF1B.

81-83. (canceled)

84. The non-transitory computer readable medium of claim 76, wherein the protein biomarkers further comprise one or more of DENND2B, COMP, CNTN2, SCARA5, CSPG4, ITGAV, SOST, SERPINA4, LILRA4, SPINK5, PINLYP, ACTN2, JAM2, FAP, TMOD4, GUCA2A, MFAP3L, DKK4, LAMA1, BAG3, SNCG, SEPTIN3, VWC2, KLRC1, ATRAID, ART3, SLITRK2, SIGLEC6, TMED4, and SLAMF7.

85-88. (canceled)

89. The non-transitory computer readable medium of claim 76, wherein the protein biomarkers further comprise one or more of CKMT1A, SEMA6C, CD2, CST5, PBXIP1, LECT2, PYY, AGRN, INSL5, CD38, PI16, CCN5, TNFRSF17, LY9, GPC1, CLMP, MEP1B, CCN1, PCDH7, SPARCL1, CRNN, PM20D1, TNFRSF12A, DSCAM, PALM, CX3CL1, MEP1A, SLURP1, APOA4, ADAMTSL5, MEPE, WFDC1, RPS10, CD300C, RIPK4, CALCB, RTBDN, ENO3, NTF3, PTPRZ1, LRP2BP, CPE, MCAM, BGN, PLB1, YAP1, TGFBI, CYBSA, EDDM3B, and SELENOP.

90-95. (canceled)

96. The non-transitory computer readable medium of claim 76, wherein the protein biomarkers further comprise one or more of ENPP6, TMEM25, GIP, CSPG5, SCGN, TMPRSS15, LAIR2, KIRREL1, NTF4, TSPAN7, ENDOU, KLK10, CCL24, GPR37, CD3D, TJP3, DKKL1, CFC1, LRRC38, GCG, AGBL2, FASLG, AHNAK2, WFIKKN2, ANXA10, HS6ST1, DUSP29, CA14, CLEC7A, PHLDB2, SCRG1, RSPO3, TOP1, TINAGL1, NCAM1, FAM3D, FLT3LG, ZP3, AGRP, ASAH2, PDGFRB, AFM, NPY, PPY, XG, MFGE8, PROS1, MEGF11, CTSO, CTLA4, CSF3R, FCAR, CTAG1A, SCPEP1, PRSS53, CRELD2, PILRA, PROC, VASH1, NOS3, BPIFB2, UPK3BL1, NOP56, JAM3, HLA-DRA, SIL1, TRPV3, EDEM2, POLR2A, CBLN1, FKBP7, CCL20, PILRB, SIRPB1, VSTM1, BST2, DLL4, C1RL, RNASET2, KCNH2, IL12RB2, FZD10, OXCT1, TREML2, GRIN2B, GFRAL, RGS8, LRPAP1, LRP2, IGSF21, DPT, HEPACAM2, MATN3, UXS1, PTTG1, BTN1A1, IL17C, SCIN, TK1, FKBP14, VWA5A, PRKG1, SV2A, PMCH, NEXN, CDCP1, DDX53, THSD1, PAK4, MMP12, FCN1, UMOD, PDIA4, IL6, BRK1, LILRA2, RBPMS2, SERPIND1, TPSG1, CEACAM5, FGF9, PPIF, RNF43, SIGLEC9, TOMM20, PDE5A, NELL1, GBA, PAEP, ERN1, PCSK7, CHCHD6, MARCO, SFTPA1, IL9, KYNU, SPINT1, LRFN2, NECTIN1, OSCAR, PZP, BPIFB1, LILRA5, CALY, RRAS, GADD45GIP1, ISM2, SCGB3A2, CEACAM6, LPP, GKN1, LRIG1, CLSPN, CXCL13, SFTPA2, COX6B1, PTGR1, RBPMS, PPT1, AOC1, PDLIM5, L3HYPDH, LONP1, APOL1, CEACAM18, FGF7, KRT14.

97. The non-transitory computer readable medium of claim 76, wherein the predictive model comprises a elastic net regression model, and wherein the predictive model achieves an area under a curve (AUC) value of at least 0.85.

98. The non-transitory computer readable medium of claim 76, wherein the predictive model comprises a support vector machine, and wherein the predictive model achieves an area under a curve (AUC) value of at least 0.84.

99. The non-transitory computer readable medium of claim 76, wherein the predictive model comprises a random forest model, and wherein the predictive model achieves an area under a curve (AUC) value of at least 0.72.

100. The non-transitory computer readable medium of claim 76, wherein the predictive model comprises a XGBoost model, and wherein the predictive model achieves an area under a curve (AUC) value of at least 0.73.

101-125. (canceled)

126. The non-transitory computer readable medium of claim 76, wherein the cancer is lung cancer.

127-150. (canceled)
